(19) United States
(12) Patent Application Publication
(10) Pub. No.: US 2018/0298377 A1
(43) Pub. Date: Oct. 18, 2018

(54) GENOMIC COMBINATORIAL SCREENING PLATFORM

(71) Applicant: THE RESEARCH FOUNDATION FOR THE STATE UNIVERSITY OF NEW YORK, Albany, NY (US)

(72) Inventors: Sasha LEVY, San Mateo, CA (US); Xianan LIU, Stony Brook, NY (US)

(21) Appl. No.: 15/767,020

(22) PCT Filed: Oct. 28, 2016
(86) PCT No.: PCT/US2016/059573

§ 371 Date: Apr. 9, 2018

Related U.S. Application Data
(60) Provisional application No. 62/248,179, filed on Oct. 29, 2015.

(51) Int. Cl. C12N 15/10; C12N 15/64; C12N 15/66

(52) U.S. Cl. CPC C12N 15/1082; C12N 15/64; C40B 40/08; C12N 2800/30; C12N 15/66

(57) ABSTRACT

The present disclosure provides methods and compositions that enable the rapid insertion of two or more combinations of genetic elements into a target cell genome, as a single copy and at a defined location. Each specific combination of genetic elements can be characterized within a single cell or in a pooled population via short-read sequencing. This technology allows extremely large combinatorial libraries of small or large DNA sequences to be rapidly constructed and screened as pools repeatedly across perturbations.

### CROSS-REFERENCE TO RELATED APPLICATIONS

This application claims the benefit of prior U.S. Provisional Application No. 62/248,179, filed Oct. 29, 2015, which is hereby incorporated by reference in its entirety.

### FIELD OF THE INVENTION

The invention relates to methods and compositions for inserting at least two DNA sequences proximate to each other in a genome and uses thereof.

### BACKGROUND

Combinatorial biological screens, such as those that assay genetic interactions between underexpressed or knocked out genes (Butland: 2008, Costanzo: 2010, Tong: 2002, Pan: 2004, Bassik: 2013), overexpressed genes (Measday: 2005), or that assay physical interactions between proteins (Ito: 2001, Uetz: 2000, Tarassov: 2008), have historically been limited in throughput by the requirement to test for interactions one-at-a-time. More recent methods assemble two or more small DNA elements onto a single plasmid and insert complex plasmid libraries into cells. The effect of each plasmid on the cell can be assayed in pools using next generation sequencing of barcodes or the DNA sequences themselves (Bassik: 2013, Wong: 2015). However, the utility of current methods to test combinations of larger DNA sequences is limited because it is necessary to assemble all elements onto a single plasmid, with practical size limits for insertion into bacterial cells, viral packaging or insertion into target cells. Furthermore, transient transfection or random insertion of plasmids into cell genomes could result in large variation in gene product copy number between cells, confounding measurements of the phenotypic effect of the combination.

Accordingly, there is an ongoing need in the art for methods and compositions to enable a rapid and comprehensive characterization of large collections of biologic combinations of small and large DNA elements at an invariant location in the cell genome. Besides circumventing size restrictions of systems that use a single plasmid, copy number variation of combinations would be reduced, resulting in less experimental error.

Described herein are methods and compositions that enable the rapid insertion of two or more combinations of genetic elements into a target cell genome, as a single copy and at a defined location. Each specific combination of genetic elements can be characterized within a single cell or in a pooled population via short-read sequencing. This technology allows extremely large combinatorial libraries of small or large DNA sequences to be rapidly constructed and screened as pools repeatedly across perturbations.

### SUMMARY OF THE INVENTION

In one embodiment, the present invention provides methods for placing at least two DNA sequences proximate to each other in a genome, the method includes: (a) providing the genome with a first site-specific recombination site; (b) recombining the first site-specific recombination site with a third site-specific recombination site compatible with the first site-specific recombination site, wherein the third site-specific recombination site is associated with a first DNA sequence, thereby forming a first hybrid recombination site associated with the first DNA sequence and a third hybrid recombination site; (c) providing the genome with a second site-specific recombination site; (d) recombining the second site-specific recombination site, with a fourth site-specific recombination site compatible with the second site-specific recombination site, wherein the fourth site-specific recombination site is associated with a second DNA sequence, thereby forming a second hybrid recombination site associated with the second DNA sequence and a fourth hybrid recombination site; (1) wherein steps (a), (b), (c), and (d) can be performed in any order; (2) wherein any two, three, or four of steps (a), (b), (c), and (d) are optionally combined into a single step; and whereby the first DNA sequence and the second DNA sequence are proximate to each other after recombining steps (b) and (d).

In another embodiment, the invention provides a kit including: a first circular DNA library containing a plurality of DNA molecules, wherein each DNA molecule contains (i) a third site-specific recombination site, (ii) a plurality of first DNA sequences, and (iii) either a first cell-selectable marker or a first portion of a split cell-selectable marker or both; and a second circular DNA library containing a plurality of DNA molecules, wherein each DNA molecule includes (i) a fourth site-specific recombination site, (ii) a plurality of second DNA sequences, and (iii) either a second cell-selectable marker or a second portion of a split cell-selectable marker or both.

As a result of the present invention, large combinatorial libraries of small or large DNA sequences can be rapidly constructed and screened as pools repeatedly across perturbations.

### DESCRIPTION OF THE FIGURES

FIG. 1 depicts an embodiment of the invention wherein a single recombinase is used to insert two proximate DNA sequences/elements into the genome.

FIG. 2 depicts an embodiment of the invention wherein two recombinases are used to insert two proximate DNA sequences/elements into the genome.

FIG. 3 depicts an embodiment of the invention wherein the third site-specific recombination site is further associated with a third DNA sequence and the fourth site-specific recombination site is further associated with a fourth DNA sequence.

FIG. 4 depicts an embodiment of the invention wherein the use of a split cell-selectable marker is used.

FIG. 5 depicts an embodiment of the invention wherein the third site-specific recombination site is further associated with a third DNA sequence and the fourth site-specific recombination site is further associated with a fourth DNA sequence; and a split cell-selectable marker is used.

FIG. 6 depicts an embodiment of the invention wherein the third site-specific recombination site is further associated with a third DNA sequence and the fourth site-specific recombination site is further associated with a fourth DNA sequence; and a cell selectable marker and split cell-selectable marker is used.

FIG. 7 depicts an embodiment of the invention wherein the genome having a firth DNA sequence and a second DNA sequence are capable of being sequenced together via paired-end sequencing.

FIG. 8 depicts an embodiment of the invention including a kit of components for performing the disclosed method of inserting two proximate DNA sequences into a genome.

FIG. 9 depicts an embodiment of the invention wherein plasmid libraries containing barcodes and associated DNA elements are sequentially inserted into a yeast genome.

FIG. 10 depicts an embodiment of the invention wherein the method is used to create a protein-protein interaction library to screen for protein-protein interactions by mating to protein fragment complementation (PCA) strains.

FIG. 11A depicts a schematic of a lineage tracking experiment in barcoded yeast with the same initial fitness. A small lineage that does not acquire a beneficial mutation (neutral, blue) will fluctuate in size due to drift before eventually being outcompeted. Rarely, a lineage will acquire a beneficial mutation (star) with a fitness effect of s (adaptive, red). In most cases, this beneficial mutation is lost to drift. If the beneficial mutants drift to a size >˜1/s (lower dotted horizontal line), the lineage will begin to grow exponentially at a rate s. Extrapolating the exponential growth to the time at which the mutation is inferred to have reach a size ˜1/s yields the establishment time (τ, dashed vertical line) which roughly corresponds to the time when the mutation occurred with an uncertainty of ˜1/s. At sizes >˜1/Ub (upper dotted horizontal line), where Ub is the total beneficial mutation rate, the lineage will acquire additional beneficial mutations. FIG. 11B depicts lineage tracking with random barcodes. Left. Sequences containing random 20 nucleotide barcodes (colors) are inserted first into a plasmid and then into a specific location in the genome. Bottom. Recombination between two partially crippled loxP sites (loxP*) integrates the plasmid into the genome and completes a URA3 selectable marker, resulting in one functional and one crippled loxP site (loxP**). The URA3 marker is interrupted by an artificial intron containing the barcode. Right. To measure relative fitness, cells are passed through growth-bottleneck cycles of ˜8 generations. Before each bottleneck, genomic DNA is extracted, lineage barcode tags are amplified using a two-step PCR protocol, and amplicons are sequenced. By inserting unique molecular identifiers (also short random barcodes, grey bars) in early cycles of the PCR, PCR duplicates of the same template molecule (purple) are detected.

FIG. 12 depicts schematic of strain constructions in the YBR209W locus. A diagram presenting the yeast strains with lox sites. Lines with arrows indicate the selection method after transformation. The sequence in the YBR209W locus are indicated.

FIG. 13 depicts schematic of construction of a large combinatorial library via sequential plasmid integration in yeast.

FIG. 14 depicts schematic of construction of a large combinatorial library via plasmid integration and mating in yeast.

FIGS. 15A-15D depict the inferred fitnesses and establishment times from lineage trajectories. (15A) Selected lineage trajectories colored according to the probability that they contain an established beneficial mutation. The decline of adaptive lineages at later times is caused by the increase of the population mean fitness (Inset). The population mean fitness is inferred from both the decline of neutral lineages (blue circles) and the growth of beneficial lineages. Shading indicates the error in mean fitness. The inferred fitnesses (15B) and establishment times (15C) from analysis of simulated trajectories correlate strongly with the known simulated values. (15D) Scatter plot of the fitness of 33 clones picked from E2 at generation 88 inferred by sequencing and pairwise competition (coloring as in (a), with outliers lightened and excluded from correlation). Error bars are 1 standard deviation.

FIGS. 16A-16B depict fitness effects and establishment times of beneficial mutations, and the population dynamics. (16A) Scatter plot of τ and s of all ˜25,000 beneficial mutations (circles) identified in E1. Circle area represents the size of the lineage at generation 88. Purple circles (dark grey) indicate lineages with mutations that occurred in the period of common growth (t<0) that were sampled into, and established in, E1 and E2. Green circles (light grey) indicate lineages that were identified as adaptive in only one replicate and likely contain mutations that arose after t=0. Lines indicate the time limits before which mutations must occur in order to establish (large dash) or be observed (small dash). These limits trail the mean fitness (solid line) by ˜1/s generations. (Inset) The spectrum of mutation rates, μ(s), as a function of fitness effect, s inferred from mutations that likely occurred after t=0. The y-axis is the mutation rate density, so the mutation rate to a range, Δs, is obtained by multiplying this by Δs. The total beneficial mutation rate to s>5% is inferred to be ˜1×10⁻⁶ and is consistent across replicates. The observed spectrum is not exponential (gray line, with the error range shaded). (16B) The distribution of the number of adaptive cells binned by their fitness over time. As the mean fitness (grey curtain) surpasses the fitness of a subpopulation, cells with that fitness begin to decline in frequency.

FIG. 17 depicts the fitness spectrum of adaptive lineages that could be identified within the first 100 generations at different frequency resolution thresholds.

FIG. 18 depicts construction of a Protein-Protein interaction Sequencing (PPiSeq) library. Primers containing a random nucleotide barcode are inserted into a common genomic location of both MAT∝ and MATa cells by homologous recombination, yielding large libraries of barcoded yeast cells. Clones from each library are picked at random and barcodes are identified by sequencing. Barcoded cells are mated to strains containing either a bait or prey protein fragment complementation construct. Diploids are sporulated and haploids containing both a barcode and a PCA construct are selected. These haploids are mated to generate diploids that contain two barcodes and both bait and prey PCA constructs. Cre-induced loxP recombination brings the two barcodes to the same chromosome, and is selected for by reconstruction of a split URA3 selectable marker. Double barcodes mark the two PCA constructs that are in each cell and are subsequently used as part of a sequencing-based pooled fitness assay to measure PPI scores.

FIGS. 19A-19C depicts lineage tracking and fitness estimation of double barcodes. (19A) The frequency trajectories of 2500 double barcoded PCA strains in the absence or presence of 0.5 μg/ml methotrexate (MTX). Frequencies are assayed every three generations during serial batch growth. Color indicates the estimated fitness relative to strains in the same pool that lack mDHFR fragments. (19B) Performance of fitness estimates on simulated data. Pearson's r=0.996. (19C) Reproducibility of fitness estimates across growth replicates. Pearson's r>0.93 in MTX.

FIG. 20 depicts PPiSeq performance. Top: Relative fitnesses of each protein fragment pair grown in the absence (black) or presence (purple) of MTX. Each protein fragment pair is assayed with 25 unique double barcodes across 3 growth replicates for a total of ˜75 fitness estimates. Asterisks indicate the mean fitness of the protein fragment pair in MTX across all measurements and PPIs are ranked according to this fitness. Bottom purple: Heat map of the significance of the fitness difference between each protein fragment pair and control strains in the same pool that lack mDHFR fragments. P-values are calculated using a Bonferroni-corrected Student's t-test. Bottom grey: the number of times each protein-protein interaction has previously been cited. Biogrid is the sum of all forms of evidence: protein fragment complementation (PCA), yeast two-hybrid (YTH), pull down/mass spectroscopy (Pulldown), and low-throughput studies (Literature).

FIGS. 21A-21C depicts Dynamic PPIs. (21A) Heatmap of PPIs across environments. All PPIs discovered here or elsewhere are shown. Colors are the fitness in each condition minus the fitness in the benign condition. Cells are arranged by unsupervised hierarchical clustering. (21B) PPI network plots of PPIs across five environments. Proteins that only interact with self are omitted. Colors are as in (21A). Edge width is proportional to the fitness and only significant edges are shown. (21C) Barplots of the log ratio of the interaction score of a perturbation over the interaction score in the benign environment as detected by three assays: PPiSeq (dark brown), split mDHFR clonal growth dynamics (light brown), and split Renilla luciferase luminescence (grey). Error bars are the standard error. *, p<0.05, Student's t-test against the benign environment.

FIG. 22A-22B depict data showing that PPiSeq is scalable. (22A) Lower bounds of the mating and loxP recombination efficiencies of a pooled mating and recombination protocol that uses ˜10¹⁰ cells per standard plate. Error bars are standard error of the mean. Each plate has the potential to generate >2×10⁷ double barcodes. (22B) Density plot of the frequencies of ˜10⁶ double barcodes that were generated by bulk mating (grey) and 2500 double barcodes that were generated by pairwise mating (purple). In both cases, the average number of reads per barcode is 67.

FIG. 23 depicts a schematic of one embodiment of the pooled competition assay. Cells are passaged through multiple growth bottleneck cycles. At each passage cells are harvested for sequencing which enables a census of the population to be taken and the relative frequencies of the genotypes to be determined.

FIG. 24 depicts histograms of the standard error of fitness estimates of high fitness (brown, x>0.07) and low fitness (grey, x<0.07) PPiSeq strains.

FIG. 25A-25C depicts validation Ftr1:Pdr5 PPI. (25A) The OD600 trajectories of the Ftr1-F[1,2]:Pdr5-F[3] split mDHFR PCA strain (purple) and a strain that lacks mDHFR fragments (grey). (25B) Barplot of the Area Under the Curve (AUC) for strains in (A). Error bars are SEM, p=2×10⁻¹¹, Student's t-test. (25C) Barplot of the Ftr1:Pdr5 split Renilla luciferase (Rluc) strain (purple) and a control that lacks any Rluc fragments (grey). Error bars are SEM, p=0.25, Student's t-test.

FIG. 26A-26B depict validation of false negatives. (26A) The OD600 trajectories of split mDHFR PCA strains Fmp45-F[1,2]:Snq2-F[3] (purple) and Tpo1-F[1,2]:Shr3-F[3] (green), and a strain that lacks mDHFR fragments (grey). (26B) Barplot of the Area Under the Curve (AUC) for strains in (26A). Error bars are SEM, * p<0.01, ** p<10⁻¹⁵, Student's t-test.

FIG. 27 depicts relative fitnesses of protein fragment pairs grown in five environments in the absence (black) or presence (purple) of MTX. Each protein fragment pair is assayed with 25 unique double barcodes across 3 growth replicates for a total of ˜75 fitness estimates (PPI score). Hollow grey circles indicate the mean fitness of the protein fragment pair in MTX across all measures. PPIs are ranked according to their fitness in the benign environment (no perturbation) and rankings are maintained between plots.

FIG. 28 depicts PPiSeq fitness correlates with protein abundance. The fitness of PPIs detected in this study or elsewhere plotted against the abundance of the least abundant protein in each PPI pair. Spearman's rho=0.68.

FIG. 29A-29B depicts the determination of the rate and removal of PCR chimeras. Most double barcode lineages are expected to be near extinction by 12 generations of growth (29A). The total number of reads for each double barcode (y-axis) was plotted against the total number of reads for each barcode 1 (BC1) multiplied by the total reads of barcode 2 (BC2, x-axis) across all conditions after 12 generations of competitive pooled growth. BC1 and BC2 frequencies are calculated by ignoring the other half of the double barcode.

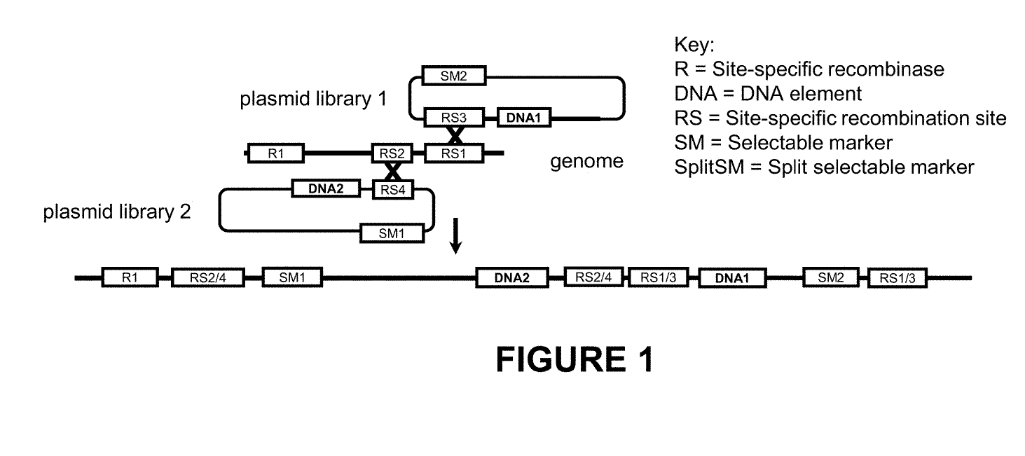

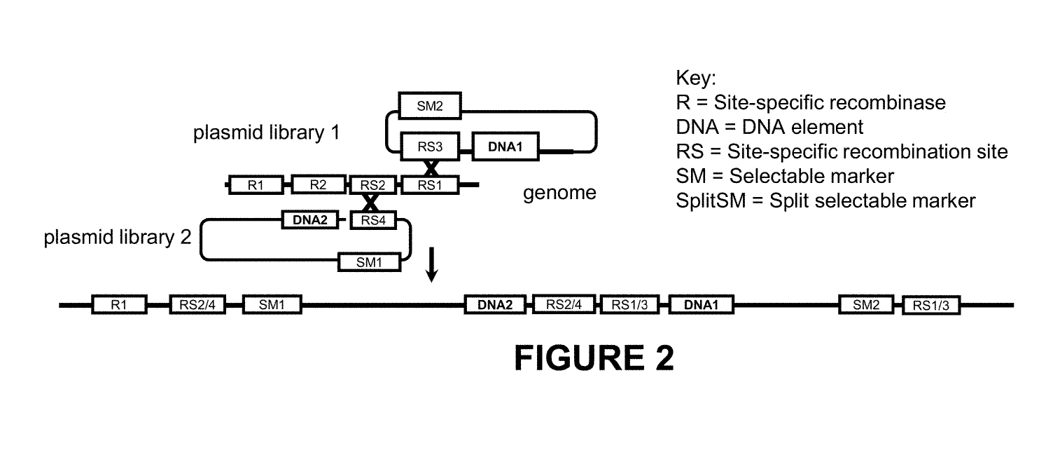

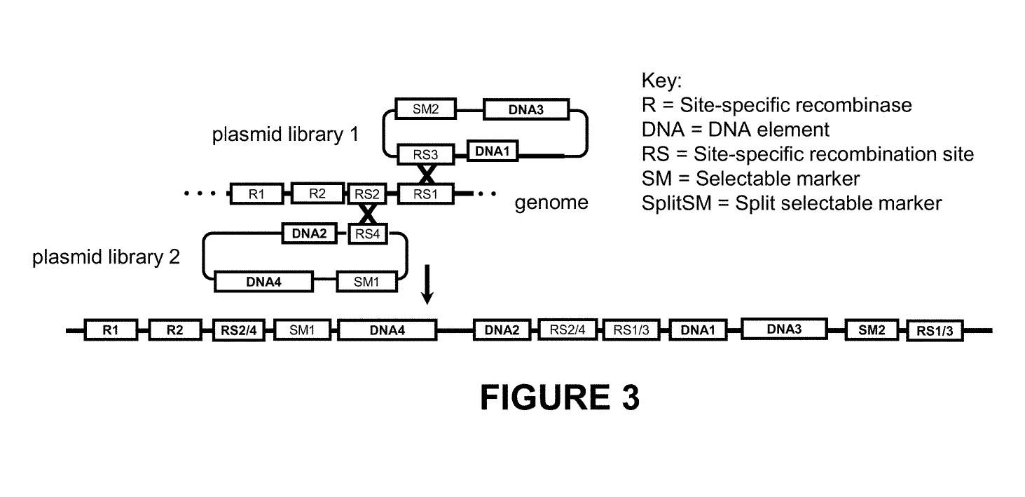

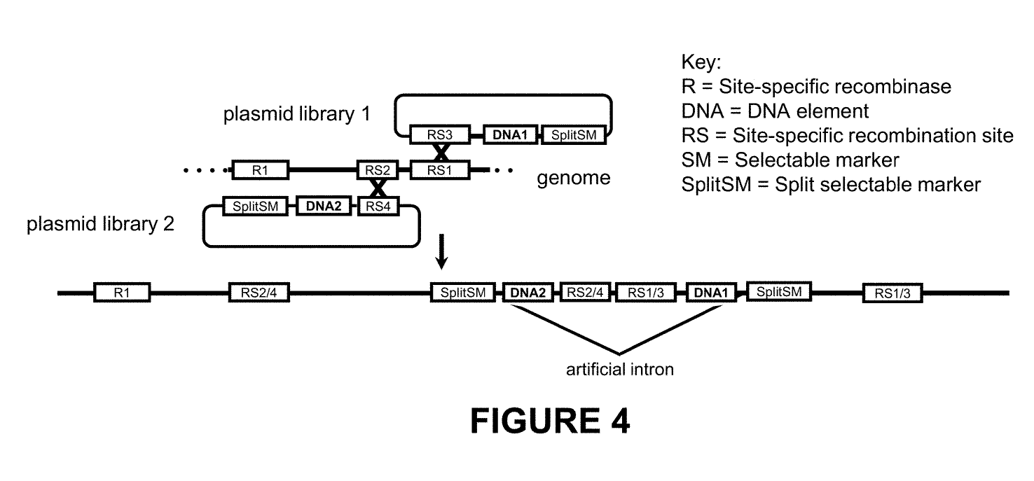

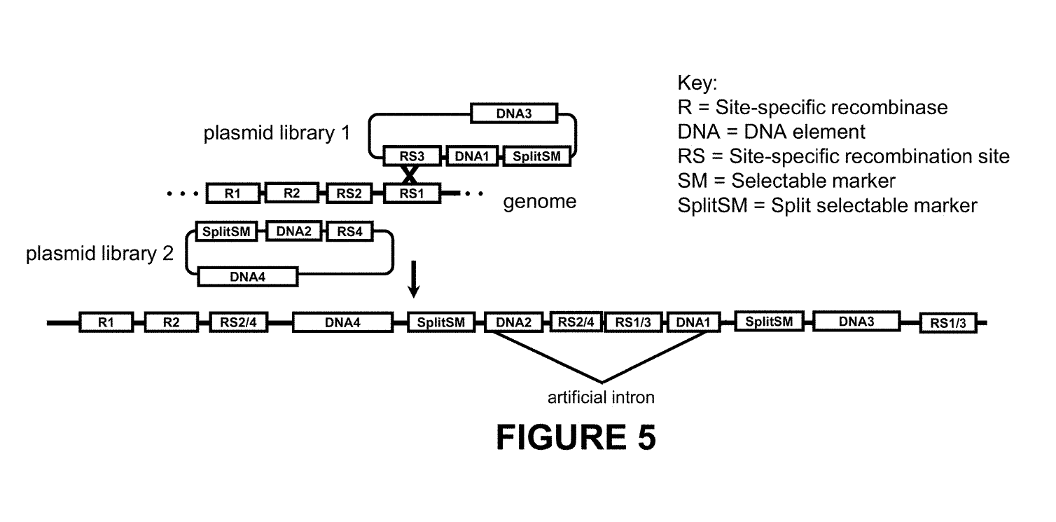

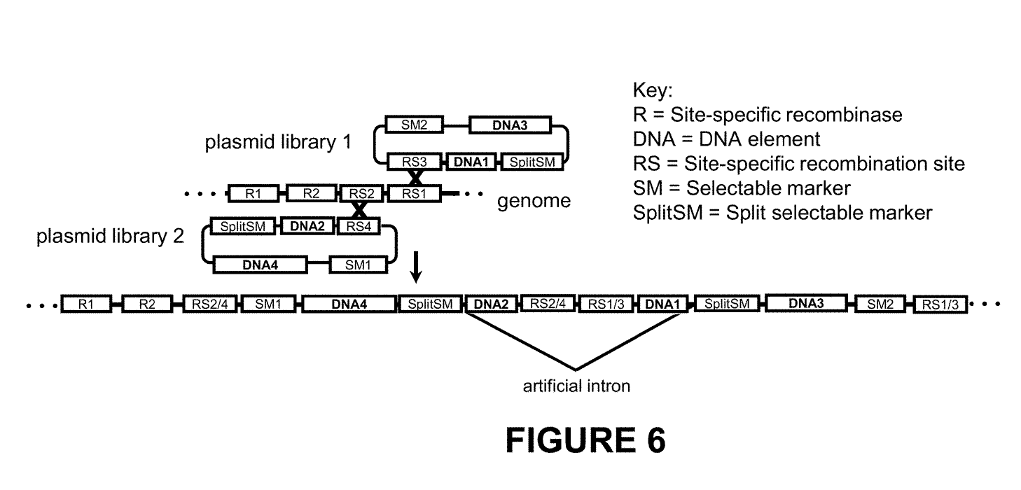

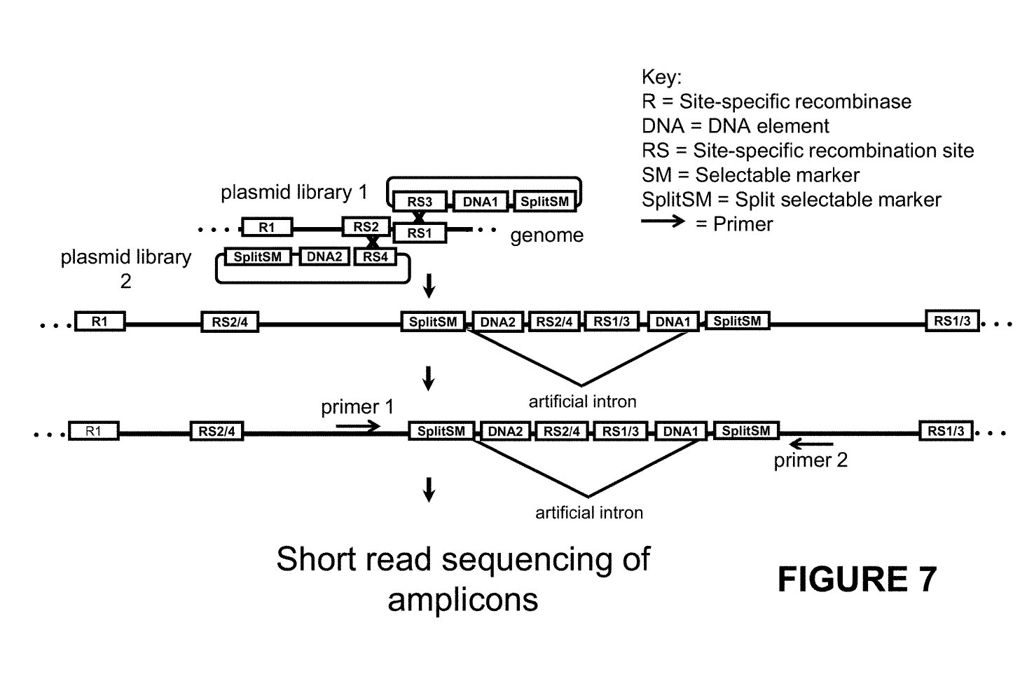

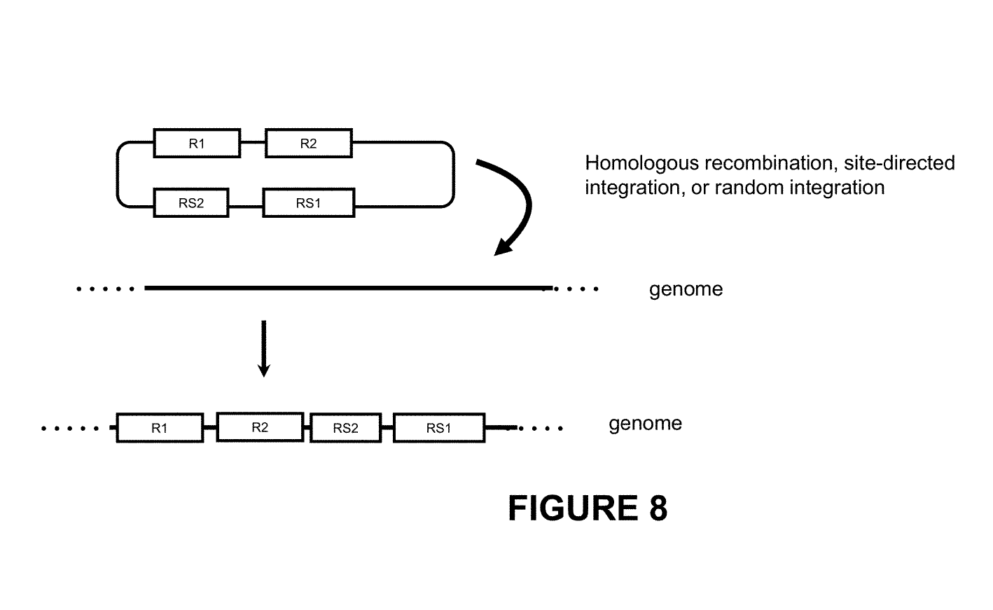

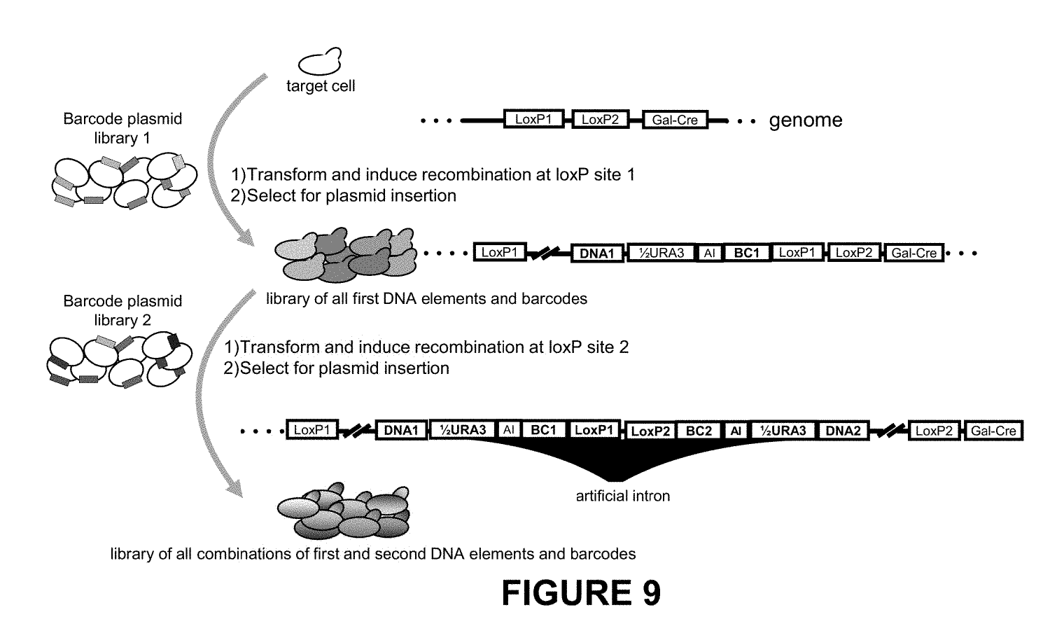

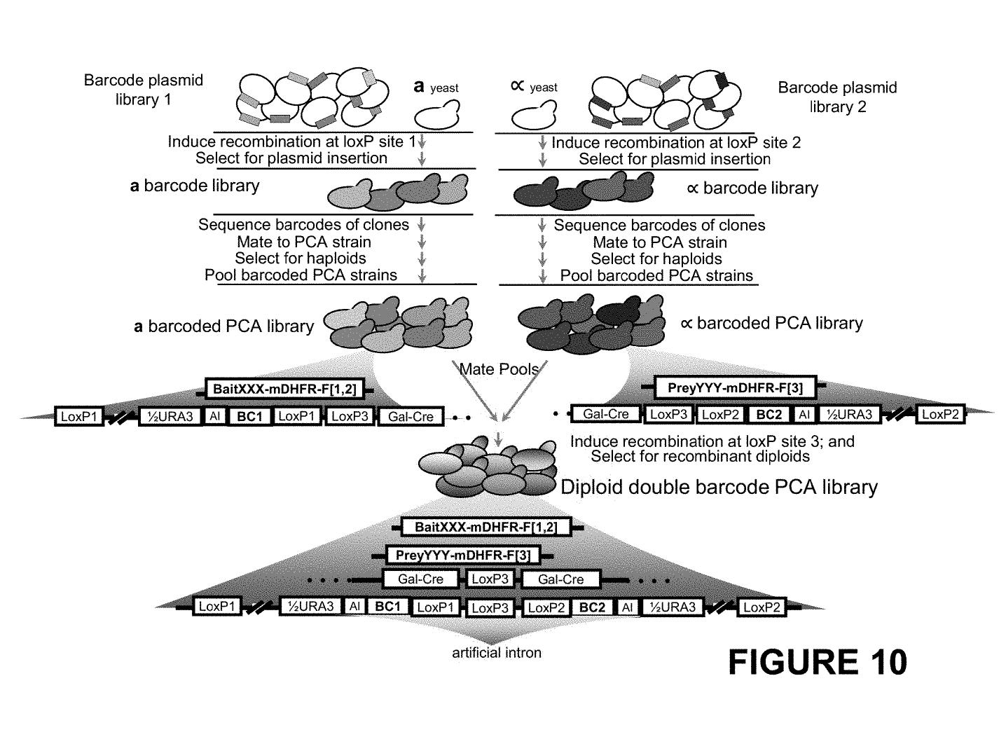

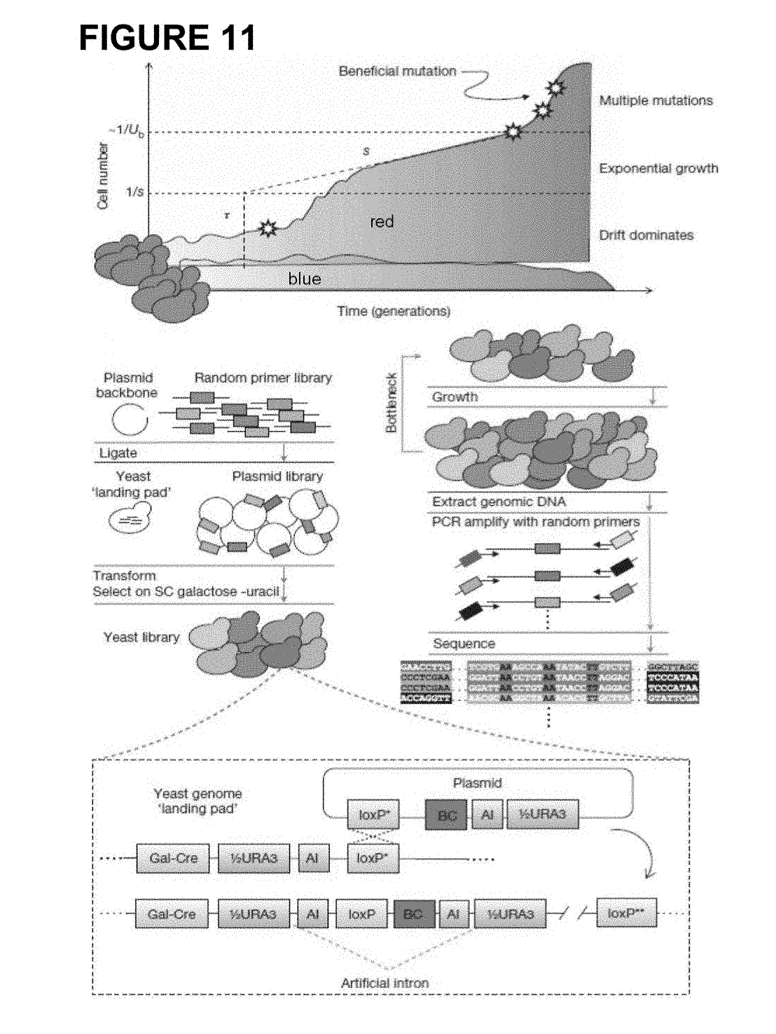

A plot that revealed a significant fraction of unexpected double barcodes remained (lower band). These unexpected double barcodes are generally confined to barcode pairs where both barcodes are abundant in the pool for other reasons. That is, they participate in a PPI (upper band), only with a different barcode partner. The most parsimonious explanation is that these double barcodes are not truly in the template pool, but rather are technical errors that result from PCR chimeras: two barcodes that stem two different templates that are merged during PCR. To remove these artifacts, this relationship is replotted except the y-axis is linear and only the lower band is plotted at BC1*BC2 frequencies greater than 10⁸ (29B).

The linear fit (red line) shows that there is a strong linear correlation between the number double barcode reads in this class and the product of the number of reads for each barcode half irrespective of its barcode partner (slope=9.36×10⁻⁸, intercept=6.14, Pearson's r=0.903). We therefore used this fit to correct all double barcode reads for PCR chimeras.

FIGS. 30A-30B depict simulated lineage trajectories (30A) and fitness estimation by likelihood maximization (30B).

FIGS. 31A-31B depict the performance fitness estimation by lineage tracking on simulated data.

FIGS. 32A-32E depict systematic errors on fitnesses. To quantify the magnitude of systematic errors in fitness, we plot all correlations between fitness inferences across all replicates for each condition.

FIGS. 33A-33E depict systematic errors on fitnesses. To quantify the magnitude of systematic errors in fitness, all correlations between fitness inferences across all replicates for each condition was plotted.

FIGS. 34A-34D iSeq platform. (34A) Schematic of the iSeq barcode locus before and after Cre-mediated recombination. Two complementary barcode constructs are introduced to the same cell on homologous chromosomes via mating. Galactose induced Cre recombination results in the two barcodes being on the same physical chromosome. Recombination events are selected for via a split URA3 marker that is only functional after recombination. (34B) First set of crosses to generate F1 strains. Two versions of each of the listed systematic deletion strains (NatMX and KanMX) are each mated to two strains with unique iSeq-compatible barcode constructs. The magic marker system is used to select for haploids of a specific mating type that contain a gene deletion and an iSeq barcode. (34C) Second set of crosses to generate F2 experimental strains. All pairwise combinations of barcoded deletion strains are next mated together, recombination at the barcode locus is induced, and double-barcode double-deletion haploids are selected following sporulation. (34D) Histograms of experimental replication. For our pilot of 9 genes, 12-16 uniquely double barcoded strains were constructed for each of the 9 possible single gene deletions (pink), and 4-8 strains were constructed for each of the 36 possible double gene deletions (turquoise).

FIGS. 35A-35F depict iSeq pooled fitness assay and reproducibility of measurements. (35A) A schematic of the iSeq pooled fitness assay. Double barcode pools are grown by serial transfer every ˜3 generations. At each transfer, relative double barcode frequencies are assayed by short-read amplicon sequencing. (35B) Representative plot of relative frequencies from a pooled fitness assay. Each line is an individual double barcode strain. Colors indicate the fitness estimate of each strain. (35C and 35D) Scatter plot of fitnesses between two biological replicates of the iSeq assay (35C) or between iSeq and a multi-well optical density based measurement (OD) (35D). Spearman's rho is shown on each plot. (35E and 35F) Frequency distributions of standard deviations of the same double barcode across three growth replicates (black), or the same double deletion across 4-8 double barcodes (grey) for iSeq (E) or OD (F) based fitness measurements.

FIGS. 36A-36C depict segregating and de novo genetic variation revealed by whole-genome sequencing. (36A) Mutations observed in F0, F1 and F2 strains. Pink bars represent gene deletion strains and turquoise bars represent control strains carrying deletions of dubious ORFs. SNP/indel frequency distributions depict the number of de novo private SNPs/indels per strain that were not observed in sequenced parental strains, but were often observed in direct descendants. Note that these SNPs in F1 strains could have been derived from private mutations present in the unsequenced iSeq barcode construct strains that F0 deletion collection strains were mated to. Aneuploidy frequency distributions depict the number of aneuploidy chromosomes present in each strain, regardless of whether or not they were observed in parental strains. (36B) For each of the strains sequenced (rows) in each of the double deletion groups, ‘WCD’ indicates identities of duplicated chromosomes, ‘SNPs’ indicates the total number of single nucleotide polymorphisms or small indels observed, and ‘Fitness’ indicates iSeq estimate in YPD. (36C) Fitnesses for each whole-genome sequenced F2 strain. Color indicates Chromosome V duplication events, and shape indicates gene reversion events in which sequencing reads mapped to one or two genic region(s) expected to be deleted. Error bars are the standard deviation of estimates across three biological replicates.

FIGS. 37A-37E depict identifying environment-dependent genetic interactions with iSeq. (37A and 37B) Scatter plot of interaction scores between two biological replicates of the iSeq assay (37A) or between iSeq and a multi-well optical density based measurement (OD) (37B). (37C) Interaction scores for individual strains carrying gene deletion pairs with a previously published positive (left) or negative (right) interaction. (37D) The genetic interaction networks in each environment. For network edges, the color represents positive (red) or negative (blue) interaction scores, the width indicates relative magnitude of each score, and dashed lines are significant changes between YPD and another environment. (37E) Genetic interaction scores of all double-barcode replicates for three double deletions in two environments. Points and error bars in 37B, 37C, and 37E are mean±SD across three growth replicates. Red dashes in 37C and 37E are median values. P-values in 37C and 37E are Wilcoxon Mann-Whitney Rank-Sum Test, and are 10% FDR corrected in 37E.

FIGS. 38A-38B depict PCR verification of integration of landing pad in mammalian cells. (37A) Integration of landing pad into mROSA26 locus in mouse 4T1 cells. (37B) Integration of landing pad into hROSA26 locus in human 293T cells. P denotes non-transfected parental 4T1 or 293T cells. CloneA is a cell lone with heterozygous integration of landing pad. Clone B is a cell clone with homozygous integration of landing pad.

FIG. 39 depicts the specificity of loxP variants. Yeast cells containing a landing pad with either a loxP site, a lox5171 site, or no lox site were transformed with plasmids containing either a loxP site, a lox5171 site, or no lox site. Transformants were counted.

FIG. 40 depicts the number of unique double barcodes per 10 ng plasmid

FIG. 41 depicts the recombination rate between loxM3W and loxW3M.

FIG. 42 depicts the mating efficiency between XLY023 and XLY024.

### DETAILED DESCRIPTION

The present disclosure provides methods for placing at least two DNA sequences proximate to each other in a genome. The genome may be from any prokaryotic or eukaryotic cell, and may be within a cell or part of a cell free system. When the genome is within a cell, the cell may be in an organism or in culture. The cell may, for example, be a yeast, a plant, an insect cell, a worm cell, an avian cell, or a mammalian cell. The mammalian cell may, for example, be a cell from a farm animal, a laboratory animal or, when the cell is in culture, a human. When the cell is in an organism, the organism may, for example be a farm animal or a laboratory animal. Some examples of farm animals include chickens, cows, goats, sheep and lambs. Some examples of laboratory animals include round worms, fruit flies, mice, rats, rabbits and monkeys.

A first site-specific recombination site is provided to the genome. Site-specific recombination sites are well known in the art. Examples of site-specific recombination sites include loxP, FRT, attP, attB, and target sites for the R recombinase of Zygosaccharomyces rouxii (RS sites). Variants of the aforementioned site-specific recombination sites and combinations thereof have also been contemplated. For example, variants of loxP include lox511, lox 5171, lox2272, M2, M3, M7, lox71, and lox66.

The genome having the above-mentioned first site-specific recombination site is recombined with a third site-specific recombination site that is compatible with the first site-specific recombination site. The third site-specific recombination site may be any recombination site that is compatible with the first site-specific recombination site. The third site-specific recombination site and the first site-specific recombination site may be recombined when both are within the genome or within a plasmid. Alternatively, the third site-specific recombination site and the first site-specific recombination site may be recombined when one is in the genome and the other is on a plasmid.

The third site-specific recombination site is associated with a first DNA sequence. As used herein, the term “associated with” means that the elements to which it refers are located on a single DNA molecule prior to the subject recombination event. For example, the third site-specific recombination site is associated with a first DNA sequence when both elements are located on the same plasmid.

The DNA molecule may be of any size that practically allows its construction, purification, amplification, and insertion into target cells. For example, the size of the DNA molecule is less than 200 kb, 150 kb, 100 kb, 50 kb, 25 kb, 10 kb, or 5 kb.

The number of bases between the third site-specific recombination site and the first DNA sequence is such that the first DNA sequence and the second DNA sequence are proximate in the genome after the recombinations.

As provided herein, recombination events between site-specific recombination sites do not include homologous recombination that can lead to higher rates of off target integrations and multiple insertion events.

A recombinase specific for the first site-specific recombination site and the third site-specific recombination site is used to induce the recombination. Recombinases are well known in the art. For example, when loxP derived recombination sites are used, Cre is a suitable recombinase. Examples of other suitable recombinases for other site-specific recombination sites include the FLP recombinase, the R recombinase of Zygosaccharomyces rouxii, the lambda integrase, the PhiC31 integrase, the Bxb1 integrase, the TnpX transposase, and combinations thereof. Variants of the aforementioned recombinases have been contemplated. Such variants include those that have increased recombinase activity as compared to the wild type recombinase, or those that have specificity for mutant/variant site-specific recombination sites. The recombinase may be located in the genome or in a plasmid. The recombinase may be under the control of an inducible promoter.

The first DNA sequence may include any desirable nucleic acid element. For example, the DNA sequence may contain barcodes, promoters, coding regions, sgRNA, gRNA, crRNA, miRNA, piRNA, siRNA, enhancers, intronic elements, and combinations thereof. The third site-specific recombination site is preferably associated with at least one cell selectable marker or a first portion of a split cell-selectable marker that confers a trait suitable for artificial selection. Cell selectable markers are well known in the art. A selectable marker is a gene introduced into a cell such as a bacterial cell or eukaryotic cells in culture. The cell selectable marker may be separated into two or more components (portions), such markers are commonly known as split cell-selectable marker (Levy: 2015).

One example of a cell selectable marker is URA3. URA3 may also serve as a split cell-selectable marker when the URA3 gene is separated into two portions, and only when both portions are expressed is a functional orotidine 5′-phosphate decarboxylase enzyme formed. As a further example, the puromycin resistance (pac) gene may be used as a split cell-selectable marker.

In one embodiment, the third-site-specific recombination site is further associated with a third DNA sequence. The third DNA sequence may include one or more cloning sites, promoters, coding regions, gRNA, crRNA, miRNA, piRNA, siRNA, enhancers, intronic elements, and combinations thereof.

As used herein, a nucleic acid barcode includes any nucleic acid sequence that can serve as a unique nucleic acid identifier. For example, when at least one nucleic acid barcode is used, it is separated from every other nucleic acid barcode sequence by a genetic distance of at least two bases. In some embodiments, the genetic distance is at least 3, 4, 5, 6, 7, 8, 9, or 10 bases.

The nucleic acid barcode includes any number of nucleotides that provides sufficient ability to be tracked by sequencing. Preferably, the nucleic acid barcodes include a minimum of 4, 5, 6, 7, 8, 9, 10, 11, 12, 13, 14, 15, 16, 17, 18, 19, 20, 30, or 50 nucleotides. The preferred maximum number of nucleotides in a nucleic acid barcode is 100 nucleotides.

In one embodiment, each nucleic acid barcode is paired with a unique third DNA sequence such that the presence of a particular nucleic acid barcode corresponds with the paired third DNA sequence.

The genome is provided with a second site-specific recombination site. The second site-specific recombination site may be, and preferably is, incompatible with the first site-specific recombination site. The genome having the second site-specific recombination site is recombined with a fourth site-specific recombination site compatible with the second site-specific recombination site.

The fourth site-specific recombination site may be any recombination site that is compatible with the second site-specific recombination site. The fourth site-specific recombination site and the second site-specific recombination site may be recombined when both are within the genome or when both are within a plasmid. Alternatively, one of the fourth site-specific recombination sites and the second site-specific recombination site is in the genome and the other is in a plasmid.

The fourth site-specific recombination site is associated with a second DNA sequence. The second DNA sequence may, for example, include nucleic acid barcodes, promoters, coding regions, sgRNA, gRNA, crRNA, miRNA, piRNA, siRNA, enhancers, intronic elements, and combinations thereof. The fourth site-specific recombination site is preferably associated with at least one cell selectable marker or a first portion of a split cell-selectable marker. In one embodiment, the fourth-site-specific recombination site is further associated with a fourth DNA sequence. The fourth DNA sequence may include one or more multiple-cloning sites, promoters, coding regions, sgRNA, gRNA, crRNA, miRNA, piRNA, siRNA, enhancers, intronic elements, and combinations thereof.

In one embodiment, each nucleic acid barcode is paired with a unique fourth DNA sequence such that the presence of a particular nucleic acid barcode corresponds with the paired fourth DNA sequence.

The site-specific recombination sites may be inserted into the genome by any method known in the art that leads to stable and specific insertion of a DNA site-specific recombination site into a genome. The site-specific recombination site may, for example, be provided to the genome by way of a DNA molecule by means of homologous recombination, or by CRISPR/CAS9-directed integration. Some examples of DNA molecules include plasmids and viruses.

The above-identified insertion or recombination steps may be performed in any order; and any two, three, or four of the above-mentioned steps may be combined into a single step. For example, a cell may be provided with a first site-specific recombination site in the genome; the third site-specific recombination site located on a plasmid along with the second site-specific recombination site and a first DNA sequence is recombined with the first site-specific recombination site; and a second plasmid including a fourth site-specific recombination site and second DNA sequence is recombined with the genome.

In another embodiment, the first site-specific recombination site and the second site-specific recombination site are inserted into the genome prior to recombination with the third site-specific recombination site and the fourth site-specific recombination site. In another embodiment, the first site-specific recombination site is recombined with the third site-specific recombination site in the genome before insertion of the second site-specific recombination site into the genome.

The recombinase used for recombining the first site-specific recombination site and third site-specific recombination site may be the same as or different from the recombinase used for recombining the second site-specific recombination site and the fourth site-specific recombination site.

The method disclosed herein provides a genome having two DNA sequences that are proximate to one another. As used herein, two DNA sequences are “proximate” to one another in a genome if both DNA sequences are capable of being sequenced together via single-end or pair-end short-read sequencing. Single-end sequencing involves sequencing DNA from only one end. Pair-end sequencing involves sequencing of both ends of a fragment. These sequencing methods continuously improve. Therefore, it is expected that the distance between two DNA sequences that are capable of being sequenced together via such methods will continuously increase (van Dijk: 2014).

According to today's most commonly used technology, for example, two DNA sequences are proximate by single-end sequencing if the total number in the first and second DNA sequence as well as the total number of nucleotides between the two DNA sequences add up to less than the typical read length. For example, two DNA sequences are proximate by singe-end sequencing if the total number in the first and second DNA sequence as well as the total number of nucleotides between the two DNA sequences is less than 20,000, 1,000, 400, 300, 200, 150, 125, 100, 50, 75, or 35 bases. Two DNA sequences are proximate by paired-end sequencing if they can be amplified by PCR and the amplicon can be practically used within the constraints of the sequencing platform. For example, two DNA sequences are proximate by paired-end sequencing if the total number in the first and second DNA sequence as well as the total number of nucleotides between the two DNA sequences add up to less than 10,000, 9000, 8000, 7000, 6000, 5000, 4000, 3000, 2000, 1000, 900, 800, 700, 600, 500, 400, 300, or 200 bases.

In the future, it is possible that two DNA sequences will be proximate if, for example, the total number of nucleotides in the first and second DNA sequence as well as the total number of nucleotides between the two DNA sequences add up to less than 100,000, 50,000, or 20,000 bases. It is furthermore contemplated that two DNA sequences will be proximate if, for example, the first and second DNA sequences are on the same chromosome.

A person of ordinary skill understands that recombination of two site-specific recombination sites results in two hybrid site-specific recombination sites at the ends of the inserted DNA element or sequence. The hybrid site-specific recombination site may be the same as or different from the original site-specific recombination sites. The hybrid site-specific recombination sites may be functional with an appropriate original site-specific recombination site and allow for further rounds of recombination; or non-functional and not allow for further rounds of recombination.

A person having ordinary skill in the art can design the insertions and recombinations of DNA described above such that the first DNA sequence and the second DNA sequence will be proximate in the genome. Such a design takes into account the total number of nucleotides in the first DNA sequence and the second DNA sequence, as well as the total of those between the two DNA sequences. The nucleotides between the two DNA sequences may, if present, include at least those in one or more of: the first hybrid recombination site and associated first DNA sequence the third hybrid recombination site and associated second DNA sequence; the second hybrid recombination site; the fourth hybrid recombination site; the number of nucleotides between any of the hybrid recombination sites and any of the associated DNA sequences; and any cell selectable markers or two or more portions of a split cell-selectable marker.

Another embodiment of the invention provides a kit of components for carrying out the above-described method. In one embodiment, the kit includes a first circular DNA library comprising a plurality of DNA molecules, wherein each DNA molecule includes (i) a third site-specific recombination site, (ii) a plurality of first DNA sequences, and (iii) either a first cell-selectable marker or a first portion of a split cell-selectable marker or both; and a second circular DNA library comprising a plurality of DNA molecules, wherein each DNA molecule includes (i) a fourth site-specific recombination site, (ii) a plurality of second DNA sequences, and (iii) either a second cell-selectable marker or a second portion of a split cell-selectable marker or both. When the first circular DNA library contains a first portion cell-selectable marker, the second circular DNA library contains a second portion of a split cell-selectable marker. As used herein, DNA molecules may be plasmids or part of a viral delivery system. As used herein, the cell-selectable marker or a portion of a split cell-selectable marker may be located anywhere on the DNA molecule.

As used herein, a “plurality” of DNA molecules includes at least 10, 100, 1,000, 10,000, 1,000,000, 10,000,000, or 100,000,000 molecules.

As used herein, “DNA sequence” includes a DNA sequence of at least 4, 15, 20, 25, 50, 100, 200, 300, 400, 500, 1000, 2000, 3000, 4000, or 5000 nucleotides.

In one embodiment, the DNA sequence includes a sequence having a maximum of 6,000, 7,000, 8,000, 9,000, 10,000, 20,000, 30,000, or 40,000 nucleotides.

Any DNA sequence may be used. For example, the first and/or second DNA sequences may include: one or more barcodes, promoters, coding regions, sgRNA, gRNA, crRNA, miRNA, piRNA, siRNA, enhancers, intronic elements, or multiple cloning sites; or combinations thereof.

In another embodiment of the invention provides a kit of components for carrying out the above-described method. In one embodiment, the kit includes a first circular DNA library comprising a plurality of DNA molecules, wherein each DNA molecule includes (i) a third site-specific recombination site, (ii) at least one first DNA sequence, and (iii) either a first cell-selectable marker or a first portion of a split cell-selectable marker or both; and a second circular DNA library comprising a plurality of DNA molecules, wherein each DNA molecule includes (i) a fourth site-specific recombination site, (ii) at least one second DNA sequence, and (iii) either a second cell-selectable marker or a second portion of a split cell-selectable marker or both. When the first circular DNA library contains a first portion cell-selectable marker, the second circular DNA library contains a second portion of a split cell-selectable marker. As used herein, DNA molecules may be plasmids or part of a viral delivery system.

The DNA molecules of the first circular DNA library may further include a third DNA sequence. The third DNA sequence may include: one or more promoters, coding regions, sgRNA, gRNA, crRNA, miRNA, piRNA, siRNA, enhancers, intronic elements, or multiple-cloning sites; or combinations thereof.

The DNA molecules of the second circular DNA library may further include a fourth DNA sequence. The fourth DNA sequence may include: one or more promoters, coding regions, sgRNA, gRNA, crRNA, miRNA, piRNA, siRNA, enhancers, intronic elements, or multiple-cloning sites; or combinations thereof.

In one embodiment, the first and/or second DNA molecule further contains one or more DNA sequences that express a site-specific recombinase.

In one embodiment, the plurality of first DNA sequences and second DNA sequences together provide more than 100, 1,000, 2,500, 5,000, 7,500, 10,000, 100, 000, 1,000,000, 10,000,000, 100,000,000, or 1,000,000,000 unique DNA sequence combinations.

In another embodiment, the sequences of a majority of the first DNA sequences and second DNA sequences, are separated from every other first DNA sequence or second DNA sequence by a genetic distance of at least two bases. In some embodiments, the genetic distance is at least 3, 4, 5, 6, 7, 8, 9, or 10 bases.

The kit optionally further contains a fifth DNA sequence having (i) a first site-specific recombination site compatible with the third site-specific recombination site (ii) a second site-specific recombination site compatible with the fourth site-specific recombination site. The first site-specific recombination site is incompatible with the second and fourth site-specific recombination sites. The second site-specific recombination site is incompatible with the first and third site-specific recombination sites. In one embodiment, the fifth DNA sequence further contains one or more DNA sequences that express a site-specific recombinase.

The first site-specific recombination site and the second site-specific recombination site are located on the fifth DNA sequence such that when the third site-specific recombination site recombines with the first site-specific recombination site; and (ii) the fourth site-specific integration recombines with the second site-specific recombination site, the first and second DNA sequences are proximate.

The fifth DNA sequence is a size that practically allows its construction, purification, amplification, and integration into the genome of target cells. For example, the size of the fifth DNA sequence is less than 200 kb, 150 kb, 100 kb, 50 kb, 25 kb, 10 kb, 5 kb, 1 kb, 500 bases, or 100 bases.

In one embodiment, the fifth DNA sequence further contains one or more DNA sequences that express a cell-selectable marker or a portion of a split cell-selectable marker or both.

In one embodiment, the fifth DNA sequence is linear or part of a third circular DNA molecule and includes flanking DNA sequences to permit insertion of the fifth DNA sequence into a genome. When the fifth DNA sequence includes a flanking DNA sequence, the flanking DNA sequence includes (i) a fifth site-specific recombination site at one flanking site and a seventh site-specific recombination site at the other flanking site, both of which are compatible with each other and with a sixth site-specific recombination site present in the genome, but which are incompatible with site-specific recombination sites one, two, three, or four; or (ii) DNA sequences that are each homologous to one of two associated DNA sequences present in the target cell genome.

In one embodiment, the fifth DNA sequence is circular and includes a fifth site-specific recombination site to permit insertion of the fifth DNA sequence into a genome. The fifth site-specific recombination site is compatible with a sixth site-specific recombination site present in the genome but incompatible with site-specific recombination sites one, two, three, or four.

In another embodiment, the fifth DNA sequence may be contained in a cell genome. Examples of cell genomes include those of yeast cells, bacterial cells, plant cells, insect cells, worm cells, avian cells, mammalian cell, or cell lines in a culture. In another embodiment, the cell genome is contained in a multicellular organism. Examples of a suitable multicellular organism include a plant, a laboratory animal, or a farm animal. Some examples of farm animals include chickens, cows, goats, sheep, and lambs. Some examples of laboratory animals include round worms, fruit flies, mice, rats, rabbits, and monkeys. In one embodiment, the genome contains one or more DNA sequences that express one or more site-specific recombinases.

The inventors have contemplated many uses of the aforementioned invention.

As one example of many uses, of the invention, the DNA sequences are part of a yeast two-hybrid (Ito: 2001, Uetz: 2000, Tavernier: 2002) or protein fragment complementation system (Galarneau: 2002, Cabantous: 2005, Tarassov: 2008). Such uses allow extremely large protein-protein interaction libraries to be cost-effectively constructed and screened as pools across drugs or other environmental perturbations.

As a second use of the invention, DNA sequences are endogenously-expressed genes, over-expressed genes or small RNAs, combinations of which can be assayed for their impact on cellular fitness or some other phenotype. For example, cell large pools could be screened for gene combinations that rescue or cause neoplastic transformation.

As a third use of the invention, DNA sequences are gene repression or knockout elements such as shRNAs or gRNAs.

As a fourth use of the invention, DNA sequences are a combination of promoters and genes, allowing for high level parallel analyses of the elements that control gene expression.

As a fifth use of the invention, DNA sequences above can be mixed and matched to study, for example, the impact of a set gene knockdowns on a set of protein-protein interactions. Indeed, once constructed, a library of DNA sequences can be easily used in combination with any other compatible library.

A sixth use of the invention is to insert large barcode libraries absent any additional DNA elements. Barcoded cell pools can be used in lineage tracking experiments to examine the dynamics of evolution, infection and cancer (Levy: 2015, Blundell: 2014, Bhang: 2015).

In the specification, numerous specific details are set forth in order to provide a thorough understanding of the present embodiments. It will be apparent, however, to one having ordinary skill in the art that the specific detail need not be employed to practice the present embodiments. In other instances, well-known materials or methods have not been described in detail in order to avoid obscuring the present embodiments.

Throughout this specification, quantities are defined by ranges, and by lower and upper boundaries of ranges. Each lower boundary can be combined with each upper boundary to define a range. The lower and upper boundaries should each be taken as a separate element.

Reference throughout this specification to “one embodiment,” “an embodiment,” “one example,” or “an example” means that a particular feature, structure or characteristic described in connection with the embodiment or example is included in at least one embodiment of the present embodiments. Thus, appearances of the phrases “in one embodiment,” “in an embodiment,” “one example,” or “an example” in various places throughout this specification are not necessarily all referring to the same embodiment or example. Furthermore, the particular features, structures or characteristics may be combined in any suitable combinations and/or sub-combinations in one or more embodiments or examples. In addition, it is appreciated that the figures provided herewith are for explanation purposes to persons ordinarily skilled in the art and that the drawings are not necessarily drawn to scale.

As used herein, the terms “comprises,” “comprising,” “includes,” “including,” “has,” “having,” or any other variation thereof, are intended to cover a non-exclusive inclusion. For example, a process, article, or apparatus that comprises a list of elements is not necessarily limited to only those elements but may include other elements not expressly listed or inherent to such process, article, or apparatus.

Further, unless expressly stated to the contrary, “or” refers to an inclusive “or” and not to an exclusive “or”. For example, a condition A or B is satisfied by any one of the following: A is true (or present) and B is false (or not present), A is false (or not present) and B is true (or present), and both A and B are true (or present).

Additionally, any examples or illustrations given herein are not to be regarded in any way as restrictions on, limits to, or express definitions of any term or terms with which they are utilized. Instead, these examples or illustrations are to be regarded as being described with respect to one particular embodiment and as being illustrative only. Those of ordinary skill in the art will appreciate that any term or terms with which these examples or illustrations are utilized will encompass other embodiments which may or may not be given therewith or elsewhere in the specification and all such embodiments are intended to be included within the scope of that term or terms. Language designating such non-limiting examples and illustrations includes, but is not limited to: “for example,” “for instance,” “e.g.,” and “in one embodiment.”

In this specification, groups of various parameters containing multiple members are described. Within a group of parameters, each member may be combined with any one or more of the other members to make additional sub-groups. For example, if the members of a group are a, b, c, d, and e, additional sub-groups specifically contemplated include any one, two, three, or four of the members, e.g., a and c; a, d, and e; b, c, d, and e; etc.

### EXAMPLES

### Example 1. Plasmid Library Construction

### 1.1. Plasmid Cloning

Plasmids pBAR1 (SEQ ID NO:108), pBAR4 (SEQ ID NO:26), and pBAR5 (SEQ ID NO:27) were cloned from the following sources by standard methods: 1) plasmid backbone/bacterial origin from pAG32; 2) natMX, kanMX, and hygMX from pAG25, pUG6, and pAG32 respectively; 3) URA3 from pSH47; and 5) artificial introns, multiple cloning sites, random barcodes and lox sites from de novo synthesis (EUROSCARF, IDT).

### 1.2 Plasmid Barcode Library Construction

Random barcodes were inserted into pBAR4 (SEQ ID NO:26) and pBAR5 (SEQ ID NO:27). Two primers containing a KpnI restriction site, a random 20 nucleotides, a unique loxP site (loxW1M or loxW2M), Table 2, and a region of homology to pBAR1 (SEQ ID NO:108) were ordered from IDT:

(SEQ ID NO: 1)

PXL005 =
5′CCAGCTGGTACCNNNNNAANNNNNTTNNNNNTTNNNNNATAACTT
CGTATAATGTATGCTATACGAACGGTAGGCGCGCCGGCCGCAAAT
3′,
and

(SEQ ID NO: 2)

PXL006 =
5′CCAGCTGGTACCNNNNNAANNNNNAANNNNNTTNNNNNTTACCGT
TCGTATAGTACACATTATACGAAGTTATGGCGCGCCGGCCGCAAAT
3′.

PXL005 contains a loxW1M site; PXL006 contains a loxW2M site. Random sequences were limited to 5 nucleotide stretches to prevent the inadvertent generation of restriction sites. The PXL005 and PXL006, paired with P23,

(SEQ ID NO: 3)

P23 = 5′GCCGAAATTGCCAGGATCAGG3′,

were used to amplify a portion of pBAR4 (SEQ ID NO:26) and pBAR5 (SEQ ID NO:27), respectively. The PCR products, pBAR4 (SEQ ID NO:26) and pBAR5 (SEQ ID NO:27) were cut with KpnI and XhoI restriction sites. To generate a HygMX-loxW1M barcode library, the digested PCR product derived from PXL005 was ligated into digested pBAR5 (SEQ ID NO:27). To generate a KanMX-loxW2M barcode library, the digested PCR product derived from PXL006 was ligated to digested pBAR4 (SEQ ID NO:26). For each ligation, ˜12-15 μg of DNA was electroporated into 10-beta electrocompetent cells (NEB). Cells were allowed to recover from electroporation in liquid LB media for 30 minutes, and plated onto 118 plates (pBAR5-W1M) or 93 (pBAR4-W2M). The loxW1M-containing plasmid library was plated at a density of ˜25,500 CFU/plate, for a total of ˜3,000,000 colonies. The loxW2M-containing plasmid library was plated at a density of ˜17,000 CFU/plate, for a total of ˜1,600,000 colonies. During the recovery period in liquid media, some fraction of the cells could have undergone a cell cycle, meaning that our true library complexity is likely to be less than the number of colonies we observe. Colonies of each library were scraped from plates and pooled in 500 ml LB-Carbenicillin. A fraction of each pool was used directly for plasmid preps to generate two plasmid libraries pBAR5-W1M and pBAR4-W2M.

### 1.3 Plasmid Open Reading Frame Library Construction

Two barcoded auxotrophic rescue libraries were generated by inserting various ORFs that rescue common yeast auxotrophies into pBAR5-W1M and pBAR4-W2M. The Met15, His3, Trp1, Leu2, Lys2 ORFs were PCR amplified from pRS421, pRS423, pRS424, pRS425, D1433 his3::LYS2 Disrupter Converter plasmids, respectively (Christianson: 1992, Brachmann: 1998, Voth: 2003). All five ORFs were inserted into pBAR4-W2M or pBAR5-W1M by Gibson assembly. Briefly, ORFs were amplified with primers that extended the amplicon 20 base pairs at the 5′ end and 21 base pairs at the 3′ end. Extended 5′ and 3′ regions are homologous to sequences in the destination plasmids flanking NheI and BclI restriction sites, respectively. Each library was linearized using the NheI and BclI restriction sites and plasmids were assembled to contain each ORF. Assembled plasmids were inserted into DH5α bacteria by KCM transformation. For each ORF insertion and for plasmids containing a barcode but no ORF, 8-10 clones were picked and Sanger sequenced to discover the unique barcode. Clones were arrayed in 96-well plates and grown in 200 ul of LB+Carbenicillin to saturation overnight. Saturated wells containing clones with the same loxP site were combined together and inoculated into 500 ml LB+Carbenicillin for plasmid preparation using the Plasmid Plus Maxi Kit (QIAGEN). Final libraries, pBAR4-W2M-AuxR and pBAR5-W1M-AuxR, containing 54 and 53 barcodes, respectively, were subsequently used to generate yeast genomic double barcode libraries.

### Example 2. Yeast Cloning

Yeast landing pad strains were constructed via four sequential gene replacements. All transformations were performed using a standard high-efficiency lithium acetate method (Gietz: 2007). First, Gal-Cre-NatMX was amplified from the plasmid pBAR1 (SEQ ID NO:108) (Levy: 2015) using the primers,

(SEQ ID NO: 4)

PEV8 =
5′GTTCTTTGCTTTTTTTCCCCAACGACGTCGAACACATTAGTCCTA
CGCACTTAACTTCGCATCTG3′,
and

(SEQ ID NO: 5)

PEV9 =
5′GCTTGCGCTAACTGCGAACAGAGTGCCCTATGAAATAGGGGAATG
CATATCATACGTAATGCTCAACCTT3′,

where underlined sequences are homologous to downstream and upstream regions of the dubious open reading frame (ORF) YBR209W, respectively. This PCR product was then transformed into two S288C derivatives, BY4741 and BY4742 (Brachmann. 1998), creating the strains SHA333 (MATa, his3Δ1, leu2Δ0, met15Δ0, ura3Δ0, ybr209w::GalCre-NatMX) and SHA319 (MATα, his3Δ1, leu2Δ0, lys2Δ0, ura3Δ0, ybr209w::GalCre-NatMX) (Table 1). Each strain was verified by PCR for successful integration.

Second, the magic marker construct, MFA1pr-HIS3-MFα1pr-LEU2 (Tong: 2004), was amplified from DNA extracted from a haploid derivative of UCC8600 (Lindstrom: 2009) using the published primers (Tong: 2004):

(SEQ ID NO: 6)

P14 = 5′GCGAACAGAGTAAACCGAA3′,
and

(SEQ ID NO: 7)

P15 = 5′GAAGGTCTGAAGGAGTTC3′.

The resulting fragment was used to replace CAN1 in SHA319 and SHA333 via homologous recombination. This insertion allows for selection of either MATa or MATα haploids via growth on synthetic complete (SC) medium containing canavanine and lacking either histidine or leucine, respectively. Correct integration was verified by PCR. Yeast strains following this replacement are SHA342 (MATa, his3Δ1, leu2Δ0, met15Δ0, ura3Δ0, ybr209w::GalCre-NatMX, can1::MFA1pr-HIS3-MFAlpha1pr-LEU2) and SHA349 (MATα, his3Δ1, leu2Δ0, lys2Δ0, ura3Δ0, ybr209w::GalCre-NatMX, can1::MFA1pr-HIS3-MFAlpha1pr-LEU2).

Third, the NatX cassette in SHA342 and SHA349 strains was replaced with URA3. The URA3 cassette was amplified from pRS426 with the following primers:

(SEQ ID NO: 8)

PXL003 =
5′ATCTGTTTAGCTTGCCTCGTCCCCGCCGGGTCACCCGGCC
AGCGACATGGAGATTGTACTGAGAGTGCAC3′,
and

(SEQ ID NO: 9)

PXL004 =
5′AACATGTTCTTTGCTTTTTTTCCCCAACGACGTCGAACAC
ATTAGTCCTACTGTGCGGTATTTCACACCG3′,

where underlined sequence correspond to sequences flanking the NatMX region. The PCR product was inserted into the genome by homologous recombination to create the XLY001 strain (MATa, his3Δ1, leu2Δ0, met15Δ0, ura3Δ0, ybr209w::GalCre-URA3, can1::MFA1pr-HIS3-MFAlpha1pr-LEU2) and XLY009 strain (MATα, his3Δ1, leu2Δ0, lys2Δ0, ura3Δ0, ybr209w::GalCre-URA3, can1::MFA1pr-HIS3-MFAlpha1pr-LEU2).

Fourth, URA3 was replaced by homologous recombination with one of three duplex ultramers containing tandem loxP sites:

(SEQ ID NO: 10)
PXL008 =
5′AGATCTGTTTAGCTTGCCTCGTCCCCGCCGGGTCACCCGGCCAGCGA
CATGGTACCGTTCGTATAATGTATGCTATACGAAGTTATTGCGCGGTG
ATCACTTATGGTACCGTTCGTATAATGTGTACTATACGAAGTTATTAGG
ACTAATGTGTTCGACGTCGTTGGGGAAAAAAAGCAAAGAACATGTTGC
C3′,
(SEQ ID NO: 11)
PXL043 =
5′AGATCTGTTTAGCTTGCCTCGTCCCCGCCGGGTCACCCGGCCAGCGA
CATGGTACCGTTCGTATAATGTATGCTATACGAAGTTATTGCGCGGTG
ATCACTTATGGTACCGTTCGTATAAAGTATCCTATACGAAGTTATTAGG
ACTAATGTGTTCGACGTCGTTGGGGAAAAAAAGCAAAGAACATGTTGC
C3′,
and
(SEQ ID NO: 12)
PXL044 =
5′AGATCTGTTTAGCTTGCCTCGTCCCCGCCGGGTCACCCGGCCAGCGA
CATGGATAACTTCGTATAAAGTATCCTATACGAACGGTATGCGCGGTG
ATCACTTATGGTACCGTTCGTATAATGTGTACTATACGAAGTTATTAGG
ACTAATGTGTTCGACGTCGTTGGGGAAAAAAAGCAAAGAACATGTTGC
C3′.

The underlined sequence corresponds to genomic sequence flanking the NatMX region. The tandem loxP sites are italicized. These oligos were transformed into XLY001 cells and integration was selected for via 5-Fluoroorotic Acid (5-FOA) counter selection of URA3. This replacement resulted in XLY003 (MATa, his3Δ1, leu2Δ0, met15Δ0, ura3Δ0, ybr209w::GalCre-loxM1W-loxM2W, can1::MFA1pr-HIS3-MFAlpha1pr-LEU2), XLY005 (MATa, his3Δ1, leu2Δ0, met15Δ0, ura3Δ0, ybr209w::GalCre-loxM1W-loxM3W, can1::MFA1pr-HIS3-MFAlpha1pr-LEU2) XLY011 (MATa, his3Δ1, leu2Δ0, met15Δ0, ura3Δ0, ybr209w::GalCre-loxW3M-loxM2W, can1::MFA1pr-HIS3-MFAlpha1pr-LEU2). The sequence of all integrated tandem loxP variants was confirmed by PCR and Sanger sequencing.

To construct strains with multiple auxotrophies that also contain the necessary elements of our interaction sequencing platform, we mated the S288C derivative BY4727 (ATCC) (MATα, his3Δ300, leu2Δ0, lys2Δ0, met15Δ0, trp1Δ63, ura3Δ0)(Brachmann: 1998), to XLY003, XLY005 and XLY011. Haploid segregants were selected to contain lys2Δ0, trp1Δ63, CAN1, the tandem loxP sites, and the correct mating type by standard methods. Selected segregants are XLY065 (MATa his3Δ1 leu2Δ0 lys2Δ0 met15Δ0 trp1Δ63 ura3Δ0 ybr209w:: GalCre-loxM1W-loxM2W), XLY058 (MATα his3Δ1 leu2Δ0 lys2Δ0 met15Δ0 trp1Δ63 ura3Δ0 ybr209w:: GalCre-loxW3M-loxM2W) and XLY059 (MATa his3Δ1 leu2Δ0 lys2Δ0 met15Δ0 trp1Δ63 ura3Δ0 ybr209w:: GalCre-loxM1W-loxM3W).

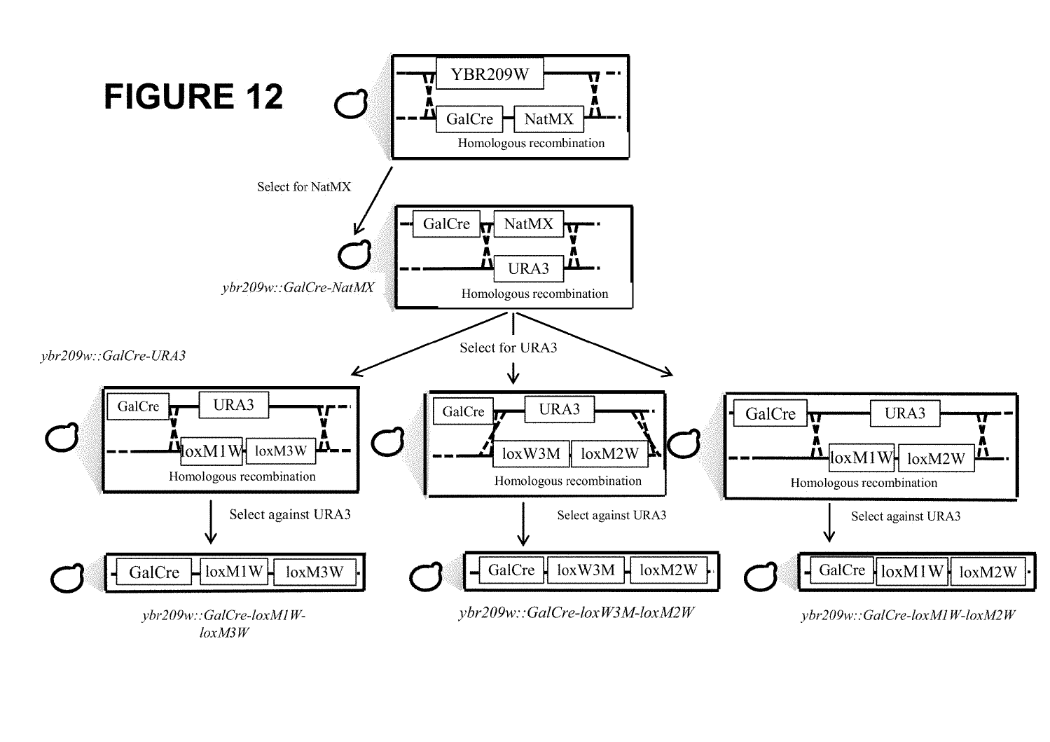

A schematic of the yeast cloning to construct the landing pad is shown in FIG. 12.

### Example 3. Specificity Tests of loxP Variants

LoxP variants loxW1W, loxW2W, and loxW3W have been reported to recombine efficiently with variants that share the same spacer region but poorly with those that do not (Lee: 1998), making these variants mutually exclusive. To test if this is true in our double barcoding systems, we performed duplicate transformations of two strains containing different tandem loxP sites, XLY005 (loxM1W-loxM3W) and XLY011(loxW3M-loxM2W), with 700 ng of single-barcode plasmids that contain no loxP site, a compatible loxP site, or an incompatible loxP site. Following transformation, cells were plated YPG (2% galactose) agar overnight. Cell lawns were replica plated onto the appropriate selectable plates to count transformation events. XLY005 was transformed with pBAR4 (SEQ ID NO:26) (no loxP), pBAR5-W1M (compatible), pBAR4-W2M (incompatible). XLY011 was transformed with pBAR5 (SEQ ID NO:27) (no loxP), pBAR5-W1M (incompatible), pBAR4-W2M (compatible). Results are depicted in FIG. 39.

### Example 4. Generation of Double Barcode Strains

### 4.1 Sequential Integration Method

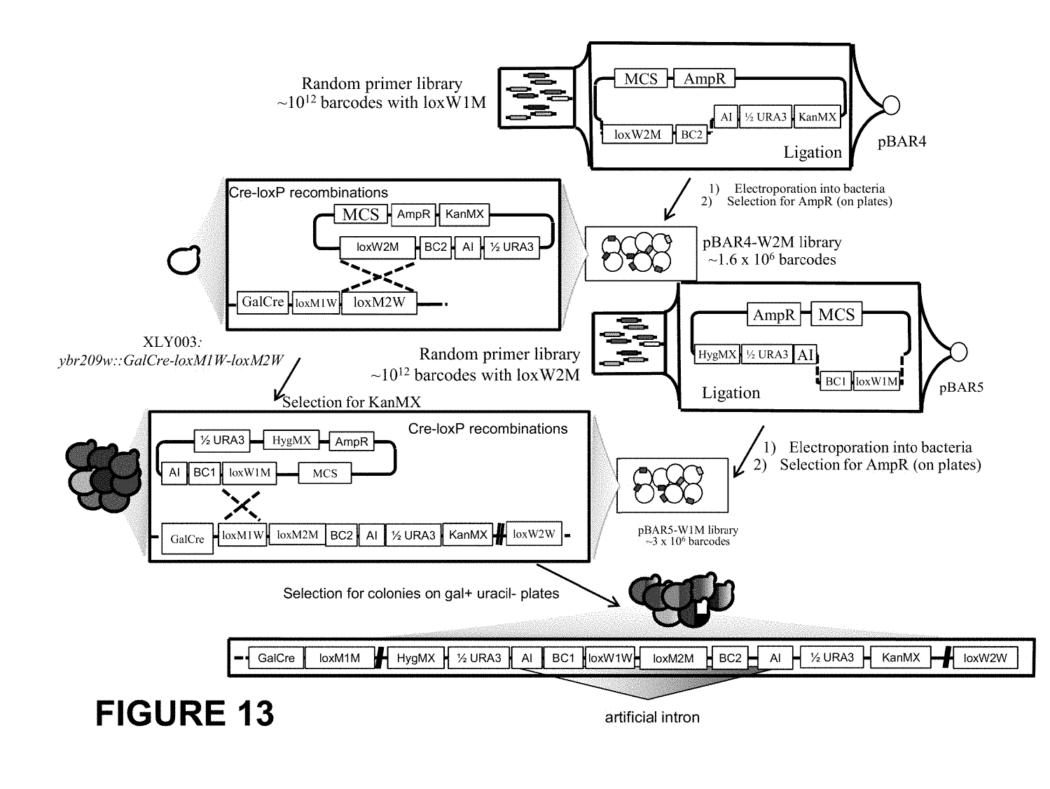

To generate double barcode strains using the sequential integration method, we first transformed XLY003 with pBAR4-W2M or pBAR4-W2M-AuxR. Transformed cells were grown overnight on YPG (2% galactose) and replica plated to YPD+G418 to select for insertion events. Plasmid insertion is irreversible because recombination between genomic loxM2W (partially crippled loxP) and plasmid loxW2M (partially crippled loxP) generates loxM2M, a non-functional loxP variant. Transformation of pBAR4 (SEQ ID NO:26) inserts first barcodes and one-half of the URA3 selectable marker at the YBR209W locus. Transformants containing multiple integrated barcoded plasmids were then pooled and transformed with pBAR5-W1M or pBAR5-W1M-AuxR. Transformation of pBAR5 (SEQ ID NO:27) inserts second barcodes and the second half of the URA3 selectable marker adjacent to the PBAR4 (SEQ ID NO:26) insertion. Cells with both plasmids inserted will have a complete the URA3 selectable marker. These cells are selected for by plating on media lacking uracil. A schematic of this process is depicted in FIG. 13.

### 4.2 Mating Method

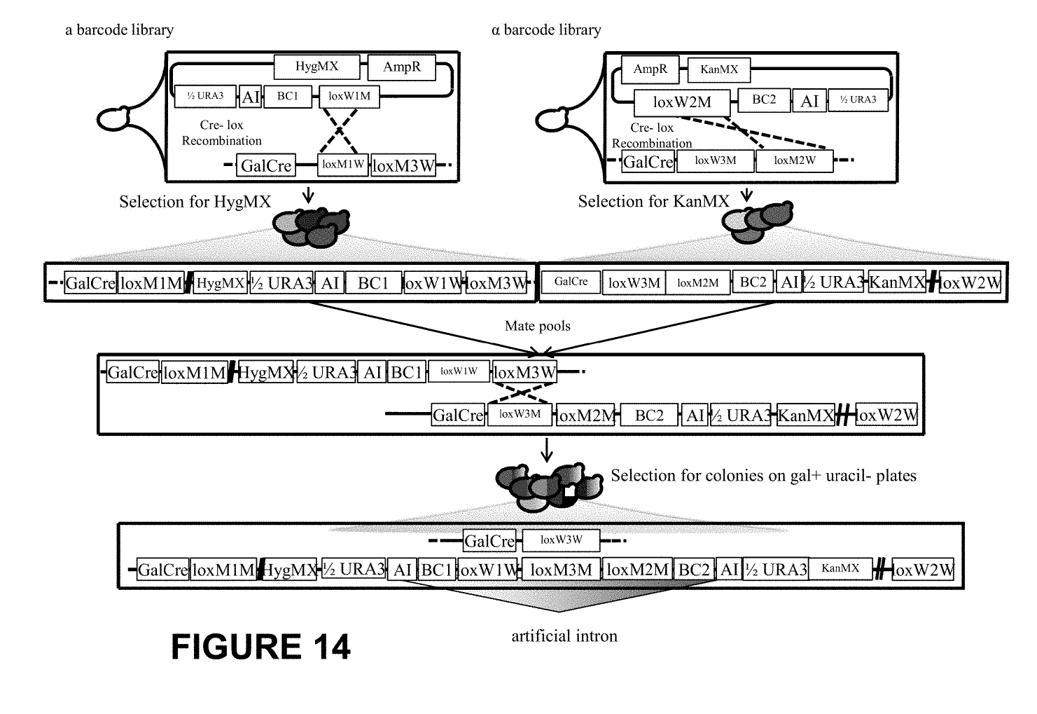

To generate double barcode strains using the mating method, we first transformed XLY005 with pBAR5-W1M or pBAR5-W1M-AuxR, and XLY011 with pBAR4-W2M or pBAR4-W2M-AuxR. Pools of transformants were mated by growing the pool to saturation in YPD, mixing equal volumes, and plating 2×10⁹ cells on YPD plates. Cell lawns were then replica plated onto SC+gal-ura plates to select for recombination between loxW3M and loxM3W on homologous chromosomes. Recombination completes the URA3 marker and brings the barcodes from pBAR4 and pBAR5 (SEQ ID NO:27) to the same chromosome, separated by three tandem loxP sites (loxW1W-loxM3M-loxM2M). A schematic of this process is depicted in FIG. 14.

### Example 5. Scalability of Double Barcoding Platforms

### 5.1 Sequential Integration Method

The number of double barcodes that can be generated by the sequential integration method is determined by the number of plasmids that can be inserted into a yeast library with a first plasmid already docked. To test the number of unique double barcodes that can be generated by this method, we first generated a yeast strain containing a single docked plasmid by integrating a single clone of pBAR4-W2M into XLY003. To test the number of second insertions, we transformed this strain with 20 μg of plasmid from a single clone of the pBAR5-W1M library. Dilutions of five replicates of transformed cells were plated on SC+gal-ura and colonies containing an integrated plasmid (those that complete the genomic URA3 gene) were counted, yielding ˜2000 transformants per μg of DNA. Based on these results, we estimate that a single plasmid maxiprep (˜1 mg of plasmid) will yield ˜2×10⁶ transformants. Results for these tests are depicted in FIG. 40.

### 5.2 Mating Method

The number of double barcodes that can be generated using the mating method depends on 1) the mating efficiency, and 2) the loxP recombination efficiency between homologous chromosomes. To estimate these efficiencies, we first generated two clonal single barcode yeast strains containing a single docked plasmid. We inserted pBAR5-W1M, containing a HygMX resistance marker, into and MATa XLY005 to create XLY023 (MATa his3Δ1 leu2Δ0 met15Δ0 ura3Δ0 ybr209w::GalCre-loxM1M-HygMX-BC-loxW1W-loxM3W can1::MFA1pr-HIS3-MFAlpha1pr-LEU2) and pBAR4-W2M, containing a KanMX resistance marker, into MATα XLY011 to create XLY024 (MATα his3Δ1 leu2Δ0 lys2Δ0 ura3Δ0 ybr209w::GalCre-loxW3M-loxM2M-BC-KanMX-loxW2W can1::MFA1pr-HIS3-MFAlpha1pr-LEU2). The two clones were grown to saturation in YPD, mixed in equal volumes, and plated overnight on YPD at a density ˜2×10⁹ cells/plate. Cells lawns were scraped and cells were counted using a Z2 particle counter (Beckman Coulter) to determine the number cell divisions the occurred on the plate (˜1.8 generations).

To estimate the mating efficiency, ˜1000, 2000, 3000, 4000, and 5000 cells of this mix were plated on YPD and YPD+Hyg+G418. All cells can grow on YPD, but only mated diploids can grow on YPD+Hyg+G418. The relative number of colonies was then used to calculate the upper and lower bound of the mating efficiency. The lower bound assumes growth of 1.8 generations following mating, while the upper bound assumes no growth following mating. Results for these tests are depicted in FIG. 42.

To test the recombination efficiency, we isolated a single diploid from the above mating, grew this clone overnight in 5 ml YPD, and plated ˜1000, 2000, 5000, and 10,000 cells on SC+gal-ura and SC-ura to count recombinants. No colonies grew on SC-ura, so the number of colonies on SC+gal-ura relative to the number of cells plated is the recombination efficiency. Results for these tests are depicted in FIG. 43.

### Example 6. Yeast Auxotrophic Rescue Library Construction

### 6.1 Sequential Insertion Method

To insert the first barcoded auxotrophic rescue plasmid library into the genome of a haploid, ˜40 μg of pBAR4-W2M-AuxR plasmid library (54 barcodes) was inserted into XLY065, resulting in ˜20,000 transformation events. Transformants were grown for 2 days on selectable media, pooled, and immediately transformed with ˜600 μg of pBAR5-W1M-AuxR. Cells were plated on 60 SC+gal-ura plates at a density of ˜5000 CFU/plate for a total of ˜300,000 transformants.

### 6.2 Mating Method

To construct a diploid double barcode library, we first transformed XLY059 (MATa) with pBAR5-W1M-AuxR and XLY058 (MATα) with pBAR4-W2M-AuxR, resulting in ˜20,000 transformants each. XLY059 and XLY058 transformants were mated on four plates as described above, generating in excess of 4×10⁷ mating events.

### 6.3 Competitive Pooled Growth Assays

Triplicate 5 ml cultures of media lacking zero (YPD and SC), one (SC-lys, SC-leu, SC-met, SC-trp, SC-his), or two (SC-lys-leu, SC-met-his, SC-his-trp, SC-lys-trp, SC-his-leu) amino acids were inoculated with 3×10⁷ cells of each auxotrophic rescue yeast barcode library. Cells were grown for five days by serial dilution, bottlenecking ˜1:8 every 24 hours. Cells grew ˜3 generations between each transfer for a total of ˜12 generations of growth. Genomic DNA from cells at each transfer was prepared using MasterPure™ Yeast DNA Purification Kit (Epicentre).

### Example 7 Double Barcode Sequencing

A two-step PCR was performed, as described (Levy: 2015) with modifications. Briefly, ˜150 ng of template per sample was amplified, which corresponds to ˜10⁷ genomes or ˜2500 copies per unique lineage tag at time zero. First, a 5-cycle PCR with OneTaq polymerase (New England Biolabs) was performed. Primers for this reaction were:

(SEQ ID NO: 13)
ACACTCTTTCCCTACACGACGCTCTTCCGATCTNNNNNNNNXXXXXTTAA
TATGGACTAAAGGAGGCTTTT,
and
(SEQ ID NO: 14)
CTCGGCATTCCTGCTGAACCGCTCTTCCGATCTNNNNNNNNXXXXXXXXX
TCGAATTCAAGCTTAGATCTGATA.

The Ns in these sequences correspond to any random nucleotide and are used in the downstream analysis to remove skew in the counts caused by PCR jack-potting. The Xs correspond to a one of several multiplexing tags, which allows different samples to be distinguished when loaded on the same sequencing flow cell. PCR products were cleaned using PCR Cleanup columns (Qiagen) and eluted into 30 ul of water. A second 23-cycle PCR was performed with high-fidelity PimestarMAX polymerase (Takara), with 25 ul of cleaned product from the first PCR as template and 50 μL total volume per tube. Primers for this reaction were the standard Illumina paired-end ligation primers:

(SEQ ID NO: 15)

AATGATACGGCGACCACCGAGATCTACACTCTTTCCCTACACGACGCTCT
TCCGATCT,
and

(SEQ ID NO: 16)

CAAGCAGAAGACGGCATACGAGATCGGTCTCGGCATTCCTGCTGAACCGC
TCTTCCGATCT.

PCR products were cleaned using PCR Cleanup columns (Qiagen). The appropriate PCR band was isolated by E-Gel agarose gel electrophoresis (Life Technologies) and quantitated by Bioanalyzer (Agilent) and Qubit fluorometry (Life Technologies). Cleaned amplicons were pooled and sequenced on a Illumina MiSeq or HiSeq using the paired end sequencing protocol. Sequencing reads were mapped to barcodes by blast using custom-written python scripts as described (Levy: 2015), allowing for ˜2 mismatches in any single barcode. Random barcodes in the primers were used to remove PCR duplicates, as described (Levy: 2015).

TABLE 1
Examples of S. cerevisiae strains
(All strains are S288C derivatives)

| Name | Genotype |
|---|---|
| BY4741 | MATα his3Δ1 leu2Δ0 met15Δ0 ura3Δ0 |
| BY4742 | MATα his3Δ1 leu2Δ0 lys2Δ0 ura3Δ0 |
| BY4727 | MATα his3Δ200 leu2Δ0 lys2Δ0 met15Δ0 |
| | trp1Δ63 ura3Δ0 |
| SHA333 | MATα his3Δ1 leu2Δ0 met15Δ0 ura3Δ0 |
| | ybr209w::GalCre-NatMX |
| SHA319 | MATα his3Δ1 leu2Δ0 lys2Δ0 ura3Δ0 |
| | ybr209w::GalCre-NatMX |
| SHA342 | MATα his3Δ1 leu2Δ0 met15Δ0 ura3Δ0 |
| | ybr209w::GalCre-NatMX |
| SHA349 | MATα his3Δ1 leu2Δ0 lys2Δ0 ura3Δ0 |
| | ybr209w::GalCre-NatMX |
| XLY001 | MATα his3Δ1 leu2Δ0 met15Δ0 ura3Δ0 |
| | ybr209w::GalCre-URA3 |
| | can1:: MFA1pr-HIS3-MFAlpha1pr-LEU2 |
| XLY009 | MATα his3Δ1 leu2Δ0 lys2Δ0 ura3Δ0 |
| | ybr209w::GalCre-URA3 |
| | can1:: MFA1pr-HIS3-MFAlpha1pr-LEU2 |
| XLY003 | MATα his3Δ1 leu2Δ0 met15Δ0 ura3Δ0 |
| | ybr209w::GalCre-lox71-lox5171/71 |
| | can1:: MFA1pr-HIS3-MFAlpha1pr-LEU2 |
| XLY005 | MATα his3Δ1 leu2Δ0 met15Δ0 ura3Δ0 |
| | ybr209w::GalCre-lox71-lox2272/71 |
| | can1:: MFA1pr-HIS3-MFAlpha1pr-LEU2 |
| XLY011 | MATα his3Δ1 leu2Δ0 lys2Δ0 ura3Δ0 |
| | ybr209w::GalCre-lox2272/66-lox5171/71 |
| | can1:: MFA1pr-HIS3-MFAlpha1pr-LEU2 |
| XLY023 | MATα his3Δ1 leu2Δ0 met15Δ0 ura3Δ0 |
| | ybr209w::GalCre-lox66/71-HygMX-BC- |
| | loxP-lox2272/71 |
| | can1:: MFA1pr-HIS3-MFAlpha1pr-LEU2 |
| XLY024 | MATα his3Δ1 leu2Δ0 lys2Δ0 ura3Δ0 |
| | ybr209w::GalCre-lox2272/66-lox5171/66/71- |
| | BC-KanMX-lox5171 |
| | can1:: MFA1pr-HIS3-MFAlpha1pr-LEU2 |
| XLY058 | MATα his3Δ1 leu2Δ0 lys2Δ0 met15Δ0 |
| | trp1Δ63 ura3Δ0 ybr209w::GalCre- |
| | lox2272/66-lox5171/71 |
| XLY059 | MATα his3Δ1 leu2Δ0 lys2Δ0 met15Δ0 |
| | trp1Δ63 ura3Δ0 ybr209w::GalCre-lox71- |
| | lox2272/71 |
| XLY065 | MATα his3Δ1 leu2Δ0 lys2Δ0 met15Δ0 |
| | trp1Δ63 ura3Δ0 ybr209w::GalCre-lox71- |
| | lox5171/71 |

TABLE 2
Examples of loxP variants, including sequences.

| | Left inverted repeat | Spacer | Right inverted repeat | |
|---|---|---|---|---|
| loxP variant | sequence | (5′-3′) | sequence | Alias |
| loxW1W | ATAACTTCGTATA | ATGTATGC | TATACGAAGTTAT | loxP |
| (SEQ ID NO: 17) | | | | |
| loxM1W | taccgTTCGTATA | ATGTATGC | TATACGAAGTTAT | lox71 |
| (SEQ ID NO: 18) | | | | |
| loxW1M | ATAACTTCGTATA | ATGTATGC | TATACGAAcggta | lox66 |
| (SEQ ID NO: 19) | | | | |
| loxW2W | ATAACTTCGTATA | ATGTgTaC | TATACGAAGTTAT | lox5171 |
| (SEQ ID NO: 20) | | | | |
| loxM2W | taccgTTCGTATA | ATGTgTaC | TATACGAAGTTAT | lox5171/71 |
| (SEQ ID NO: 21) | | | | |
| loxW2M | ATAACTTCGTATA | ATGTgTaC | TATACGAAcggta | lox5171/66 |
| (SEQ ID NO: 22) | | | | |
| loxW3W | ATAACTTCGTATA | AaGTATcC | TATACGAAGTTAT | lox2272 |
| (SEQ ID NO: 23) | | | | |
| loxM3W | taccgTTCGTATA | AaGTATcC | TATACGAAGTTAT | lox2272/71 |
| (SEQ ID NO: 24) | | | | |
| loxW3M | ATAACTTCGTATA | AaGTATcC | TATACGAAcggta | lox2272/66 |
| (SEQ ID NO: 25) | | | | |

### Example 8. Lineage Tracking with Random Barcodes

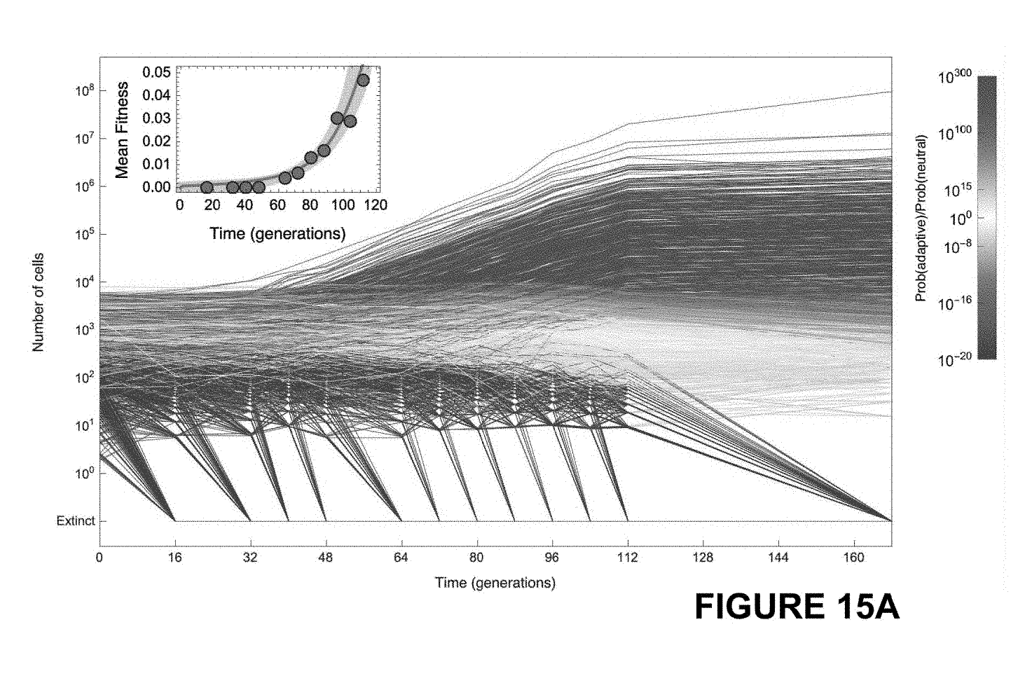

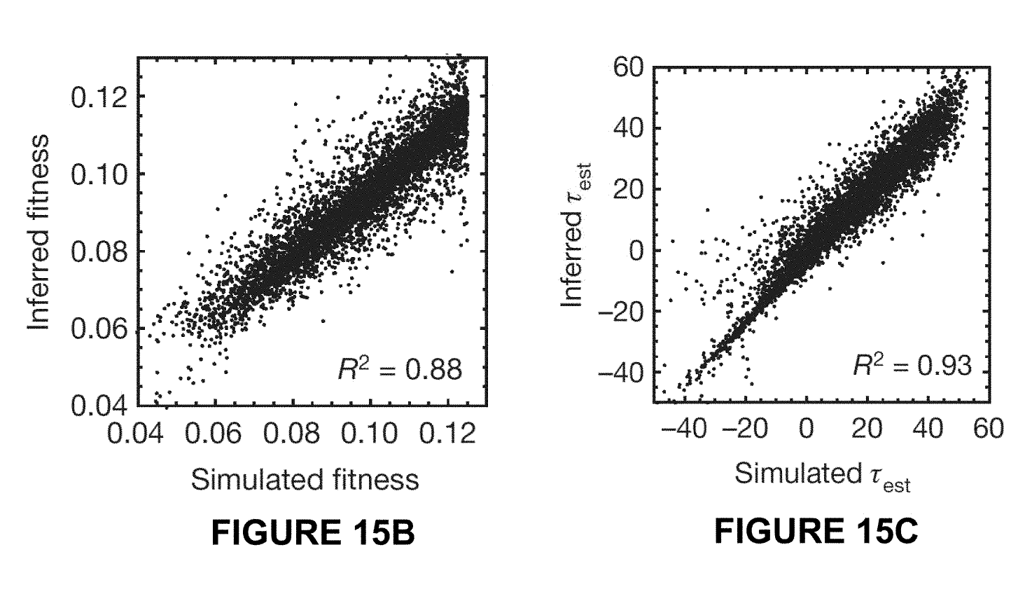

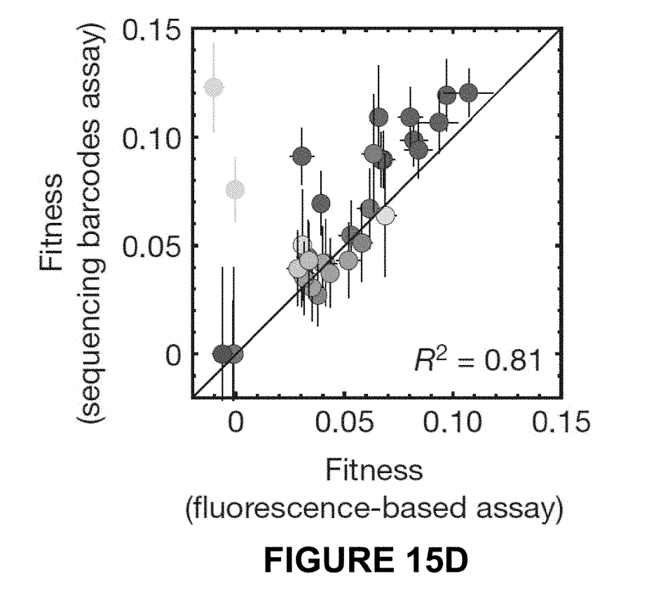

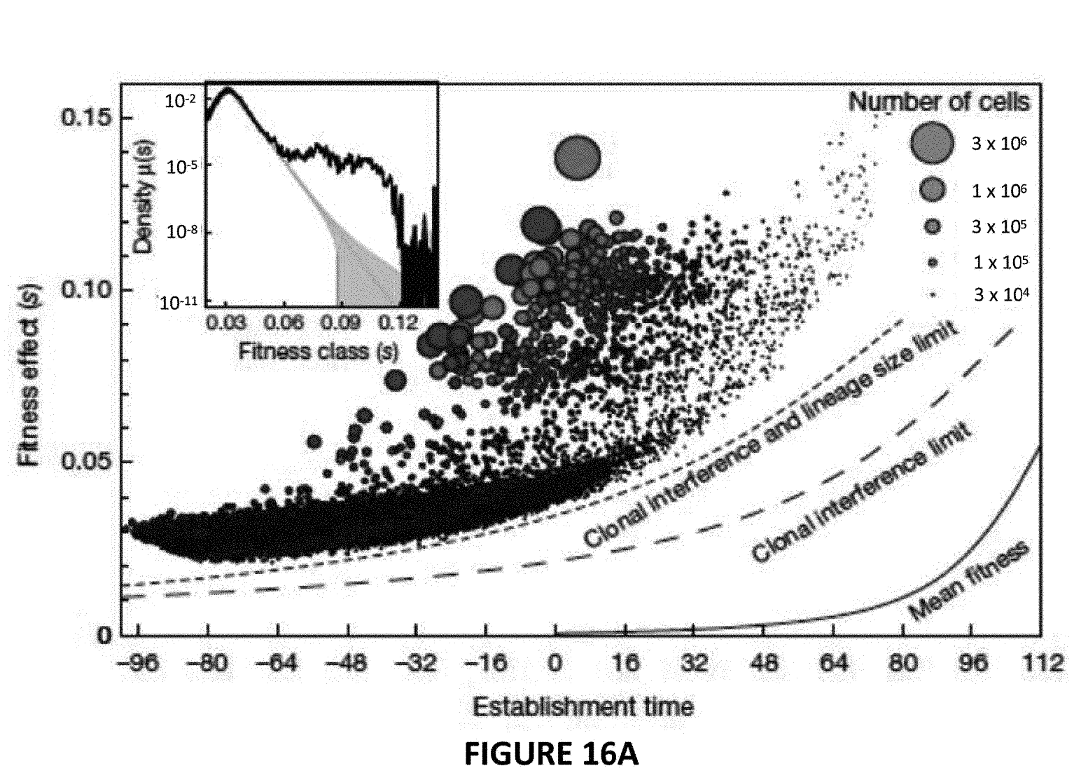

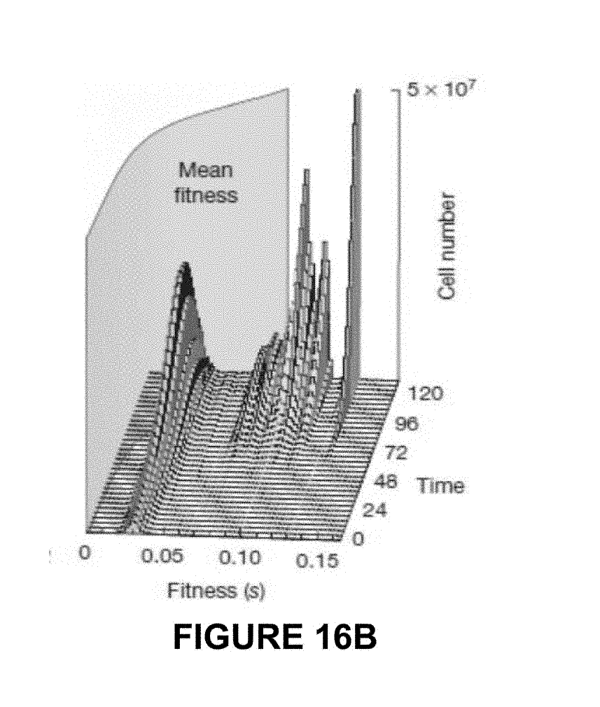

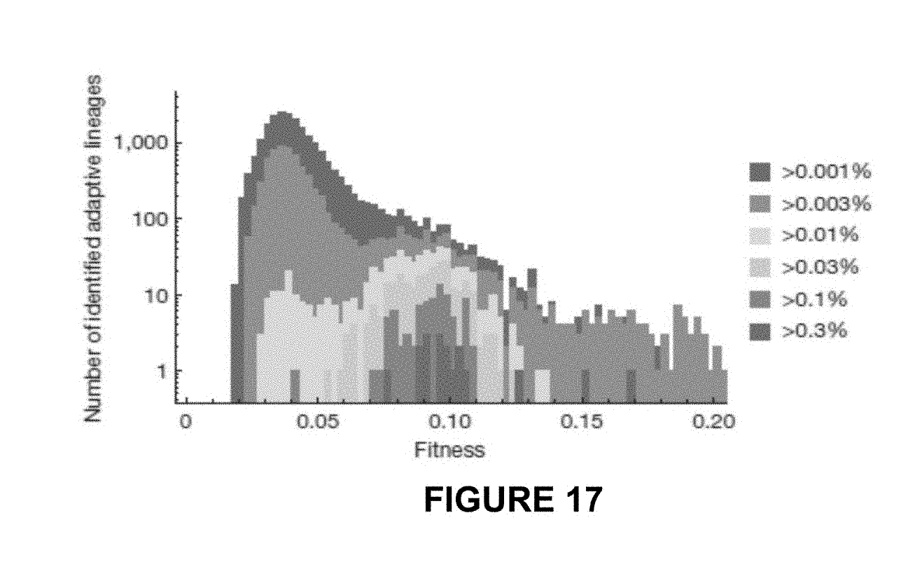

Approximately 0.5 million random barcodes were introduced into yeast and this pool was evolved under laboratory conditions to observe the evolutionary dynamics of all barcoded lineages (See FIGS. 14-17). Lineage tracking was used to discover the approximate time of occurrence (establishment time) and the fitness effect of ˜20,000 adaptive mutations. (Levy: 2015).

### Example 9. A Scalable Double-Barcode Sequencing Platform for Characterization of Dynamic Protein-Protein Interactions

A highly scalable and robust method to identify and quantitatively score dynamic PPIs that called Protein-Protein interaction Sequencing (PPiSeq) is provided herein to shown. The PPiSeq platform combines PCA, a new genomic double-barcoding technology, time-course barcode sequencing of competing cell pools, and an analytical framework to precisely call fitnesses from barcode lineage trajectories. We use these tools to examine the interactions between ˜100 protein pairs at high replication and across five environments. In a benign environment, the ability for PPiSeq to identify PPIs is on par with existing assays. In addition, PPiSeq finds that a large fraction of PPIs change across environments, many of which could be validated by other PPI assays. Finally, PPiSeq is capable of generating libraries exceeding 10⁹ double barcodes and could potentially be used to simultaneously assay the entire protein interactome in a single experiment.

### Results

### The PPiSeq Platform

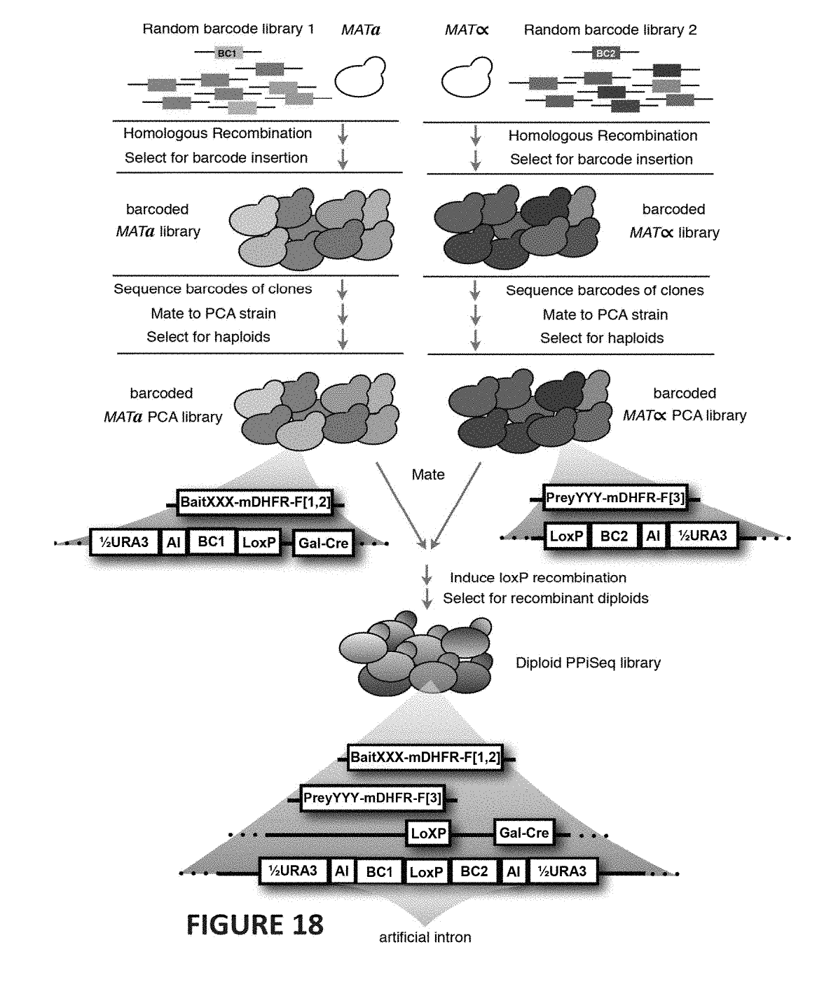

A general interaction Sequencing platform (iSeq) is developed. Barcodes that are adjacent to a loxP recombination site are introduced at a common chromosomal location in closely related MATα and MAT∝ haploids. Barcodes are placed on opposite sides of the loxP site in each sex such that mating and Cre induction causes recombination between homologous chromosomes, resulting in a barcode-loxP-barcode configuration on one chromosome (See FIG. 18). This event is selected for by loxP recombination-induced reassembly of a split URA3 marker (Levy: 2015). A double barcode unambiguously identifies both parents of a cross in highly complex cell pools, with each barcode half being in close enough proximity to allow the pair to be sequenced together by short-read sequencing. Next, double barcode strains are grown in pools, relative double barcode frequencies are assayed at several times, and their trajectories are used in combination with a global maximum likelihood method to estimate the relative fitness of each strain. While iSeq could in theory be used to study interactions between any two genetic elements (e.g. gene knockouts, point mutations, or engineered CRISPR constructs), here we use iSeq in combination with the DHFR PCA system to construct a Protein-Protein interaction Sequencing (PPiSeq) platform (FIG. 18).

### PPiSeq is Accurate and Highly Reproducible

To test the reproducibility of PPiSeq and compare it to existing PPI assays, 9 bait and 9 prey split mDHFR PCA strains were selected and 5 different barcodes were added to each. PCA constructs were chosen to encompass a number of previously-discovered PPIs. We also added 5 different barcodes to two control strains that do not contain a mDHFR. Haploid barcoded PCA strains were next pairwise mated and pooled to generate a library of 2500 double barcode (PPiSeq) strains, with each of the 100 genotypes being represented by 25 unique double barcodes.

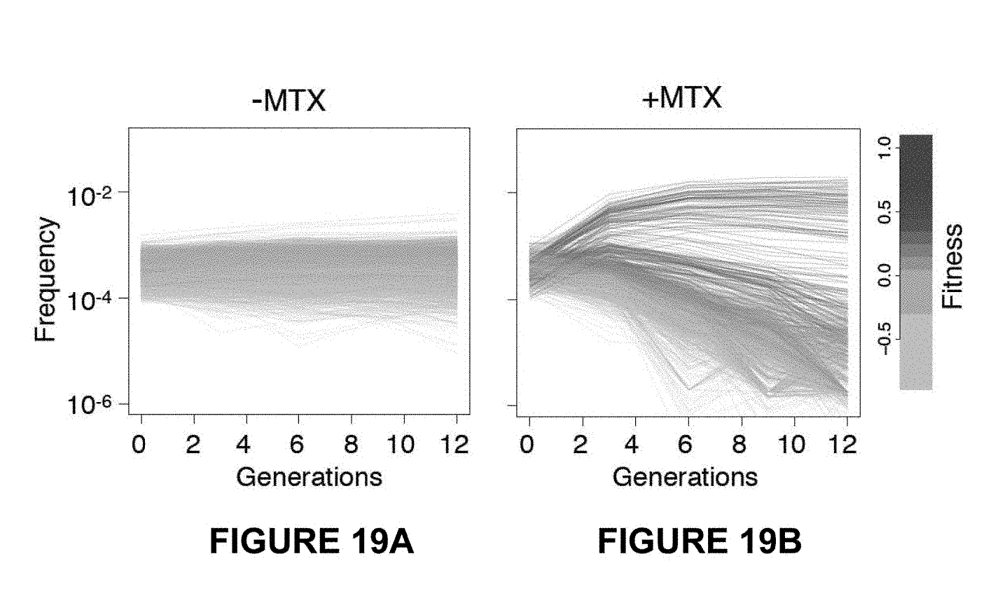

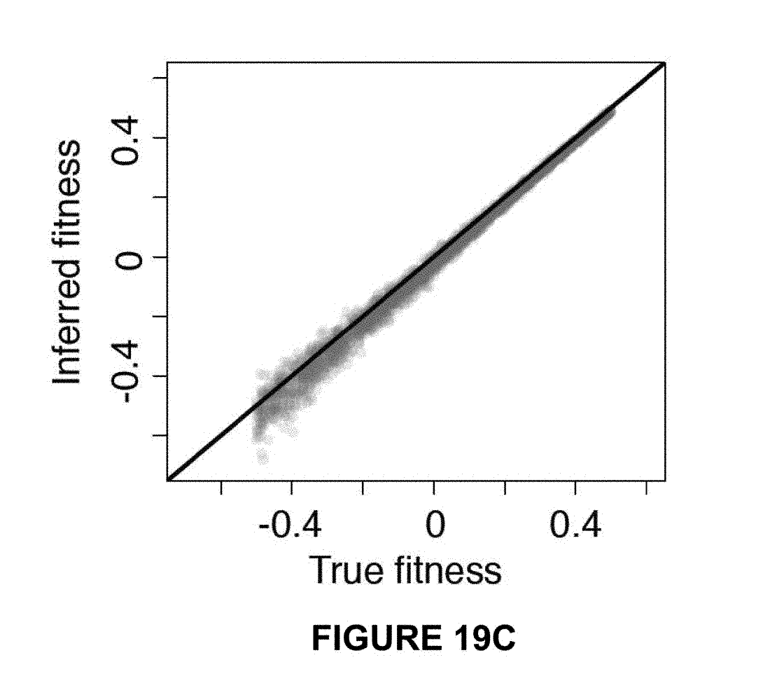

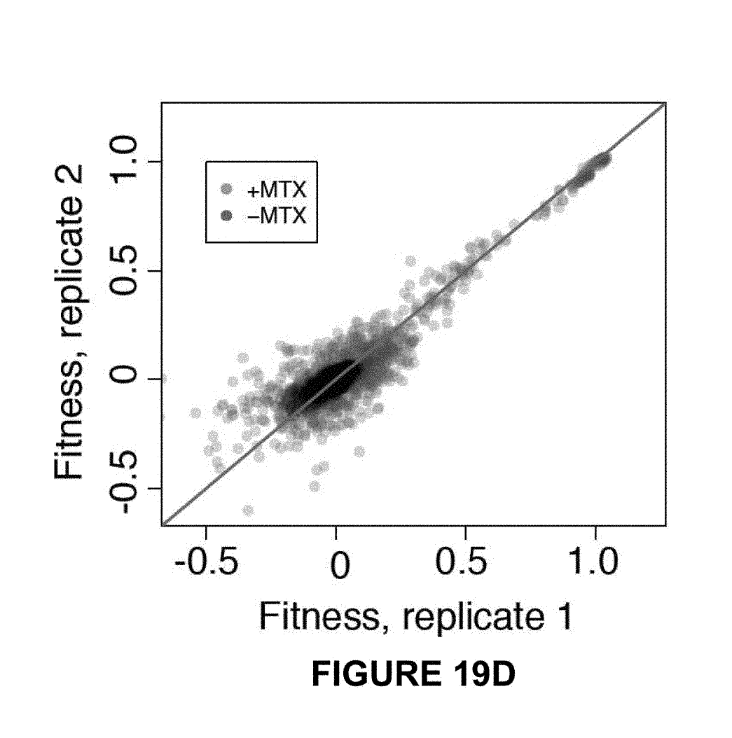

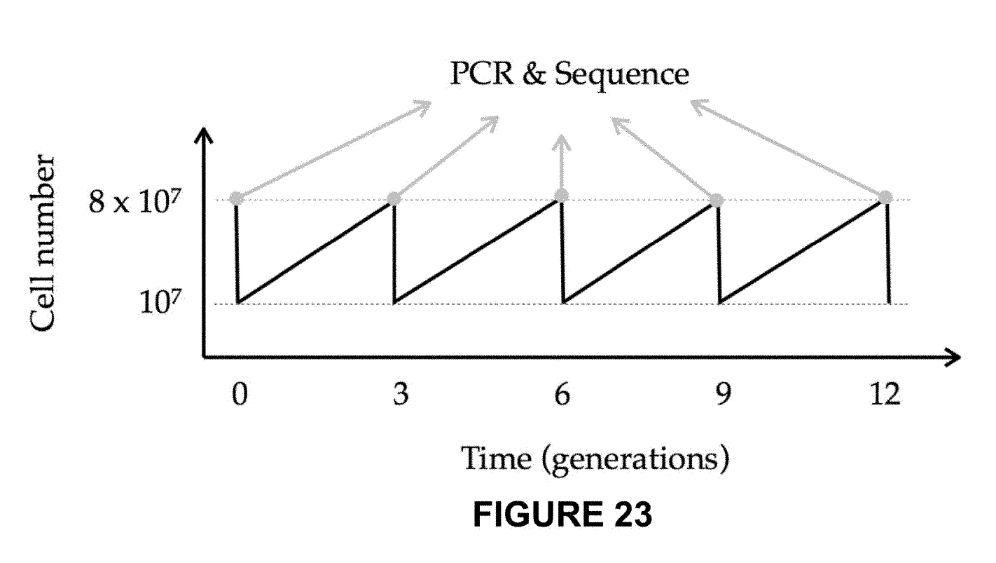

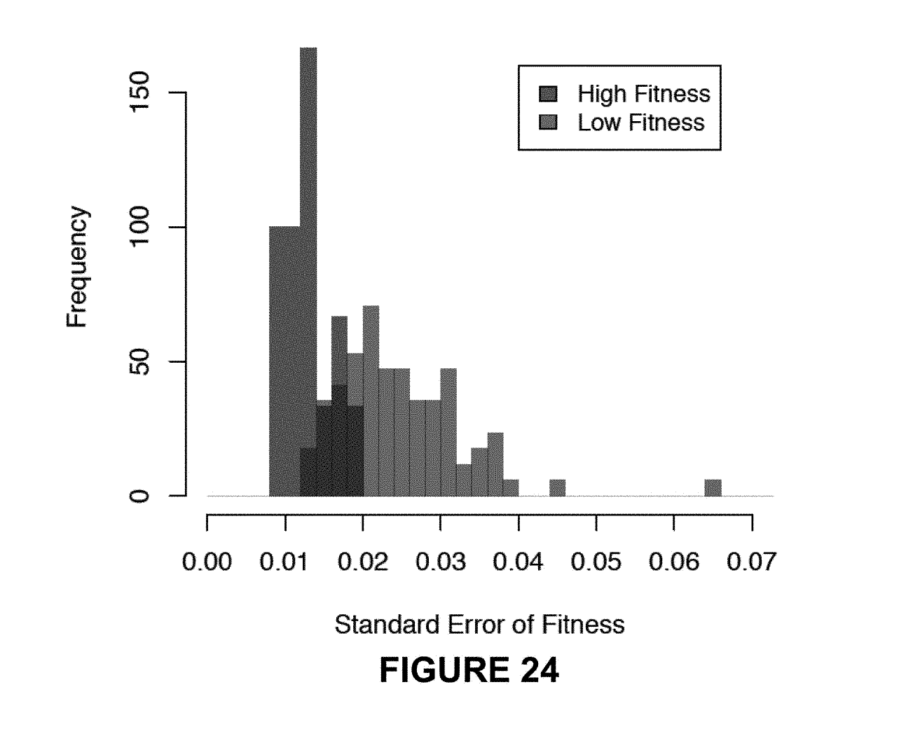

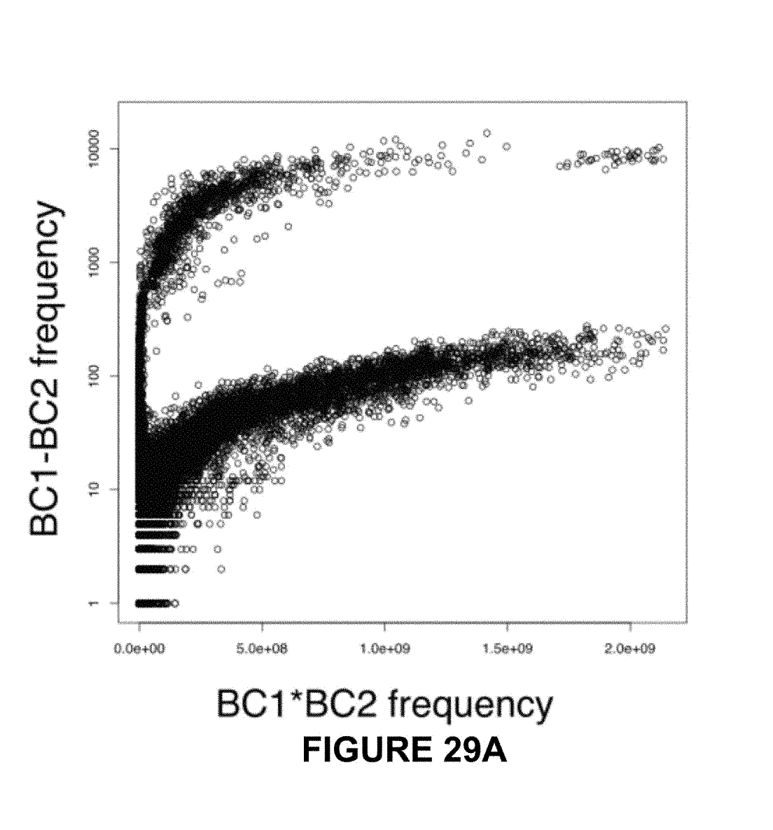

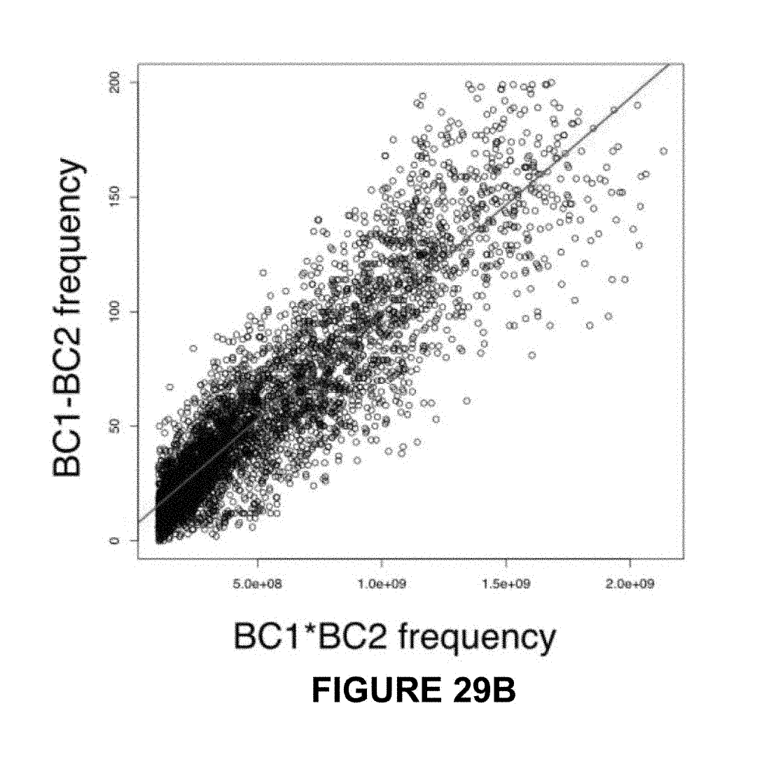

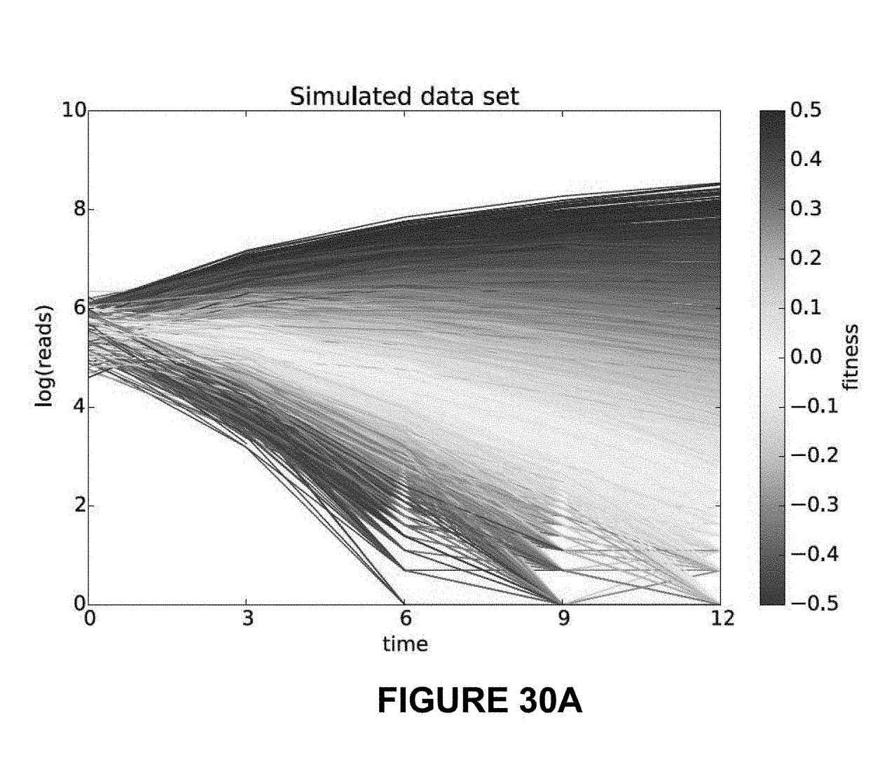

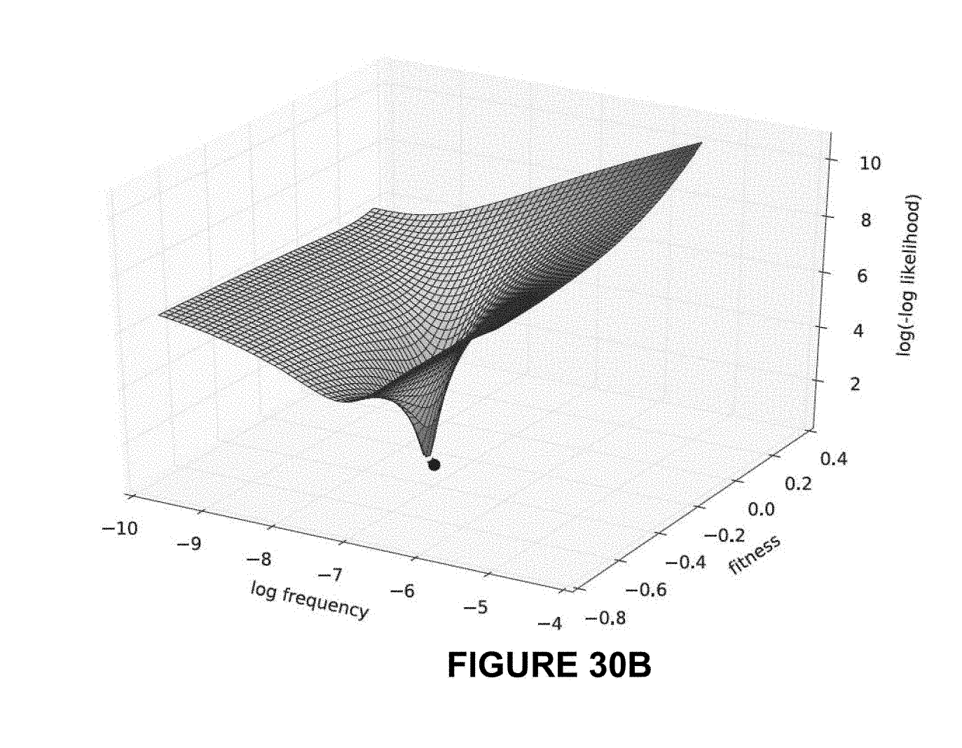

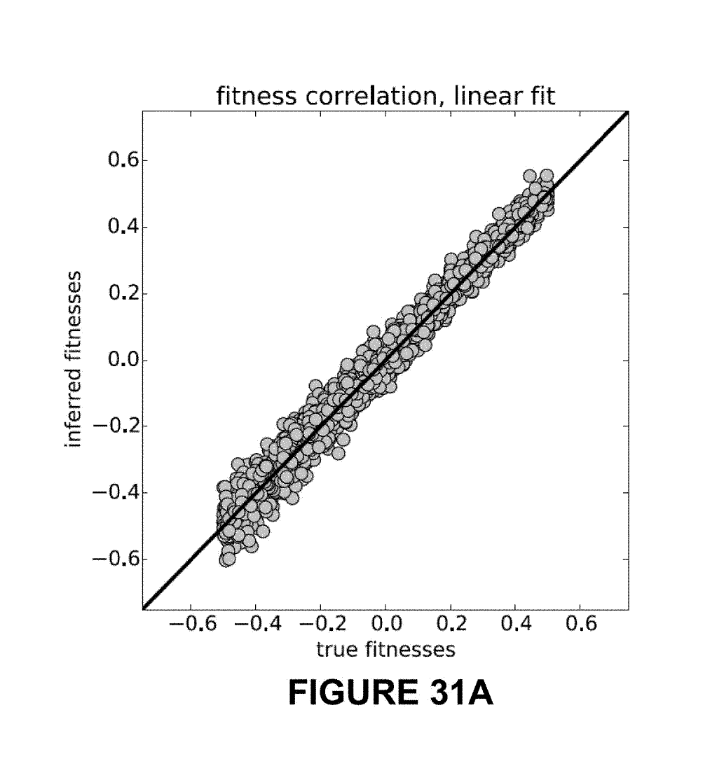

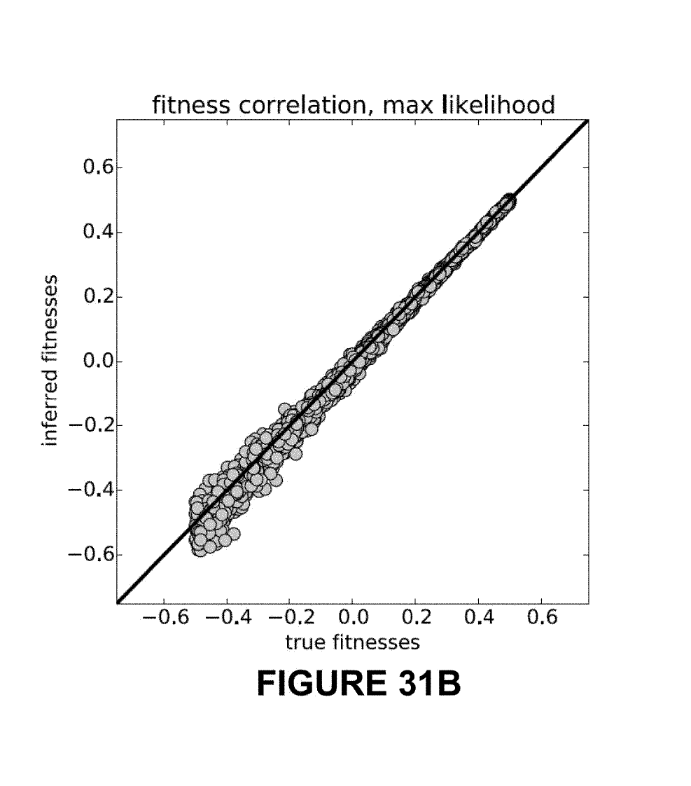

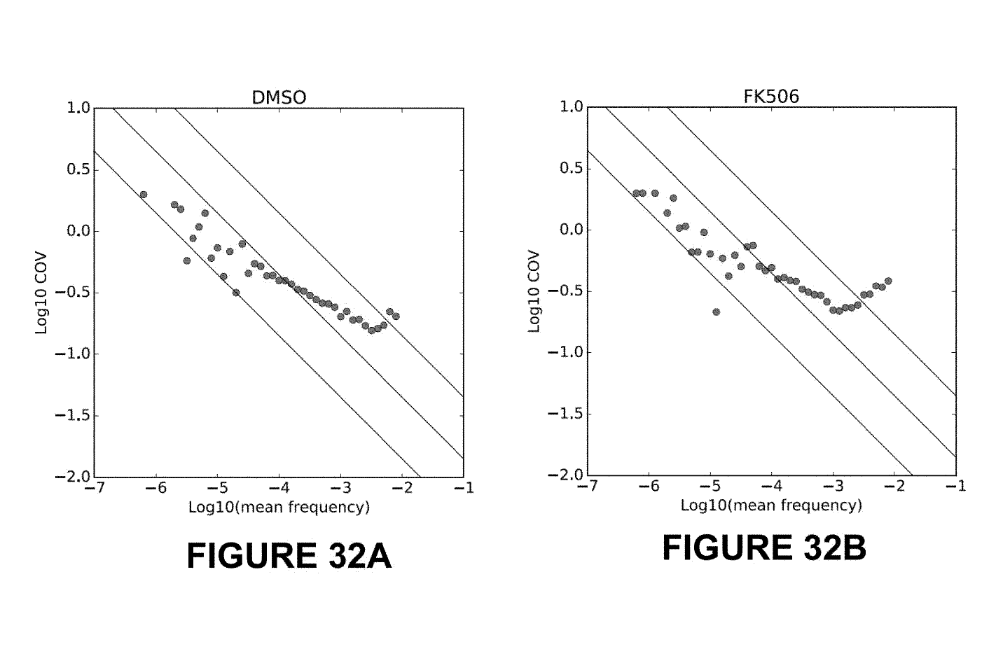

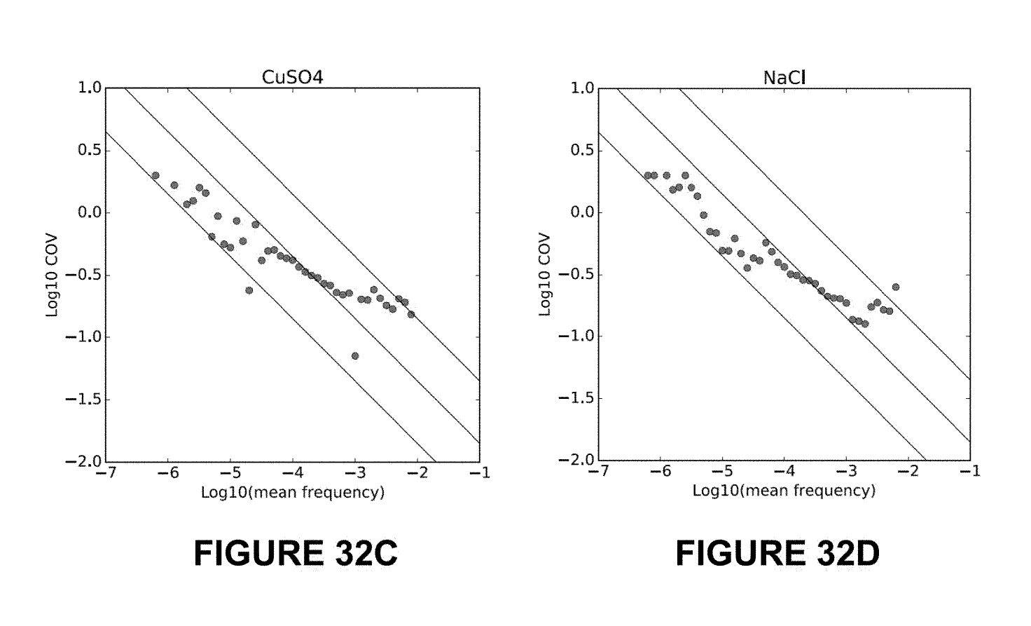

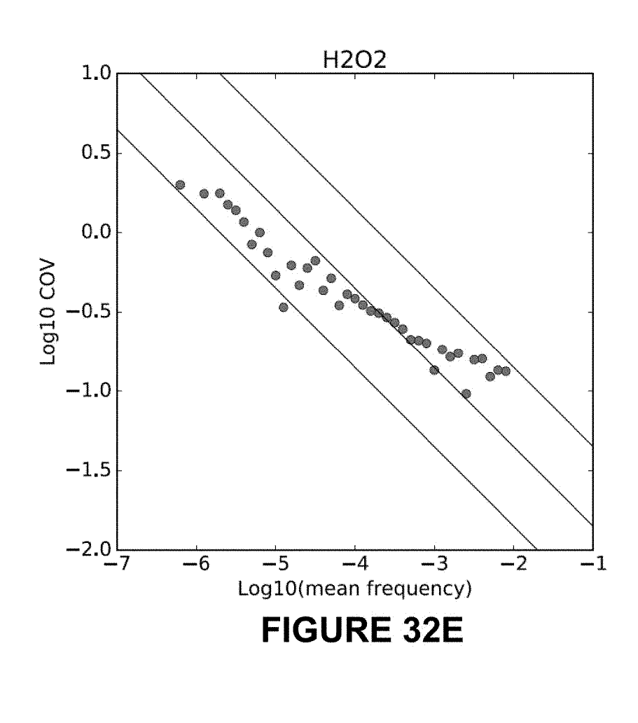

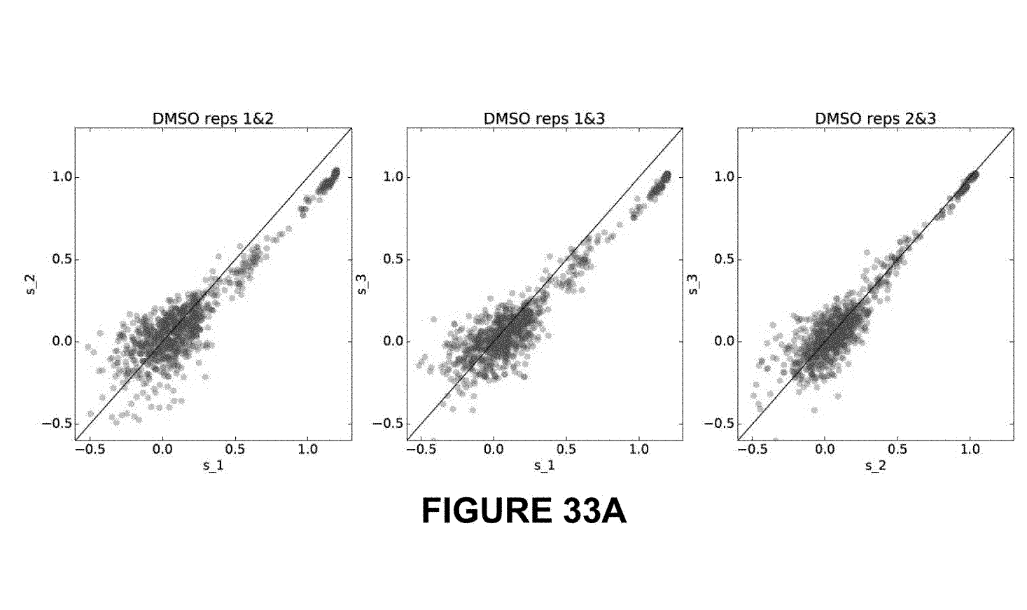

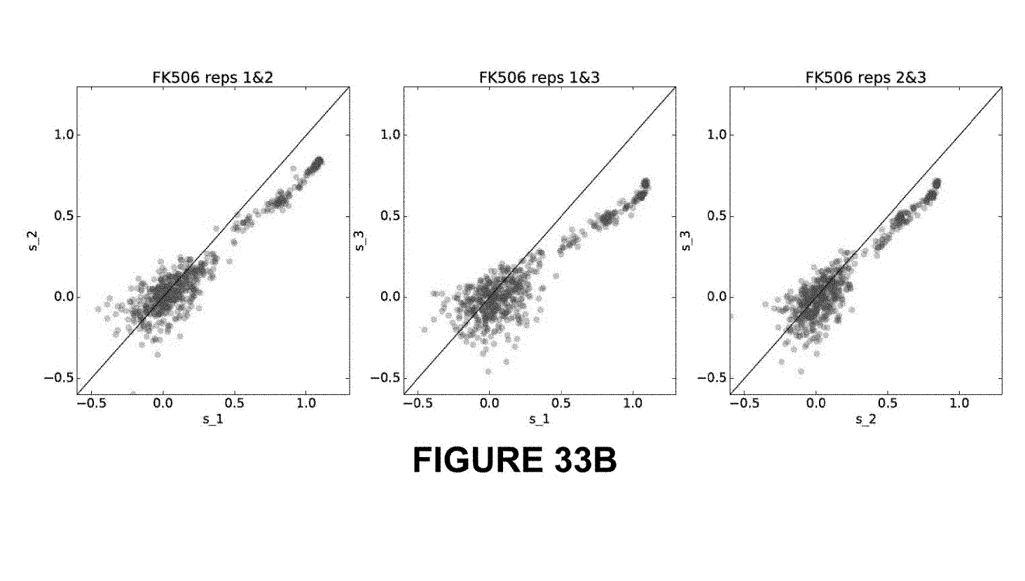

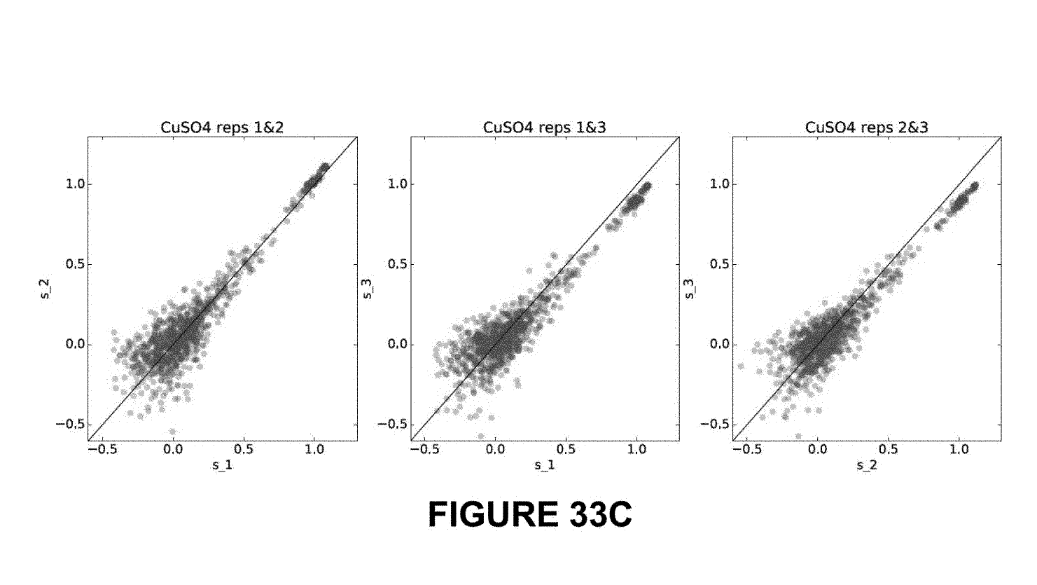

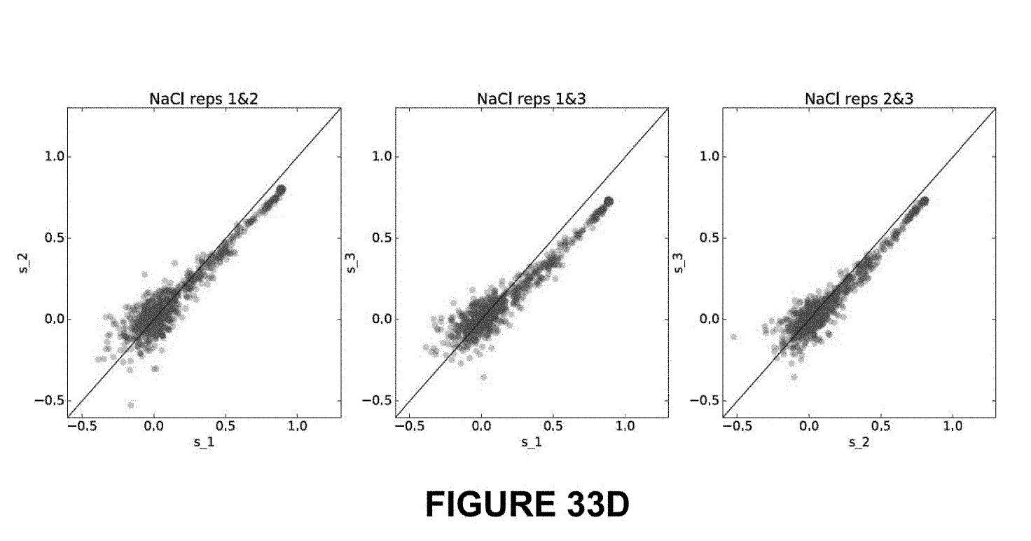

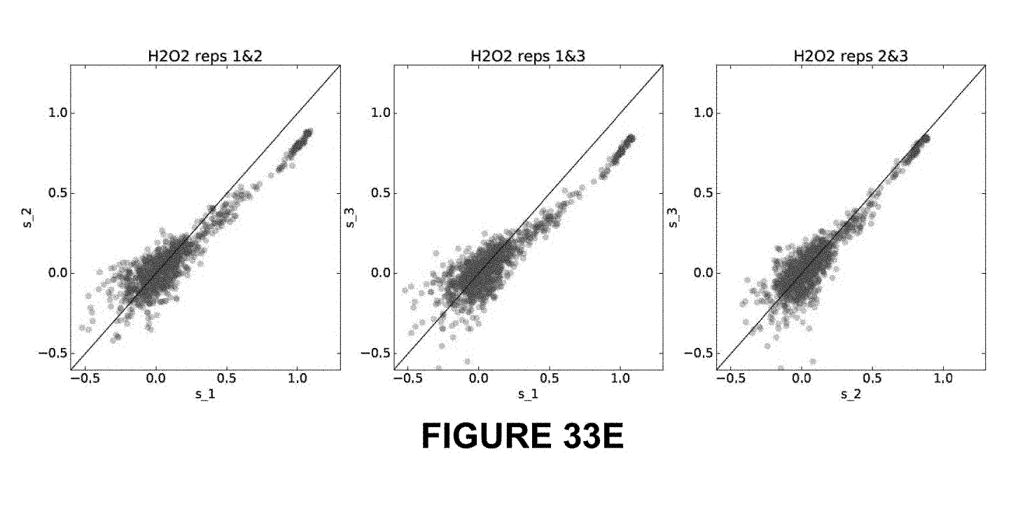

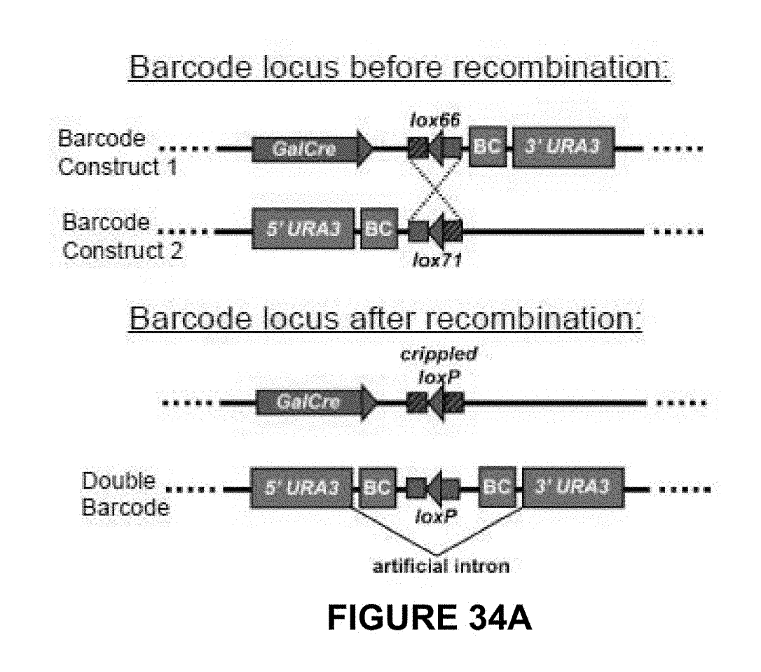

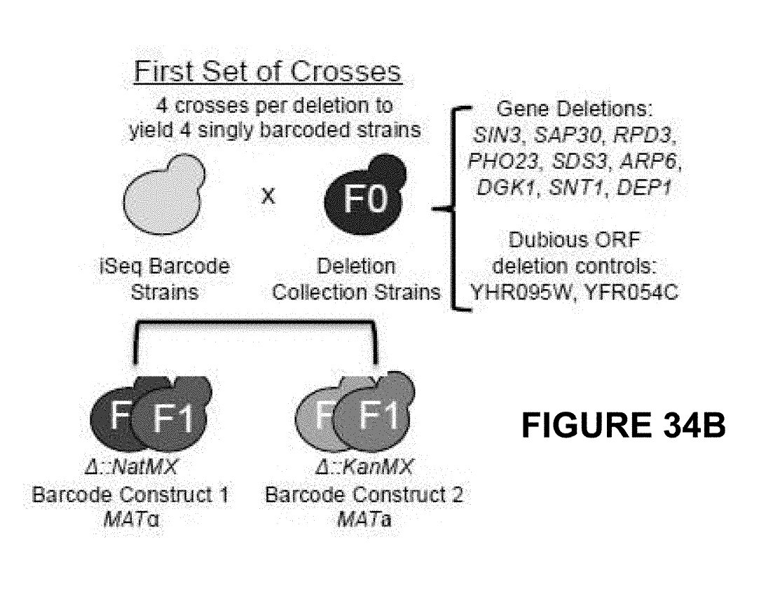

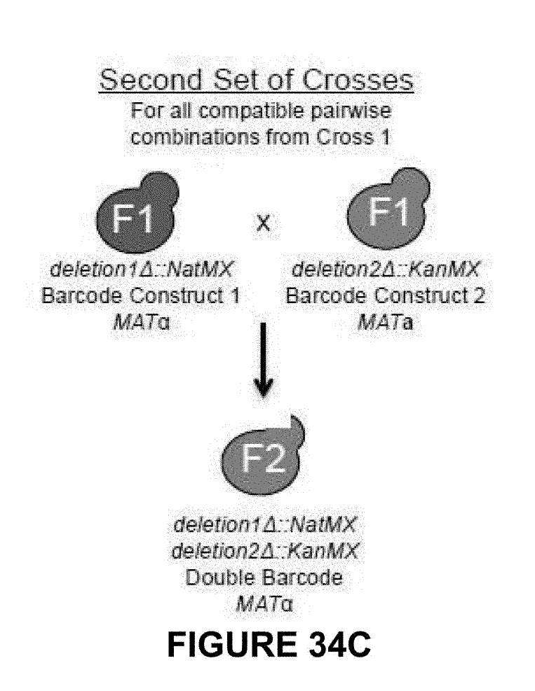

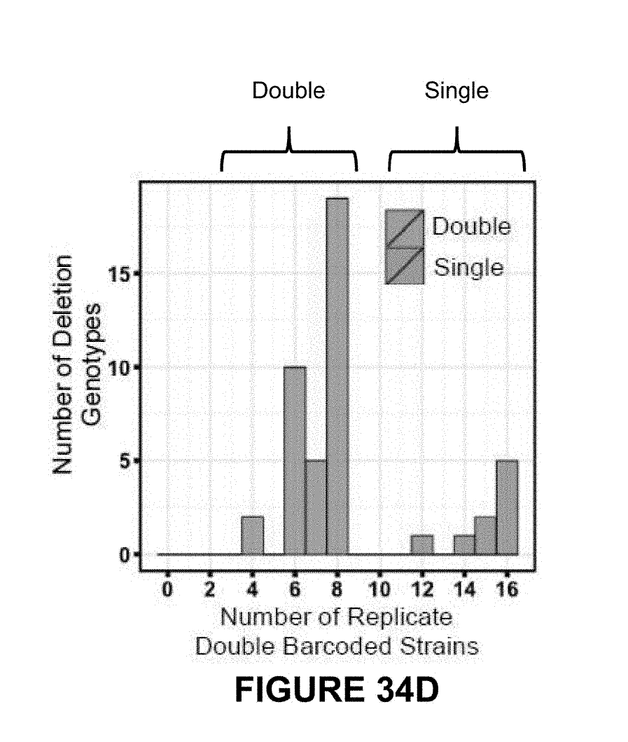

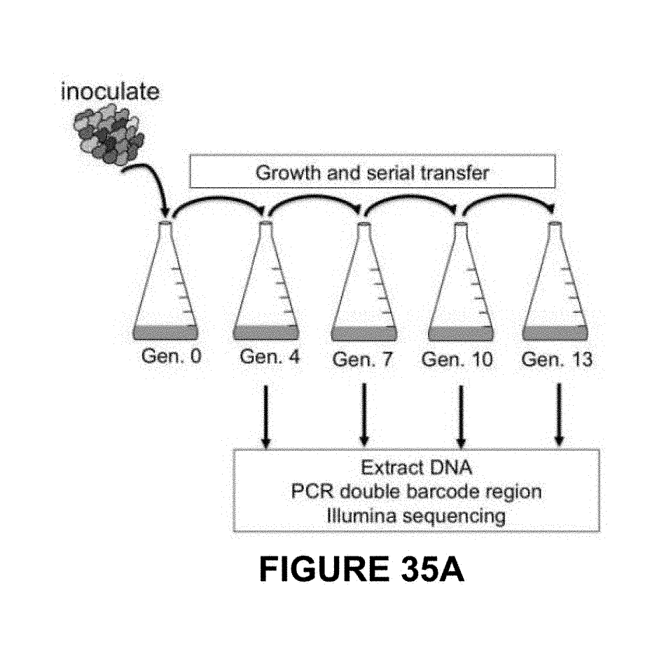

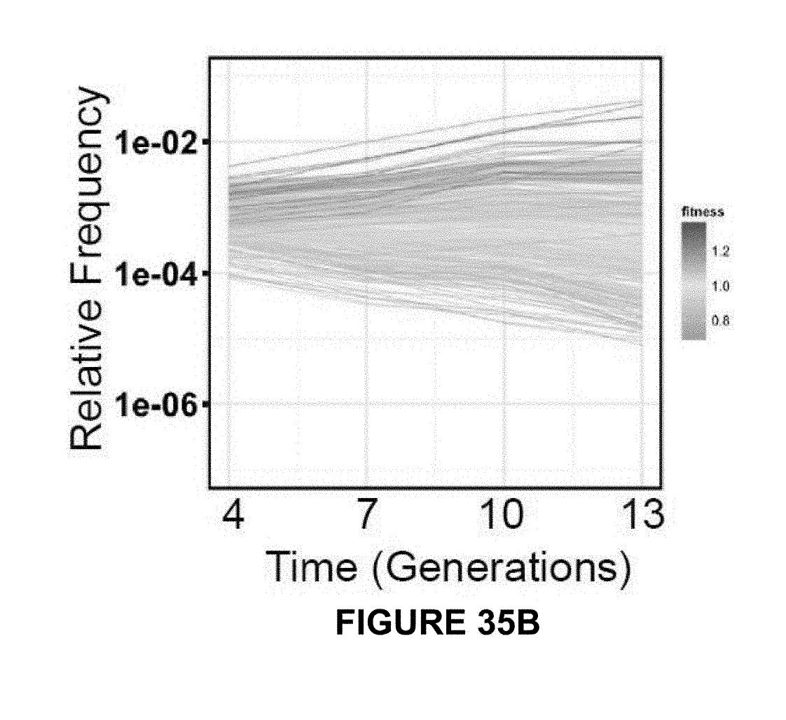

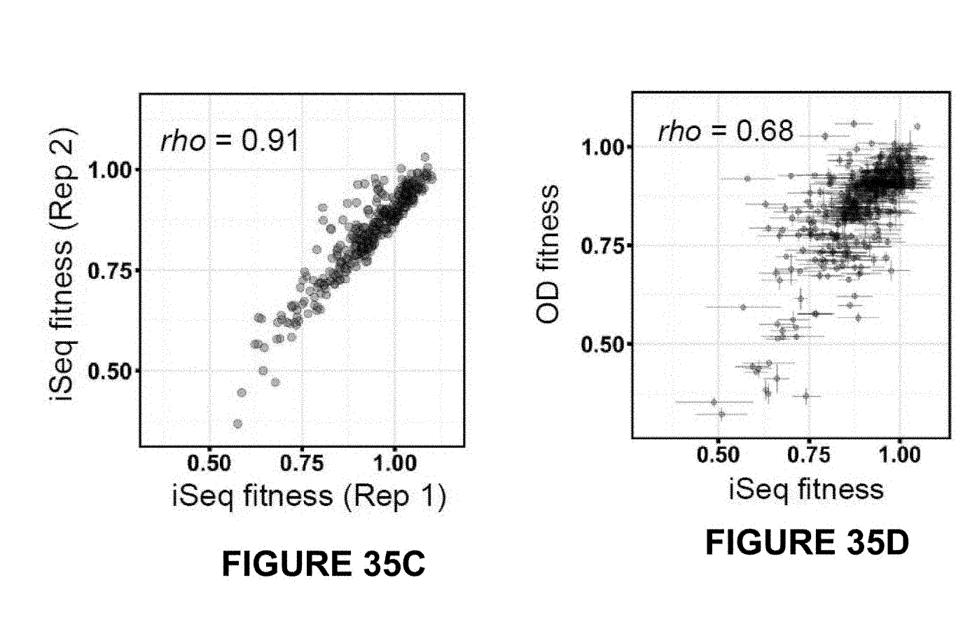

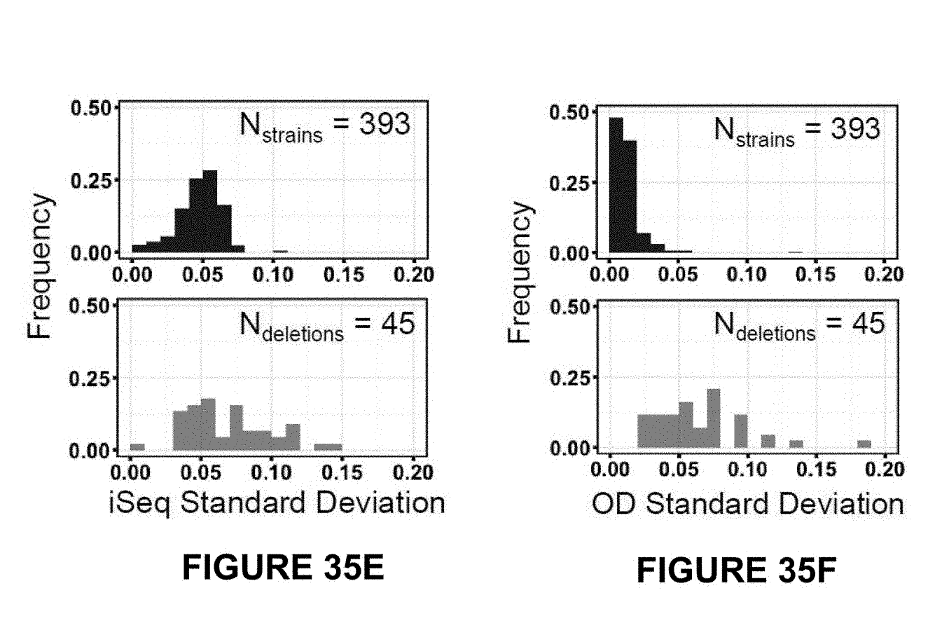

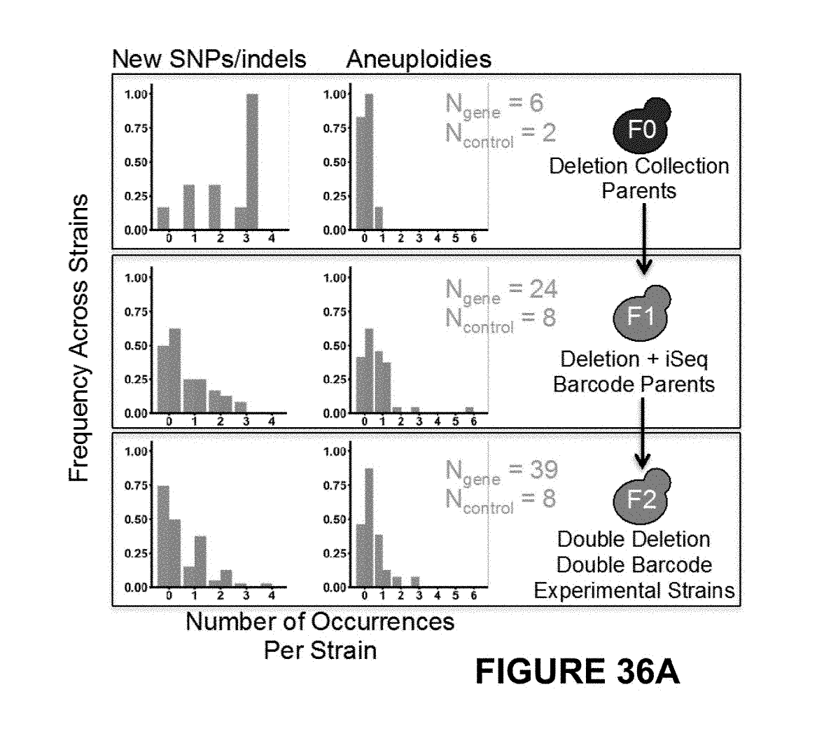

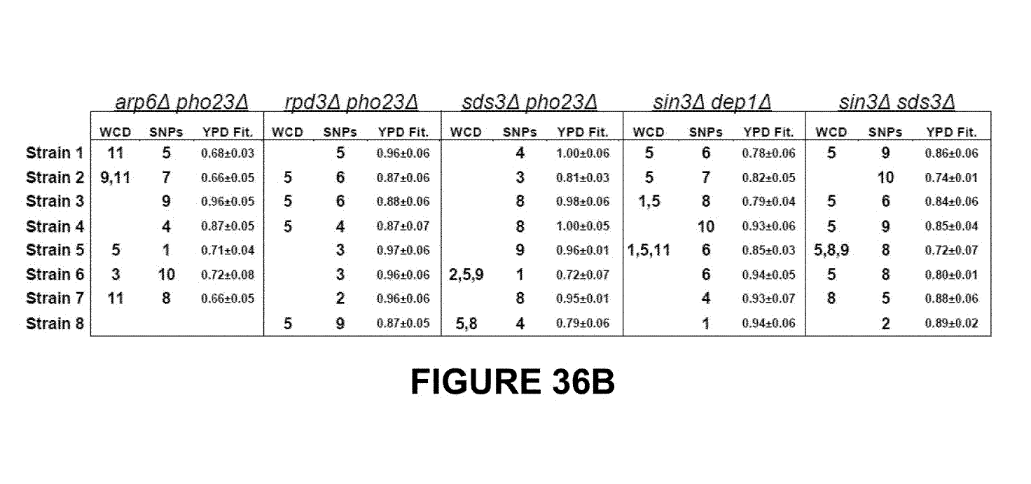

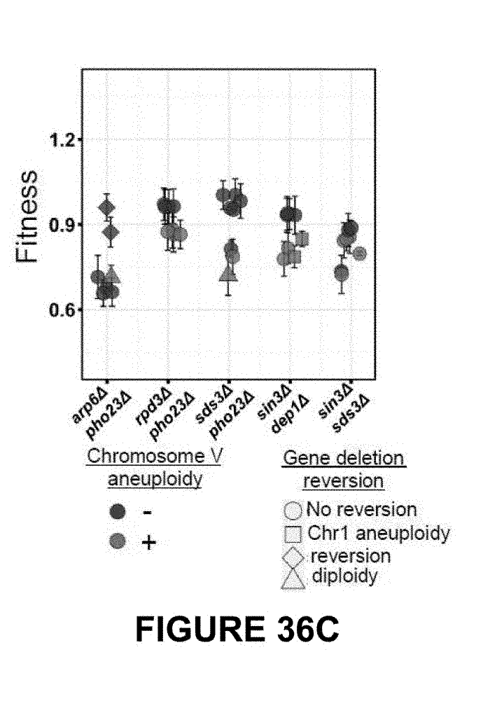

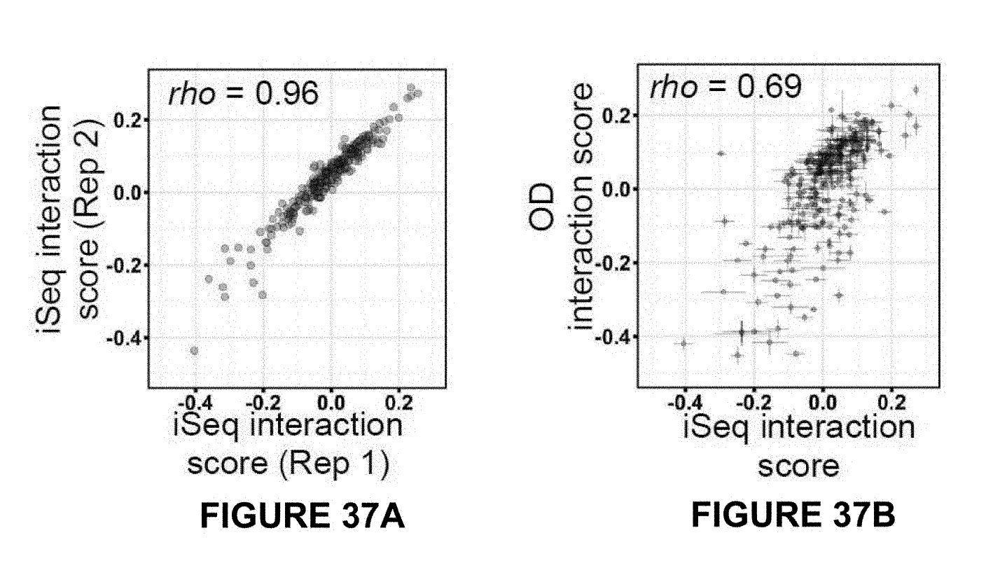

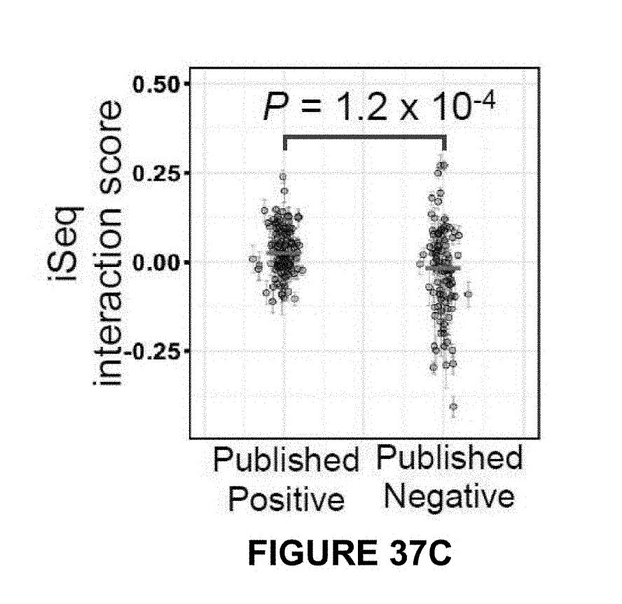

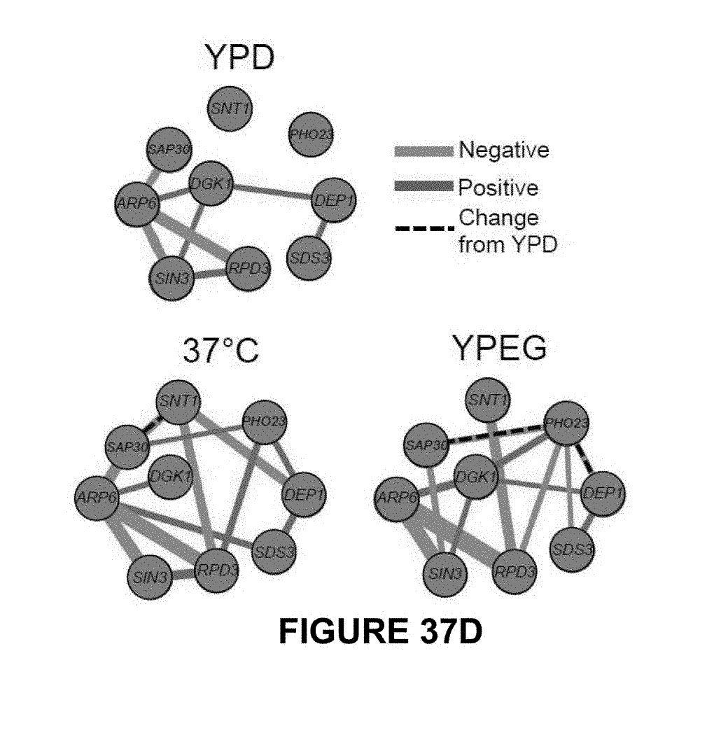

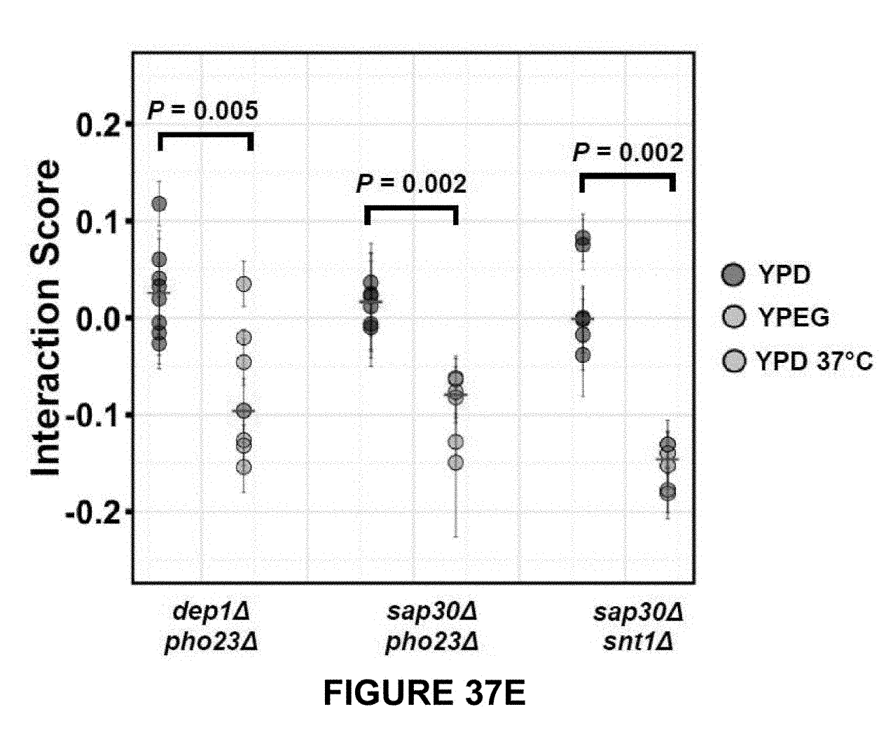

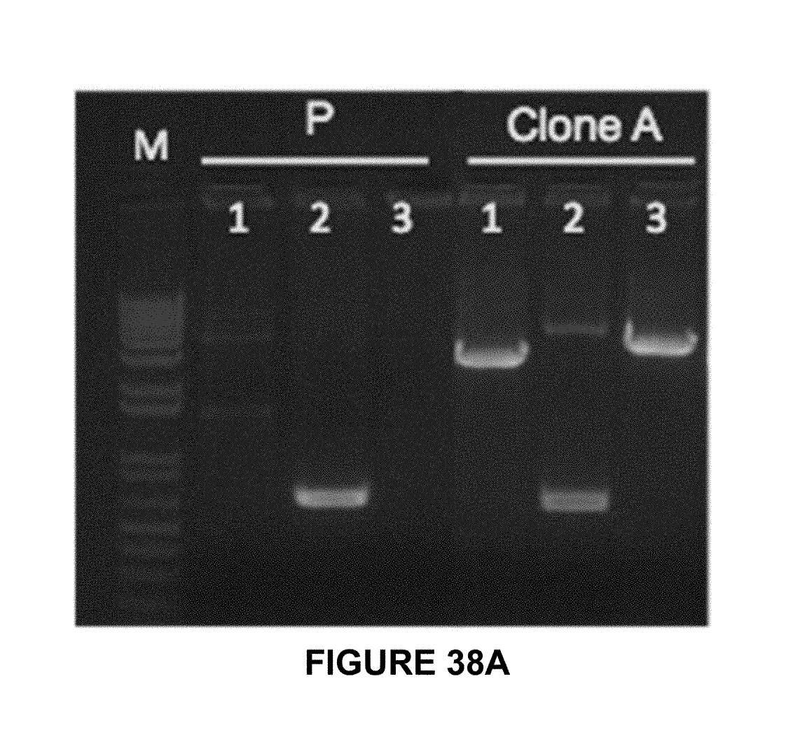

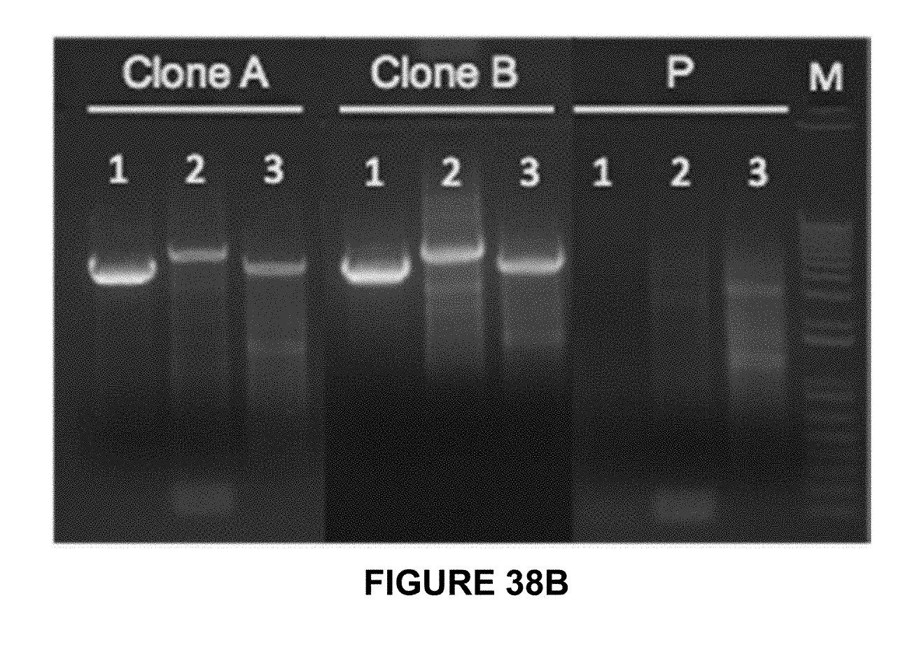

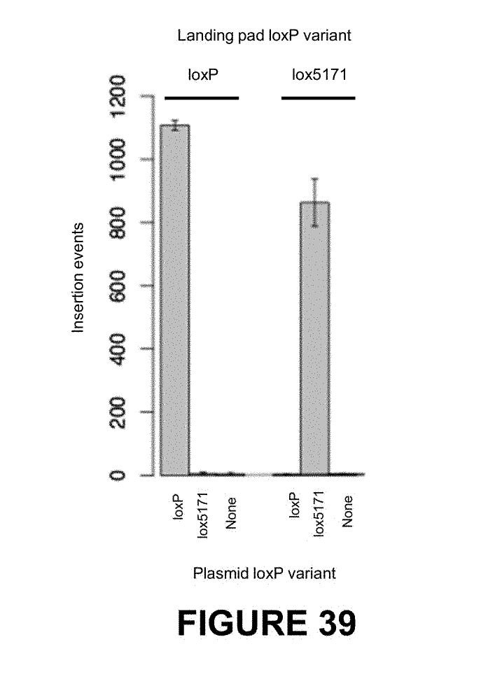

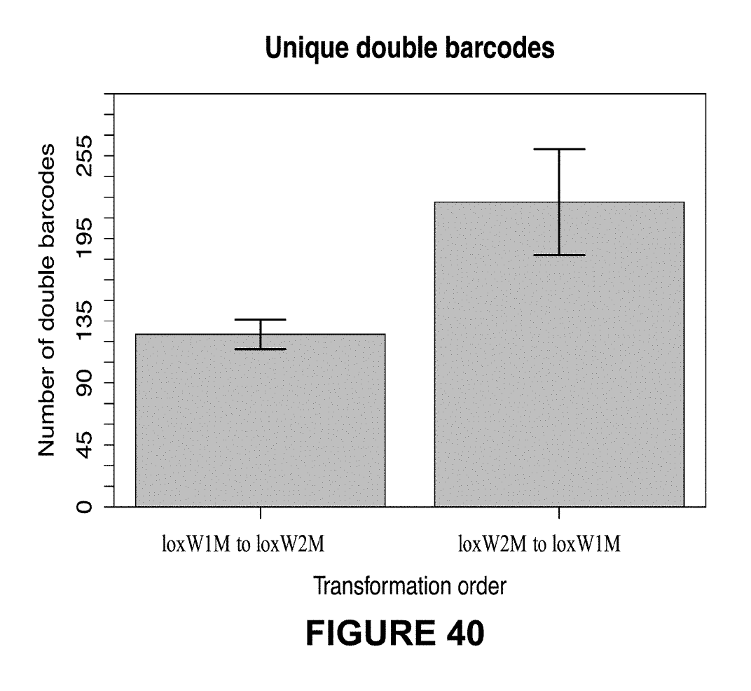

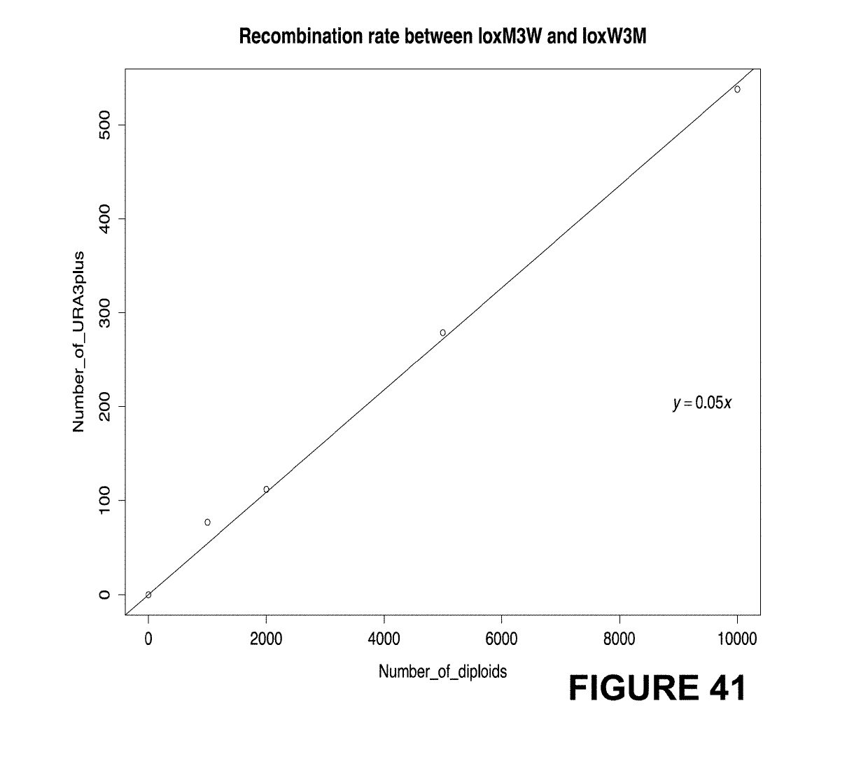

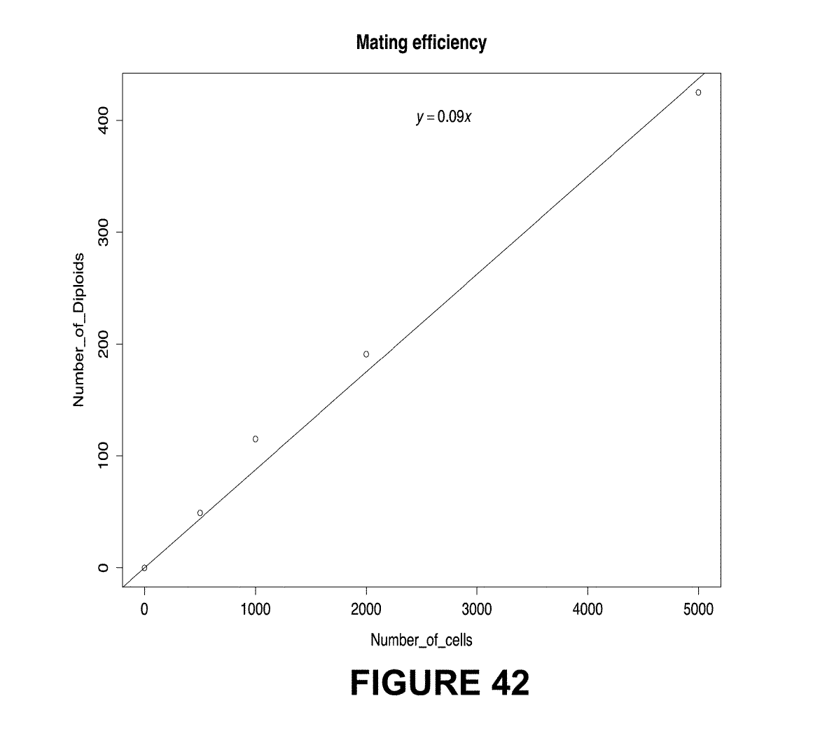

A pooled growth and bar-seq assay was developed that is capable of robustly measuring the relative fitness of all strains in the pool. We expected that as low fitness PPiSeq strains drop out of the population, the frequency trajectories of a higher fitness strain will begin to “bend” as its competition gets tougher (green lines, FIG. 19B). The dynamics of this competition depends on the abundances and relative fitnesses of all strains in the pool, and will therefore change if the composition of the pool changes. Because of this, barcode frequencies at a single time point do not provide a constant measure of fitness across conditions. We therefore monitored relative barcode frequencies over several early time points. We grew the PPiSeq pool in triplicate in standard yeast media and the presence or absence of a low concentration of MTX for ˜12 generations in serial batch culture, diluting 1:8 every 24 hours (˜3 generations, FIG. 23). To allow for fitness measurements of all strains, we chose a low concentration of MTX (0.5 μg/ml, 200-fold lower concentration than traditional PCA) where even strains lacking mDHFR will grow slowly. Double barcodes were sequenced at each dilution (every 3 generations). Reads representing putative PCR chimeras, double barcodes where each double barcode half stems from a different template, occurred at a low but predictable frequency (0.2%, FIGS. 29A-29B) and could confound our results. The expected number of PCR chimeras was subtracted from each double barcode count and generated lineage trajectories with these corrected counts (FIGS. 19A-19B). In the absence of MTX, most PPiSeq strains do not change in frequency over time. However, in the presence of MTX, most strains are driven close to extinction by 12 generations, while others with higher fitness rise in frequency or have a slower decline. Higher fitness indicates that protein-mDHFR-fragment pairs within that strain interact to generate complete and functional mDHFR reporter proteins that, in turn, allow the strain to grow faster in the presence of low amounts of MTX.

To robustly calculate the fitness of each trajectory, a maximum likelihood strategy was used (see below for detailed explanation of Fitness estimation by lineage tracking). Briefly, we make a first fitness estimate of each strain using a simple log-linear regression over the early time points. Based on these fitnesses and the initial relative frequencies of each double barcode, we estimate the expected trajectory of each double barcode and compare this to the measured trajectory under a noise model that accounts for experimental errors (Levy: 2015). We next make small changes to our fitness estimates, repeat this comparison, accept updated fitness estimates if they better fit the data (higher likelihood), and perform this procedure iteratively until fitness estimates are stable (maximized likelihood). To make fitnesses comparable between replicates, or across different barcode pools or environments, we define a strain's fitness relative to the control strain that lacks any mDHFR fragments, whose fitness is set to zero. We find that this procedure performs extremely well on simulated data with parameters similar to our pooled growth experiments (Pearson's r=0.996), and across replicate growth experiments (Pearson's r>0.91 between all MTX(+) replicates). Fitness estimates are generally more accurate for higher fitness strains (those putatively identifying a PPI) because these trajectories are unlikely to fall to low frequencies where counting noise of sequencing reads will be high.

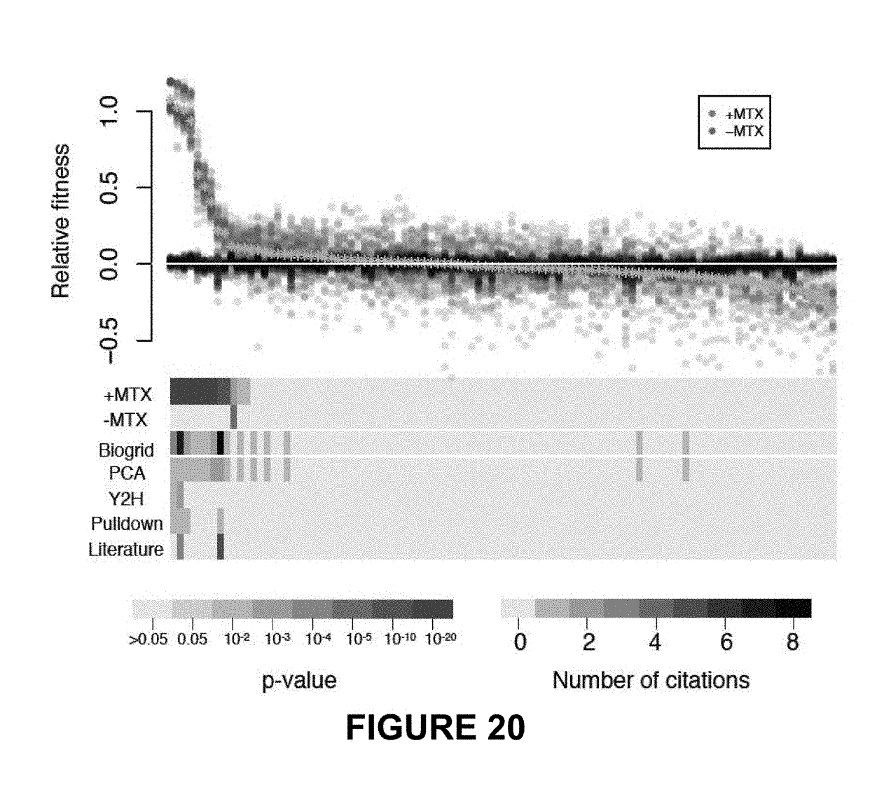

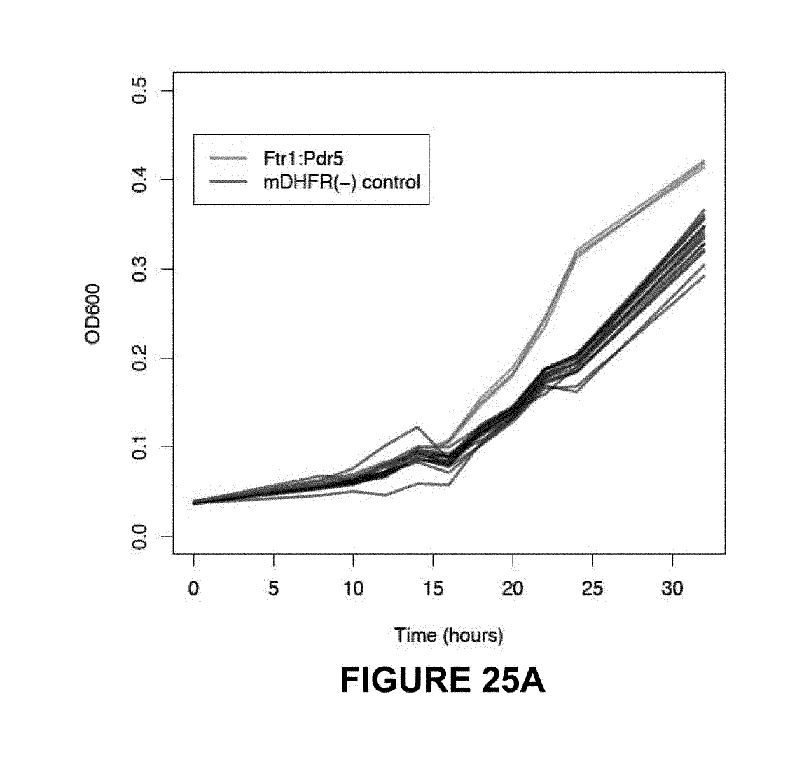

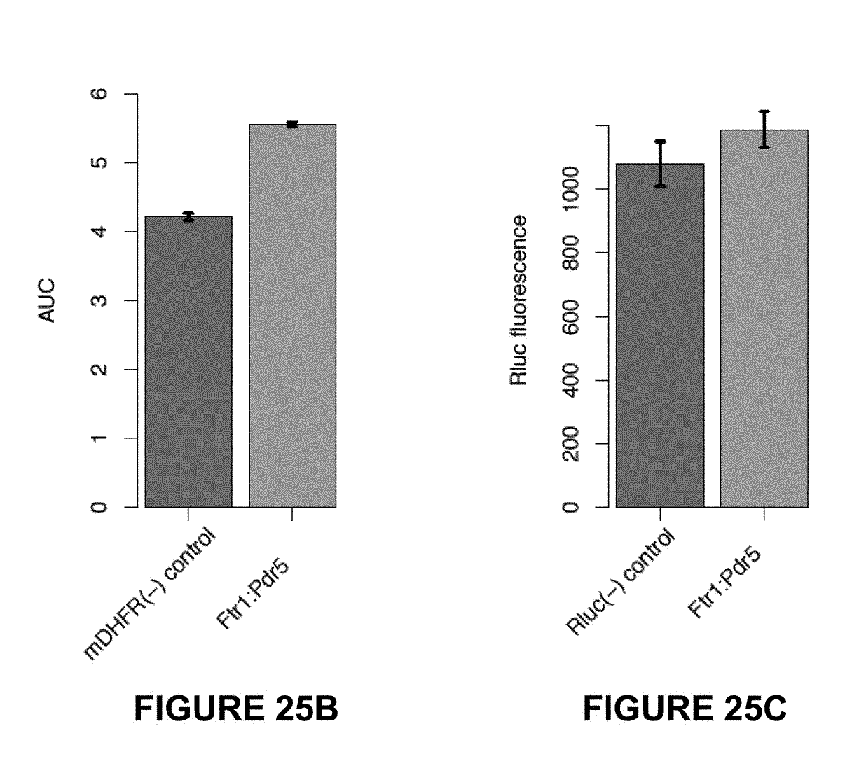

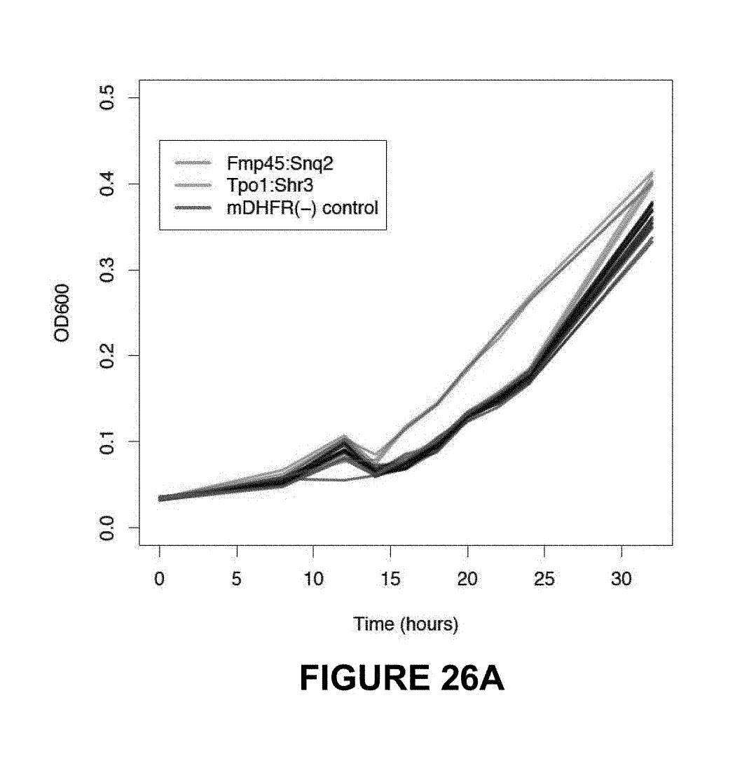

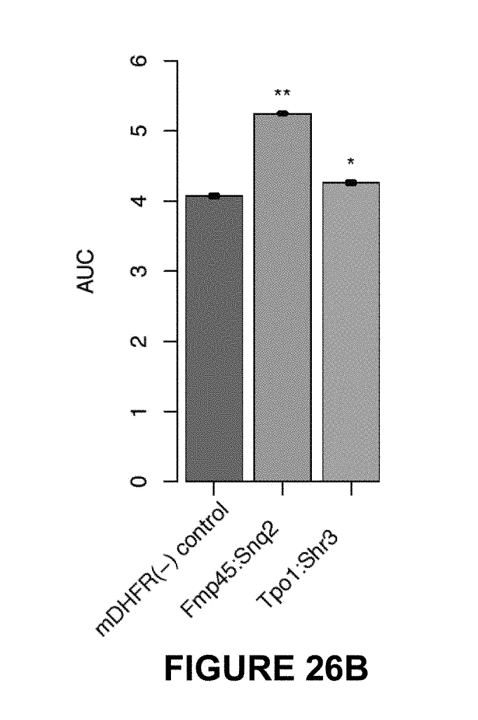

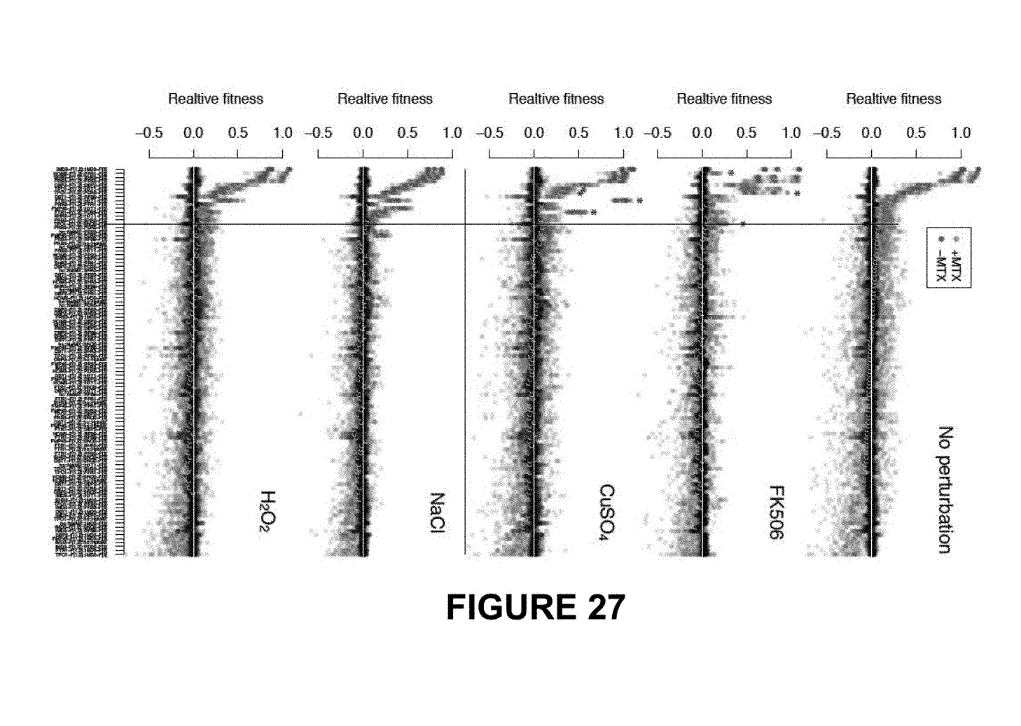

The fitness for each PPI across all ˜75 replicate estimates (˜25 double barcodes per PPI, 3 replicate growth experiments) was compared in the presence or absence of MTX (FIG. 27). Standard errors on fitness are low (typically, SEM<0.05 in MTX(+), with higher fitness PPIs having the lowest errors (SEM<0.02 in MTX(+) for PPIs with fitness >0.07). The fitness values of each PPI was compared against the fitness values of the control strains lacking mDHFR in both MTX(+) and MTX(−) conditions (FIG. 20). As expected in MTX(−), almost none of the strains differ significantly in fitness from the control. The single exception is Prs3-F[1,2]:Fpr1-F[3], which displayed a small but highly significant fitness advantage (fitness=0.04, p-value<2×10⁻⁶, Bonferroni corrected one-sided Student's t-test) that is perhaps due to an adaptive mutation that occurred in the parental PCA strain prior to barcoding. After removing the Prs3:Fpr1 strain from consideration, 11 significant PPIs in MTX(+) were found, 10 that have been previously identified, and one that is new, Ftr1:Pdr5 (fitness=0.10, p-value<0.002). Ftr1:Pdr5 was validated by two additional assays. First, we tracked the optical density (OD600) of Ftr1:Pdr5 PPiSeq strains and the mDHFR(−) control strains grown in isolation in MTX(+) media and found that Ftr1:Pdr5 strains rise in optical density faster (p-value<2×10⁻¹¹, Student's t-test, FIG. 25). Second, we performed a less sensitive split Renilla luciferase (Rluc) PCA assay and found that Ftr1:Pdr5 has a consistently higher (but not significant) fluorescence when compared to control cells (p-value=0.24, Student's t-test). As discussed below, the Rluc PCA assay finds a significant Ftr1:Pdr5 interaction in an alternative environment (p-value<0.02 in 200 μM copper sulfate, Student's t-test), strongly suggesting that our finding in this benign environment is not a false positive.

Our PPiSeq assay missed five putative PPIs that had been discovered by traditional PCA. Three (Shr3:Hxt1, Tpo1:Snq2, and Fmp45:Pdr5) showed elevated but not significant fitness increases in MTX(+) (0.10, 0.08, and 0.06, respectively). As discussed below, PPiSeq does find all of these interactions to be significant in at least one perturbation environment, suggesting that these PPIs are sensitive to the environment and that environmental differences between PPiSeq and traditional PCA may impact their detection. The remaining two PPIs (Fmp45:Snq2 and Tpo1:Shr3) could not be detected by PPiSeq in any environment, but could be validated as being PPIs using isolated growth and optical density tracking over 32 hours of growth. Notably, differences in optical density between Tpo1:Shr3 and control strains only began to appear around 25 hours of growth, likely caused by a change in Tpo1 localization following the diauxic shift, suggesting that our current 24 hour growth-bottleneck regime is not sensitive to PPIs that are specific to this later growth phase and that longer growth-bottleneck cycles may capture additional PPIs.

Overall, the ability of PPiSeq to detect PPIs appears to be on par with existing PPI assays; in this test set, PPiSeq discovered 10 PPIs that have been described by other assays, 1 new PPI validated here, 0 false positives, and 5 false negatives. When considering other environments, PPiSeq accuracy improves to 14 PPIs discovered and only 2 false negatives. However, in contrast with previous high-throughput assays, detected PPIs span a reproducible range of positive fitnesses. Growth rate of PCA strains in MTX has previously been found to correlate with the number of functional mDHFR molecules per cell, suggesting that fitness differences in our assay are founded in differences in the abundance, localization, or binding of the interacting proteins.

### PPiSeq Detects Dynamic PPIs

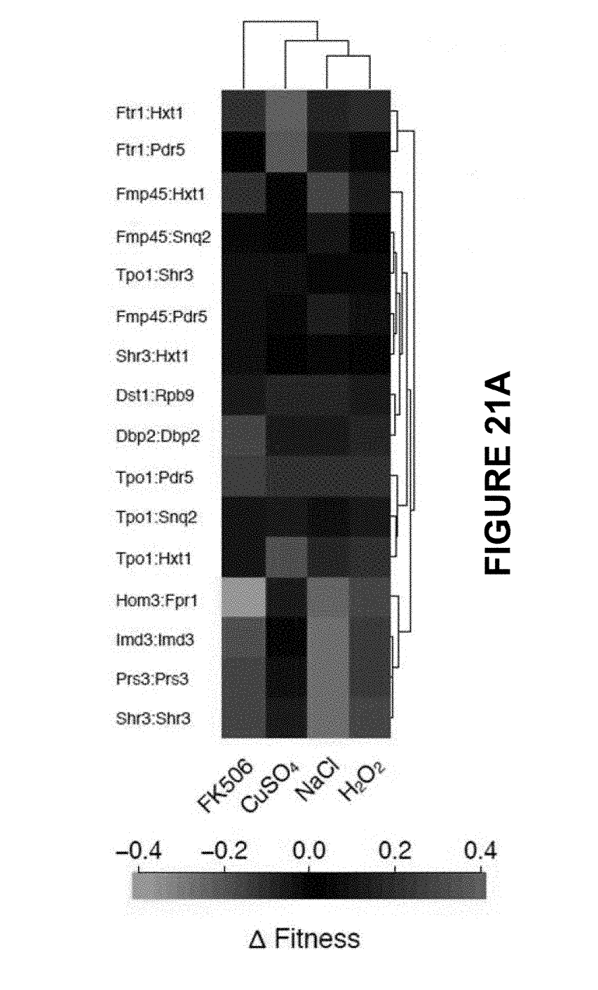

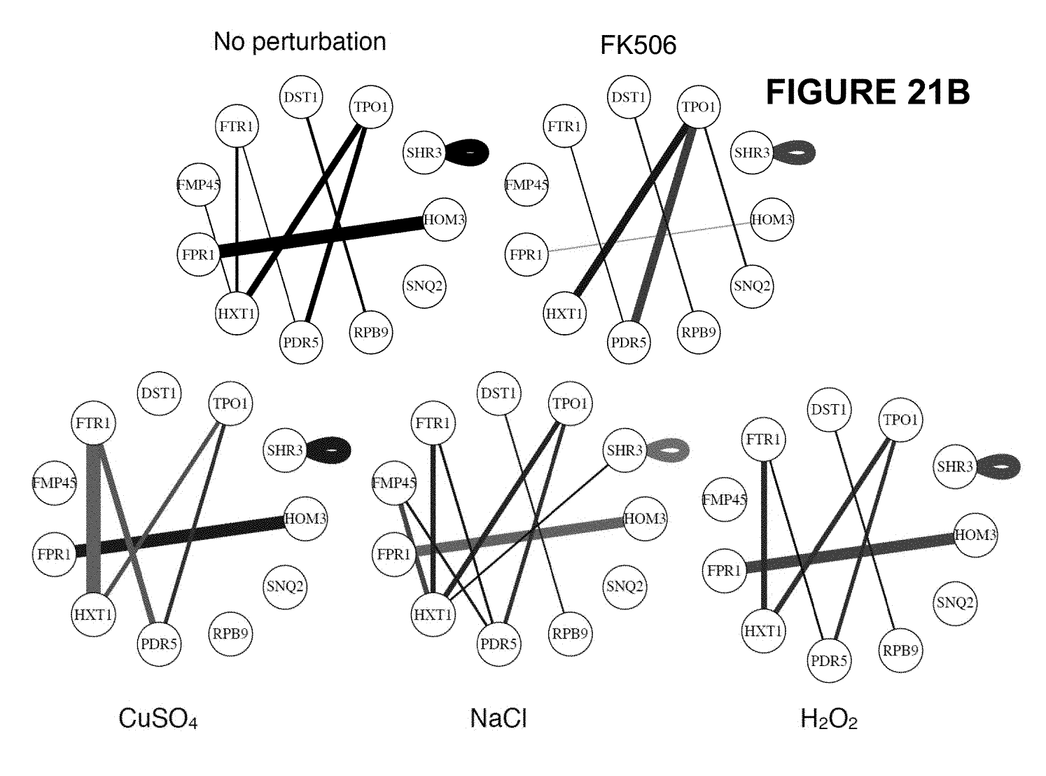

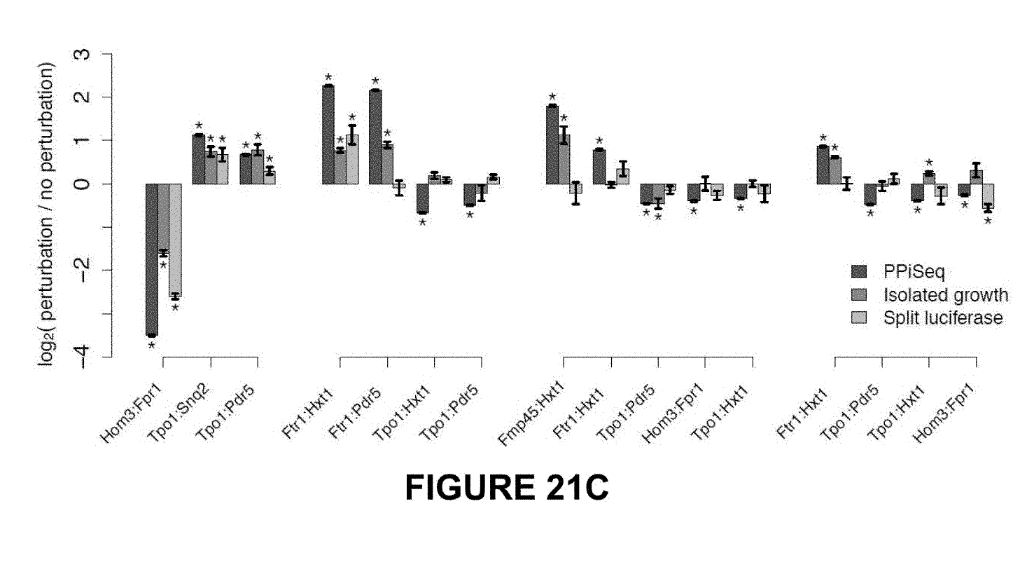

One advantage of using a pooled growth and bar-seq approach for detecting PPIs is that, once a barcoded PCA pool is constructed, it is trivial to re-test the entire interaction space across perturbations in order to detect PPIs that are dynamic. Here, we grew the pool of 2500 PPiSeq strains in triplicate in MTX(−) and MTX(+) media supplemented with one of four additional perturbagens: 0.001% hydrogen peroxide (oxidative stress), 175 mM sodium chloride (high salt), 200 μM copper sulfate (high copper), and 50 μM of FK506, an inhibitor of calcineurin function in yeast. The fitness of each strain was calculated in each environment relative to the mDHFR(−) control strain using the maximum likelihood strategy described above. As expected, major fitness differences between strains within each MTX(+) environment were found, but not within the MTX(−) environments (FIG. 21A-21C). Surprisingly, 86% of detected PPIs significantly changed in fitness in a least one perturbation relative to the benign environment (12 of 14, p<0.05, Bonferroni corrected Student's t-test) and 50% were undetectable by our assay in at least one environment (7 of 14, p>0.05, Bonferroni corrected one-sided Student's t-test). To validate these changes, 16 PPI-environment combinations were selected where fitness was significantly different from the benign environment, and assayed each by both optical density tracking and Rluc PCA (FIG. 21C). 9 of 16 dynamic PPIs could be validated by at least one method.

A number of factors appear to underlie PPI changes across environments. One expected change is the interaction between the aspartate kinase Hom3 and the peptidyl-prolyl cis-trans isomerase Fpr1 in FK506, which has been previously found to physically disrupt this interaction. Our assay does still detect the Hom3:Fpr1 PPI in FK506, however fitness is diminished ˜10-fold (p<10⁻⁵⁹). Other dynamic PPIs appear to be due, at least in part, to changes in protein expression. For example, FK506 has been shown to result in increased expression of the polyamine transporter TPO1, and the multidrug transporters SNQ2 and PDR5, and, in agreement with previous findings, we find higher fitnesses in FK506 for both the Tpo1:Pdr5 and Tpo1:Snq2 PPIs (p<10⁻¹⁶ and p<0.01, respectively). Second, high copper has been found to result in increased expression of the iron permease FTR1, and we find higher fitnesses for interactions between Ftr1 and both the glucose transporter Hxt1 (p<10⁻¹⁸) and the multidrug transporter Pdr5 (p<0.05). Third, high salt has been found to increase expression of the glucose transporter HXT1, and we find a higher fitness for the interaction between Hxt1 and the integral membrane protein Fmp45 (p<10⁻²⁴). Still other dynamic PPIs may be due to changes in protein localization. For example, both TPO1 and PDR5 increase in mRNA expression in high salt (4.7- and 2.7-fold, respectively), yet the fitness of the Tpo1:Pdr5 PPiSeq strain decreases (p<10⁻¹¹). This contradiction appears to be resolved by the finding that Pdr5, but not Tpo1, becomes depleted from the plasma membrane in high salt.

### PPiSeq is Scalable

At least 500,000 uniquely barcoded strains can be tracked in parallel in a single cell pool. Furthermore, we found that for the majority of barcodes, errors in frequencies are consistent with counting noise stemming from finite read depths, rather than some other factor in the experimental protocol (See below for Analysis of errors). Given exponentially declining sequencing costs, it is therefore possible that several million double barcodes could be assayed in parallel. In order for our PPiSeq platform to reach these scales, two criteria must be met. First, PPiSeq must be capable of generating a large number of double barcode strains by pooled mating. Although it is technically possible to probe extremely large interaction spaces by pairwise mating in ordered arrays, the cost and time required to do so is high, and this requirement would greatly reduce the flexibility and scalability of the platform. Second, the distribution of initial double barcode frequencies must be of a form that allows the fitness of most strains in the pool to be measured at reasonable sequencing depths. A distribution where many double barcodes are missing or are present at low frequencies would result in a large fraction of uncharacterized interactions.

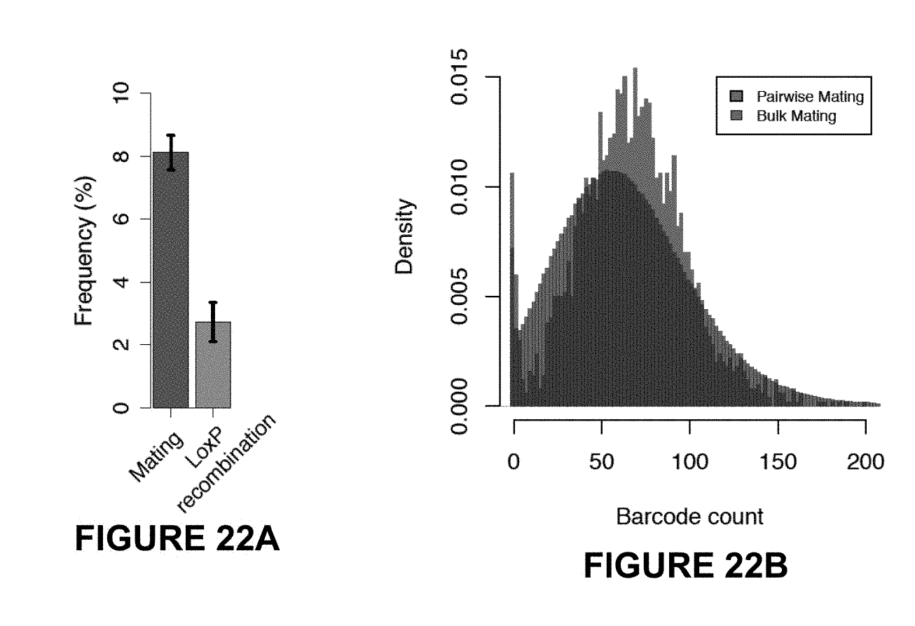

To test how many unique double barcodes could be realistically generated by pooled mating, we developed a protocol that mates ˜10¹⁰ haploids on a standard agar plate, and then selects for diploid double barcode recombinants (See Methods section below). Based on experimental tests, we estimated the lower bounds of the frequency of mating (8%) and loxP recombination (2%) of this protocol, and predicted that at least 2×10⁷ (i.e. 10¹⁰×8.1%×2.7%) unique double barcoded diploids are generated per plate (FIG. 22A and Mating and loxP recombination efficient estimates below). Based on this performance, we estimate that double barcode library sizes exceeding 10⁹ could be achieved by a single investigator (˜50 mating plates).

We next compared the initial double barcode frequency distribution of a large bulk mating (˜1 million double barcodes possible across 5 mating plates) to the smaller pairwise mating we used to generate the PPiSeq strains above (2500 double barcodes possible) and found that the two protocols resulted in similar barcode frequency distributions (FIG. 22B). At an average sequencing depth of ˜67 reads/barcode (See comparison between bulk and pairwise mating below), bulk and pairwise mating protocols detect a similar number of double barcodes at low and moderate frequencies (>98% at >1 read, >95% at >10 reads), suggesting that even moderate read depths will be sufficient to characterize most double barcodes in the pool.

### DISCUSSION

We describe a highly parallel Protein-Protein interaction Sequencing (PPiSeq) assay that is sensitive, accurate, and graded. Importantly, PPiSeq provides a quantitative score (fitness) for each PPI that is robust to changes in the environment or pool constituents. Furthermore, both library construction and fitness assays are performed in large cell pools, making the platform highly scalable. PPiSeq is therefore a powerful new platform for protein-interactome-scale investigations of dynamic PPIs.

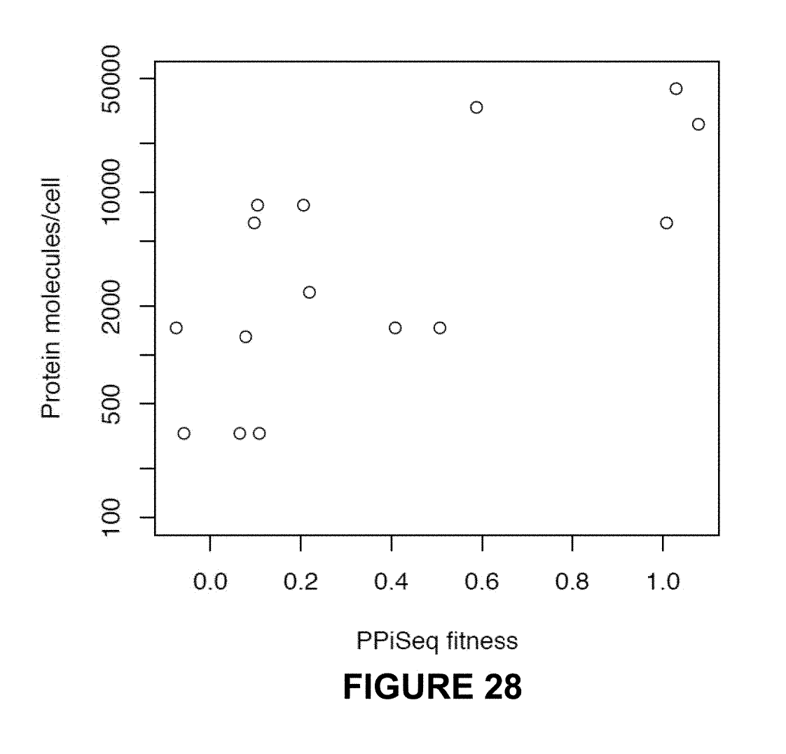

The growth of each PCA strain is known to correlate with the number of reconstituted mDHFR reporter proteins per cell, which, in turn could be influenced by several factors including the abundance of each interacting protein, the binding affinity, and the extent of co-localization of each binding pair. Protein abundances appear to have a large influence on fitness. For the 16 PPIs in our test set, fitness correlates reasonably well with the abundance of the least abundant interaction partner (FIG. 28, Spearman's rho=0.68). Additionally, many of the changes in fitness across environments that we detect co-vary with changes in mRNA expression of one or both interacting partners that are reported in the literature. However, other factors are likely to be important as well. For example, a recent proteome-wide screen found that nearly as many proteins change in localization as change in abundance when cells are exposed to hydroxyurea. In our test set, we find one example where a change in localization appears to be driving a PPI change (Tpo1:Pdr5). However, we do caution that these interpretations are made with previously published data that may contain important differences in experimental conditions. An unbiased and systematic characterization of the factors underlying the dynamic protein interactome will therefore require combining PPiSeq with genome-scale mRNA abundance, protein abundance, and protein localization studies under the same conditions.

For cells treated with FK506, PPiSeq not only detects a change in the PPI target of the drug, Hom3:Fpr1, but also changes in other PPIs such as Tpo1:Snq2 and Tpo1:Pdr5. In this case, additional changes appear to be caused by a specific cellular response to the drug, as each of these proteins are efflux transporters. However, dynamic PPIs that are a response to global changes in the cell physiology or that are due to off-target binding of a drug may also be likely. Avoiding off-target effects, as well as a systems level understanding of a drug's effect on the cell, are often the primary concerns of drug development. Because of the ease by which large numbers of PPIs can be quantitatively screened across many perturbations in relatively small volumes of media, PPiSeq therefore provides a powerful new tool for high-throughput drug screening.

More generally, iSeq provides a new framework for performing large-scale interaction screens. Because strain construction and scoring can be performed in cell pools, instead of one-by-one, a major throughput limitation to interaction screens has been removed. Furthermore, iSeq can be used to investigate combinations of any two genetic elements, such a gene knockouts or engineered constructs, and will therefore have broad utility beyond PPI screens.

### Methods

### Construction of Plasmid Backbones.

pBAR1 (SEQ ID NO:108), pBAR4 (SEQ ID NO:26) and pBAR5 (SEQ ID NO:27) were cloned from the following sources (all available from EUROSCARF) by standard methods: 1) plasmid backbone/bacterial origin from pAG32, 2) kanMX from pUG6, 3) Gal-Cre from pSH63, 4) URA3 from pSH47, 5) artificial intron, random barcodes and loxP sites were synthesized de novo (IDT).

### Construction of Plasmid Random Barcode Libraries.

Random barcodes were inserted into pBAR4 (SEQ ID NO:26) and pBAR5 (SEQ ID NO:27) by ligation. Primers containing a KpnI restriction site, a random 20 nucleotides, lox71 or lox66 sites, and a region of homology to the plasmids were ordered from IDT using the “hand mixed” option:

(SEQ ID NO: 31)

P84 (lox66) =
CCAGCTGGTACCNNNNNAANNNNNTTNNNNNTTNNNNNATAACTTCGTAT
AGCATACATTATACGAACGGTA GGCGCGCCGGCCGCAAAT,
and

(SEQ ID NO: 32)

P85 (lox71) =
CCAGCTGGTACCNNNNNAANNNNNTTNNNNNTTNNNNNATAACTTCGTAT
AGCATACATTATACGAACGGTA GGCGCGCCGGCCGCAAAT.

Random sequences were limited to 5 nucleotide stretches to prevent the inadvertent generation of restriction sites. To construct the pBAR4 (SEQ ID NO:26) plasmid library, P85 and P23 (GCCGAAATTGCCAGGATCAGG) (SEQ ID NO:3) primers were used to amplify a portion of pBAR1 (SEQ ID NO:108). Both the PCR product and pBAR4 (SEQ ID NO:26) were cut with KpnI and XhoI restriction sites and ligated together to generate plasmids containing a lox71 site and a random barcode. Ligation products were inserted into DH10B cells (Life Technologies) by electroporation, allowed to recover from electroporation in liquid media for 30 minutes, and plated onto 12 LB-Ampicillin plates at a density of ˜6000 CFU/plate, a total of ˜72,000 colonies. During the recovery period in liquid media, some fraction of the cells could have undergone a cell cycle, meaning that our true library complexity is likely to be less than the number of colonies we observe. Colonies were pooled in 900 ml LB-Ampicillin and a fraction of the pool was used directly for plasmid preps to generate the plasmid library (pBAR4-L1). Similar methods were used with P84 (lox66) and pBAR5 (SEQ ID NO:27) to construct pBAR5-L1, a library containing ˜120,000 barcodes. The final barcoded plasmid libraries are pBAR4_L1 and pBAR5_L1. pBAR4_L1 contains a partially crippled loxP site (lox66), the barcode region, the 3′ end of URA3 gene preceded by part of an artificial intron and the KanMX dominant drug resistant marker. pBAR5_L1 contains a complementary partially crippled loxP site (lox71), the barcode region, the 5′ end of URA3 gene followed by part of an artificial intron, and the KanMX dominant drug resistant marker.

### Construction of Barcode Acceptor Strains.

Barcode acceptor strains are derived from BY4741 (MATa, his3Δ1, leu2Δ0, met15Δ0, ura3Δ0) and BY4742 (MATα, his3Δ1, leu2Δ0, lys2Δ0, ura3Δ0). First, Gal-Cre and NatMX was inserted the YBR209W locus in opposite orientations via homologous recombination. Disruption of YBR209W has no impact on fitness. For the BY4741 insertion, pBAR1 (SEQ ID NO:108) sequence was amplified with the following primers:

(SEQ ID NO: 33)

P102 =
GCTTGCGCTAACTGCGAACAGAGTGCCCTATGAAATAGGGGAATGCGCAC
TTAACTTCGCATCTG,
and

(SEQ ID NO: 34)

P103 =
GTTCTTTGCTTTTTTTCCCCAACGACGTCGAACACATTAGTCCTACATAT
CATACGTAATGCTCAACCTT.

Underlined sequences correspond to sequences flanking the dubious open reading frame, YBR209W. The PCR product, containing Gal-Cre and the NatMX selectable marker, was inserted into the genome by homologous recombination. For BY4742, Gal-Cre-NatMX was placed in the opposite orientation using the following primers:

(SEQ ID NO: 4)

PEV8 =
GTTCTTTGCTTTTTTTCCCCAACGACGTCGAACACATTAGTCCTACGCAC
TTAACTTCGCATCTG,
and

(SEQ ID NO: 5)

PEV9 =
GCTTGCGCTAACTGCGAACAGAGTGCCCTATGAAATAGGGGAATGCATAT
CATACGTAATGCTCAACCTT.

Second, we PCR amplified the dual magic marker (MFapr1-HIS3-MF 1pr-LEU2) from strain UCC8600 10-12, and inserted it at the CAN1 locus in both the BY4741 and BY4742 derivative. The promoters MFa1pr and MF 1pr are only active in MATa and MATα haploids, respectively. Populations of CAN1/can1:: MFApr1-HIS3-MF 1pr-LEU2 diploids can be easily converted to either MATa or MATα haploids by growing on media containing canavanine (for selection against diploids) but lacking histidine or leucine, respectively. Final barcode acceptor strains are SHA345 (MATa, his3Δ1, leu2Δ0, met15Δ0, ura3Δ0 ybr209w::(F)GalCre-NatMX, can1::MFApr1-HIS3-MF 1pr-LEU2) and SHA349 (MATα, his3Δ1, leu2Δ0, lys2Δ0, ura3Δ0, ybr209w::(R)GalCre-NatMX can1::MFApr1-HIS3-MF 1pr-LEU2), where F and R represent opposite orientations relative to the centromere.

### Construction of Yeast Random Barcode Libraries.

The barcode region of pBAR4_L1 and pBAR5_L1 were PCR amplified with P40, and PEV8 and PEV9, respectively.

(SEQ ID NO: 35)

P40 =
CAACCTGAAGTCTAGGTCCTATT.

PCR products from pBAR4_L1 (containing lox66-Barcode-3′URA3-KanMX) and pBAR5_L1 (containing lox71-Barcode-5′URA3-KanMX) were integrated by homologous recombination into SHA345 and SHA349, respectively, replacing the NatMX marker to yield SHA345+BC (MATa, his3Δ, leu2Δ, met15Δ, ura3Δ, ybr209w::GalCre-lox66-Barcode-3′URA3-KanMX, can1::MFa1pr-HIS3-MF 1pr-LEU2) and SHA349+BC (MATα, his3Δ, leu2Δ, lys2Δ, ura3Δ, ybr209w::KanMX-5′URA3-Barcode-lox71-GalCre, can1::MFa1pr-HIS3-MF 1pr-LEU2). Transformants were picked and arrayed into 96-well plates for storage and further characterization. Each SHA345+BC and SHA349+BC strain was assayed for growth on YDP+kanamycin (for KanMX), YPD+nourseothricin (for loss of NatMX). Additionally, each strain was mated to a complementary tester strain, and plated on CM+galactose−uracil to test for a functional barcode-loxP-1/2URA3 construct. Barcoded strains that passed quality, we next Sanger sequenced at the barcode locus to identify the random barcode sequence. Strains that contain the same barcode were removed from the plate arrays. To check for errors in the library, we next employed an arrayed mating strategy whereby arrayed SHA345+BC plates were pairwise mated to arrayed SHA349+BC plates. Arrayed matings were plated CM+galactose−uracil to select for diploids that have undergone Cre-lox recombination to generate double barcodes. The diploids were pooled, double barcodes from these pools were PCR amplified with a plate specific primer pair, and multiple plate matings were sequenced together on an Illumina MiSeq (see below). Unexpected double barcode reads (which indicate that there was an error in Sanger sequencing or arraying, or a well contained a mix of multiple barcodes) was used to prune the barcode libraries. In total, we generated a verified library 1137 MATa SHA345+BC and 844 MATa haploid barcode strains.

### Haploid PPiSeq Library Construction.

Nine haploid strains expressing PCA hybrid proteins of interest tagged with the N-terminal portion of mDHFR (HOM3-F[1,2]-NatMX, DST1-F[1,2]-NatMX, TPO1-F[1,2]-NatMX, FMP45-F[1,2]-NatMX, FTR1-F[1,2]-NatMX, IMD3-F[1,2]-NatMX, DBP2-F[1,2]-NatMX, SHR3-F[1,2]-NatMX, PRS3-F[1,2]-NatMX) and one negative control strain (ho::NatMX) were each mated with five different SHA349+BC strains. Similarly, nine haploid strains expressing PCA hybrid proteins of interest tagged with the C-terminal portion of mDHFR (FPR1-F[3]-HphMX, RPB9-F[3]-HphMX, SNQ2-F[3]-HphMX, PDR5-F[3]-HphMX, HXT1-F[3]-HphMX, IMD3-F[3]-HphMX, DBP2-F[3]-HphMX, SHR3-F[3]-HphMX, PRS3-F[3]-HphMX) and one negative control strain (ho::HphMX) were each mated with five different SHA345+BC strains. The haploid PCA strains were described in (Tarassov: 2008) and are commercially available at Dharmacon. Diploids were selected on YPD+G418+nourseothricin or YPD+G418+hygromycin B, respectively. The resulting diploids (i.e. two sets of 50 strains) were then sporulated by growing them overnight in YPD to saturation in 96-well microtiter plates at 100 μl per culture, and on the following day washing the pellets twice with water and resuspending the pellets in ‘enriched sporulation media’ (Remy: 2001). The sporulation cultures were incubated in 96-well microtiter plates at 24° C. with continuous shaking at 200 rpm. Spore counts were about 10-20% after one week. 10 μl of every culture was then transferred into 5 ml of YNB+ammonium sulfate+dextrose+leucine+uracil+G418+nourseothricin to select for MATα haploids with a barcode, GENE-F[1,2]::NatMX (and MET+, LYS+) or YNB+ammonium sulfate+dextrose+histidine+uracil+G418+hygromycin B to select for MATα haploids with a barcode, GENE-F[3]::HphMX (and MET+, LYS+) and grown for 3 days to saturation.

### Pairwise Diploid PPiSeq Library Construction.

PPiSeq haploids were systematically mated to create 50×50=2500 diploid strains using standard protocols on a Singer ROTOR HDA robot. Diploid strains were selected on YPD+nourseothricin+hygromycin B. Expression of the Cre-recombinase and strains that successfully recombined their loxP sites were then selected on CSM-uracil+galactose media. A frozen stock of the pool was created by washing the 2500 strains off the agar plates using YPD+15% glycerol and storing aliquots at −80° C.

### Pooled Growth Assays

An aliquot of the frozen pairwise-mated double barcoded PCA pool was thawed and grown overnight by inoculating 200 μl into 20 ml of YNB+ammonium sulfate+dextrose+histidine+leucine. At late log phase (OD600=1.89), four aliquots of 1 ml each were harvested, pelleted by centrifugation, and stored as time-0 samples at −80° C. A 48-well plate was then inoculated with YNB+ammonium sulfate+dextrose+histidine+leucine media (700 μl) with or without 0.5 μg/ml methotrexate and the pool at a starting OD600=0.0525. The media was supplemented with one of the following components: DMSO (final at 0.5%), FK506 (final at 50 μM), hydrogen peroxide (final at 0.001%), sodium chloride (final at 175 mM), or copper sulfate (final at 200 μM). Every condition was assayed in triplicate. Every 3 generations (i.e. at 3, 6, 9, and 12 pool generations), 600 μl were harvested, pelleted by centrifugation and then stored at −80° C. 70 μl were inoculated into fresh media of the same type (i.e. with or without methotrexate and containing the same component). Genomic DNA was then extracted from all 124 samples using the YeaStar Genomic DNA Kit (Zymo Research), and double barcodes were PCR-amplified using the Q5 High-Fidelity 2× Master Mix (NEB) according to manufacturer instructions. PCR was performed with barcoded up and down sequencing primers (multiplexing tags) that produce a double index to uniquely identify each sample. PCR products were confirmed by agarose gel electrophoresis. After PCR, samples were combined and bead cleaned with Thermo Scientific Sera-Mag Speed Beads Carboxylate-Modified particles. Sequencing was performed on an Illumina HiSeq 2500 with 25% PhiX DNA. The PhiX DNA was necessary to increase the read complexity for proper calibration of the instrument.

### Double Barcode Sequence Analysis.

Barcode reads were processed with custom written software in Python and R as described (Levy: 2015), with modifications. Briefly, sequences were parsed to isolate the two barcode regions (38 base pairs each), sorted by their multiplexing tags (see above), and removed if they failed to pass any of three quality filters: 1) The average Illumina quality score for both barcode regions must be greater than 30, 2) the first barcode must match the regular expression ‘\D*?(.ACC|T.CC|TA.C|TAC.)\D{4,7}?AA\D{4,7}?AA\D{4,7}?TT\D{4,7}?(.TAA|A.AA|A T.A|ATA.)\D*|\D*?GTACTAACGGCTAATTTGGTGCCCA\D*’, and 3) the second barcode must match the regular expression ‘\D*?(.TAT|T.AT|TT.T|TTA.)\D{4,7}?AA\D{4,7}?AA\D{4,7}?TT\D{4,7}?(.GTA|G.TA|GG.A|GGT.)\D*’. A BLAST database containing all expected double barcodes (76 bases each) was constructed and each read was blasted (word size=11, reward=1, penalty=−2) against this database. Double barcode reads that blasted at an e<10⁻²⁸ (˜2 mismatches) to an expected double barcode were summed to calculate as an initial estimate of the read number of each double barcode in each condition.

### Comparisons to Existing PPI Studies

Interaction data was downloaded from the Biogrid (S. cerevisiae version 3.4.131). PPIs we sorted based on the form of evidence: Protein Fragment Complementation (PCA), Yeast Two Hybrid (YTH), Affinity Pull-Down Assays (Pulldown), and other lower-throughput methods in the literature.

### Significance Test for Dynamic PPIs

The fitness of each double barcode strain in each environment was determined as described in below. Fitnesses for a given PPI were compared across environments using a two-sided Student's t-test Bonferroni corrected for 400 tests.

### PPI Scoring by Isolated Growth Optical Density Dynamics

Haploid PCA strains were streaked from frozen stocks onto YPD to recover isolated colonies. MATα PCA strains harboring BAIT-DHFR F[1,2]-NatMX were mated one-by-one to MATα PREY-DHFR[3]-HphMX PCA strains in YPD liquid media. A control diploid strain that lacks DHFR was generated by mating a barcoded MATα ho::NatMX strain with a barcoded MATα ho::HphMx strain. Following 12 h of mating, cells were plated onto YPD+nourseothricin+hygromycin B agar and grown for 48 h at 30° C. to select for diploids. One colony of each diploid was inoculated into YPD+nourseothricin+hygromycin B liquid media, grown for 12 h at 30° C., and then stored in 15% glycerol at −80° C. Cells were streaked from frozen stocks onto YPD and grown for 48 h at 30° C. Three isolated colonies of each strain were suspended in sterilized water and counted. For each replicate, 6.4×10⁴ cells were inoculated into 150 ul of media in black-walled, clear-bottom 96-well plates (Nunc #265301). Media was synthetic dextrose supplemented with standard concentrations of the amino acids histidine, leucine, and uracil, plus methotrexate (0.5 μg/ml) and one of the following perturbagens: DMSO (final at 0.5%), FK506 (final at 50 μM), hydrogen peroxide (final at 0.001%), sodium chloride (final at 175 mM), or copper sulfate (final at 200 μM). Plates were sealed with foil (Costar #6570) and shaken at 1,300 rpm (DTS4, Elmi) at 30° C. The optical density (OD units at 600 nm) of each microwell culture was recorded (F500, Tecan) at 0, 8, 10, 12, 14, 16, 18, 20, 22, 24, and 32 h. The area under the curve (AUC) was calculated as the sum of all OD readings before saturation (32 h) for each strain in each environment. The relative fitness for a strain in a specific condition was quantified with following equation: (AUC_{target strain}−AUC_{control strain})_{condition} (AUC_{target strain}−AUC_{control strain})_{DMSO}.

### Split Luciferase Protein Fragment Complementation Assay

To construct Renilla luciferase (Rluc) PCA strains, we replaced the DHFR fragments with Rluc PCA fragments in haploid DHFR PCA strains (Tarassov: 2008) via homologous recombination. The Rluc-F[1]-NatMX homologous recombination cassette was PCR amplified from the pAG25-linker-Rluc F[1]-NatMx plasmid (Malleshaiah: 2010), and the Rluc-F[2]-HphMX cassette was PCR amplified from the pAG32-linker-Rluc F[2]-HphMx plasmid (Malleshaiah: 2010). We used the same pair of primers for the amplification of both homologous recombination cassettes. The forward primer (GGCGGTGGCGGATC-AGGAGGC) (SEQ ID NO:29) anneals to the linker sequence in pAG25-linker-Rluc F[1]-NatMx or PAG32-linker-Rluc F[2]-HphMX. The reverse primer (TTCGACACTGGATGGCGGCGTTAG) (SEQ ID NO:30) anneals to the 3′ end of the TEF terminator region of NatMX or HphMX. To increase the recombination efficiency for some genes, it was necessary to add an additional 40 bp to the forward primer that matches gene-specific sequence upstream of the stop codon. In all cases, MATa PCA (DHFR F[1,2]-NatMX) strains were transformed with the Rluc F[2]-HphMX cassettes and MATα PCA (DHFR F[3]-HphMX) strains were transformed with the Rluc F[1]-NatMX cassettes. Transformants were selected by plating on YPD plus the appropriate antibiotic, and proper incorporation of the Rluc PCA cassette was validated by PCR. Next, MATa PCA strains harboring BAIT-Rluc-F[1]-NatMX were mated one-by-one to MATα PREY-Rluc-F[2]-HphMX strains in YPD liquid media. Following 12 h of mating, cells were plated onto YPD+nourseothricin+hygromycin B agar and grown for 48 h at 30° C. to select for diploids. One colony of each diploid was inoculated into YPD+nourseothricin+hygromycin B liquid media, grown for 12 h at 30° C., and then stored in 15% glycerol at −80° C.

Triplicate fresh colonies of each diploid Rluc PCA strain were grown in 5 ml synthetic dextrose media supplemented with standard concentrations of histidine, leucine, and uracil at 30° C. for 24 h, then diluted 1:32 into 5 ml of the same media supplemented DMSO (0.5%), FK506 (50 μM), hydrogen peroxide (0.001%), sodium chloride (175 mM), or copper sulfate (200 μM). Cells were grown for 24 h at 30° C., diluted 1:32 again into fresh media containing the same supplement, and grown for another 6 h. Cells were counted, and 1-2×10⁷ cells were pelleted, and resuspended in 180 ul phosphate-buffered saline (PBS), pH 7.2 containing 1 mM EDTA. Cells were transferred to white 96-well flat bottom plates (Greiner bio-one #655075). The luciferase substrate, benzyl coelenterazine (Nanolight #301), was diluted 1:10 from the stock (2 mM in absolute ethanol) using 1×PBS, and 20 ul of diluted substrate was added to each sample (to a final concentration of 20 μM). A Centro LB 960 microplate luminometer (Berthold Technologies) was used to measure the Rluc PCA signal, which was integrated for 10 seconds. Changes in luminescence in response to a specific condition were calculated by the following equation: luminescence_{condition}/luminescence_{DMSO}.

### Pooled Construction of a Large Double Barcode Library

iSeq-barcoded haploid MATa (1137 SHA345+BC strains) and MATα (844 SHA349+BCs strains) strains were grown to saturation (48 h at 30° C.) in 100 uL YPD+G418 in 96-well plates. Clones of the same mating type were pooled to generate the MATα and MATa barcode pools, and stored in 15% glycerol aliquots at −80° C. The frozen barcode pools were thawed completely at room temperature, and 1.35×10⁹ cells of the MATα pool and 2.9×10⁹ cells of MATa pool were each inoculated into 200 ml YPD+G418 and grown for 20 h at 30° C. A cell count of each pool was taken, the two pools were combined at equal cell densities, and this mixed pool was streaked onto 6 YPD plates at a density of 10¹⁰ cells/plate to mate. Cells were grown on YPD for 24 h at 30° C., and then all plates were scraped and pooled in water. The number of cells in this pool was counted and ˜3.3×10¹⁰ cells (⅓ of all the cells) were plated onto 30 SC-Met-Lys plates at equal cell densities. Cells were incubated for 48 h at 30° C. and then replicated onto another 30 SC-Met-Lys plates. After another 48 h incubation at 30° C., cells were scraped from the 30 SC-Met-Lys plates and pooled in water. All the cells (4.2×10¹⁰) were spun down, resuspended with 1 L SC+Gal−Ura, and grown for 48 h at 30° C. Then cells were counted and 100 mL (˜8.2×10⁹ cells) was inoculated into 1 L SC-Ura media and grown for 48 h at 30° C. to further enrich for loxP recombinants. Finally, all the cells were collected to form the pooled diploid barcode library.

### Sequencing of Bulk Mated Double Barcode Pools

Genomic DNA of the pooled diploid PPiSeq library and pooled diploid barcode library was extracted using the MasterPure Yeast DNA Purification Kit (Epicentre # MPY80200). To completely remove RNAs, extra RNase treatment, DNA precipitation with isopropanol, and washing with 70% ethanol were added after the recommended protocol from the manufacturer. Double barcode amplicons were generated using a two-step PCR protocol (Levy: 2015). Briefly, a 5-cycle PCR with OneTag polymerase (New England Biolabs) was performed in 6 reactions (˜500 ng template and 50 μl total volume per reaction) for the diploid PPiSeq library amplifying ˜80,000 copies per unique lineage tag, and 60 reactions for the large double barcode library amplifying ˜1000 copies per unique lineage tag. The PCR products were then pooled and purified with PCR Cleanup columns (Qiagen) at 6 PCR reactions per column A second 21-cycle (diploid PPiSeq library) or 23-cycle PCR (diploid barcode library) was performed with high-fidelity PrimerSTAR Max polymerase (Takara) in 3 reactions for the diploid PPiSeq library and 30 reactions for the large double barcode library, with 15 μl of cleaned product from the first PCR as template and 50 μl total volume per tube. PCR products from all reaction tubes were pooled and purified using a PCR Cleanup column (Qiagen) and eluted into 50 μL of water. The appropriate PCR band was isolated by E-Gel agarose gel electrophoresis (Life Technologies) and quantitated by Qubit fluorometry (Life Technologies). Sequencing was performed on an Illumina HiSeq 2500 with 25% PhiX DNA spike-in. The PhiX DNA was necessary to increase the read complexity for proper calibration of the instrument.

### Fitness Estimation by Lineage Tracking

We use the corrected double barcode reads at 0, 3, 6, 9, and 12 generations to estimate the fitness of each double barcode PPiSeq strain in each condition and replicate. In competition assays, the “fitness” is defined as a relative growth rate: the relative increase in frequency per unit time of one genotype over another. Here, we measure relative to a “null” strain with no PCA constructs (ho::NatMX/ho::HphMX), whose fitness is then defined to be x=0. Using the frequency of each double barcode to infer the fitness, x, of each lineage (between time points t and t+δt) relative to this null strain is then straightforward:

$$x = \overline{x} ( t ) + \frac{ln ( f ( t + δ t ) ) - ln ( f ( t ) )}{δ t} \qquad ( 1 )$$

where x̅ is the mean fitness of the population, defined as

$$\overline{x} ( t ) = {\sum }_{lineages i} {x}_{i} {f}_{i} . \qquad ( 2 )$$

Because of the differences in fitness between strains, the mean fitness can change substantially over short periods of time, even at the very beginning of the assay. Accurate inferences of fitness from frequency data must take this changing mean fitness into account.

Linear regressions can have high errors of fitness. The simplest way of estimating the relative fitnesses would be to perform a linear regression on the (log) relative frequencies. However in most situations, a linear regression performs poorly because, as the mean fitness of the population increases, trajectories begin to curve and linear regression will no longer accurately capture the true relative growth rates (FIG. 19B). Sometimes, if the mean fitness does not increase significantly early on, restricting analysis to the first two time points allows linear regression to perform reasonably well. However, the rate at which the mean fitness changes depends strongly on the pool of genotypes being tested and the environment in which they are grown, so this method cannot be generalized. Additionally, subtle fitness differences will often go undetected when restricted to just two time points because the noise around any one time may be high. Incorporation of additional time points (when the mean fitness is changing) therefore has the potential to significantly decrease fitness estimate errors.

A maximum likelihood method to reduce fitness errors. To improve fitness estimates over linear regression, we use a maximum likelihood algorithm to infer relative fitnesses. Our algorithm maximizes:

Probability(relative frequency data fitness estimates & initial frequency estimates) (3)

The advantage of such an approach is that it makes use of all the data. As we show in the comparisons to simulated data sets this approach can significantly improve fitness estimates: reducing the errors on high fitness genotypes by an order-of-magnitude under conditions similar to our experiment. Improvements of our likelihood maximization process over a linear fit will, of course, depend on the environment, the pool of genotypes being tested, and the sampling frequency.

Interactions though the mean fitness. One key subtlety in performing any optimization to determine the “best” fitness estimates is that one cannot optimize each lineage independently. A change in the estimate for the fitness of lineage 1, say, impacts the likelihoods of all other lineages, particularly if lineage 1 is very fit. We discuss this subtlety in steps 10-12 of the algorithm below in reference to how best to update guesses to search for the maximum likelihood position.

What functional form should be chosen for the likelihood function? In general there are a number of stochastic processes that determine the relative frequency inferred from unique sequencing reads given an initial frequency and fitness. These include sampling at the sequencer (i.e. finite read depth), PCR amplification noise and noise inherent to the growth process of the cells and sampling at bottlenecks (“genetic drift”). In the data considered here the population size (N≈10⁷) is far larger than the read depth at a typical time point (R≈5×10⁵). Therefore sampling at the sequencer dominates the noise with genetic drift adding a very minor correction to this (see below “Errors on frequency”). We therefore assume changes in relative frequency from time point to time point are deterministic, with all noise introduced at the sequencing stage. Extending our algorithm to include other forms of noise would be straightforward. We have found that:

$$ln P ( r \vert f ) \approx \frac{1}{2} ln ( \frac{{( Df )}^{1 / 2}}{4 π κ {r}^{3 / 2}} ) - \frac{{( \sqrt{r} - \sqrt{DF} )}^{2}}{κ} \qquad ( 4 )$$

is an accurate functional form for the noise, so we use this in our likelihood estimates. Here, is a (free) noise parameter O(1) that can be fit from the data. Of particular importance is that this form has an exponential rather than Gaussian tail.

### Algorithm

1. Start by making an initial guess at the initial frequencies f and fitnesses x for all lineages (these are vectors whose entries are the values for the first, second lineage etc. . . . down to the 2,500th lineage). A good guess at the initial frequencies comes from looking at the relative frequency of the lineages at t₀:

$${f}_{i} = \frac{{r}_{i} ( {t}_{0} )}{D ( {t}_{0} )} \qquad ( 5 )$$

where rᵢ is the number of reads on the ith lineage and D the read depth (both at t=0). A reason-able first guess for the fitnesses comes from performing a linear regression on the log-transformed trajectories:

$${x}_{i} = \frac{ln ( {f}_{i} ( t + Δ t ) ) - ln ( {f}_{i} ( t ) )}{Δ t} \qquad ( 6 )$$

2. Given these initial guesses we want to calculate the likelihood of the data under the assumption that competition between lineages is only via the mean fitness and that no lineages accumulate any additional beneficial mutations, so that fitnesses remain constant in time.
3. Use the fitnesses xᵢ and initial frequencies f(t₀) to estimate the initial mean fitness x̅ (t₀)

x̅(t₀)=x·f(t₀) (7)

4. Use the fitness xᵢ and the initial mean fitness x̅(t₀) to predict the frequencies at the next time point:

fᵢ(t₀+Δt)=fᵢ(t₀)exp[(xᵢ−x̅(t₀))Δt] (8)

5. Recalculate the new mean fitness at this later time point:

x̅(t₀+Δt)=x·f(t₀+Δt) (7)

6. Iterate this procedure until the frequencies of all lineages at all time points are predicted (as well as mean fitness trajectory):

{f(t₀),f(t₁) . . . f(tₖ)} and x̅(t) (10)

### See FIGS. 30A and 30B.

7. The (log) probability distribution across reads, r, given some read depth, D, and true frequency, f, of the lineage is calculated using

$$ln P ( r \vert f ) \approx \frac{1}{2} ln ( \frac{{( Df )}^{1 / 2}}{4 π κ {r}^{3 / 2}} ) - \frac{{( \sqrt{r} - \sqrt{DF} )}^{2}}{κ} \qquad ( 11 )$$

where κ is the noise parameter which is O(1) and can be obtained by fitting.
8. The log likelihood of the data given the model is then obtained by summing over all time points. The total likelihood L of all data given the guesses across all lineages is then obtained by summing across all lineages. This value L is a function of x and f(t₀), which are our “guesses”.

L(x,f(t₀)) (12):

9. The aim is to maximize this likelihood by making small changes to our guesses and accepting those that increase the likelihood. However, because of the interaction through the mean fitness, it is extremely inefficient to make random steps away from the current guess and re-evaluate the likelihood each time as some optimization algorithms would implement. The inefficiency comes from the fact that any change to any fitness requires re-calculating the likelihood for all other lineages because of the interaction through the mean fitness.
10. Instead, we implement a “smart” guess by realizing that the interaction through the mean fitness is rather weak. What this means in practice is that maximizing the likelihood of each lineage independently, assuming that the mean fitness does not change, should be a good approximation to the true maximum likelihood guess and hence should be a sensible next guess. We therefore choose this the way of updating our guesses for frequency and fitness.
11. Once this new guess is made, the trajectories are calculated in a way that is self-consistent with the predicted mean fitness as outlined in steps 3-6. If the guess increases the likelihood, it is accepted.
12. This process is repeated until the algorithm converges (no steps can increase the likelihood further).
13. The final guesses for the frequency and fitness vectors are then assigned to me the maximum likelihood guesses.
14. This algorithm is not guaranteed to converge to the global maximum since it is deterministic rather than stochastic. However, by examining a large number of likelihood surfaces (as shown in FIG. 30B) we found no cases where the algorithm was trapped in a local maximum (because landscapes are smooth). We verified this with simulations discussed below.

Applying the maximum likelihood algorithm above to a simulated data set with 2500 lineages results in accurate inferences of the fitness. The algorithm improves upon linear regression substantially, particularly for lineages with positive fitness. Lineages with (x>0) typically are measured across all 5 time points. Here the fact our algorithm uses all the data is important: it reduces the errors in fitness by an order of magnitude (from ±0.1 down to ±0.01). For lineages with negative fitness the improvement is more modest. Lineages with low fitness are typically pushed to low frequencies rapidly and the first two time points are therefore the most informative. It is therefore hard to improve substantially on the linear regression method which itself uses only the first two time points. We observe however that this is some improvement for lineages with moderately negative fitness −0.3<x<0. Here fitness errors come down by about a factor of two (from ±0.1 to about ±0.05)

Comparison to simulated data set. To verify that this algorithm does indeed work well and to quantify the improvement it affords over a simple linear regression we ran it on a simulated data set (FIGS. 30 and 31). The simulated data set was closely modeled on the experimental set-up.

### Specifically:

1. Two vectors (of length 2,500) are created to serve as the true initial frequencies F and true fitnesses X.
2. The initial frequencies F are drawn from a Gaussian distribution with mean μ= 1/2500=4×10⁻⁴ and standard deviation 6=8×10⁻⁵ with each entry being forced to be positive.
3. The fitnesses X are drawn from a distribution with density ρ(x)=exp(−|x|) where the range is restricted to being in the interval −0.5<X<0.5. This distribution means that most lineages have small fitness, while also ensuring there will also be lineages at the extremes of the range.
4. The frequencies of each lineage at subsequent time points are calculated via:

$${F}_{i} ( t + 1 ) = {F}_{i} ( t ) exp ( {X}_{i} - \overline{X} ( t ) ) + η \sqrt{\frac{{F}_{i} ( t )}{N}} \qquad ( 13 )$$

where the first term is the deterministic change in frequency due to fitness differences and the second term are stochastic changes due to genetic drift. X̅ is the mean fitness X·F and η is a random variate from a Gaussian distribution with zero mean and unit variance which is used for the stochastic elements of genetic drift. Using this procedure frequency data is generated for each lineage out to 12 generations.
5. Every 3 generations we generate read counts by Poisson sampling the frequencies at a mean coverage of 200/lineage=500,000 total reads (typical of the data).

See FIGS. 31A AND 31B.

### Analysis of Errors

Errors on frequency measurement. The errors in frequency measurements for the vast majority of bar-codes are characterized by counting noise i.e. noise where the variance is proportional to the mean. To validate this, we looked at frequencies of the same barcode measured across different replicates. If the noise is counting noise, then the standard deviation (i.e. typical error) in the frequency in replicate 1, say, should be:

$$δ {f}_{1} ∼ \sqrt{\frac{f}{{R}_{1}}} \qquad ( 14 )$$

hence if we plot the magnitude of the difference in estimated frequencies between the two replicates divided by the mean frequency (the “coefficient of variantion”) then

$$\frac{\vert {f}_{1} - {f}_{2} \vert}{\overline{f}} ∼ \frac{1}{\sqrt{f}} \vert \frac{1}{\sqrt{{R}_{1}}} - \frac{1}{\sqrt{{R}_{2}}} \vert \qquad ( 15 )$$

so counting noise behavior can be validated by checking that, as a function of the mean frequency, the coefficient of variation declines as 1/\sqrt{f}. The constant of proportionality should be a small multiple of 1/√R where R is the sequencing depth. In the plot below we validate this by plotting the coefficient of variation in frequency between replicates as a function of mean frequency on log-log axes, on which a 1/\sqrt{f} scaling will have a gradient of −½. For barcodes at low frequency (<0.1%), their scaling broadly agrees with that predicted by counting noise with a coefficient between 1-3 (FIG. 32). The error in frequency of these barcodes is therefore dominated by the noise that comes from finite read depth. Barcodes present at higher frequencies (>0.1%) begin to deviate from this scaling. Barcodes at higher frequency likely have non-negligible contributions from other noise processes such as PCR and DNA prep noise as well as a likely contribution from biological noise. As discussed below, there are also sources of systematic errors which disproportionately affect high-frequency barcodes. We note however, that the errors associated with high-frequency barcodes are nonetheless generally much smaller than those of low frequency barcodes.

Systematic errors on fitnesses. To quantify the magnitude of systematic errors in fitness, we plot all correlations between fitness inferences across all replicates for each condition (FIG. 33). In most conditions we find a consistent story: high fitness barcodes in one of the three replicates typically demonstrate systematic differences in relative fitness with magnitudes up to ±0.15. Interestingly these systematic effects only influence the high-fitness strains. Low fitness strains have no noticeable systematic effects (i.e. they are scattered symmetrically around the x=y). The most likely explanation for these systematic effects are due to estimations of the “mean fitness” over the last few time points. A slight underestimation of the mean fitness at late times, for example, would cause the estimates for all high fitness barcodes to be underestimated too. Such systematic effects influence the high-fitness barcodes more than the low fitness ones because information from the later time points affects their estimates more (since they are at higher frequency at late times). Another plausible reason for these systematic effects is that the handful of high fitness strains that dominate the population at late times can modestly change the environment in which pooled growth is happening. This is consistent with the lack of systematic effects observed in previous pooled growth studies which start with higher complexity pools and where no one strain increases enough to dominate the population. We hypothesize therefore that systematic effects will be reduced as the PPiSeq platform is scaled up. In this case, any one strain will constitute a small fraction of the pool and therefore makes it less plausible it can change the environment significantly.

Mating and loxP Recombination Efficient Estimates

### Mating Efficiency Estimation

A mating efficiency test between barcoded PCA strains was performed in quadruplicate. Barcoded MATa and MATα PCA pools were each grown in 50 ml YPD liquid media to saturation. The two pools were combined, and 1×10¹⁰ cells were plated onto a single YPD plate to mate. Cells were grown for 24 h at 30° C. and the cell lawn was scraped into 10 ml of water. A cell count was taken to determine the total growth on the plate (˜1.7-fold growth). Cells were spread onto plates YPD+CloNat+Hygromycin plates at densities of 1000, 2000, and 5000 cells/plate to estimate the number of diploids on the mating plate. Following a 48 h growth at 30° C., colonies on each plate were counted and a linear regression was fit to this data. However, a single mating event may result in several observed diploids because some growth occurs on a mating plate, meaning that early mating events may be counted more than once. Thus, to generate a more conservative estimate of the mating efficiency, we divided the number of observed diploids by the fold increase in the number of cells on the mating plate (˜1.7). This procedure is likely to be an underestimate of the true mating efficiency for two reasons: 1) it assumes that all diploids are generated before cell outgrowth, while it is likely that some are generated after one or more haploid cell divisions, and 2) it assumes that diploids undergo the same number of cell divisions as haploids, yet mating takes ˜4 hours, meaning that haploids are likely to undergo more cell divisions during the outgrowth on the mating plate. Nevertheless, the lower bound of the mating efficiency reported in FIG. 22A is the most useful measure for the ultimate scalability of the assay.

### LoxP Recombination Efficiency Estimation

A loxP recombination efficiency test was performed on four randomly picked clones from a pooled mating between iSeq-barcoded PCA strains (above). Each clone was grown in 5 ml YPD+Nat+Hyg liquid media for 24 h at 30° C., spun down, and resuspended into 3.2 ml of YPG liquid media at a cell concentration of ˜2×10⁸ cells/ml to induce Gal-Cre mediated loxP recombination. Cells were grown for 24 h at 30° C., and a cell count was taken to calculate the fold increase in cells in the recombination media (˜1.7-fold growth). Cells were plated at three densities (500, 1000, and 2000 cells/plate) on SC-Ura agar and incubated for 48 h at 30° C. Each plate was counted and a linear regression was fit to this data to estimate the total number of recombinant cells. Similar to mating frequency estimations described above, a single recombination event may result in several observed recombinants because some growth occurs in the recombination media. Thus, to generate a lower bound of the recombination efficiency, we divided the number of observed diploids by the fold increase in the number of cells in the recombination media. Results are depicted in FIG. 22A.

### Comparison Between Bulk and Pairwise Mating

Pairwise mated libraries were sequenced at a higher depth than bulk mated libraries (−200 reads per barcode and −67 reads per barcode, respectively). The compare barcode frequency distributions at similar read depths, we sampled pairwise mating reads (without replacement) to −67 reads per barcode. Shown in FIG. 22B. Other sampling attempts did not significantly change the results or conclusions.

### Example 10. A Double-Barcode Method for Detecting Dynamic Genetic Interactions in Yeast

An interaction Sequencing platform (iSeq) is developed and applied to measuring genetic interactions. The key innovation of iSeq is a system that recombines two barcodes that exist on homologous chromosomes such that they are brought into close proximity on the same physical chromosome in vivo to form a double barcode (FIG. 34A). iSeq accurately assays the fitness of each uniquely marked strain in the pool by monitoring double barcode frequencies over several growth bottleneck cycles using a quantitative double-barcode amplicon sequencing and counting protocol. In this study, we demonstrate the utility of iSeq, by using it to measure the GIs between all pairwise combinations of nine deletions across three environments at high replication. For any given clonally derived double barcode strain, we show that fitness measurements and iSeq interaction scores are highly reproducible across biological replicates and find several new environment-dependent GIs. However, we find low reproducibility between different double barcode strains ostensibly carrying the same double deletions, which cannot be explained by measurement error. By whole-genome sequencing of the experimental strains, we find that segregating variation and de novo mutations that occur during strain construction can have large effects on genetic interaction scores.

### Results

The iSeq Platform

The iSeq platform includes a novel double-barcoding technology combined with a pooled fitness assay. The double-barcoding technology uniquely identifies both parents of a mating event. While iSeq could be used to study interactions between any two genomes or genetic elements, here we use iSeq in combination with gene deletion strains to assay interactions between pairwise combinations of deletions over three environments. Our system functions by first introducing loxP recombination sites at a common chromosomal location in both MATα and MATα haploids. Barcodes are placed on opposite sides of the loxP sites such that mating and Cre induction causes recombination between homologous chromosomes, resulting in a barcode-loxP-barcode configuration on one chromosome (FIG. 34A). Because these double barcodes are unlikely to dissociate during genomic DNA preparation and are in close enough proximity to be sequenced by short-read single-end or paired-end sequencing, pools of double barcode strains can subsequently be assayed using standard pooled barcode sequencing approaches. See for example (Pan: 2004).

Experimental Design: Genes and Controls Chosen for iSeq Validation

To validate this approach, a group of 9 genes was selected and used iSeq to measure the genetic interactions between the 36 possible gene pair combinations. To assess iSeq across a range of values, the genotypes in this set were chosen to include a range of published quantitative interaction scores. Furthermore, seven of the gene pairs have no published interaction, providing negative controls as well as the possibility of detecting novel environment-dependent genetic interactions upon growth in new conditions. By “marking” each of these gene deletions with four different iSeq barcodes, up to eight independently constructed strains were generated for each double mutant assayed, thus providing a high level of biological replication.

Single mutant controls, required for interaction score estimates, were generated via the same protocol as their double mutant counterparts, ensuring that all experimental strains carried iSeq double barcodes and the same set of markers. When generating single mutants, we used dubious ORF deletions as placeholders for the second gene deletion. The two dubious ORFs YHR095W and YFR054C were chosen, are not expressed, have no fitness defect when deleted under the conditions in which they have been tested, and have no reported genetic interactions in the BioGRID database. Thus, strains carrying one gene deletion and one dubious ORF gene deletion should be reasonable proxies for single mutants. In total, we assayed multiple replicates of 36 double, and 9 single gene deletions.

Construction of iSeq Deletion Strains

To generate deletion strains carrying the double-barcoding system we first constructed two yeast iSeq barcode libraries (288 strains each, in the same MATα starting strain) by replacing the dubious open reading frame (ORF) YBR209W with one of two complementary plasmid-derived constructs via homologous recombination. The YBR209W site has been used successfully as an integration site for heterologous genetic elements, and its transcript is not expressed and its absence does not significantly affect fitness.

MATa strains derived from the systematic deletion collection (Winzeler: 1999) that carry either a NatMX or a KanMX selectable marker at the deletion locus (F0 haploids) were selected and mated to MATα clones from each barcode library. Resulting diploids were sporulated and the magic marker system (Tong: 2004) was used to select MATa or MATα haploid clones containing both the iSeq barcode and either a KanMX or NatMX marked deletion, respectively (F1 haploids, FIG. 34B). After selection, and for each clone, the mating type was verified and the iSeq barcode sequence identified. In total we barcoded each of the 9 gene deletions and 2 dubious ORF deletions with 4 different single iSeq barcodes, 2 barcodes for each version of the deletion (KanMX or NatMX) (FIG. 34B).

To construct double-barcoded double-deletion strains, we mated all pairwise combinations of KanMX and NatMX strains, induced recombination at the iSeq barcode locus, sporulated, eliminated diploids by zymolyase digestion and then selected haploid clones (F2 haploids, FIG. 34C). After all matings, each double gene deletion is represented by up to 8 unique iSeq double barcodes, and each single gene deletion, that brings together a gene deletion with a dubious ORF deletion, is represented by up to 16 double barcodes (FIG. 34D). Finally, 8 double-barcoded control strains, each intended to represent a wild-type phenotype, were generated by bringing together two dubious ORF deletions. In total, 393 double-barcoded strains, 257 double gene deletions, and 136 single gene deletions were generated.

### Pooled Fitness Estimates of Double-Barcode Double-Deletion Strains

All 393 double-barcode haploid strains were pooled and mixed this pool with a pool of the 8 putative wild-type control strains at a ratio of 50:50. We combined pools in this way so that at least 50% of cells start with approximately wild-type fitness, thereby minimizing the effects of strain-strain interactions between different mutant genotypes during pooled growth. We propagated this combined pool by serial batch culture in YPD at 30° C. at an effective population size of 8×10⁹, bottlenecking 1:8 at each transfer (FIG. 35A, every 3 generations). This design, which samples at multiple and relatively frequent time points, was chosen for three reasons. First, multiple measurements increase the sensitivity to detect subtle fitness differences between strains. Second, measurements every few generations enable accurate estimates of low fitness genotypes that are rapidly driven to extinction. Third, this large population size was required for our DNA extraction and barcode sequencing protocol, such that sufficient material could be extracted for barcode sequencing. At each bottleneck, we extracted genomic DNA and then sequenced the double barcodes to estimate the relative frequency of each strain in the population (FIGS. 35A-35B). The slope of a log-linear regression of the change in frequency relative to wild-type over the four time points was used as the measure of fitness for each double barcoded strain. For each double barcoded strain, fitness measurements were highly reproducible across biological replicates (FIG. 35C, Spearman's rho=0.91-0.97, P<2.2×10⁻¹⁶). The possibility that our pooled fitness assay might have larger errors on lower fitness genotypes was investigated, as those genotypes could be quickly driven to low frequencies where sampling errors have a larger effect. No significant association between fitness and standard deviation of fitness in our assay was found (Spearman's rho=−0.07, P=0.19), with the least fit double barcode still having a low fitness error (s=0.49+0.11) in YPD. Greater errors on a small subset of low fitness strains in the two other conditions tested was discovered, as in these conditions, these strains are typically driven below our detection limit after just two time points.

To validate the fitness obtained by iSeq, and to determine whether pooling strains had an effect on strain fitness, we next compared iSeq fitness measurements to those from a standard growth assay. Each strain was grown in an individual well of a multi-well plate, optical-density based growth curves were generated and the maximum exponential growth rate was used as a proxy for fitness.

Exponential growth rate might not be expected to correlate highly with fitness during sequential batch growth since potentially important growth dynamics when entering or leaving saturation are not captured in sequential batch growth. Nevertheless, we find a significant positive correlation between the two methods indicating that potential strain-strain interactions during pooled growth had little to no effect on our fitness estimates (FIG. 35D, Spearman's rho=0.68, P<2.2×10⁻¹⁶, N=391 strains).

However, despite the reproducibility of the fitness estimates for any given double barcode across replicate cultures, and its concordance with a secondary measure of fitness, there was variability in fitness between strains carrying different double barcodes but the same putative gene deletions. The median SD of fitness for the same double barcode measured across independent cultures is 0.049, while the median SD of fitness of strains with different barcodes but the same deletions is 0.063 (FIG. 35E). This high variability across strains was also observed in our independent OD-based measure of fitness, indicating it was not an artifact of measuring the fitness in pooled format (FIG. 35F).

### Influence of Genetic Background on Fitness

The fitness varied when comparing strains carrying identical gene deletions but unique double barcodes (FIGS. 35E-35F). This variation between double barcodes may be caused either by segregating genetic variation in the parental strains and/or by de novo mutations that occurred during the growth, mating, or sporulation steps of strain construction. To investigate this possibility, whole genome sequencing was performed on 10 F0_{BC}, 6 F0, 24 F1 and 39 F2 strains that were related by descent (FIGS. 34B-34C). 8 control F2 strains were sequenced, which each carry two dubious ORF deletions, and their corresponding F0 and F1 parental strains, in order to help determine for any mutations that did arise, whether they arose due to the strain generation protocol or due to the presence of a gene deletion that causes a severe fitness defect.

A subset of strains from the gene deletion collection has been shown to carry both aneuploidies and suppressor mutations. Thus, as the sequenced F0 strains were derived from the deletion collection, we first looked for mutations present in these strains. In 7 of the 8 F0 strains, we observed between one and three private SNPs that were not observed in any other strains except direct descendants (FIG. 36A), with similar numbers observed between the gene and control groups. Only one aneuploidy was observed, in the PHO23 deletion strain, on Chromosome XI.

The mutations present in the 24 F1 strains carrying one gene deletion and one iSeq barcode (FIG. 34B) were studied. Surprisingly, aneuploidy was extremely common, with 14 strains having an extra copy of at least one chromosome, and of those, 12 strains carried an extra copy of Chromosome V. We also observed aneuploidy in 3 of the 8 F1 control strains (FIG. 36A), indicating the aneuploidies were not a response to a specific gene deletion, but more likely a general result of the strain generation procedure. In addition to aneuploidy, we found that 15 of the 32 F1 strains had accumulated between 1 and 3 new SNPs during the first cross and selection, with similar numbers observed in both gene and dubious ORF controls (FIG. 36A). Some fraction of the SNPs first observed in F1 strains may have originated from the unsequenced iSeq barcode construct strains to which the F0 deletion strains were crossed. However, as we describe below, similar numbers of de novo private SNPs were observed in the F2 strains, suggesting that the fraction of SNPs originating in unsequenced barcode construct strains is small.

Next the genomes of the 39 F2 strains where analyzed, which were generated after the second round of mating and were used in the pooled fitness assay (See FIG. 34C). First, as with the F1 strains, aneuploidy was common (FIG. 36B). Of the 39 sequenced F2 strains, 21 had at least one chromosome duplicated (54%) and of these, as with the F1 strains, chromosome V was the most likely to be duplicated (16 of 21 strains). The strains aneuploid for chromosome V generally had lower fitness than strains with the same gene deletions but no aneuploidy (FIG. 36C). A duplication of chromosome V was also observed in one of the eight F2 control strains, indicating these aneuploidies can occur in the absence of gene deletions. In total, 25 of 30 aneuploidies observed in the 21 F2 strains appeared to be inherited, as the aneuploidies were also observed in at least one related F1 parent. Aneuploidies also appeared to be lost, of the 7 crosses where both F1 parents carried the same duplicated chromosome, in 3 cases F2 progeny did not have the aneuploidy.

Second, by examining the coverage in the genic regions that we expected to be deleted, we observed that 6 of the 39 sequenced F2 strains actually carried a copy of one or both of their two intended gene deletions. In two cases, aneuploidy of chromosome I yielded a heterozygous DEPT gene deletion. Two other cases (in putative arp6Δpho23Δ and sds3Δpho23Δ strains) contained reads mapping to the expected gene deletions, as well as several heterozygous SNPs, suggesting that they are diploids that somehow managed to survive digestion by zymolyase and haploid selection via the magic marker system. The two remaining cases contained reads mapping to the PHO23 ORF, even though it was intended to be deleted, but no evidence of either aneuploidy or diploidy. A rare recombination event reinstated the PHO23 sequence after the second mating step to a strain carrying a wild-type PHO23. These reversions did not always lead to an increase in fitness as compared to other strains in the same group, as they often coincided with other events such as aneuploidy (FIG. 36C). None of the F0 deletion collection strains yielded sequencing reads at their gene deletions, while two F1 strains did, due to aneuploidy (in DEP1 or SDS3), indicating these gene reinstatement events can occur after just one round of mating. No read coverage was ever observed in any of the 27 sequenced dubious ORF deletions (F0, F1, and F2), suggesting that these reversion events may be selected for because they result in increased fitness.

Finally, there were a total of 62 unique SNPs and small indels segregating across the 39 F2 double deletion strains sequenced. The analysis of the sequenced parent strains indicates that approximately ⅓ of these were first observed in the deletion collection, ˜⅓ after the first cross, and ˜⅓ after the second cross. The total number of SNPs observed per double mutant strain ranged from 1 to 10, with a median of 6 (FIG. 36B), and the number per strain did not vary significantly across the five double deletion groups (P=0.45, Kruskal-Wallis Rank Sum Test). We also observed 4 to 10 SNPs in our 8 control F2 strains, illustrating similar numbers of mutations accumulate in the absence of gene deletions. A majority (56%) of the SNPs and indels either fall in intergenic regions, result in synonymous changes, or result in amino acid changes predicted to be tolerated. However, 18% resulted in frameshifts, premature stop codons, or non-synonymous changes predicted to affect protein function. There was no significant enrichment for any GO terms for the genes hit by SNPs and indels with functional consequences; however, this might be due to the small sample size. Regardless, segregating variation likely underlies some of the differences in fitness observed for different double barcoded strains carrying the same gene deletions.

### Interaction Score Estimates Using Double Barcodes

Despite the genetic variation present in our strains, we were still able to calculate an interaction score for each strain using our fitness data. An interaction score, E, is defined as the difference between the observed double mutant fitness, and its expected value based on the product of the fitnesses of the two corresponding single mutants. Using this definition, we find that interaction scores for each double barcode strain are highly reproducible between biological replicates (FIG. 37A, Spearman's rho=0.96-0.98, P<2.2×10⁻¹⁶, N=255 strains) and correlate with interaction scores derived from the maximum exponential growth rate of single and double mutants (FIG. 37B, Spearman's rho=0.69, P<2.2×10⁻¹⁶, N=255 strains). However, high variance between double barcodes that represent the same putative double knockout genotype was found. The median SD of interaction scores across strains with identical gene deletions is 0.072, 2.5-fold higher than the variance of each double barcode across biological replicates (median SD=0.072 vs. SD=0.028, P=2.2×10⁻¹⁶, Wilcoxon Rank-Sum Test, N=36 gene deletion pairs and 255 strains).

The interactions identified herein was compared with those collected through literature curation (Stark: 2006). It is noted however, that these published interactions are generally derived from colony growth on plates, and some interactions can be condition-specific, such that they are only observable either during growth in liquid, or when assayed on plates. Of the 36 gene pairs we tested, 14 have a reported negative genetic interaction, 15 a positive reported interaction and 7 have no reported interaction. Our scores for interactions in strains in the positive group were significantly different from those in the negative group (FIG. 37C, P=1.2×10⁻⁴, Wilcoxon Rank-Sum Test), suggesting that despite the scores being generated from different experimental conditions, and the known genetic variation our strains, there are similar observable trends.

To compare iSeq interaction scores to those previously reported from large-scale systematic screens, we calculated a mean interaction score for each double deletion (4-8 double barcodes per double gene deletion with 3 replicate growth experiments each). Interaction scores derived from iSeq weakly correlate with those derived from two previous studies (Collins: 2007; Costanzo: 2010) (Collins: Spearman's rho=0.36, P=0.063, N=28 gene pairs; and Costanzo: Spearman's rho=0.38, P=0.005, N=33 gene pairs). As discussed above, complete agreement is not necessarily expected between different assays because they are performed in different growth conditions.

Measurement of Differential Interactions Using iSeq

Two additional pooled fitness assays we performed on our set of strains—one in heat stress (YPD 37° C.) and one in a non-fermentable carbon source (ethanol and glycerol, YPEG). As we observed in rich medium, fitness and interaction score estimates in the two new growth conditions were highly reproducible across replicate cultures (Spearman's rho=0.97-0.99, P<2.2×10⁻¹⁶, fitness median SD=0.027, interaction score median SD=0.024), while there was only a weak negative correlation between fitness and the SD of fitness across replicate cultures.

To determine whether there are changes in interaction scores across conditions, we first called significant interactions in each of the three conditions using 95% confidence intervals. Though many changes in sign and magnitude of interaction scores were observed between YPD and the two alternate conditions, a total of three gene pairs changed interaction score in a statistically significant manner (FIG. 37E, P≤0.005, N=6-8 strains, Wilcoxon Rank-Sum Test, 10% FDR). Two gene deletion pairs (dep1Δpho23Δ and sap30Δpho23Δ) had no interaction in YPD but interact negatively in YPEG. One other gene deletion pair (sap30Δsnt1Δ) changed from no significant interaction in YPD at 30° C., to a negative genetic interaction in YPD at 37° C.

### DISCUSSION

A new double barcode interaction sequencing technology (iSeq) was developed that can be used to quantitatively examine pairwise genetic interactions. iSeq's double barcoding system allowed us to use pooled serial batch growth and high-throughput sequencing to measure the fitness of hundreds of double deletion strains simultaneously, an approach previously only possible with pools of single deletion strains, or double deletions carrying a common deletion. Our method produces extremely reproducible fitness and GI estimates for the same double barcode across replicate pooled growth experiments. Furthermore, the pooled iSeq fitness and GI scores correlate well with measurements made during individual growth, indicating pooled growth does not confound our results. At current rates, considering an average coverage of 100 reads per strain for each of five time points and 50% of the pool made up of a WT control strain, we estimate a sequencing cost of S0.02 per GI per replicate per environment, and these costs will fall at the same rate as sequencing.

In one embodiment, iSeq can be applied to the measurement of interactions between a larger group of genes is to modify the strain generation protocol. By implementing robotics to automate matings, pinnings and selections on plates, one could relatively easily cross iSeq BC library strains (carrying single iSeq barcodes) to deletion collection strains by SGA. Double-barcode, double deletion strains could then be generated via another round of SGA, or, for increased throughput, via pooled matings. In contrast to our pilot study, strains generated from this modified protocol would likely consist of many segregants, perhaps yielding measurements more comparable with previous studies, but inhibiting one from observing differences between independently constructed strains. These two contrasting approaches illustrate iSeq's flexibility, and we believe its applications will extend far beyond GI studies to any experiment aimed at uniquely identifying the origins of selected progeny derived from up to 10⁶ individual crosses.

Importantly, we illustrated iSeq's utility to measure variance between individual clonally derived strains with the same presumptive genotype by assaying several replicate strains in parallel. Performing iSeq with 4-8 independent constructs of the same double deletion, we found a high variance in both fitnesses and GI scores. The median correlation value for comparisons between our 8 replicate strains per double gene deletion was 0.42, similar to previous reports of 0.2 to 0.5 (Schuldiner: 2005; Jasnos: 2007; Dodgson: 2016). However, ours is the first study, to our knowledge, to use whole genome sequencing to investigate the underlying genetic variation that might confound GI measurements and lead to relatively low reproducibility. Our observation of new aneuploidies and SNPs after the first round of mating means mutations can accumulate very quickly, even during standard strain generation protocols requiring a single mating step. Furthermore, these new mutations occurred prior to the Gal-induced Cre activity, and were also observed in dubious ORF deletion carrying controls, leading us to believe they were not an artifact specific to the deletion strains we chose, or the barcoding system itself.

However, several factors could limit the bearing of our mutational findings on previous GI studies. First, to select haploids, our study used the magic marker construct carrying the MFA/MFalpha promoters which is more leaky and prone to diploidization than the construct with the STE2/STE3 promoters. Further experimental work would be required to directly compare rates of aneuploidy accumulation using either construct. However, it is also possible that the deletions we chose to examine have higher than average rates of mutation or chromosome segregation defects. Indeed, four of the double deletions we sequenced contain at least one gene shown to be involved in chromosome maintenance (SIN3, SDS3, and RPD3) (Wahba: 2011).

Additionally, we chose a set of deletions with generally severe fitness effects, which might be more likely to accumulate additional fitness-altering mutations. Consequently, we did observe a slightly elevated accumulation of aneuploidies and SNPs in our strains carrying gene deletions compared to those carrying dubious ORF deletions (FIG. 36A). Finally, our strains also went through one additional round of mating and selection compared with standard interaction studies, which provided more opportunity for mutations to arise and segregate across our experimental strains.

Regarding the specific mutations we observed in our strains, despite the fact that aneuploidy typically results in a growth defect, in some cases it can provide an advantage during stress and even help overcome the loss of a gene (Vernon: 2008; Pavelka: 2010; Yona: 2012; Liu: 2015). In our experiments, we find that chromosome V duplication was commonly observed in strains resulting from both the first and second rounds of mating and haploid selection, which conferred a growth advantage. The magic marker locus we used to select for haploids of a desired mating type (can1Δ::MFA1pr-HIS3-MFα1pr-LEU2), is located on chromosome V. It functions by expressing His3 or Leu2 under a MATa-dependent or MATα-dependent promoter, respectively. Thus, an extra copy of the magic marker locus created by duplication may produce more His3 or Leu2, providing a benefit during selection on media lacking histidine or leucine. In our pooled growth assays, however, we found that chromosome V duplication typically correlates with a decrease in fitness, suggesting that the selective advantage only occurs during strain construction. We lacked the statistical power to determine if rarer aneuploidies or SNPs also correlate with fitness. Of particular concern is that some of these variants may be deletion-specific suppressor mutations; these have been found in the deletion collection (Teng: 2013), and have been found to establish after only a few generations of growth (Szamecz: 2014). In our sequencing, we observed five cases of an aneuploidy of a chromosome rescuing a gene deletion.

There are several potential solutions to reduce the amount of segregating genetic variation and de novo mutations that is likely leading to the poor reproducibility of genetic interaction screens. To address the common chromosome V aneuploidy we observe (in 41% of sequenced strains), one potential solution would be to include, at the magic marker locus, a gene that can be tolerated in no more than two copies in the haploid (including one copy at the endogenous locus), such as CDC14 (Moriya: 2006). Alternatively, using the STE2/STE3 driven magic marker, or having the construct on a plasmid rather than genomically integrated may reduce the rates of accumulation of chromosome V aneuploidy. However, it is clear that not all genetic variation could be controlled in this manner A possible alternative approach, to minimize the generation of confounding genetic variation, would be to minimize the number of generations deletion strains undergo between the introduction of the gene deletion(s) and the fitness measurements. For example, inducible CRISPR/Cas9 systems that knockdown selected gene targets are available (Gilbert: 2013; Mans: 2015; Senturk: 2015; Smith: 2016), and these could be used in conjunction with iSeq, by integrating gRNAs at the same time and location as barcodes in order to generate inducible double knockdowns. This strategy could also be employed to search for interactions that include essential genes. Thus, a CRISPR/Cas9 approach combined with the iSeq double barcoding principle, is likely to provide a system by which to expand our view of genetic interaction networks from one that is static (one environment) to one that is dynamic (many environments).

### Materials and Methods:

### Yeast Barcode Library Construction

Two complementary barcode libraries, consisting of 288 clones each, were generated in a MATα starting strain derived from BY4742 (MATα ura3Δ0 leu2Δ0 his3Δ1 lys2Δ0) (Brachmann: 1998). This starting strain also carries the magic marker construct (Tong: 2004), which allows for selection of either MATa or MATα haploids via growth on synthetic complete (SC) media containing canavanine and lacking either histidine or leucine respectively. The barcode construct in each strain of each library sits at the dubious ORF YBR209W, and consists of a DNA barcode with 20 random nucleotides, a HygMX selectable marker, and either the 5′ half of the URA3 selectable marker and lox71 in the 5′ library, or the 3′ half of the URA3 selectable marker and lox66 in the 3′ library.

### Double-Barcoded Double-Deletion Yeast Strain Generation

Haploid gene deletion strains, carrying either KanMX or NatMX marked deletions, were derived from the diploid heterozygous deletion collection (Tong: 2001; Pan: 2004) for the following genes and dubious ORFs: ARP6, SAP30, SDS3, PHO23, SIN3, DGK1, SNT1, DEP1, RPD3, YHR095W and YFR054C. Each of the 11 deletion strains marked with KanMX was mated to two unique strains from the 5′ barcode construct carrying yeast library. NatMX marked deletion strains were each mated to two strains from the 3′ barcode construct carrying yeast library. Resulting diploid strains from each cross, and carrying a deletion and the barcode construct, were sporulated and plated for haploid single colonies.

To obtain strains carrying two gene deletions and both complementary barcode constructs, all pairwise combinations of singly barcoded deletion strain were mated. In each resulting diploid, Cre-mediated recombination was induced at the barcode locus by growing on SC+2% Galactose−Ura at 30° C. for 2 days. Cells were sporulated, and unsporulated diploids were digested using zymolyase as described (Herman: 1997) before selecting single haploid colonies.

### Pooled Growth

The 393 barcoded single and double gene deletion strains were frogged from frozen glycerol stocks to 1 mL liquid YPD in 2 mL 96-well plates, and placed at 30° C. After 3 days of growth, all strains were pooled, glycerol was added to a final concentration of 17% and aliquots were stored at −80° C. for future inoculations. The 8 barcoded WT control strains, generated from the matings of two dubious ORF barcoded deletion strains, were grown 0/N in liquid YPD, pooled, glycerol added and aliquots were stored at −80° C. for future inoculations.

The pooled fitness assay was carried out in 3 growth conditions: YPD, YPD 37° C. and YPEG (YP+2% EtOH, 2% Glycerol). The alternate conditions were chosen because in the Saccharomyces Genome Database, 7 of 9 of the single gene deletions are annotated as heat sensitive, and 4 of 9 have decreased respiratory growth.

For pooled growth fitness estimates, the double barcoded WT and double barcoded mutant pools were mixed at a 50:50 cellular ratio. For YPD, YPD 37° C., and YPEG cultures, 1.5625×10⁹, 6.25×10⁸, 6.78×10⁹ cells of this mixture were respectively used to inoculate 100 mL liquid of media in a 500 mL flask, in triplicate. The cells were cultured shaking at 230 rpm at 30° C. or 37° C. Every 24 hr, for a total of 8 time points, 12.5 mL culture were transferred to 87.5 mL fresh medium, and placed back in the incubator. At each transfer, the remaining overnight cultures were split into two 50 mL tubes, spun down and re-suspended in a 5 mL solution of 0.9M Sorbitol, 0.1M EDTA, 0.1M Tris-HCl pH 7.5 for DNA extractions.

### Barcode Sequencing

Barcode sequencing was done as previously described (Levy: 2015). Briefly, genomic DNA was extracted by spooling. A 2-step PCR was carried out on 14.4 μg genomic DNA to amplify the barcoded region, add multiplexing tags and add Illumina paired-end sequencing adaptors. Four initial time points were pooled and sequenced on the Illumina MiSeq.

Remaining libraries were pooled and paired-end sequencing was performed over 4 lanes on the Illumina HiSeq 2000 (10, 11, 20, and 23 libraries per lane). Additionally, 21 libraries were resequenced on one lane on Illumina HiSeq 2000 to test for sequencing noise.

Custom Python scripts were used to de-multiplex the time points from the Illumina data and to determine the number of reads matching each known double barcode in the pool at each time point.

### Fitness and Genetic Interaction Estimates

To estimate the fitness of each strain in the pool, barcode counts at each of the first four time points, were normalized for each strain by first dividing by the total number of counts at that time point to get a relative frequency. These frequencies were then normalized to the change in WT frequency, and then subsequently divided by the relative frequency at the first time point. After taking the natural logarithm of each of these normalized frequencies, a least squares linear regression was fit using the 1 m function in R, using a predefined intercept of 0. The fitness estimate for each strain was then defined as 1+m, where m is the slope of the fitted line.

To estimate quantitative genetic interaction scores, we calculated the deviation, ε, of the observed fitness of each double mutant strain (fᵢⱼ) in the pool from the expected fitness, based on the product of the observed fitness of the single mutant strains, fi and fj, as:

ε=fij−(fi×fj)

Fitness and interaction score estimates for each experimental strain across each replicate were calculated. To call interaction scores as significantly positive or negative, a 95% confidence interval was calculated around the mean score from the 4-8 strains with identical pairs of gene deletions.

### Optical Density Fitness Estimates

393 barcoded strains were streaked for single colonies on YPD. A single colony was used to inoculate a 2 mL overnight YPD culture. For three replicates of each strain, 2 μL of this O/N culture were used to inoculate 98 μL YPD in a 96-well plate. This plate was placed in the TECAN (GENios) and OD595 was taken every 15 minutes for 90 cycles, or 180 cycles for exceptionally slow growing strains.

To estimate fitness of each strain, the region of the curve during exponential growth was found for each strain by fitting a linear regression to each window of 10 time points, across all 90 total time points (90 total windows). This windowing method was employed to adjust for the fact that not all strains started at the same OD, and to avoid choosing arbitrary threshold values within which to calculate the doubling time. The fitted line corresponding to the window with the maximum slope, and therefore maximum growth rate, was used to calculate a doubling time for each strain. Fitness estimates were calculated by dividing the doubling time of a WT strain (generated above) that was included on the plate by the doubling time of the experimental strain (St Onge: 2007).

### Whole-Genome Sequencing

Strains were streaked for single colonies from frozen stocks, and grown up overnight in YPD at 30° C. Genomic DNA was isolated with the YeaStar Genomic DNA Kit (Zymo Research). Libraries for Illumina sequencing were constructed in 96-well format as previously described (Kryazhimskiy: 2014), pooled and analyzed for quality using Bioanalyzer (Agilent Technologies) and Qubit (Life Technologies) and sequenced on one lane of Illumina HiSeq 2000. Reads were trimmed for adaptors, quality and minimum length with cutadapt 1.7.1 (Martin: 2011). Reads were mapped to the reference genome with BWA version 0.7.10-r789 (Li: 2009a). And variants were called with GATK's Unified Genotyper v.3.3.0 (McKenna: 2010). Significant changes in copy number were discovered using the CNV-Seq package (Xie: 2009). SIFT was used to predict the protein function tolerance of amino acid changes resulting from SNPs verified by visual inspection using samtools tview and mpileup (Kumar: 2009; Li: 2009).

### Example 11: Tandem Barcoded Plasmid Integration and Sequencing in Mammalian Cells

I. Integration of Landing Pad into the ROSA26 Locus

A mouse and human tandem integration landing pad was designed and inserted it at the ROSA26 locus in each cell type. ROSA26 is “safe harbor” locus in the mammalian genome. Transgenes located at this site are unlikely to interfere with expression of endogenous genes and are presumably expressed in every cell type.

### 1. Construction of Landing Pad Plasmid

The landing pad plasmid pXYZ8 (SEQ ID NO: 95) includes the following major elements: two loxP variants, a Tamoxifen-inducible Cre recombinase and a drug resistant marker PGKpuropA flanked by the two FRT sites.

pXYZ8 (SEQ ID NO: 95) was constructed in three steps:

First, plasmids pXYZ1 (SEQ ID NO: 91) and pXYZ7 (SEQ ID NO: 94) were constructed from the following sources by standard methods: 1) plasmid backbone/bacterial origin from pUC19 (SEQ ID NO: 90), 2) PGK promoter, Puro R from MSCV-Puro (Clontech), 3) EFS promoter from plasmid lentiCRISPR-EGFP sgRNA4 (Addgene#51763), and 4) ERT2CreERT2 and pA from pCAG-ERT2CreERT2 (Addgene#13777).

Second, a landing pad element containing two loxP variant recombination sites (loxM3W and loxM1W), two FRT recombination sites, and an R recombination site was synthesized by IDT and integrated into pIDTUC-Amp plasmid (Integrated DNA Technologies, IDT) at EcoRV site to create pXYZ5 (SEQ ID NO: 92).

Third, The PGKpuropA and EFS-ERT2CreERT2 pA cassettes were sequentially cloned into pXYZ5 (SEQ ID NO: 92) by Gibson assembly: 1) PGKpuropA was amplified from pXYZ1 (SEQ ID NO: 91), and inserted between restriction sites NdeI and HpaI of pXYZ5 (SEQ ID NO: 92) to generate pXYZ6 (SEQ ID NO: 93) EFS-ERT2CreERT2 pA was amplified from pXYZ7 (SEQ ID NO: 94), and cloned into restriction site NotI of pXYZ6 (SEQ ID NO: 93) to generate pXYZ8. Because PGKpuropA is flanked by the two FRT sites, it can be excised out by FLP-FRT recombination at a downstream step.

### 2. Construction of Landing Pad Donor Plasmids

Donor plasmids containing the landing pad flanked by homology arms were constructed in two steps.

First, two plasmids containing ROSA26 homology arms (˜3 kb each) were constructed. pXYZ9 (SEQ ID NO: 96) contains mouse ROSA26 sequences, and pXYZ17 (SEQ ID NO: 98) contains human ROSA26 sequences. Any sequence of interest then can be easily inserted into pXYZ9 (SEQ ID NO: 96) or pXYZ17 (SEQ ID NO: 98) to construct different donor plasmids.

The left arm and right arms of mouse ROSA26 (mROSA26) were amplified from genomic DNA of 4T1 cells (ATCC® CRL-2539™) using the primers,

Left:
(SEQ ID NO: 36)
PXYZ007F = 5′ CAGGTCGACTCTAGAGGATCCTCGTCGTCTGATTGG
CTCT3′,
and
(SEQ ID NO: 37)
PXYZ007R = 5′ accagttatccctaGGAGGGACTCATTTAATATTAG
TCC3′.
Right:
(SEQ ID NO: 38)
PXYZ008F = 5′ ctagggataacagggtAATGAGCTATTAAGGCTTTT
TGTC3′,
and
(SEQ ID NO: 39)
PXYZ008R = 5′ GAGCTCGGTACCCGGGGATCCTCAAAAGAACCACTG
AGTA3′.

The left arm and right arms of human ROSA26 (hROSA26) were amplified from the genomic DNA from 293T celU (ATCC®CRL-3216™) using the primers,

Left:
PXYZ0011F =
(SEQ ID NO: 40)
5′ CAGGTCGACTCTAGAGGATCCGGGAGTACACACTCTCCTAAAA3′,
and
PXYZ0023R =
(SEQ ID NO: 41)
5′ attaccagttatccctaCATGGAGGCGATGACGAGATCA3′.
Right:
PXYZ0024F =
(SEQ ID NO: 42)
5′ tagggataacagggtaatAGTCGCTTCTCGATTATGGGCG3′,
and
PXYZ0012R =
(SEQ ID NO: 43)
5′ GAGCTCGGTACCCGGGGATCACCTGACCTGCAAGTTTCCAAAA3′.

Underlined sequences are homologous to 3′ and 5′ ends of linearized pUC19 (SEQ ID NO: 90) vector cut by BamHI. Sequences in italics are partial reverse complements of each other, contain the I-SceI restriction site and eventually form a cloning site to insert the landing pad. To generate the ROSA26 homology plasmids, purified left arm and right arm amplicons were mixed with pUC19 (SEQ ID NO: 90) cut with BamHI for Gibson assembly. The resulting plasmids are pXYZ9 (SEQ ID NO: 96) (mouse ROSA26,) and pXYZ17 (SEQ ID NO: 98) (human ROSA26).

To construct mouse donor plasmid pXYZ10 (SEQ ID NO: 97), the landing pad was amplified from pXYZ8 (SEQ ID NO: 95) using the primers:

(SEQ ID NO: 44)

PXYZ009F =
5′ TGAGTCCCTCCTAGGGATAAGACAGATCGACACTGCTCGA3′,
and

(SEQ ID NO: 45)

PXYZ009R =
5′ CTTAATAGCTCATTACCCTGGCTCGTCCAGAACTGATCCA3′,

where underlined sequences are homologous to the 3′ and 5′ ends of linearized pXYZ9 (SEQ ID NO: 96) cut by I-SceI. Purified PCR product derived from PXY009F and PXYZ009R was mixed with I-SceI digested pXYZ9 (SEQ ID NO: 96) for Gibson assembly to generate the donor plasmid pXYZ10 (SEQ ID NO: 97).

To construct human donor plasmid pXYZ18 (SEQ ID NO: 99), the landing pad was amplified from pXYZ8 (SEQ ID NO: 95) using the primers,

(SEQ ID NO: 46)

PXYZ0025F =
5′ TCGCCTCCATGTAGGGATAAGACAGATCGACACTGCTCGA3′,
and

(SEQ ID NO: 47)

PXYZ0025R =
5′ GAGAAGCGACTATTACCCCTGGCTCGTCCAGAACTGATCCA3′,

where underlined sequences are homologous to 3′ and 5′ end of linearized pXYZ17 (SEQ ID NO: 98) cut by I-SceI. Purified PCR product derived from PXY0025F and PXYZ0025R was mixed with I-SceI digested pXYZ17 for Gibson assembly to generate the donor plasmid pXYZ18 (SEQ ID NO: 99).

### 3. Integration of the Landing Pad at the Genomic ROSA26 Locus in Mammalian Cells

### 3.1 Construction of Cas9-sgRNA Plasmid.

3.11 sgRNA Design

We used CRISPR-mediated homology dependent repair (HDR) was used to achieve the integration of landing pad into the ROSA26 locus. Single guide RNA (sgRNA) guides nuclease Cas9 to cleave the target genomic locus, and then the donor plasmid containing homology arms acts as a template for repair the double strand breaks (DSBs). sgRNAs targeting the first intron of ROSA26 locus of mROSA26 and hROSA26 were identified using the CRISPR Design Tool (www.tools.genome-engineering.org).

3.12 sgRNA Cloning

sgRNA guide sequences were cloned into pX330-Cas9 (Addgene #42230, a vector containing Cas9 and the sgRNA scaffold) to generate plasmids that cut the ROSA26 locus.

For each sgRNA, a double stranded guide sequence flanked on either end by a cut BbsI restriction site was generated by annealing two synthesized oligos.

Oligo sequences for mROSA26 sgRNA are:

PFC001 F

(SEQ ID NO: 48)

5′ caccGCCCCTATAAAAGAGCTATTA3′,
and
PFC001 R

(SEQ ID NO: 49)

5′ aaacTAATAGCTCTTTTATAGGGGc3′.

Oligo sequences for hROSA26 sgRNA are:

PXYZ0022F

(SEQ ID NO: 50)

5′ caccgAATCGAGAAGCGACTCGACA3′,
and
PXYZ0022R

(SEQ ID NO: 51)

5′ aaacTGTCGAGTCGCTTCTCGATTc3′.

Underlined sequences are guide sequences provided by CRISPR Design Tool, and the lowercase letters indicate the BbsI overhangs for downstream ligation. Each oligo pair was annealed, and then ligated into the BbsI site in pX330-Cas9 (Ran: 2013).

3.2 Co-transfection of the Cas9-sgRNA plasmid and donor plasmid into mammalian cells.
3.21 For easily transfected cells (e.g. 293T, a Human Embryonic Kidney epithelial cell), 3-5×10⁵ cells were seeded in 6 cm dish on the day before transfection. Cell density was 50-80% confluent on the day of transfection. Cells were transfected with 1 ug of the specific Cas9-sgRNA plasmid and 1 ug of pXYZ18 (SEQ ID NO: 99) by standard lipid transfection methods, such as lipofectamine (Thermofisher).
3.22 For difficult to transfect cells (e.g. 4T1, Mouse Breast Tumor Epithelial cells), 2 μg of the specific Cas9-sgRNA plasmid and 2 μg of pXYZ10 (SEQ ID NO: 97) were electroporated into 1-2×10⁶ cells via 2b or 4D-Nucleofector (Amaxa).
3.3 Puromycin selection.

Approximately 24 h after transfection, cells were trypsinized and passed from a 6 cm dish to a 10 cm dish or from a 10 cm dish to a 15 cm dish. The next day, 1.5 μg/ml (for 293T) and 3 μg/ml (for 4T1) puromycin was added to the media. Cells were grown for 3-4 days, which was sufficient for puromycin selection.

### 4. Removal of the Puromycin Resistance Marker

To remove FRT-flanked PGKpuropA, cells were transfected with pCAG-Flpe:GFP (Addgene #13788), which contains a modified version of the Flp recombinase, Flpe. The next day, GFP positive cells were sorted by flow cytometry into 96-well plates such that each well contains a single cell. All wells were inspected to confirm that each contained a single colony ˜10 days after sorting.

### 5. Validation of Integration and PGKpuropA Removal

To check for proper integration of landing pad and removal of PGKpuropA, we isolated genomic DNA from each clonal cell line, and then genotyped each by PCR.

Integration at one end (upstream) in mouse cells was validated using the primers:

(SEQ ID NO: 52)

PXYZ0026F =
5′ GAGGGTCAGCGAAAGTAGCT3′,
and

(SEQ ID NO: 53)

PXYZ0027R2 =
5′ TCGAGCAGTGTCGATCTGTC3′.

Upstream integration in human cells was validated using the primers:

(SEQ ID NO: 54)

PXYZ0027F3 =
5′ GTGGGTATTCTCTGCTTTAGTC3′,
and

(SEQ ID NO: 55)

PXYZ0027R3 =
5′ CCGTAGGTAGTCACGCAACT3′.

Both forward primers prime the upstream region of the ROSA26 left arm, and both reverse primers prime the 5′ end of landing pad. Correct integration results in ˜3 kb band, but there is no band in non-transfected parental cells (FIG. 38, lane 1).

Downstream integration in mouse cells was validated using the primers:

(SEQ ID NO: 56)

PXYZ0030F2 =
5′ TGGATCAGTTCTGGACGAGC3′,
and

(SEQ ID NO: 57)

PXYZ0030R =
5′ GGAGCCATTCAGTGTTCACTAT3′.

Downstream integration in human cells was validated using the primers:

(SEQ ID NO: 58)

PXYZ0030F1 =
5′ CCAGTCATAGCTGTCCCTCT3′,
and

(SEQ ID NO: 59)

PXYZ0031R2 =
5′ GGACCCTGAAGTCTCTCTCCCA3′.

Both forward primers prime the 3′ end of landing pad, and both reverse primers prime the downstream region of ROSA26 right arm. Correct integration results in ˜3 kb band, but there is no band in non-transfected parental cells (FIG. 38, lane 3).

Heterozygosity of integration and PGKpuropA removal in human cells was validated using the primers:

(SEQ ID NO: 60)

PXYZ0029F3 = 5′ GTGATCTCGTCATCGCCTCCA3′,
and

(SEQ ID NO: 61)

PXYZ0029R3 = 5′ ACCAAGTTAGCCCCTTAAGCCT3′.

Heterozygosity of integration and puromycin removal in mouse cells was validated using the primers:

(SEQ ID NO: 62)

PXYZ0028F3 = 5′ GTCTGCAGCCATTACTAAACAT3′,
and

(SEQ ID NO: 63)

PXYZ0028R1 = 5′ CCCTTGGTTCTAAAGATACCACA.

Both forward primers prime the ROSA26 left arm, and both reverse primers prime the ROSA26 right arm.

Heterozygous integration results in two bands: In 4T1 cells, the wild-type mROSA26 locus (˜700 bp) and the integrated mROSA26 locus (˜5 kb, FIG. 38A, lane2 of clone A). In 293T cells, the wild-type hROSA26 locus (˜80 bp) and the integrated hROSA26 locus (˜4.3 kb, FIG. 38B, lane 2 of clone A).

Homozygous integration results in only one ˜4.3 kb band in 293T cells (FIG. 38B, lane 2 of clone B).

II. Barcoded Library Construction

Plasmid libraries compatible with the tandem integration landing pad were constructed to contain a loxP variant, a barcode and at least one drug resistance marker.

### 1. Construction of Drug Resistance Marker Cassettes

Plasmids containing the cassettes of different drug resistance markers or GFP: pXYZ23 (SEQ ID NO: 101), pXYZ24 (SEQ ID NO: 102), pXYZ25 (SEQ ID NO: 103), PXYZ26 (SEQ ID NO: 104), and pXYZ27 (SEQ ID NO: 105) were constructed by ligating a drug resistance markers or a GFP cassette into vector pCDNA3.1 (SEQ ID NO: 100) LIC (Addgene #30124), downstream of the CMV promoter.

PuroR was amplified from pXYZ1 (SEQ ID NO: 91) using the primers:

(SEQ ID NO: 64)
PXYZ0031F =
5′CCCaagcttGCCGCCACCATGACCGAGTACAAGCC3′,
and
(SEQ ID NO: 65)
PXYZ0031R = 5′GCCtctagaGCTAGCTTGCCAAACCTACA3′.

HygroR was amplified from MSCV-Hygro (Clontech) using the primers:

(SEQ ID NO: 66)
PXYZ0032F = 5′ CCCaagcttGCCGCCACCATGAAAAAGCCT3′,
and
(SEQ ID NO: 67)
PXYZ0032R = 5′ GCCtctagaCTTGTTCGGTCGGCATCTAC3′.

BlastiR was amplified from pLenti-6.3-V5 (Thermo Fisher) using the primers:

(SEQ ID NO: 68)
PXYZ0033F =
5′ CCCaagcttGCCGCCACCATGGCCAAGCCTTTGTC3′,
and
(SEQ ID NO: 69)
PXYZ0033R = 5′ GCCtctagaGTACCGAGCTCGAATTGTGC3′.

ZeoR was amplified from pBabe-HAZ (Addgene#17383) using the primers:

(SEQ ID NO: 70)
PXYZ0034F =
5′ CCCaagcttGCCGCCACCATGGCCAAGTTGACCAGTGCC3′,
and
(SEQ ID NO: 71)
PXYZ0034R = 5′ GCCtctagaCCAAACCTACAGGTGGGGT3′.

GFP was amplified from pCAGFlpe:GFP (Addgene#13788) using the primers:

(SEQ ID NO: 72)
PXYZ0035F = 5′ CCCaagcttGTCGCCACCATGGTGAGCAA3′,
and
(SEQ ID NO: 73)
PXYZ0035R = 5′ GCCtctagaGGAGTGCGGCCGCTTTACTT3′.

Underlined sequences are “Kozak consensus sequences” that improve translation efficiency, and the lowercase letters denote restriction sites. PCR products derived from each primer pair were digested with HindIII and XbaI, and ligated into linearized pCDNA3.1 (SEQ ID NO: 100) LIC cut by HindIII and XbaI.

### 2. Construction of Plasmid Backbone for Barcode Libraries

Two plasmids, BXL061 (SEQ ID NO: 107) and BXL064 (SEQ ID NO: 106), were constructed to form backbones for generation of complementary mammalian barcode libraries.

BXL061 (SEQ ID NO: 107) was constructed with the following steps: 1) pBAR4 (SEQ ID NO:26) was digested with NcoI and HpaI. A fragment that contains bacterial ampicillin resistance gene (AmpR), replication origin (ori) was purified. 2) Three oligonucleotides (pXL141, pXL142, and pXL143) were added to the DNA fragment from step 1 by Gibson Assembly to form two unique homing endonuclease sites (I-SceI and I-CeuI) and a multiple cloning site (MCS2).

(SEQ ID NO: 74)
pXL141 =
5′AAcagatcttgactgattatcTAGGGATAACAGGGTAATTAACTATA
ACGGTCCTAAGGTAGCGAGGGCCCATC3′.
(SEQ ID NO: 75)
pXL142 =
5′TAGCGAGGGCCCATCGATTGGCCATCGCGAATGCATCACGTGCTG
CAGCAGCTGGAGCTC3′.
(SEQ ID NO: 76)
pXL143 =
5′GCAGCAGCTGGAGCTCCCGCGGCCTGCAGGTACGTAAGGCCTAA
CCTGCATTAATGAATCG3′.

BXL064 (SEQ ID NO: 106) was constructed with the following steps: 1) pBAR3 was digested with PciI and a fragment containing AmpR and the ori was purified. 2) Three oligonucleotides (pXL142, pXL144, and pXL145) were inserted into the DNA fragment from step 1 by Gibson Assembly to form the same two homing endonuclease sites and a multiple cloning site (MCS2). 3) To form a second multiple cloning site (MCS1), the Gibson assembled construct from step 2 was digested with KpnI and NotI and ligated with double strand oligonucleotide that was formed by annealing pXLmcs and pXLmcs-r-m.

(SEQ ID NO: 77)
pXL144 =
5′GCTGGCCTTTTGCTCATAGGGATAACAGGGTAATTAACTATAACGGTC
CTAAGGTAGCGAGGGCCCATC3′.
(SEQ ID NO: 78)
pXL145 =
5′GCAGCAGCTGGAGCTCCCGCGGCCTGCAGGTACGTAAGGCCTTGGAT
GTATGTTAATATGG3′.
(SEQ ID NO: 79)
pXLmcs =
5′GGCCGCTTAATTAACAATTGGCTAGCCCCGGGGCATGCGGCGCCACTA
GTTGATCACGTACGCCTAGGTCTAGAC3′.
(SEQ ID NO: 80)
pXLmcs-r-m =
5′TCGAGTCTAGACCTAGGCGTACGTGATCAACTAGTGGCGCCGCATGCC
CCGGGGCTAGCCAATTGTTAATTAAGC3′.

LoxP variants loxW3M and loxW1M were inserted into vector BXL064 (SEQ ID NO: 106) and BXL061 (SEQ ID NO: 107), respectively.

### 3. Addition of Selectable Drug Markers

Drug resistance markers are used for selection of successful genomic integration of barcoded plasmids. PuroR and HygroR were added into BXL064 (SEQ ID NO: 106) and BXL061 (SEQ ID NO: 107), respectively, at MCS1 site using the following methods:

The CMV-PuroR-pA and CMV-HyroR-pA cassettes were amplified from pXYZ23 (SEQ ID NO: 101) and pXYZ24 (SEQ ID NO: 102) using the primers:

(SEQ ID NO: 81)

PXYZ0036F =
5′GCGTACGTGATCAACTAGTGGAGATCTCCCGATCCCCTAT3′,
and

(SEQ ID NO: 82)

PXYZ0036R =
5′TTAATTAACAATTGGCTAGCGCTGGCAAGTGTAGCGGTCA3′.

Underlined sequences are homologous to the 3′ and 5′ ends of linearized BXL064 (SEQ ID NO: 106) and BXL061 (SEQ ID NO: 107) cut by SpeI and NheI.

BXL064 (SEQ ID NO: 106) and BXL061 (SEQ ID NO: 107) were digested with NheI and SpeI. Purified PCR product CMV-PuroR-pA was mixed with linearized BXL064 (SEQ ID NO: 106), and Purified PCR product CMV-HyroR-pA was mixed with linearized BXL061 (SEQ ID NO: 107) for Gibson assembly, generating pXYZ28 (SEQ ID NO: 109) and pXYZ29 (SEQ ID NO: 110).

### 4. Insertion of Barcodes

Random barcodes were inserted into pXYZ28 (SEQ ID NO: 109) and pXYZ29 (SEQ ID NO: 110).

First, inserts containing a random 20 nucleotides and a unique loxP site (lox W3M or lox W1M) were generated by amplifying plasmid pBAR1 (SEQ ID NO:108) with primers P23 and either PXYZBC001 or PXYZBC002.

(SEQ ID NO: 3)
P23= 5′ GCCGAAATTGCCAGGATCAGG3′.
(SEQ ID NO: 83)
PXYZBC001 =
5′CCAGCTGGTACCNNNNNAANNNNNTTNNNNNTTNNNNNATAACTTCGT
ATAAaGTATcCTATACGAAcggtaGGCGCGCCGGCCGCAAAT3′.
(SEQ ID NO: 84)
PXYZBC002 =
5′CCAGCTGGTACCNNNNNAANNNNNAANNNNNTTNNNNNTtaccgTTCGT
ATAGCATACATTATACGAAGTTATGGCGCGCCGGCCGCAAAT3′.

Underlined sequences are loxP variants lox W3M (PXYZBC001) and lox W1M (PXYZBC002).

pXYZ28 (SEQ ID NO: 109), and pXYZ29 (SEQ ID NO: 110) were linearized by KpnI and XhoI. To generate a PuroR-loxW3M barcode library, PCR product derived from PXYZBC001 and P23 was digested by KpnI and XhoI and ligated into linearized pXYZ28 (SEQ ID NO: 109). To generate a HygroR-loxW1M barcode library, PCR product derived from PXYZBC002 and P23 was digested and ligated into linearized pXYZ29 (SEQ ID NO: 110). Ligation products were transformed into bacteria using standard methods, resulting in ˜100,000 barcode insertion events per plasmid.

### 5. Addition of the “Payload”

We next inserted different genetic elements (e.g. selection markers, sgRNA or open reading frames) into each barcode library (pXYZ28-W3M (SEQ ID NO: 111) and pXYZ29-W1M (SEQ ID NO: 112)) at a multicloning site (MCS2). Each payload will therefore be barcoded.

As one example, we inserted a second drug resistance selection marker or GFP into the pXYZ28-W3M (SEQ ID NO: 111) and pXYZ29-W1M (SEQ ID NO: 112) libraries at the MCS2 site by the following methods:

The CMV-BlastiR-pA, CMV-ZeoR-pA and CMV-GFP-pA cassettes were amplified using the primers:

(SEQ ID NO: 85)
PXYZ0038F = 5′TCGATTGGCCATCGCGAATGGGAGATCTCCCGATC
CCCTAT3′,
and
(SEQ ID NO: 86)
PXYZ0038R = 5′AGCTGCTGCAGCACGTGATGGCTGGCAAGTGTAGC
GGTCA3′.

The SV40-neoR-pA cassette was amplified using the primers:

(SEQ ID NO: 87)
PXYZ0039F = 5′TCGATTGGCCATCGCGAATGCGCGAATTAATTCTGT
GGAATGT3′,
and
(SEQ ID NO: 88)
PXYZ0039R = 5′AGCTGCTGCAGCACGTGATGAGGTCGACGGTATACA
GACAT3′.

Underlined sequences are homologous to the 3′ and 5′ ends of linearized pXYZ28-W3M (SEQ ID NO: 111) and pXYZ29-W1M (SEQ ID NO: 112) cut by BsmI. pXYZ28-W3M (SEQ ID NO: 111) and pXYZ29-W1M (SEQ ID NO: 112) were digested with BsmI. Purified PCR products were mixed with linearized pXYZ28-W3M (SEQ ID NO: 111) for Gibson assembly assay to construct library pXYZ28-W3M-BlastiR (SEQ ID NO: 113), pXYZ28-W3M-ZeoR (SEQ ID NO: 114), pXYZ28-W3M-neoR (SEQ ID NO: 115), pXYZ28-W3M-GFP (SEQ ID NO: 116).

Purified PCR products were mixed with linearized pXYZ29-W1M (SEQ ID NO: 112) for Gibson assembly assay to construct library pXYZ29-W1M-BlastiR (SEQ ID NO: 117), pXYZ29-W1M-ZeoR (SEQ ID NO: 118), pXYZ29-W1M-neoR (SEQ ID NO: 119), and pXYZ29-W1M-GFP (SEQ ID NO: 120). When the total number of payloads is small (e.g. <100), each selected transformant is likely to contain a unique barcode because the initial barcoded library complexity is high (˜100,000 barcodes).

III. Tandem Integration of Barcoded Plasmid Libraries at the Landing Pad

On day 1, equal concentrations of pXYZ28-W3M (SEQ ID NO: 111), pXYZ28-W3M-BlastiR (SEQ ID NO: 113), pXYZ28-W3M-ZeoR (SEQ ID NO: 114), pXYZ28-W3M-neoR (SEQ ID NO: 115), and pXYZ28-W3M-GFP (SEQ ID NO: 116) were electroporated into 1-2×10⁶ cells via 2b- or 4D-Nucleofector (Amaxa) and plated on 60 mm dishes. On day 2, cells were transferred to 100 mm dishes and cultured in the medium containing 1 μmol 4-Hydroxytamoxifen (4-OHT). 24 h post 4-OHT induction, we changed the medium, and 1.5 μg/ml puromycin was added to the medium. Cells were grown for 3-4 days, which was sufficient for puromycin selection. Cells with successful integration of the first library into the loxM3W site were then transfected with the second library containing equal concentrations of pXYZ29-W1M (SEQ ID NO: 112), pXYZ29-W1M-BlastiR (SEQ ID NO: 117), pXYZ29-W1M-ZeoR (SEQ ID NO: 118), pXYZ29-W1M-neoR (SEQ ID NO: 119), pXYZ29-W1M-GFP (SEQ ID NO: 120) by electroporation and plated on 60 mm dishes. Cells were transferred to 100 mm dishes at around 24 h post transfection. The next day, 800 μg/ml Hygromycin was added to the medium. Cells were grown for 3-4 days, which was sufficient for Hygromycin selection.

IV. Double Barcode Sequencing in Mammalian Cells

Cells were harvested, and genomic DNA was extracted. To reduce the complexity of DNA template during barcode PCR, genomic DNA sufficient to contain ˜500 copies of each double barcode was first digested with restriction endonuclease I-SceI (New England Biolabs) overnight at 37° C. Then, size selection for the barcode region was performed using SPRIselect beads (Beckman Coulter). Because the double barcodes region is flanked by two rare I-SceI sites, it is likely to be the only short DNA fragment recovered following size selection. To precipitate large genomic DNA fragments, we added 0.6× volume ratio (beads/sample) of beads. The supernatant, which contains the short double barcode DNA fragments, was removed from the beads and then we added 1.2× volume ratio of beads to precipitate the short double barcode DNA fragments to the beads. Double barcodes were eluted from the beads with water. A two-step PCR was performed using the size selected DNA, as described with modifications. First, a 3-cycle PCR with OneTaq polymerase (New England Biolabs) was performed. Primers for this reaction were:

(SEQ ID NO: 13)
5′ACACTCTTTCCCTACACGACGCTCTTCCGATCTNNNNNNNNXXXXXTT
AATATGGACTAAAGGAGGCTTTT3′,
and
(SEQ ID NO: 14)
5′CTCGGCATTCCTGCTGAACCGCTCTTCCGATCTNNNNNNNNXXXXXXX
XXTCGAATTCAAGCTTAGATCTGATA3′.

The Ns in these sequences correspond to any random nucleotide and are used in the downstream analysis to remove skew in the counts caused by PCR jackpotting. The Xs correspond to one of several multiplexing tags, which allow different samples to be distinguished when loaded on the same sequencing flow cell. PCR products were purified using SPRIselect beads with 1× volume ratio. A second 23-cycle PCR was performed with high-fidelity PrimeSTAR HS polymerase (Takara). Primers for this reaction were Illumina paired-end ligation primers:

(SEQ ID NO: 15)
pE1 =
5′AATGATACGGCGACCACCGAGATCTACACTCTTTCCCTACACGACGCT
CTTCCGATCT3′,
and
(SEQ ID NO: 16)
pE2 =
5′CAAGCAGAAGACGGCATACGAGATCGGTCTCGGCATTCCTGCTGAACC
GCTCTTCCGATCT3′.

PCR products were cleaned using SPRIselect beads with 1× volume ratio, and quantitated by Bioanalyzer (Agilent) and Qubit fluorometry (Life Technologies). Cleaned amplicons were pooled and sequenced on an Illumina MiSeq or HiSeq using paired end sequencing.

V. Integration of Two Plasmids that Each Contain a Portion of the Puromycin Gene Integrated into a Landing Pad in a Mammalian Cell Genome.

SEQ ID NO:121 depicts integration of two plasmids that each contain a portion of the puromycin gene integrated into a landing pad at the ROSA26 locus in mammalian cells. Both portions of the puromycin gene together provide puromycin resistance. Bases 5124-6654 include the two portions of the puromycin gene separated by an artificial intron that contains two barcodes and two loxP variants. The remaining sequence includes the up- and down-stream ROSA26 sequence, the two plasmid sequences, and other elements of the landing pad that include inducible Cre.

While there have been described what are presently believed to be the preferred embodiments of the present invention, those skilled in the art will realize that other and further changes and modifications may be made thereto without departing from the spirit of the invention, and it is intended to claim all such modifications and changes as come within the true scope of the invention.

TABLE 3
DNA sequence name and corresponding SEQ ID NO.

| DNA SEQUENCE | SEQ ID NO. |
|---|---|
| pBAR1 | 108 |
| pBAR4 | 26 |
| pBAR5 | 27 |
| pIDTUCAmp | 89 |
| pUC19 | 90 |
| pXYZ1 | 91 |
| pXYZ5 | 92 |
| pXYZ6 | 93 |
| pXYZ7 | 94 |
| pXYZ8 | 95 |
| pXYZ9 | 96 |
| pXYZ10 | 97 |
| pXYZ17 | 98 |
| pXYZ18 | 99 |
| pCDNA3.1 | 100 |
| pXYZ23 | 101 |
| pXYZ24 | 102 |
| pXYZ25 | 103 |
| pXYZ26 | 104 |
| pXYZ27 | 105 |
| pXYZ28 | 109 |
| pXYZ28-W3M | 111 |
| pXYZ28-W3M-BlastiR | 113 |
| pXYZ28-W3M-ZeoR | 114 |
| pXYZ28-W3M-neoR | 115 |
| pXYZ28-W3M-GFP | 116 |
| pXYZ29 | 110 |
| pXYZ29-W1M | 112 |
| pXYZ29-W1M-BlastiR | 117 |
| pXYZ29-W1M-ZeoR | 118 |
| pXYZ29-W1M-neoR | 119 |
| pXYZ29-W1M-GFP | 120 |
| BXL061 | 107 |
| BXL064 | 106 |
| Split Puromycin | 121 |

### INCORPORATION OF SEQUENCE LISTING

Incorporated herein by reference in its entirety is the Sequence Listing for the above-identified Application. The Sequence Listing is disclosed on a computer-readable ASCII text file titled “Sequence_Listing_178-435_PCT.txt”, created on Oct. 28, 2016. The sequence.txt file is 318 KB in size.

### REFERENCES

Bassik M. C., Kampmann M., Lebbink R. J., Wang S., Hein M. Y., Poser I., Weibezahn J., Horlbeck M. A., Chen S., Mann M., Hyman A. A., LeProust E. M., McManus M. T., Weissman J. S., A systematic mammalian genetic interaction map reveals pathways underlying ricin susceptibility. 2013 Cell 152: 909-922.
Bhang H.-E. C., Ruddy D. A., Krishnamurthy Radhakrishna V., Caushi J. X., Zhao R., Hims M. M., Singh A. P., Kao I., Rakiec D., Shaw P., Balak M., Raza A., Ackley E., Keen N., Schlabach M. R., Palmer M., Leary R. J., Chiang D. Y., Sellers W. R., Michor F., Cooke V. G., Korn J. M., Stegmeier F., Studying clonal dynamics in response to cancer therapy using high-complexity barcoding. 2015 Nat Med 21: 440-448.
Blundell J. R., Levy S. F., Beyond genome sequencing: Lineage tracking with barcodes to study the dynamics of evolution, infection, and cancer. 2014 Genomics. 104(6 Pt A):417-30.
Brachmann, C, Cost, G., Boeke, J. Designer deletion strains derived from Saccharomyces cerevisiae S288C: a useful set of strains and plasmids for PCR-mediated gene disruption and other applications. 1998 Yeast, 14: 115-32.
Butland G., Babu M., Díaz-Mejía J. J., Bohdana F., Phanse S., Gold B., Yang W., Li J., Gagarinova A. G., Pogoutse O., Mori H., Wanner B. L., Lo H., Wasniewski J., Christopoulos C., Ali M., Venn P., Safavi-Naini A., Sourour N., Caron S., Choi J.-Y., Laigle L., Nazarians-Armavil A., Deshpande A., Joe S., Datsenko K. A., Yamamoto N., Andrews B. J., Boone C., Ding H., Sheikh B., Moreno-Hagelsieb G., Greenblatt J. F., Emili A., eSGA: E. coli synthetic genetic array analysis. 2008 Nat. Methods 5: 789-795.
Cabantous S., Terwilliger T. C., Waldo G. S., Protein tagging and detection with engineered self-assembling fragments of green fluorescent protein. 2005 Nat Biotechnol 23: 102-107.
Christianson, T. R., Sikorski, R., Dante, M., Schero, J. and Hieter, P. Multifunctional yeast high copy-number shuttle vectors. 1992 Gene 110, 119-122.
Collins S. R., Schuldiner M., Krogan N. J., Weissman J. S., A strategy for extracting and analyzing large-scale quantitative epistatic interaction data. 2006 Genome Biol. 7: R63.
Costanzo M., Baryshnikova A., Bellay J., Kim Y., Spear E. D., Sevier C. S., Ding H., Koh J. L. Y., Toufighi K., Mostafavi S., Prinz J., St Onge R. P., VanderSluis B., Makhnevych T., Vizeacoumar F. J., Alizadeh S., Bahr S., Brost R. L., Chen Y., Cokol M., Deshpande R., Li Z., Lin Z.-Y., Liang W., Marback M., Paw J., San Luis B.-J., Shuteriqi E., Tong A. H. Y., van Dyk N., Wallace I. M., Whitney J. A., Weirauch M. T., Zhong G., Zhu H., Houry W. A., Brudno M., Ragibizadeh S., Papp B., Pal C., Roth F. P., Giaever G., Nislow C., Troyanskaya O. G., Bussey H., Bader G. D., Gingras A.-C., Morris Q. D., Kim P. M., Kaiser C. A., Myers C. L., Andrews B. J., Boone C., The genetic landscape of a cell. 2010 Science 327: 425-431.
Dodgson S. E., Kim S., Costanzo M., Baryshnikova A., Morse D. L., Kaiser C. A., Boone C., Amon A., Chromosome-Specific and Global Effects of Aneuploidy in Saccharomyces cerevisiae. 2016 Genetics 202: 1395-1409.
Galarneau A., Primeau M., Trudeau L. E., β-Lactamase protein fragment complementation assays as in vivo and in vitro sensors of protein-protein interactions. 2002 Nature 20: 619-622.
Gietz R D, Schiestl R H. Nat Protoc., Large-scale high-efficiency yeast transformation using the LiAc/SS carrier DNA/PEG method. 2007; 2(1):38-41.
Gilbert L. A., Larson M. H., Morsut L., Liu Z., Brar G. A., Torres S. E., Stern-Ginossar N., Brandman O., Whitehead E. H., Doudna J. A., Lim W. A., Weissman J. S., Qi L. S., CRISPR-mediated modular RNA-guided regulation of transcription in eukaryotes. 2013 Cell 154: 442-451.
Herman P. K., Rine J., Yeast spore germination: a requirement for Ras protein activity during re-entry into the cell cycle. 1997 EMBO J. 16: 6171-6181.
Ito T., Chiba T., Ozawa R., Yoshida M., Hattori M., Sakaki Y., A comprehensive two-hybrid analysis to explore the yeast protein interactome. 2001 Proc. Natl. Acad. Sci. U.S.A. 98: 4569-4574.
Jasnos L., Korona R., Epistatic buffering of fitness loss in yeast double deletion strains. 2007 Nat. Genet. 39: 550-554.
Kryazhimskiy S., Rice D. P., Jerison E. R., Desai M. M., Microbial evolution. Global epistasis makes adaptation predictable despite sequence-level stochasticity. 2014 Science 344: 1519-1522.
Kumar P., Henikoff S., Ng P. C., Predicting the effects of coding non-synonymous variants on protein function using the SIFT algorithm. 2009 Nat Protoc 4: 1073-1081.
Lee, G.; Saito, I., Role of nucleotide sequences of loxP spacer region in Cre-mediated recombination. 1998 Gene, 216, 55-65.
Levy S. F., Blundell J. R., Venkataram S., Petrov D. A., Fisher D. S., Sherlock G., Quantitative evolutionary dynamics using high-resolution lineage tracking. 2015 Nature 519: 181-186.
Li H., Durbin R., Fast and accurate short read alignment with Burrows-Wheeler transform. 2009a Bioinformatics 25: 1754-1760.
Li H., Handsaker B., Wysoker A., Fennell T., Ruan J., Homer N., Marth G., Abecasis G., Durbin R., 1000 Genome Project Data Processing Subgroup, The Sequence Alignment/Map format and SAMtools. 2009 Bioinformatics 25: 2078-2079.
Lindstrom, D. L. & Gottschling, D. E., The mother enrichment program: a genetic system for facile replicative life span analysis in Saccharomyces cerevisiae. 2009 Genetics 183, 413-22-1SI-13 SI.
Liu G., Yong M. Y. J., Yurieva M., Srinivasan K. G., Liu J., Lim J. S. Y., Poidinger M., Wright G. D., Zolezzi F., Choi H., Pavelka N., Rancati G., Gene Essentiality Is a Quantitative Property Linked to Cellular Evolvability. 2015 Cell 163: 1388-1399.
Mans R., van Rossum H. M., Wijsman M., Backx A., Kuijpers N. G. A., van den Broek M., Daran-Lapujade P., Fronk J. T., van Maris A. J. A., Daran J.-M. G., CRISPR/Cas9: a molecular Swiss army knife for simultaneous introduction of multiple genetic modifications in Saccharomyces cerevisiae. 2015 FEMS Yeast Res. 15.
Malleshaiah M K, Shahrezaei V, Swain P S, Michnick S W. The scaffold protein Ste5 directly controls a switch-like mating decision in yeast. 2010 Nature. 465(7294):101-5.
Martin M., Cutadapt removes adapter sequences from high-throughput sequencing reads. 2011 EMBnet.journal. 17: 10.
McKenna A., Hanna M., Banks E., Sivachenko A., Cibulskis K., Kernytsky A., Garimella K., Altshuler D., Gabriel S., Daly M., DePristo M. A., The Genome Analysis Toolkit: a MapReduce framework for analyzing next-generation DNA sequencing data. 2010 Genome Res. 20: 1297-1303.
Measday V., Baetz K., Guzzo J., Yuen K., Kwok T., Sheikh B., Ding H., Ueta R., Hoac T., Cheng B., Pot I., Tong A., Yamaguchi-Iwai Y., Boone C., Hieter P., Andrews B., Systematic yeast synthetic lethal and synthetic dosage lethal screens identify genes required for chromosome segregation. 2005 Proc. Natl. Acad. Sci. U.S.A. 102: 13956-13961.
Moriya H., Shimizu-Yoshida Y., Kitano H., In vivo robustness analysis of cell division cycle genes in Saccharomyces cerevisiae. 2006 PLoS Genet. 2: e111.
Pan X., Yuan D. S., Xiang D., Wang X., Sookhai-Mahadeo S., Bader J. S., Hieter P., Spencer F., Boeke J. D., A robust toolkit for functional profiling of the yeast genome. 2004 Molecular Cell 16: 487-496.
Pavelka N., Rancati G., Zhu J., Bradford W. D., Saraf A., Florens L., Sanderson B. W., Hattem G. L., Li R., Aneuploidy confers quantitative proteome changes and phenotypic variation in budding yeast. 2010 Nature 468: 321-325.
Schuldiner M., Collins S. R., Thompson N. J., Denic V., Bhamidipati A., Punna T., Ihmels J., Andrews B., Boone C., Greenblatt J. F., Weissman J. S., Krogan N. J., Exploration of the function and organization of the yeast early secretory pathway through an epistatic miniarray profile. 2005 Cell 123: 507-519.
Senturk S., Shirole N. H., Nowak D. D., Corbo V., Vaughan A., Tuveson D. A., Trotman L. C., Kepecs A., Stegmeier F., Sordella R., A rapid and tunable method to temporally control Cas9 expression enables the identification of essential genes and the interrogation of functional gene interactions in vitro and in vivo. 2015 bioRxiv 023366.
Smith J. D., Suresh S., Schlecht U., Wu M., Wagih O., Peltz G., Davis R. W., Steinmetz L. M., Parts L., St Onge R. P., Quantitative CRISPR interference screens in yeast identify chemical-genetic interactions and new rules for guide RNA design. 2016 Genome Biol. 17: 45.
Stark, C. Breitkreutz B J, Reguly T, Boucher L, Breitkreutz A, Tyers M., BioGRID: a general repository for interaction datasets. 2006 Nucleic Acids Res 34, D535-9.
St Onge R. P., Mani R., Oh J., Proctor M., Fung E., Davis R. W., Nislow C., Roth F. P., Giaever G., Systematic pathway analysis using high-resolution fitness profiling of combinatorial gene deletions. 2007 Nat. Genet. 39: 199-206.
Szamecz B., Boross G., Kalapis D., Kovacs K., Fekete G., Farkas Z., Lázár V., Hrtyan M., Kemmeren P., Groot Koerkamp M. J. A., Rutkai E., Holstege F. C. P., Papp B., Pál C., The genomic landscape of compensatory evolution. 2014 PLoS Biol. 12: e1001935.
Tarassov K., Messier V., Landry C. R., Radinovic S., Sema Molina M. M., Shames I., Malitskaya Y., Vogel J., Bussey H., Michnick S. W., An in vivo map of the yeast protein interactome. 2008 Science 320: 1465-1470.
Tavernier J., Eyckerman S., Lemmens I., Van der Heyden J., Vandekerckhove J., Van Ostade X., MAPPIT: a cytokine receptor-based two-hybrid method in mammalian cells. 2002 Clinical Experimental Allergy 32: 1397-1404.
Teng X., Dayhoff-Brannigan M., Cheng W.-C., Gilbert C. E., Sing C. N., Diny N. L., Wheelan S. J., Dunham M. J., Boeke J. D., Pineda F. J., Hardwick J. M., Genome-wide consequences of deleting any single gene. 2013 Mol. Cell 52: 485-494.
Tong A., Drees B., Nardelli G., Bader G., Brannetti B., Castagnoli L., Evangelista M., Ferracuti S., Nelson B., Paoluzi S., Quondam M., Zucconi A., Hogue C., Fields S., Boone C., Cesareni G., A combined experimental and computational strategy to define protein interaction networks for peptide recognition modules. 2002 Science 295: 321-4.
Tong A. H., Evangelista M., Parsons A. B., Xu H., Bader G. D., Pagé N., Robinson M., Raghibizadeh S., Hogue C. W., Bussey H., Andrews B., Tyers M., Boone C., Systematic genetic analysis with ordered arrays of yeast deletion mutants. 2001 Science 294: 2364-2368.
Tong, A.; Lesage, G.; Bader, G.; Ding, H.; Xu, H.; Xin, X.; Young, J.; Berriz, G.; Brost, R.; Chang, M.; Chen, Y.; Cheng, X.; Chua, G.; Friesen, H.; Goldberg, D.; Haynes, J.; Humphries, C.; He, G.; Hussein, S.; Ke, L.; Krogan, N.; Li, Z.; Levinson, J.; Lu, H.; Menard, P.; Munyana, C.; Parsons, A.; Ryan, O.; Tonikian, R.; Roberts, T.; Sdicu, A.; Shapiro, J.; Sheikh, B.; Suter, B.; Wong, S.; Zhang, L.; Zhu, H.; Burd, C.; Munro, S.; Sander, C.; Rine, J.; Greenblatt, J.; Peter, M.; Bretscher, A.; Bell, G.; Roth, F.; Brown, G.; Andrews, B.; Bussey, H.; Boone, C., Global mapping of the yeast genetic interaction network. 2004 Science, 303, 808-13.
Uetz P., Giot L., Cagney G., Mansfield T. A., Judson R. S., Knight J. R., Lockshon D., Narayan V., Srinivasan M., Pochart P., Qureshi-Emili A., Li Y., Godwin B., Conover D., Kalbfleisch T., Vijayadamodar G., Yang M., Johnston M., Fields S., Rothberg J. M., A comprehensive analysis of protein-protein interactions in Saccharomyces cerevisiae. 2000 Nature 403: 623-627.
van Dijk, E. L.; Auger, H.; Jaszczyszyn, Y.; Thermes, C., Ten years of next-generation sequencing technology. 2014 Trends in Genetics, 30, 418-426.
Vernon M., Lobachev K., Petes T. D., High rates of “unselected” aneuploidy and chromosome rearrangements in tell mec1 haploid yeast strains. 2008 Genetics 179: 237-247.
Voth W P, Jiang Y W, Stillman D J., New ‘marker swap’ plasmids for converting selectable markers on budding yeast gene disruptions and plasmids. 2003 Yeast August; 20(11):985-93.
Wahba L., Amon J. D., Koshland D., Vuica-Ross M., RNase H and multiple RNA biogenesis factors cooperate to prevent RNA:DNA hybrids from generating genome instability. 2011 Mol. Cell 44: 978-988.
Winzeler E. A., Shoemaker D. D., Astromoff A., Liang H., Anderson K., Andre B., Bangham R., Benito R., Boeke J. D., Bussey H., Chu A. M., Connelly C., Davis K., Dietrich F., Dow S. W., Bakkoury El M., Foury F., Friend S. H., Gentalen E., Giaever G., Hegemann J H., Jones T., Laub M., Liao H., Liebundguth N., Lockhart D. J., Lucau-Danila A., Lussier M., M'Rabet N., Menard P., Mittmann M., Pai C., Rebischung C., Revuelta J. L., Riles L., Roberts C. J., Ross-MacDonald P., Scherens B., Snyder M., Sookhai-Mahadeo S., Storms R. K., Véronneau S., Voet M., Volckaert G., Ward T. R., Wysocki R., Yen G. S., Yu K., Zimmermann K, Philippsen P., Johnston M., Davis R. W., Functional characterization of the S. cerevisiae genome by gene deletion and parallel analysis. 1999 Science 285: 901-906.
Wong A. S. L., Choi G. C. G., Cheng A. A., Purcell O., Lu T. K., Massively parallel high-order combinatorial genetics in human cells. 2015 Nat Biotechnol 33: 952-961.
Xie C., Tammi M. T., CNV-seq, a new method to detect copy number variation using high-throughput sequencing. 2009 BMC Bioinformatics 10: 80
Yona A. H., Manor Y. S., Herbst R. H., Romano G. H., Mitchell A., Kupiec M., Pilpel Y., Dahan O., Chromosomal duplication is a transient evolutionary solution to stress. 2012 Proc. Natl. Acad. Sci. U.S.A. 109: 21010-21015.

1. A method for placing at least two DNA sequences proximate to each other in a genome, the method comprising:
(a) providing the genome with a first site-specific recombination site;
(b) recombining the first site-specific recombination site with a third site-specific recombination site compatible with the first site-specific recombination site, wherein the third site-specific recombination site is associated with a first DNA sequence, thereby forming a first hybrid recombination site associated with the first DNA sequence, and a second hybrid recombination site;
(c) providing the genome with a second site-specific recombination site;
(d) recombining the second site-specific recombination site, with a fourth site-specific recombination site compatible with the second site-specific recombination site, wherein the fourth site-specific recombination site is associated with a second DNA sequence, thereby forming a third hybrid recombination site associated with the second DNA sequence, and a fourth hybrid recombination site;
(1) wherein steps (a), (b), (c), and (d) can be performed in any order;
(2) wherein any two, three, or four of steps (a), (b), (c), and (d) are optionally combined into a single step; and
whereby the first DNA sequence and the second DNA sequence are proximate to each other after recombining steps (b) and (d).

2. The method of claim 1, wherein the genome is in a cell.

3. The method of claim 1, wherein the first site-specific recombination site and the third site-specific recombination site are recombined with a recombinase specific for the first site-specific recombination site and the third site-specific recombination site.

4. The method of claim 1, wherein the second site-specific recombination site and the fourth site-specific recombination site are recombined with a recombinase specific for the second site-specific recombination site and the fourth site-specific recombination site.

5. The method of claim 1, wherein the first site-specific recombination site and the second site-specific recombination site are provided to the genome by means of a plasmid.

6. The method of claim 1, wherein the third site-specific recombination site associated with the first DNA sequence is on a plasmid, and is recombined with the first site-specific recombination site on the genome.

7. The method of claim 1, wherein the fourth site-specific recombination site associated with the second DNA sequence is on a plasmid, and is recombined with the first site-specific recombination site on the genome.

8. The method of claim 1, wherein the first site-specific recombination site and/or the second site-specific recombination site are selected from the group consisting of loxP, FRT, attP, attB, target sites for the R recombinase of Zygosaccharomyces rouxii (RS sites), variants thereof, and combinations thereof.

9. The method of claim 8, wherein the first site-specific recombination site and the second site-specific recombination site are incompatible with each other.

10. The method of claim 1, wherein the third site-specific recombination site is further associated with a third DNA sequence and/or the fourth site-specific recombination site is further associated with a fourth DNA sequence.

11. The method of claim 10, wherein the third DNA sequence and/or the fourth DNA sequence are selected from the group consisting of multiple-cloning sites, promoters, coding regions, sgRNA, gRNA, crRNA, miRNA, piRNA, siRNA, enhancers, intronic elements, and combinations thereof.

12. The method of claim 1, wherein the third site-specific recombination site is associated with a first portion of a split cell-selectable marker and the fourth site-specific recombination site is associated with a second portion of a split cell-selectable marker; wherein the first portion of a split cell-selectable marker and the second portion of a split cell-selectable marker are co-expressed in the genome to permit selection.

13. The method of claim 1, wherein the first DNA sequence and/or the second DNA sequence independently comprise a minimum of 4 nucleotides.

14. The method of claim 1, wherein the first DNA sequence and/or the second DNA sequence independently comprise a maximum of 300 nucleotides.

15. The method of claim 14, wherein the first DNA sequence and/or the second DNA sequence are selected from the group consisting of nucleic acid barcodes, promoters, coding regions, sgRNA, gRNA, crRNA, miRNA, piRNA, siRNA, enhancers, intronic elements, and combinations thereof.

16. The method of claim 1, wherein the first DNA sequence and the second DNA sequence are capable of being sequenced together via single-end or paired-end short-read sequencing.

17. The method of claim 1, wherein the method is performed on a large scale basis to create a library of genomes comprising at least two DNA sequences proximate to each other.

18. A kit comprising:
a first circular DNA library comprising a plurality of DNA molecules, wherein each DNA molecule comprises (i) a third site-specific recombination site, (ii) a plurality of first DNA sequences, and (iii) either a first cell-selectable marker or a first portion of a split cell-selectable marker or both; and
a second circular DNA library comprising a plurality of DNA molecules, wherein each DNA molecule comprises (i) a fourth site-specific recombination site, (ii) a plurality of second DNA sequences, and (iii) either a second cell-selectable marker or a second portion of a split cell-selectable marker or both.

19. A kit according to claim 18, further comprising:
a fifth DNA sequence comprising (i) a first site-specific recombination site compatible with the third site-specific recombination site (ii) a second site-specific recombination site compatible with the fourth site-specific recombination site; and
wherein the first site-specific recombination site is incompatible with the second site-specific recombination site;
wherein the third site-specific recombination site is incompatible with the second and fourth site-specific recombination sites;
wherein the fourth site-specific recombination site is incompatible with the first and third site-specific recombination sites;
wherein the fifth DNA sequence has a size that when the third site-specific recombination site recombines with the first site-specific recombination site; and (ii) the fourth site-specific integration recombines with the second site-specific recombination site, the first and second DNA sequences are proximate; and
with the proviso that when the first circular DNA library comprises a first portion of a cell-selectable marker and the second circular DNA library comprises a second portion of a split cell-selectable marker; the first portion and the second portion function to provide a functional selectable marker when both portions are co-expressed in a genome.

20.-23. (canceled)

24. The kit according to claim 18, wherein the fifth DNA sequence comprises: (i) flanking sequences that are homologous to a DNA sequence present on the genome; (ii)) a fifth site-specific recombination site at one flanking site and a seventh site-specific recombination site at the other flanking site, both of which are compatible with each other and with a sixth site-specific recombination site present in the genome; or (iii) a circular DNA molecule comprising a fifth site-specific recombination site compatible with a sixth site-specific recombination site present on the genome;
wherein the fifth, sixth, and seventh site-specific recombination sites are incompatible with site-specific recombination sites one, two, three, or four.

25.-36. (canceled)


SEQUENCE LISTING (121 sequences)

SEQ ID NO: 1 - DNA, 90 bp, Artificial Sequence
  Synthetic nucleotide sequence
  misc_feature (13)..(17): n is a, c, g, or t
  misc_feature (20)..(24): n is a, c, g, or t
  misc_feature (27)..(31): n is a, c, g, or t
  misc_feature (34)..(38): n is a, c, g, or t
ccagctggtaccnnnnnaannnnnttnnnnnttnnnnnataacttcgtataatgtatgctatacgaacggtaggcgcgccggccgcaaat

SEQ ID NO: 2 - DNA, 91 bp, Artificial Sequence
  Synthetic nucleotide sequence
  misc_feature (13)..(17): n is a, c, g, or t
  misc_feature (20)..(24): n is a, c, g, or t
  misc_feature (27)..(31): n is a, c, g, or t
  misc_feature (34)..(38): n is a, c, g, or t
ccagctggtaccnnnnnaannnnnaannnnnttnnnnnttaccgttcgtatagtacacattatacgaagttatggcgcgccggccgcaaat

SEQ ID NO: 3 - DNA, 21 bp, Artificial Sequence
  Synthetic nucleotide squence
gccgaaattgccaggatcagg

SEQ ID NO: 4 - DNA, 65 bp, Artificial Sequence
  Synthetic nucleotide sequence
gttctttgctttttttccccaacgacgtcgaacacattagtcctacgcacttaacttcgcatctg

SEQ ID NO: 5 - DNA, 70 bp, Artificial Sequence
  Synthetic nucleotide sequence
gcttgcgctaactgcgaacagagtgccctatgaaataggggaatgcatatcatacgtaatgctcaacctt

SEQ ID NO: 6 - DNA, 19 bp, Artificial Sequence
  Synthetic nucleotide sequence
gcgaacagagtaaaccgaa

SEQ ID NO: 7 - DNA, 18 bp, Artificial Sequence
  Synthetic nucleotide sequence
gaaggtctgaaggagttc

SEQ ID NO: 8 - DNA, 70 bp, Artificial Sequence
  Synthetic nucleotide sequence
atctgtttagcttgcctcgtccccgccgggtcacccggccagcgacatggagattgtactgagagtgcac

SEQ ID NO: 9 - DNA, 70 bp, Artificial Sequence
  Synthetic nucleotide sequence
aacatgttctttgctttttttccccaacgacgtcgaacacattagtcctactgtgcggtatttcacaccg

SEQ ID NO: 10 - DNA, 193 bp, Artificial Sequence
  Synthetic nucleotide sequence
agatctgtttagcttgcctcgtccccgccgggtcacccggccagcgacatggtaccgttcgtataatgtatgctatacgaagttattgcgcggtgatcacttatggtaccgttcgtataatgtgtactatacgaagttattaggactaatgtgttcgacgtcgttggggaaaaaaagcaaagaacatgttgcc

SEQ ID NO: 11 - DNA, 193 bp, Artificial Sequence
  Synthetic nucleotide sequence
agatctgtttagcttgcctcgtccccgccgggtcacccggccagcgacatggtaccgttcgtataatgtatgctatacgaagttattgcgcggtgatcacttatggtaccgttcgtataaagtatcctatacgaagttattaggactaatgtgttcgacgtcgttggggaaaaaaagcaaagaacatgttgcc

SEQ ID NO: 12 - DNA, 193 bp, Artificial Sequence
  Synthetic nucleotide sequence
agatctgtttagcttgcctcgtccccgccgggtcacccggccagcgacatggataacttcgtataaagtatcctatacgaacggtatgcgcggtgatcacttatggtaccgttcgtataatgtgtactatacgaagttattaggactaatgtgttcgacgtcgttggggaaaaaaagcaaagaacatgttgcc

SEQ ID NO: 13 - DNA, 71 bp, Artificial Sequence
  Synthetic sequence
  misc_feature (34)..(46): n is a, c, g, or t
acactctttccctacacgacgctcttccgatctnnnnnnnnnnnnnttaatatggactaaaggaggctttt

SEQ ID NO: 14 - DNA, 74 bp, Artificial Sequence
  Synthetic sequence
  misc_feature (34)..(50): n is a, c, g, or t
ctcggcattcctgctgaaccgctcttccgatctnnnnnnnnnnnnnnnnntcgaattcaagcttagatctgata

SEQ ID NO: 15 - DNA, 58 bp, Artificial Sequence
  Synthetic sequence
aatgatacggcgaccaccgagatctacactctttccctacacgacgctcttccgatct

SEQ ID NO: 16 - DNA, 61 bp, Artificial Sequence
  Synthetic sequence
caagcagaagacggcatacgagatcggtctcggcattcctgctgaaccgctcttccgatct

SEQ ID NO: 17 - DNA, 34 bp, Artificial Sequence
  Artificial sequence
ataacttcgtataatgtatgctatacgaagttat

SEQ ID NO: 18 - DNA, 34 bp, Artificial Sequence
  Synthetic sequence
taccgttcgtataatgtatgctatacgaagttat

SEQ ID NO: 19 - DNA, 34 bp, Artificial Sequence
  Synthetic sequence
ataacttcgtataatgtatgctatacgaacggta

SEQ ID NO: 20 - DNA, 34 bp, Artificial Sequence
  Synthetic sequence
ataacttcgtataatgtgtactatacgaagttat

SEQ ID NO: 21 - DNA, 34 bp, Artificial Sequence
  Synthetic sequence
taccgttcgtataatgtgtactatacgaagttat

SEQ ID NO: 22 - DNA, 34 bp, Artificial Sequence
  Synthetic sequence
ataacttcgtataatgtgtactatacgaacggta

SEQ ID NO: 23 - DNA, 34 bp, Artificial Sequence
  Synthetic sequence
ataacttcgtataaagtatcctatacgaagttat

SEQ ID NO: 24 - DNA, 34 bp, Artificial Sequence
  Synthetic sequence
taccgttcgtataaagtatcctatacgaagttat

SEQ ID NO: 25 - DNA, 34 bp, Artificial Sequence
  Synthetic sequence
ataacttcgtataaagtatcctatacgaacggta

SEQ ID NO: 26 - DNA, 5149 bp, Artificial Sequence
  Synthetic sequence
gaacgcggccgcttaattaacaattggctagccccggggcatgcggcgccactagttgatcacgtacgcctaggtctagactcgagtcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcggccggcgcgcccttaagcaggagggtaccgatatcagatctaagcttgaattcgaatttttactaacaaatggtattatttataacagatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattaccggtcacccggccagcgacatggaggcccagaataccctccttgacagtcttgacgtgcgcagctcaggggcatgatgtgactgtcgcccgtacatttagcccatacatccccatgtataatcatttgcatccatacattttgatggccgcacggcgcgaagcaaaaattacggctcctcgctgcagacctgcgagcagggaaacgctcccctcacagacgcgttgaattgtccccacgccgcgcccctgtagagaaatataaaaggttaggatttgccactgaggttcttctttcatatacttccttttaaaatcttgctaggatacagttctcacatcacatccgaacataaacaaccatgggtaaggaaaagactcacgtttcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggcaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagtactgacaataaaaagattcttgttttcaagaacttgtcatttgtatagtttttttatattgtagttgttctattttaatcaaatgttagcgtgatttatattttttttcgcctcgacatcatctgcccagatgcgaagttaagtgcgcagaaagtaatatcatgcgtcaatcgtatgtgaatgctggtcgctatactgctgtcgattcgatactaacgccgccatccagtgtcgaaaacgagctctaaggttgagcattacgtatgatatgtccatgtacaataattaaatatgaattaggagaaagacttagcttcttttcgggtgatgtcacttaaaaactccgagaataatatataataagagaataaaatattagttattgaataagaactgtaaatcagctggcgttagtctgctaatggcagcttcatcttggtttattgtagcatgaatcatatttgcctttttttcctgtaattcaatgattcttgcttctatactatcctcaatgcaaaaccttgtgggcccgttaacctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatggacatattgtcgttagaacgcggctacaattaatacataaccttatgtatcatacacatacgatttaggtgacactata

SEQ ID NO: 27 - DNA, 4990 bp, Artificial Sequence
  Synthetic sequence
tatagtgtcacctaaatcgtatgtgtatgatacataaggttatgtattaattgtagccgcgttctaacgacaatatgtccatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcaggttaacgggcccaatagttttgccagcggaattccacttgcaattacataaaaaattccggcggtttttcgcgtgtgactcaatgtcgaaatacctgcctaatgaacatgaacatcgcccaaatgtatttgaagacccgctgggagaagttcaagatatataagtaacaagcagccaatagtataaaaaaaaatctgagtttattacctttcctggaatttcagtgaaaaactgctaattatagagagatatcacagagttactcactaatggagctctcagtactgacaataaaaagattcttgttttcaagaacttgtcatttgtatagtttttttatattgtagttgttctattttaatcaaatgttagcgtgatttatattttttttcgcctcgacatcatctgcccagatgcgaagttaagtgcgcagaaagtaatatcatgcgtcaatcgtatgtgaatgctggtcgctatactgctgtcgattcgatactaacgccgccatccagtgtcgaaaacttattcctttgccctcggacgagtgctggggcgtcggtttccactatcggcgagtacttctacacagccatcggtccagacggccgcgcttctgcgggcgatttgtgtacgcccgacagtcccggctccggatcggacgattgcgtcgcatcgaccctgcgcccaagctgcatcatcgaaattgccgtcaaccaagctctgatagagttggtcaagaccaatgcggagcatatacgcccggagccgcggcgatcctgcaagctccggatgcctccgctcgaagtagcgcgtctgctgctccatacaagccaaccacggcctccagaagaagatgttggcgacctcgtattgggaatccccgaacatcgcctcgctccagtcaatgaccgctgttatgcggccattgtccgtcaggacattgttggagccgaaatccgcgtgcacgaggtgccggacttcggggcagtcctcggcccaaagcatcagctcatcgagagcctgcgcgacggacgcactgacggtgtcgtccatcacagtttgccagtgatacacatggggatcagcaatcgcgcatatgaaatcacgccatgtagtgtattgaccgattccttgcggtccgaatgggccgaacccgctcgtctggctaagatcggccgcagcgatcgcatccatggcctccgcgaccggctgcagaacagcgggcagttcggtttcaggcaggtcttgcaacgtgacaccctgtgcacggcgggagatgcaataggtcaggctctcgctgaattccccaatgtcaagcacttccggaatcgggagcgcggccgatgcaaagtgccgataaacataacgatctttgtagaaaccatcggcgcagctatttacccgcaggacatatccacgccctcctacatcgaagctgaaagcacgagattcttcgccctccgagagctgcatcaggtcggagacgctgtcgaacttttcgatcagaaacttctcgacagacgtcgcggtgagttcaggctttttacccataggcccagaataccctccttgacagtcttgacgtgcgcagctcaggggcatgatgtgactgtcgcccgtacatttagcccatacatccccatgtataatcatttgcatccatacattttgatggccgcacggcgcgaagcaaaaattacggctcctcgctgcagacctgcgagcagggaaacgctcccctcacagacgcgttgaattgtccccacgccgcgcccctgtagagaaatataaaaggttaggatttgccactgaggttcttctttcatatacttccttttaaaatcttgctaggatacagttctcacatcacatccgaacataaacaaccccatgtcgctggccgggtgaccgattcggtaatctccgaacagaaggaagaacgaaggaaggagcacagacttagattggtatatatacgcatatgtagtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatgtatgttaatatggactaaaggaggcttttgtcgacggatccgatatcggtaccctcctgcttaagggcgcgccggccgcaaattaaagccttcgagcgtcccaaaaccttctcaagcaaggttttcagtataatgttacatgcgtacacgcgtctgtacagaaaaaaaagaaaaatttgaaatataaataacgttcttaatactaacataactataaaaaaataaatagggacctagacttcaggttgtctaactccttccttttcggttagagcggatgtggggggagggcgtgaatgtaagcgtgacataactaattacatgactcgagtctagacctaggcgtacgtgatcaactagtggcgccgcatgccccggggctagccaattgttaattaagcggccgcgttc

SEQ ID NO: 28 - DNA, 26 bp, Artificial Sequence
  Synthetic sequence
  misc_feature (1)..(5): n is a, c, g, or t
  misc_feature (8)..(12): n is a, c, g, or t
  misc_feature (15)..(19): n is a, c, g, or t
  misc_feature (22)..(26): n is a, c, g, or t
nnnnnaannnnnaannnnnttnnnnn

SEQ ID NO: 29 - DNA, 21 bp, Artificial Sequence
  Synthetic sequence
ggcggtggcggatcaggaggc

SEQ ID NO: 30 - DNA, 24 bp, Artificial Sequence
  Synthetic sequence
ttcgacactggatggcggcgttag

SEQ ID NO: 31 - DNA, 90 bp, Artificial Sequence
  Synthetic sequence
  misc_feature (13)..(17): n is a, c, g, or t
  misc_feature (20)..(24): n is a, c, g, or t
  misc_feature (27)..(31): n is a, c, g, or t
  misc_feature (34)..(38): n is a, c, g, or t
ccagctggtaccnnnnnaannnnnttnnnnnttnnnnnataacttcgtatagcatacattatacgaacggtaggcgcgccggccgcaaat

SEQ ID NO: 32 - DNA, 90 bp, Artificial Sequence; identical to SEQ ID NO: 31
  Synthetic sequence
  misc_feature (13)..(17): n is a, c, g, or t
  misc_feature (20)..(24): n is a, c, g, or t
  misc_feature (27)..(31): n is a, c, g, or t
  misc_feature (34)..(38): n is a, c, g, or t

SEQ ID NO: 33 - DNA, 65 bp, Artificial Sequence
  Synthetic sequence
gcttgcgctaactgcgaacagagtgccctatgaaataggggaatgcgcacttaacttcgcatctg

SEQ ID NO: 34 - DNA, 70 bp, Artificial Sequence
  Synthetic sequence
gttctttgctttttttccccaacgacgtcgaacacattagtcctacatatcatacgtaatgctcaacctt

SEQ ID NO: 35 - DNA, 23 bp, Artificial Sequence
  Synthetic sequence
caacctgaagtctaggtcctatt

SEQ ID NO: 36 - DNA, 40 bp, Artificial Sequence
  Synthetic sequence
caggtcgactctagaggatcctcgtcgtctgattggctct

SEQ ID NO: 37 - DNA, 40 bp, Artificial Sequence
  Synthetic sequence
accctgttatccctaggagggactcatttaatattagtcc

SEQ ID NO: 38 - DNA, 40 bp, Artificial Sequence
  Synthetic sequence
ctagggataacagggtaatgagctattaaggctttttgtc

SEQ ID NO: 39 - DNA, 40 bp, Artificial Sequence
  Synthetic sequence
gagctcggtacccggggatcctcaaaagaaccactgagta

SEQ ID NO: 40 - DNA, 43 bp, Artificial Sequence
  Synthetic sequence
caggtcgactctagaggatccgggagtacacactctcctaaaa

SEQ ID NO: 41 - DNA, 40 bp, Artificial Sequence
  Synthetic sequence
attaccctgttatccctacatggaggcgatgacgagatca

SEQ ID NO: 42 - DNA, 40 bp, Artificial Sequence
  Synthetic sequence
tagggataacagggtaatagtcgcttctcgattatgggcg

SEQ ID NO: 43 - DNA, 43 bp, Artificial Sequence
  Synthetic sequence
gagctcggtacccggggatcacctgacctgcaagtttccaaaa

SEQ ID NO: 44 - DNA, 40 bp, Artificial Sequence
  Synthetic sequence
tgagtccctcctagggataagacagatcgacactgctcga

SEQ ID NO: 45 - DNA, 40 bp, Artificial Sequence
  Synthetic sequence
cttaatagctcattaccctggctcgtccagaactgatcca

SEQ ID NO: 46 - DNA, 40 bp, Artificial Sequence
  Synthetic sequence
tcgcctccatgtagggataagacagatcgacactgctcga

SEQ ID NO: 47 - DNA, 41 bp, Artificial Sequence
  Synthetic sequence
gagaagcgactattacccctggctcgtccagaactgatcca

SEQ ID NO: 48 - DNA, 25 bp, Artificial Sequence
  Synthetic sequence
caccgcccctataaaagagctatta

SEQ ID NO: 49 - DNA, 25 bp, Artificial Sequence
  Synthetic sequence
aaactaatagctcttttataggggc

SEQ ID NO: 50 - DNA, 25 bp, Artificial Sequence
  Synthetic sequence
caccgaatcgagaagcgactcgaca

SEQ ID NO: 51 - DNA, 25 bp, Artificial Sequence
  Synthetic sequence
aaactgtcgagtcgcttctcgattc

SEQ ID NO: 52 - DNA, 20 bp, Artificial Sequence
  Synthetic sequence
gagggtcagcgaaagtagct

SEQ ID NO: 53 - DNA, 20 bp, Artificial Sequence
  Synthetic sequence
tcgagcagtgtcgatctgtc

SEQ ID NO: 54 - DNA, 22 bp, Artificial Sequence
  Synthetic sequence
gtgggtattctctgctttagtc

SEQ ID NO: 55 - DNA, 20 bp, Artificial Sequence
  Synthetic sequence
ccgtaggtagtcacgcaact

SEQ ID NO: 56 - DNA, 20 bp, Artificial Sequence
  Synthetic sequence
tggatcagttctggacgagc

SEQ ID NO: 57 - DNA, 22 bp, Artificial Sequence
  Synthetic sequence
ggagccattcagtgttcactat

SEQ ID NO: 58 - DNA, 20 bp, Artificial Sequence
  Synthetic sequence
ccagtcatagctgtccctct

SEQ ID NO: 59 - DNA, 22 bp, Artificial Sequence
  Synthetic sequence
ggaccctgaagtctctctccca

SEQ ID NO: 60 - DNA, 21 bp, Artificial Sequence
  Synthetic sequence
gtgatctcgtcatcgcctcca

SEQ ID NO: 61 - DNA, 22 bp, Artificial Sequence
  Synthetic sequence
accaagttagccccttaagcct

SEQ ID NO: 62 - DNA, 22 bp, Artificial Sequence
  Synthetic sequence
gtctgcagccattactaaacat

SEQ ID NO: 63 - DNA, 23 bp, Artificial Sequence
  Synthetic sequence
cccttggttctaaagataccaca

SEQ ID NO: 64 - DNA, 35 bp, Artificial Sequence
  Synthetic sequence
cccaagcttgccgccaccatgaccgagtacaagcc

SEQ ID NO: 65 - DNA, 29 bp, Artificial Sequence
  Synthetic sequence
gcctctagagctagcttgccaaacctaca

SEQ ID NO: 66 - DNA, 30 bp, Artificial Sequence
  Synthetic sequence
cccaagcttgccgccaccatgaaaaagcct

SEQ ID NO: 67 - DNA, 29 bp, Artificial Sequence
  Synthetic sequence
gcctctagacttgttcggtcggcatctac

SEQ ID NO: 68 - DNA, 35 bp, Artificial Sequence
  Synthetic sequence
cccaagcttgccgccaccatggccaagcctttgtc

SEQ ID NO: 69 - DNA, 29 bp, Artificial Sequence
  Synthetic sequence
gcctctagagtaccgagctcgaattgtgc

SEQ ID NO: 70 - DNA, 39 bp, Artificial Sequence
  Synthetic sequence
cccaagcttgccgccaccatggccaagttgaccagtgcc

SEQ ID NO: 71 - DNA, 28 bp, Artificial Sequence
  Synthetic sequence
gcctctagaccaaacctacaggtggggt

SEQ ID NO: 72 - DNA, 29 bp, Artificial Sequence
  Synthetic sequence
cccaagcttgtcgccaccatggtgagcaa

SEQ ID NO: 73 - DNA, 29 bp, Artificial Sequence
  Synthetic sequence
gcctctagaggagtgcggccgctttactt

SEQ ID NO: 74 - DNA, 75 bp, Artificial Sequence
  Synthetic sequence
aacagatcttgactgatttttctagggataacagggtaattaactataacggtcctaaggtagcgagggcccatc

SEQ ID NO: 75 - DNA, 60 bp, Artificial Sequence
  Synthetic sequence
tagcgagggcccatcgattggccatcgcgaatgcatcacgtgctgcagcagctggagctc

SEQ ID NO: 76 - DNA, 61 bp, Artificial Sequence
  Synthetic sequence
gcagcagctggagctcccgcggcctgcaggtacgtaaggcctaacctgcattaatgaatcg

SEQ ID NO: 77 - DNA, 69 bp, Artificial Sequence
  Synthetic sequence
gctggccttttgctcatagggataacagggtaattaactataacggtcctaaggtagcgagggcccatc

SEQ ID NO: 78 - DNA, 61 bp, Artificial Sequence
  Synthetic sequence
gcagcagctggagctcccgcggcctgcaggtacgtaaggccttggatgtatgttaatatgg

SEQ ID NO: 79 - DNA, 75 bp, Artificial Sequence
  Synthetic sequence
ggccgcttaattaacaattggctagccccggggcatgcggcgccactagttgatcacgtacgcctaggtctagac

SEQ ID NO: 80 - DNA, 75 bp, Artificial Sequence
  Synthetic sequence
tcgagtctagacctaggcgtacgtgatcaactagtggcgccgcatgccccggggctagccaattgttaattaagc

SEQ ID NO: 81 - DNA, 40 bp, Artificial Sequence
  Synthetic sequence
gcgtacgtgatcaactagtggagatctcccgatcccctat

SEQ ID NO: 82 - DNA, 40 bp, Artificial Sequence
  Synthetic sequence
ttaattaacaattggctagcgctggcaagtgtagcggtca

SEQ ID NO: 83 - DNA, 90 bp, Artificial Sequence
  Synthetic sequence
  misc_feature (13)..(17): n is a, c, g, or t
  misc_feature (20)..(24): n is a, c, g, or t
  misc_feature (27)..(31): n is a, c, g, or t
  misc_feature (34)..(38): n is a, c, g, or t
ccagctggtaccnnnnnaannnnnttnnnnnttnnnnnataacttcgtataaagtatcctatacgaacggtaggcgcgccggccgcaaat

SEQ ID NO: 84 - DNA, 91 bp, Artificial Sequence
  Synthetic sequence
  misc_feature (13)..(17): n is a, c, g, or t
  misc_feature (20)..(24): n is a, c, g, or t
  misc_feature (27)..(31): n is a, c, g, or t
  misc_feature (34)..(38): n is a, c, g, or t
ccagctggtaccnnnnnaannnnnaannnnnttnnnnnttaccgttcgtatagcatacattatacgaagttatggcgcgccggccgcaaat

SEQ ID NO: 85 - DNA, 41 bp, Artificial Sequence
  Synthetic sequence
tcgattggccatcgcgaatgggagatctcccgatcccctat

SEQ ID NO: 86 - DNA, 40 bp, Artificial Sequence
  Synthetic sequence
agctgctgcagcacgtgatggctggcaagtgtagcggtca

SEQ ID NO: 87 - DNA, 43 bp, Artificial Sequence
  Synthetic sequence
tcgattggccatcgcgaatgcgcgaattaattctgtggaatgt

SEQ ID NO: 88 - DNA, 41 bp, Artificial Sequence
  Synthetic sequence
agctgctgcagcacgtgatgaggtcgacggtatacagacat

SEQ ID NO: 89 - DNA, 2752 bp, Artificial Sequence
  Synthetic sequence
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccaaatgcggtgtgaaataccgcacagatgcgtaaggagaaaataccgcatcaggcgccattcgccattcaggctgcgcaactgttgggaagggcgatcggtgcgggcctcatcgctattacgccagctggcgaaagggggatgtgctgcaaggcgattaagttgggtaacgccagggttttcccagtcacgacgttgtaaaacgacggccagtgcaacgcgatgacgatggatagcgattcatcgatgagctgacccgatcgcggccgccggagggttgcgtttgagacgggcgacagatatcagttctggaccagcgagctgtgctgcgacgcgtggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctctttcgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgtcgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactctacctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 90 - DNA, 2686 bp, Artificial Sequence
  Synthetic sequence
gcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttgcatgcctgcaggtcgactctagaggatccccgggtaccgagctcgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaaga

SEQ ID NO: 91 - DNA, 4496 bp, Artificial Sequence
  Synthetic sequence
ggatccccgggtaccgagctcgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttgcatgcctgcaggtcgactctagaggatccgatctctcgaggttaacgaattctaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacctgcagcccaagcttaccatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgacgcccgccccacgacccgcagcgcccgaccgaaaggagcgcacgaccccatgcatcgataaaataaaagattttatttagtctccagaaaaaggggggaatgaaagaccccacctgtaggtttggcaagctagcgtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgccatatgctggctgccatgaacaaaggtggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatgaagatccctcgacctg

SEQ ID NO: 92 - DNA, 3251 bp, Artificial Sequence
  Synthetic sequence
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccaaatgcggtgtgaaataccgcacagatgcgtaaggagaaaataccgcatcaggcgccattcgccattcaggctgcgcaactgttgggaagggcgatcggtgcgggcctcatcgctattacgccagctggcgaaagggggatgtgctgcaaggcgattaagttgggtaacgccagggttttcccagtcacgacgttgtaaaacgacggccagtgcaacgcgatgacgatggatagcgattcatcgatgagctgacccgatcgccgccgccggagggttgcgtttgagacgggcgacagatgacagatcgacactgctcgaaagttcagatgtgcggcgagttgcgtgactacctacgggtaacagtttcttaccgttcgtataaagtatcctatacgaagttatttatggcagggtgaaacgcaggtcgccagctaccgttcgtataatgtatgctatacgaagttatctttcggcggtgaaattatcgatgagcgtggtggttatgccgatcgcgtctccacgtgcatcaaggcgcgccattgatgcggccgcatctagaagttcctattccgaagttcctattctctagaaagtataggaacttcgcagaatcatatggaatgacttggttgagttaactgttagtagaagttcctattccgaagttcctattctctagaaagtataggaacttcggatcagtttaaacagtctgacgagatcatatcactgtggacgttgatgaaagaatacgttattctttcatcaaatcgtgtcgtggatcagttctggacgagcatcagttctggaccagcgagctgtgctgcgactcgtggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgtcgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactctacctttttcaatattattgaagcatttatcagggttattgtccatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 93 - DNA, 5036 bp, Artificial Sequence
  Synthetic sequence
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccaaatgcggtgtgaaataccgcacagatgcgtaaggagaaaataccgcatcaggcgccattcgccattcaggctgcgcaactgttgggaagggcgatcggtgcgggcctcatcgctattacgccagctggcgaaagggggatgtgctgcaaggcgattaagttgggtaacgccagggttttcccagtcacgacgttgtaaaacgacggccagtgcaacgcgatgacgatggatagcgattcatcgatgagctgacccgatcgccgccgccggagggttgcgtttgagacgggcgacagatgacagatcgacactgctcgaaagttcagatgtgcggcgagttgcgtgactacctacgggtaacagtttcttaccgttcgtataaagtatcctatacgaagttatttatggcagggtgaaacgcaggtcgccagctaccgttcgtataatgtatgctatacgaagttatctttcggcggtgaaattatcgatgagcgtggtggttatgccgatcgcgtctccacgtgcatcaaggcgcgccattgatgcggccgcatctagaagttcctattccgaagttcctattctctagaaagtataggaacttcgcagaatcatagatctctcgaggttaacgaattctaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacctgcagcccaagcttaccatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgacgcccgccccacgacccgcagcgcccgaccgaaaggagcgcacgaccccatgcatcgataaaataaaagattttatttagtctccagaaaaaggggggaatgaaagaccccacctgtaggtttggcaagctagcgtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgccatatgctggctgccatgaacaaaggtggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatgaagatccctcgacctgaactgttagtagaagttcctattccgaagttcctattctctagaaagtataggaacttcggatcagccgcggcagtctgacgagatcatatcactgtggacgttgatgaaagaatacgttattctttcatcaaatcgtgtcgtggatcagttctggacgagcatcagttctggaccagcgagctgtgctgcgactcgtggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgtcgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactctacctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 94 - DNA, 6468 bp, Artificial Sequence
  Synthetic sequence
gatccccgggtaccgagctcgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttgcatgcctgcaggtcgactctagaggatcgggacagcagagatccactttggcgccggctcgagtggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtgtcgtgacgcggggcaaagaattcccgggtgagccgccaccatggctggagacatgagagctgccaacctttggccaagcccgctcatgatcaaacgctctaagaagaacagcctggccttgtccctgacggccgaccagatggtcagtgccttgttggatgctgagccccccatactctattccgagtatgatcctaccagacccttcagtgaagcttcgatgatgggcttactgaccaacctggcagacagggagctggttcacatgatcaactgggcgaagagggtgccaggctttgtggatttgaccctccatgatcaggtccaccttctagaatgtgcctggctagagatcctgatgattggtctcgtctggcgctccatggagcacccagtgaagctactgtttgctcctaacttgctcttggacaggaaccagggaaaatgtgtagagggcatggtggagatcttcgacatgctgctggctacatcatctcggttccgcatgatgaatctgcagggagaggagtttgtgtgcctcaaatctattattttgcttaattctggagtgtacacatttctgtccagcaccctgaagtctctggaagagaaggaccatatccaccgagtcctggacaagatcacagacactttgatccacctgatggccaaggcaggcctgaccctgcagcagcagcaccagcggctggcccagctcctcctcatcctctcccacatcaggcacatgagtaacaaaggcatggagcatctgtacagcatgaagtgcaagaacgtggtgcccctctatgacctgctgctggaggcggcggacgcccaccgcctacatgcgcccactagccgtggaggggcatccgtggaggagacggaccaaagccacttggccactgcgggctctacttcatcgcattccttgcaaaagtattacatcacgggggaggcagagggtttccctgccacagctgtcgacaatttactgaccgtacaccaaaatttgcctgcattaccggtcgatgcaacgagtgatgaggttcgcaagaacctgatggacatgttcagggatcgccaggcgttttctgagcatacctggaaaatgcttctgtccgtttgccggtcgtgggcggcatggtgcaagttgaataaccggaaatggtttcccgcagaacctgaagatgttcgcgattatcttctatatcttcaggcgcgcggtctggcagtaaaaactatccagcaacatttgggccagctaaacatgcttcatcgtcggtccgggctgccacgaccaagtgacagcaatgctgtttcactggttatgcggcggatccgaaaagaaaacgttgatgccggtgaacgtgcaaaacaggctctagcgttcgaacgcactgatttcgaccaggttcgttcactcatggaaaatagcgatcgctgccaggatatacgtaatctggcatttctggggattgcttataacaccctgttacgtatagccgaaattgccaggatcagggttaaagatatctcacgtactgacggtgggagaatgttaatccatattggcagaacgaaaacgctggttagcaccgcaggtgtagagaaggcacttagcctgggggtaactaaactggtcgagcgatggatttccgtctctggtgtagctgatgatccgaataactacctgttttgccgggtcagaaaaaatggtgttgccgcgccatctgccaccagccagctatcaactcgcgccctggaagggatttttgaagcaactcatcgattgatttacggcgctaaggatgactctggtcagagatacctggcctggtctggacacagtgcccgtgtcggagccgcgcgagatatggcccgcgctggagtttcaataccggagatcatgcaagctggtggctggaccaatgtaaatattgtcatgaactatatccgtaacctggatagtgaaacaggggcaatggtgcgcctgctggaagatggcgatctcgagccatctgctggagacatgagagctgccaacctttggccaagcccgctcatgatcaaacgctctaagaagaacagcctggccttgtccctgacggccgaccagatggtcagtgccttgttggatgctgagccccccatactctattccgagtatgatcctaccagacccttcagtgaagcttcgatgatgggcttactgaccaacctggcagacagggagctggttcacatgatcaactgggcgaagagggtgccaggctttgtggatttgaccctccatgatcaggtccaccttctagaatgtgcctggctagagatcctgatgattggtctcgtctggcgctccatggagcacccagtgaagctactgtttgctcctaacttgctcttggacaggaaccagggaaaatgtgtagagggcatggtggagatcttcgacatgctgctggctacatcatctcggttccgcatgatgaatctgcagggagaggagtttgtgtgcctcaaatctattattttgcttaattctggagtgtacacatttctgtccagcaccctgaagtctctggaagagaaggaccatatccaccgagtcctggacaagatcacagacactttgatccacctgatggccaaggcaggcctgaccctgcagcagcagcaccagcggctggcccagctcctcctcatcctctcccacatcaggcacatgagtaacaaaggcatggagcatctgtacagcatgaagtgcaagaacgtggtgcccctctatgacctgctgctggaggcggcggacgcccaccgcctacatgcgcccactagccgtggaggggcatccgtggaggagacggaccaaagccacttggccactgcgggctctacttcatcgcattccttgcaaaagtattacatcacgggggaggcagagggtttccctgccacagcttgatagcggccgcactcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgccatatgctggctgccatgaacaaaggtggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatgaagatccctcgacctgc

SEQ ID NO: 95 - DNA, 8862 bp, Artificial Sequence
  Synthetic sequence
ggccgcatctagaagttcctattccgaagttcctattctctagaaagtataggaacttcgcagaatcatagatctctcgaggttaacgaattctaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacctgcagcccaagcttaccatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgacgcccgccccacgacccgcagcgcccgaccgaaaggagcgcacgaccccatgcatcgataaaataaaagattttatttagtctccagaaaaaggggggaatgaaagaccccacctgtaggtttggcaagctagcgtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgccatatgctggctgccatgaacaaaggtggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatgaagatccctcgacctgaactgttagtagaagttcctattccgaagttcctattctctagaaagtataggaacttcggatcagccgcggcagtctgacgagatcatatcactgtggacgttgatgaaagaatacgttattctttcatcaaatcgtgtcgtggatcagttctggacgagcatcagttctggaccagcgagctgtgctgcgactcgtggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgtcgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactctacctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccaaatgcggtgtgaaataccgcacagatgcgtaaggagaaaataccgcatcaggcgccattcgccattcaggctgcgcaactgttgggaagggcgatcggtgcgggcctcatcgctattacgccagctggcgaaagggggatgtgctgcaaggcgattaagttgggtaacgccagggttttcccagtcacgacgttgtaaaacgacggccagtgcaacgcgatgacgatggatagcgattcatcgatgagctgacccgatcgccgccgccggagggttgcgtttgagacgggcgacagatgacagatcgacactgctcgaaagttcagatgtgcggcgagttgcgtgactacctacgggtaacagtttcttaccgttcgtataaagtatcctatacgaagttatttatggcagggtgaaacgcaggtcgccagctaccgttcgtataatgtatgctatacgaagttatctttcggcggtgaaattatcgatgagcgtggtggttatgccgatcgcgtctccacgtgcatcaaggcgcgccattgatgcggccgggacagcagagatccactttggcgccggctcgagtggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtgtcgtgacgcggggcaaagaattcccgggtgagccgccaccatggctggagacatgagagctgccaacctttggccaagcccgctcatgatcaaacgctctaagaagaacagcctggccttgtccctgacggccgaccagatggtcagtgccttgttggatgctgagccccccatactctattccgagtatgatcctaccagacccttcagtgaagcttcgatgatgggcttactgaccaacctggcagacagggagctggttcacatgatcaactgggcgaagagggtgccaggctttgtggatttgaccctccatgatcaggtccaccttctagaatgtgcctggctagagatcctgatgattggtctcgtctggcgctccatggagcacccagtgaagctactgtttgctcctaacttgctcttggacaggaaccagggaaaatgtgtagagggcatggtggagatcttcgacatgctgctggctacatcatctcggttccgcatgatgaatctgcagggagaggagtttgtgtgcctcaaatctattattttgcttaattctggagtgtacacatttctgtccagcaccctgaagtctctggaagagaaggaccatatccaccgagtcctggacaagatcacagacactttgatccacctgatggccaaggcaggcctgaccctgcagcagcagcaccagcggctggcccagctcctcctcatcctctcccacatcaggcacatgagtaacaaaggcatggagcatctgtacagcatgaagtgcaagaacgtggtgcccctctatgacctgctgctggaggcggcggacgcccaccgcctacatgcgcccactagccgtggaggggcatccgtggaggagacggaccaaagccacttggccactgcgggctctacttcatcgcattccttgcaaaagtattacatcacgggggaggcagagggtttccctgccacagctgtcgacaatttactgaccgtacaccaaaatttgcctgcattaccggtcgatgcaacgagtgatgaggttcgcaagaacctgatggacatgttcagggatcgccaggcgttttctgagcatacctggaaaatgcttctgtccgtttgccggtcgtgggcggcatggtgcaagttgaataaccggaaatggtttcccgcagaacctgaagatgttcgcgattatcttctatatcttcaggcgcgcggtctggcagtaaaaactatccagcaacatttgggccagctaaacatgcttcatcgtcggtccgggctgccacgaccaagtgacagcaatgctgtttcactggttatgcggcggatccgaaaagaaaacgttgatgccggtgaacgtgcaaaacaggctctagcgttcgaacgcactgatttcgaccaggttcgttcactcatggaaaatagcgatcgctgccaggatatacgtaatctggcatttctggggattgcttataacaccctgttacgtatagccgaaattgccaggatcagggttaaagatatctcacgtactgacggtgggagaatgttaatccatattggcagaacgaaaacgctggttagcaccgcaggtgtagagaaggcacttagcctgggggtaactaaactggtcgagcgatggatttccgtctctggtgtagctgatgatccgaataactacctgttttgccgggtcagaaaaaatggtgttgccgcgccatctgccaccagccagctatcaactcgcgccctggaagggatttttgaagcaactcatcgattgatttacggcgctaaggatgactctggtcagagatacctggcctggtctggacacagtgcccgtgtcggagccgcgcgagatatggcccgcgctggagtttcaataccggagatcatgcaagctggtggctggaccaatgtaaatattgtcatgaactatatccgtaacctggatagtgaaacaggggcaatggtgcgcctgctggaagatggcgatctcgagccatctgctggagacatgagagctgccaacctttggccaagcccgctcatgatcaaacgctctaagaagaacagcctggccttgtccctgacggccgaccagatggtcagtgccttgttggatgctgagccccccatactctattccgagtatgatcctaccagacccttcagtgaagcttcgatgatgggcttactgaccaacctggcagacagggagctggttcacatgatcaactgggcgaagagggtgccaggctttgtggatttgaccctccatgatcaggtccaccttctagaatgtgcctggctagagatcctgatgattggtctcgtctggcgctccatggagcacccagtgaagctactgtttgctcctaacttgctcttggacaggaaccagggaaaatgtgtagagggcatggtggagatcttcgacatgctgctggctacatcatctcggttccgcatgatgaatctgcagggagaggagtttgtgtgcctcaaatctattattttgcttaattctggagtgtacacatttctgtccagcaccctgaagtctctggaagagaaggaccatatccaccgagtcctggacaagatcacagacactttgatccacctgatggccaaggcaggcctgaccctgcagcagcagcaccagcggctggcccagctcctcctcatcctctcccacatcaggcacatgagtaacaaaggcatggagcatctgtacagcatgaagtgcaagaacgtggtgcccctctatgacctgctgctggaggcggcggacgcccaccgcctacatgcgcccactagccgtggaggggcatccgtggaggagacggaccaaagccacttggccactgcgggctctacttcatcgcattccttgcaaaagtattacatcacgggggaggcagagggtttccctgccacagcttgatagcggccgcactcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgccatatgctggctgccatgaacaaaggtggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatgaagatccctcgacctgcgatccccgggtaccgagctgcctgcaggtcgactctagaggatc

SEQ ID NO: 96 - DNA, 8738 bp, Artificial Sequence
  Synthetic sequence
gatccccgggtaccgagctcgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttgcatgcctgcaggtcgactctagaggatcctcgtcgtctgattggctctcggggcccagaaaactggcccttgccattggctcgtgttcgtgcaagttgagtccatccgccggccagcgggggcggcgaggaggcgctcccaggttccggccctcccctcggccccgcgccgcagagtctggccgcgcgcccctgcgcaacgtggcaggaagcgcgcgctgggggcggggacgggcagtagggctgagcggctgcggggcgggtgcaagcacgtttccgacttgagttgcctcaagaggggcgtgctgagccagacctccatcgcgcactccggggagtggagggaaggagcgagggctcagttgggctgttttggaggcaggaagcacttgctctcccaaagtcgctctgagttgttatcagtaagggagctgcagtggagtaggcggggagaaggccgcacccttctccggaggggggaggggagtgttgcaatacctttctgggagttctctgctgcctcctggcttctgaggaccgccctgggcctgggagaatcccttccccctcttccctcgtgatctgcaactccagtctttctagaagatgggcgggagtcttctgggcaggcttaaaggctaacctggtgtgtgggcgttgtcctgcaggggaattgaacaggtgtaaaattggagggacaagacttcccacagattttcggttttgtcgggaagttttttaataggggcaaataaggaaaatgggaggataggtagtcatctggggttttatgcagcaaaactacaggttattattgcttgtgatccgcctcggagtattttccatcgaggtagattaaagacatgctcacccgagttttatactctcctgcttgagatccttactacagtatgaaattacagtgtcgcgagttagactatgtaagcagaattttaatcatttttaaagagcccagtacttcatatccatttctcccgctccttctgcagccttatcaaaaggtattttagaacactcattttagccccattttcatttattatactggcttatccaacccctagacagagcattggcattttccctttcctgatcttagaagtctgatgactcatgaaaccagacagattagttacatacaccacaaatcgaggctgtagctggggcctcaacactgcagttcttttataactccttagtacactttttgttgatcctttgccttgatccttaattttcagtgtctatcacctctcccgtcaggtggtgttccacatttgggcctattctcagtccagggagttttacaacaatagatgtattgagaatccaacctaaagcttaactttccactcccatgaatgcctctctcctttttctccatttataaactgagctattaaccattaatggtttccaggtggatgtctcctcccccaatattacctgatgtatcttacatattgccaggctgatattttaagacattaaaaggtatatttcattattgagccacatggtattgattactgcttactaaaattttgtcattgtacacatctgtaaaaggtggttccttttggaatgcaaagttcaggtgtttgttgtctttcctgacctaaggtcttgtgagcttgtattttttctatttaagcagtgctttctcttggactggcttgactcatggcattctacacgttattgctggtctaaatgtgattttgccaagcttcttcaggacctataattttgcttgacttgtagccaaacacaagtaaaatgattaagcaacaaatgtatttgtgaagcttggtttttaggttgttgtgttgtgtgtgcttgtgctctataataatactatccaggggctggagaggtggctcggagttcaagagcacagactgctcttccagaagtcctgagttcaattcccagcaaccacatggtggctcacaaccatctgtaatgggatctgatgccctcttctggtgtgtctgaagaccacaagtgtattcacattaaataaataaatcctccttcttcttcttttttttttttttaaagagaatactgtctccagtagaatttactgaagtaatgaaatactttgtgtttgttccaatatggtagccaataatcaaattactctttaagcactggaaatgttaccaaggaactaatttttatttgaagtgtaactgtggacagaggagccataactgcagacttgtgggatacagaagaccaatgcagactttaatgtcttttctcttacactaagcaataaagaaataaaaattgaacttctagtatcctatttgtttaaactgctagctttacttaacttttgtgcttcatctatacaaagctgaaagctaagtctgcagccattactaaacatgaaagcaagtaatgataattttggatttcaaaaatgtagggccagagtttagccagccagtggtggtgcttgcctttatgcctttaatcccagcactctggaggcagagacaggcagatctctgagtttgagcccagcctggtctacacatcaagttctatctaggatagccaggaatacacacagaaaccctgttggggaggggggctctgagatttcataaaattataattgaagcattccctaatgagccactatggatgtggctaaatccgtctacctttctgatgagatttgggtattattttttctgtctctgctgttggttgggtcttttgacactgtgggctttctttaaagcctccttcctgccatgtggtctcttgtttgctactaacttcccatggcttaaatggcatggctttttgccttctaagggcagctgctgagatttgcagcctgatttccagggtggggttgggaaatctttcaaacactaaaattgtcctttaattttttttttaaaaaatgggttatataataaacctcataaaatagttatgaggagtgaggtggactaatattaaatgagtccctcctagggataacagggtaatgagctattaaggctttttgtcttatacttaactttttttttaaatgtggtatctttagaaccaagggtcttagagttttagtatacagaaactgttgcatcgcttaatcagattttctagtttcaaatccagagaatccaaattcttcacagccaaagtcaaattaagaatttctgacttttaatgttaatttgcttactgtgaatataaaaatgatagcttttcctgaggcagggtctcactatgtatctctgcctgatctgcaacaagatatgtagactaaagttctgcctgcttttgtctcctgaatactaaggttaaaatgtagtaatacttttggaacttgcaggtcagattcttttataggggacacactaagggagcttgggtgatagttggtaaaatgtgtttcaagtgatgaaaacttgaattattatcaccgcaacctactttttaaaaaaaaaagccaggcctgttagagcatgcttaagggatccctaggacttgctgagcacacaagagtagttacttggcaggctcctggtgagagcatatttcaaaaaacaaggcagacaaccaagaaactacagttaaggttacctgtctttaaaccatctgcatatacacagggatattaaaatattccaaataatatttcattcaagttttcccccatcaaattgggacatggatttctccggtgaataggcagagttggaaactaaacaaatgttggttttgtgatttgtgaaattgttttcaagtgatagttaaagcccatgagatacagaacaaagctgctatttcgaggtctcttggtttatactcagaagcacttctttgggtttccctgcactatcctgatcatgtgctaggcctaccttaggctgattgttgttcaaataaacttaagtttcctgtcaggtgatgtcatatgatttcatatatcaaggcaaaacatgttatatatgttaaacatttgtacttaatgtgaaagttaggtctttgtgggtttgatttttaattttcaaaacctgagctaaataagtcatttttacatgtcttacatttggtggaattgtataattgtggtttgcaggcaagactctctgacctagtaaccctacctatagagcactttgctgggtcacaagtctaggagtcaagcatttcaccttgaagttgagacgttttgttagtgtatactagtttatatgttggaggacatgtttatccagaagatattcaggactatttttgactgggctaaggaattgattctgattagcactgttagtgagcattgagtggcctttaggcttgaattggagtcacttgtatatctcaaataatgctggccttttttaaaaagcccttgttctttatcaccctgttttctacataatttttgttcaaagaaatacttgtttggatctccttttgacaacaatagcatgttttcaagccatattttttttcctttttttttttttttttggtttttcgagacagggtttctctgtatagccctggctgtcctggaactcactttgtagaccaggctggcctcgaactcagaaatccgcctgcctctgcctcctgagtgccgggattaaaggcgtgcaccaccacgcctggctaagttggatattttgttatataactataaccaatactaactccactgggtggatttttaattcagtcagtagtcttaagtggtctttattggcccttcattaaaatctactgttcactctaacagaggctgttggtactagtggcacttaagcaacttcctacggatatactagcagattaagggtcagggatagaaactagtctagcgttttgtatacctaccagctttatactaccttgttctgatagaaatatttcaggacatctagagtgtactataaggttgatggtaagcttataaggaacttgaaagtggagtaactactccatttctctgaggggagaattaaaatttttgaccaagtgttgttgagccactgagaatggtctcagaacataacttcttaaggaaccttcccagattgccctcaacactgcaccacatttggtcctgcttgaacattgccatggctcttaaagtcttaattaagaatattaattgtgtaattattgtttttcctcctttagatcattccttgaggacaggacagtgcttgtttaaggctatatttctgctgtctgagcagcaacaggtcttcgagatcaacatgatgttcataatcccaagatgttgccatttatgttctcagaagcaagcagaggcatgatggtcagtgacagtaatgtcactgtgttaaatgttgctatgcagtttggatttttctaatgtagtgtaggtagaacatatgtgttctgtatgaattaaactcttaagttacaccttgtataatccatgcaatgtgttatgcaattaccattttaagtattgtagctttctttgtatgtgaggataaaggtgtttgtcataaaatgttttgaacatttccccaaagttccaaattataaaaccacaacgttagaacttatttatgaacaatggttgtagtttcatgcttttaaaatgcttaattattcaattaacaccgtttgtgttataatatatataaaactgacatgtagaagtgtttgtccagaacatttcttaaatgtatactgtctttagagagtttaatatagcatgtcttttgcaacatactaacttttgtgttggtgcgagcaatattgtgtagtcattttgaaaggagtcatttcaatgagtgtcagattgttttgaatgttattgaacattttaaatgcagacttgttcgtgttttagaaagcaaaactgtcagaagctttgaactagaaattaaaaagctgaagtatttcagaagggaaataagctacttgctgtattagttgaaggaaagtgtaatagcttagaaaatttaaaaccatatagttgtcattgctgaatatctggcagatgaaaagaaatactcagtggttcttttgag

SEQ ID NO: 97 - DNA, 14848 bp, Artificial Sequence
  Synthetic sequence
gatccccgggtaccgagctcgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttgcatgcctgcaggtcgactctagaggatcctcgtcgtctgattggctctcggggcccagaaaactggcccttgccattggctcgtgttcgtgcaagttgagtccatccgccggccagcgggggcggcgaggaggcgctcccaggttccggccctcccctcggccccgcgccgcagagtctggccgcgcgcccctgcgcaacgtggcaggaagcgcgcgctgggggcggggacgggcagtagggctgagcggctgcggggcgggtgcaagcacgtttccgacttgagttgcctcaagaggggcgtgctgagccagacctccatcgcgcactccggggagtggagggaaggagcgagggctcagttgggctgttttggaggcaggaagcacttgctctcccaaagtcgctctgagttgttatcagtaagggagctgcagtggagtaggcggggagaaggccgcacccttctccggaggggggaggggagtgttgcaatacctttctgggagttctctgctgcctcctggcttctgaggaccgccctgggcctgggagaatcccttccccctcttccctcgtgatctgcaactccagtctttctagaagatgggcgggagtcttctgggcaggcttaaaggctaacctggtgtgtgggcgttgtcctgcaggggaattgaacaggtgtaaaattggagggacaagacttcccacagattttcggttttgtcgggaagttttttaataggggcaaataaggaaaatgggaggataggtagtcatctggggttttatgcagcaaaactacaggttattattgcttgtgatccgcctcggagtattttccatcgaggtagattaaagacatgctcacccgagttttatactctcctgcttgagatccttactacagtatgaaattacagtgtcgcgagttagactatgtaagcagaattttaatcatttttaaagagcccagtacttcatatccatttctcccgctccttctgcagccttatcaaaaggtattttagaacactcattttagccccattttcatttattatactggcttatccaacccctagacagagcattggcattttccctttcctgatcttagaagtctgatgactcatgaaaccagacagattagttacatacaccacaaatcgaggctgtagctggggcctcaacactgcagttcttttataactccttagtacactttttgttgatcctttgccttgatccttaattttcagtgtctatcacctctcccgtcaggtggtgttccacatttgggcctattctcagtccagggagttttacaacaatagatgtattgagaatccaacctaaagcttaactttccactcccatgaatgcctctctcctttttctccatttataaactgagctattaaccattaatggtttccaggtggatgtctcctcccccaatattacctgatgtatcttacatattgccaggctgatattttaagacattaaaaggtatatttcattattgagccacatggtattgattactgcttactaaaattttgtcattgtacacatctgtaaaaggtggttccttttggaatgcaaagttcaggtgtttgttgtctttcctgacctaaggtcttgtgagcttgtattttttctatttaagcagtgctttctcttggactggcttgactcatggcattctacacgttattgctggtctaaatgtgattttgccaagcttcttcaggacctataattttgcttgacttgtagccaaacacaagtaaaatgattaagcaacaaatgtatttgtgaagcttggtttttaggttgttgtgttgtgtgtgcttgtgctctataataatactatccaggggctggagaggtggctcggagttcaagagcacagactgctcttccagaagtcctgagttcaattcccagcaaccacatggtggctcacaaccatctgtaatgggatctgatgccctcttctggtgtgtctgaagaccacaagtgtattcacattaaataaataaatcctccttcttcttcttttttttttttttaaagagaatactgtctccagtagaatttactgaagtaatgaaatactttgtgtttgttccaatatggtagccaataatcaaattactctttaagcactggaaatgttaccaaggaactaatttttatttgaagtgtaactgtggacagaggagccataactgcagacttgtgggatacagaagaccaatgcagactttaatgtcttttctcttacactaagcaataaagaaataaaaattgaacttctagtatcctatttgtttaaactgctagctttacttaacttttgtgcttcatctatacaaagctgaaagctaagtctgcagccattactaaacatgaaagcaagtaatgataattttggatttcaaaaatgtagggccagagtttagccagccagtggtggtgcttgcctttatgcctttaatcccagcactctggaggcagagacaggcagatctctgagtttgagcccagcctggtctacacatcaagttctatctaggatagccaggaatacacacagaaaccctgttggggaggggggctctgagatttcataaaattataattgaagcattccctaatgagccactatggatgtggctaaatccgtctacctttctgatgagatttgggtattattttttctgtctctgctgttggttgggtcttttgacactgtgggctttctttaaagcctccttcctgccatgtggtctcttgtttgctactaacttcccatggcttaaatggcatggctttttgccttctaagggcagctgctgagatttgcagcctgatttccagggtggggttgggaaatctttcaaacactaaaattgtcctttaattttttttttaaaaaatgggttatataataaacctcataaaatagttatgaggagtgaggtggactaatattaaatgagtccctcctagggataagacagatcgacactgctcgaaagttcagatgtgcggcgagttgcgtgactacctacgggtaacagtttcttaccgttcgtataaagtatcctatacgaagttatttatggcagggtgaaacgcaggtcgccagctaccgttcgtataatgtatgctatacgaagttatctttcggcggtgaaattatcgatgagcgtggtggttatgccgatcgcgtctccacgtgcatcaaggcgcgccattgatgcggccgggacagcagagatccactttggcgccggctcgagtggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtgtcgtgacgcggggcaaagaattcccgggtgagccgccaccatggctggagacatgagagctgccaacctttggccaagcccgctcatgatcaaacgctctaagaagaacagcctggccttgtccctgacggccgaccagatggtcagtgccttgttggatgctgagccccccatactctattccgagtatgatcctaccagacccttcagtgaagcttcgatgatgggcttactgaccaacctggcagacagggagctggttcacatgatcaactgggcgaagagggtgccaggctttgtggatttgaccctccatgatcaggtccaccttctagaatgtgcctggctagagatcctgatgattggtctcgtctggcgctccatggagcacccagtgaagctactgtttgctcctaacttgctcttggacaggaaccagggaaaatgtgtagagggcatggtggagatcttcgacatgctgctggctacatcatctcggttccgcatgatgaatctgcagggagaggagtttgtgtgcctcaaatctattattttgcttaattctggagtgtacacatttctgtccagcaccctgaagtctctggaagagaaggaccatatccaccgagtcctggacaagatcacagacactttgatccacctgatggccaaggcaggcctgaccctgcagcagcagcaccagcggctggcccagctcctcctcatcctctcccacatcaggcacatgagtaacaaaggcatggagcatctgtacagcatgaagtgcaagaacgtggtgcccctctatgacctgctgctggaggcggcggacgcccaccgcctacatgcgcccactagccgtggaggggcatccgtggaggagacggaccaaagccacttggccactgcgggctctacttcatcgcattccttgcaaaagtattacatcacgggggaggcagagggtttccctgccacagctgtcgacaatttactgaccgtacaccaaaatttgcctgcattaccggtcgatgcaacgagtgatgaggttcgcaagaacctgatggacatgttcagggatcgccaggcgttttctgagcatacctggaaaatgcttctgtccgtttgccggtcgtgggcggcatggtgcaagttgaataaccggaaatggtttcccgcagaacctgaagatgttcgcgattatcttctatatcttcaggcgcgcggtctggcagtaaaaactatccagcaacatttgggccagctaaacatgcttcatcgtcggtccgggctgccacgaccaagtgacagcaatgctgtttcactggttatgcggcggatccgaaaagaaaacgttgatgccggtgaacgtgcaaaacaggctctagcgttcgaacgcactgatttcgaccaggttcgttcactcatggaaaatagcgatcgctgccaggatatacgtaatctggcatttctggggattgcttataacaccctgttacgtatagccgaaattgccaggatcagggttaaagatatctcacgtactgacggtgggagaatgttaatccatattggcagaacgaaaacgctggttagcaccgcaggtgtagagaaggcacttagcctgggggtaactaaactggtcgagcgatggatttccgtctctggtgtagctgatgatccgaataactacctgttttgccgggtcagaaaaaatggtgttgccgcgccatctgccaccagccagctatcaactcgcgccctggaagggatttttgaagcaactcatcgattgatttacggcgctaaggatgactctggtcagagatacctggcctggtctggacacagtgcccgtgtcggagccgcgcgagatatggcccgcgctggagtttcaataccggagatcatgcaagctggtggctggaccaatgtaaatattgtcatgaactatatccgtaacctggatagtgaaacaggggcaatggtgcgcctgctggaagatggcgatctcgagccatctgctggagacatgagagctgccaacctttggccaagcccgctcatgatcaaacgctctaagaagaacagcctggccttgtccctgacggccgaccagatggtcagtgccttgttggatgctgagccccccatactctattccgagtatgatcctaccagacccttcagtgaagcttcgatgatgggcttactgaccaacctggcagacagggagctggttcacatgatcaactgggcgaagagggtgccaggctttgtggatttgaccctccatgatcaggtccaccttctagaatgtgcctggctagagatcctgatgattggtctcgtctggcgctccatggagcacccagtgaagctactgtttgctcctaacttgctcttggacaggaaccagggaaaatgtgtagagggcatggtggagatcttcgacatgctgctggctacatcatctcggttccgcatgatgaatctgcagggagaggagtttgtgtgcctcaaatctattattttgcttaattctggagtgtacacatttctgtccagcaccctgaagtctctggaagagaaggaccatatccaccgagtcctggacaagatcacagacactttgatccacctgatggccaaggcaggcctgaccctgcagcagcagcaccagcggctggcccagctcctcctcatcctctcccacatcaggcacatgagtaacaaaggcatggagcatctgtacagcatgaagtgcaagaacgtggtgcccctctatgacctgctgctggaggcggcggacgcccaccgcctacatgcgcccactagccgtggaggggcatccgtggaggagacggaccaaagccacttggccactgcgggctctacttcatcgcattccttgcaaaagtattacatcacgggggaggcagagggtttccctgccacagcttgatagcggccgcactcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgccatatgctggctgccatgaacaaaggtggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatgaagatccctcgacctgcgatccccgggtaccgagctgcctgcaggtcgactctagaggatcggccgcatctagaagttcctattccgaagttcctattctctagaaagtataggaacttcgcagaatcatagatctctcgaggttaacgaattctaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacctgcagcccaagcttaccatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgacgcccgccccacgacccgcagcgcccgaccgaaaggagcgcacgaccccatgcatcgataaaataaaagattttatttagtctccagaaaaaggggggaatgaaagaccccacctgtaggtttggcaagctagcgtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgccatatgctggctgccatgaacaaaggtggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatgaagatccctcgacctgaactgttagtagaagttcctattccgaagttcctattctctagaaagtataggaacttcggatcagccgcggcagtctgacgagatcatatcactgtggacgttgatgaaagaatacgttattctttcatcaaatcgtgtcgtggatcagttctggacgagccagggtaatgagctattaaggctttttgtcttatacttaactttttttttaaatgtggtatctttagaaccaagggtcttagagttttagtatacagaaactgttgcatcgcttaatcagattttctagtttcaaatccagagaatccaaattcttcacagccaaagtcaaattaagaatttctgacttttaatgttaatttgcttactgtgaatataaaaatgatagcttttcctgaggcagggtctcactatgtatctctgcctgatctgcaacaagatatgtagactaaagttctgcctgcttttgtctcctgaatactaaggttaaaatgtagtaatacttttggaacttgcaggtcagattcttttataggggacacactaagggagcttgggtgatagttggtaaaatgtgtttcaagtgatgaaaacttgaattattatcaccgcaacctactttttaaaaaaaaaagccaggcctgttagagcatgcttaagggatccctaggacttgctgagcacacaagagtagttacttggcaggctcctggtgagagcatatttcaaaaaacaaggcagacaaccaagaaactacagttaaggttacctgtctttaaaccatctgcatatacacagggatattaaaatattccaaataatatttcattcaagttttcccccatcaaattgggacatggatttctccggtgaataggcagagttggaaactaaacaaatgttggttttgtgatttgtgaaattgttttcaagtgatagttaaagcccatgagatacagaacaaagctgctatttcgaggtctcttggtttatactcagaagcacttctttgggtttccctgcactatcctgatcatgtgctaggcctaccttaggctgattgttgttcaaataaacttaagtttcctgtcaggtgatgtcatatgatttcatatatcaaggcaaaacatgttatatatgttaaacatttgtacttaatgtgaaagttaggtctttgtgggtttgatttttaattttcaaaacctgagctaaataagtcatttttacatgtcttacatttggtggaattgtataattgtggtttgcaggcaagactctctgacctagtaaccctacctatagagcactttgctgggtcacaagtctaggagtcaagcatttcaccttgaagttgagacgttttgttagtgtatactagtttatatgttggaggacatgtttatccagaagatattcaggactatttttgactgggctaaggaattgattctgattagcactgttagtgagcattgagtggcctttaggcttgaattggagtcacttgtatatctcaaataatgctggccttttttaaaaagcccttgttctttatcaccctgttttctacataatttttgttcaaagaaatacttgtttggatctccttttgacaacaatagcatgttttcaagccatattttttttcctttttttttttttttttggtttttcgagacagggtttctctgtatagccctggctgtcctggaactcactttgtagaccaggctggcctcgaactcagaaatccgcctgcctctgcctcctgagtgccgggattaaaggcgtgcaccaccacgcctggctaagttggatattttgttatataactataaccaatactaactccactgggtggatttttaattcagtcagtagtcttaagtggtctttattggcccttcattaaaatctactgttcactctaacagaggctgttggtactagtggcacttaagcaacttcctacggatatactagcagattaagggtcagggatagaaactagtctagcgttttgtatacctaccagctttatactaccttgttctgatagaaatatttcaggacatctagagtgtactataaggttgatggtaagcttataaggaacttgaaagtggagtaactactccatttctctgaggggagaattaaaatttttgaccaagtgttgttgagccactgagaatggtctcagaacataacttcttaaggaaccttcccagattgccctcaacactgcaccacatttggtcctgcttgaacattgccatggctcttaaagtcttaattaagaatattaattgtgtaattattgtttttcctcctttagatcattccttgaggacaggacagtgcttgtttaaggctatatttctgctgtctgagcagcaacaggtcttcgagatcaacatgatgttcataatcccaagatgttgccatttatgttctcagaagcaagcagaggcatgatggtcagtgacagtaatgtcactgtgttaaatgttgctatgcagtttggatttttctaatgtagtgtaggtagaacatatgtgttctgtatgaattaaactcttaagttacaccttgtataatccatgcaatgtgttatgcaattaccattttaagtattgtagctttctttgtatgtgaggataaaggtgtttgtcataaaatgttttgaacatttccccaaagttccaaattataaaaccacaacgttagaacttatttatgaacaatggttgtagtttcatgcttttaaaatgcttaattattcaattaacaccgtttgtgttataatatatataaaactgacatgtagaagtgtttgtccagaacatttcttaaatgtatactgtctttagagagtttaatatagcatgtcttttgcaacatactaacttttgtgttggtgcgagcaatattgtgtagtcattttgaaaggagtcatttcaatgagtgtcagattgttttgaatgttattgaacattttaaatgcagacttgttcgtgttttagaaagcaaaactgtcagaagctttgaactagaaattaaaaagctgaagtatttcagaagggaaataagctacttgctgtattagttgaaggaaagtgtaatagcttagaaaatttaaaaccatatagttgtcattgctgaatatctggcagatgaaaagaaatactcagtggttcttttgag

SEQ ID NO: 98 - DNA, 8741 bp, Artificial Sequence
  Synthetic sequence
gatccccgggtaccgagctcgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttgcatgcctgcaggtcgactctagaggatcgggagtacacactctcctaaaaagctcagcacactccactcagctggatcctccccaatcacaagtatagctatgtaacatctgtccaacaaagaaatgtaaatatgaccttggtcacaaatatgtcatctaaaaacttgtttagtcaactgatgaaggtaattagcacagaacttggggattcgttcagtacctatgtgcaggaaggaaactggagaaatgaacttaaatatagacaaagcccagtggaacccagaggtctgggaaagaaatgagttaccggtgaatacattgcacaagtaacatgagaaagcagaaaatgcaggtcatacacgcacccctgacccagaccagcagagctgactgcagcatccatatccaagagaaagaccactgacgcccaagaagtgagacaagcaaggactctatagaatcaattagcatagaaggggctttcccaacagtttaactttccctctcatgcgatttacctacttgaaccagggctctttcctacactcctcttcacattcccgacttacacgcagagggaaagagaattcataaagggaatatttttctgcctttgaagatattctcacaagatcgttctccacgcccaaggcaagtaaaacgacacaatctggctcaactccaggctcgaaccctacacattcaacgaggctatctcagacacgctgtggcacacgccacggggagccagagaacgtgtggtgggggtggcgaaggtaatgcctttgggaagcagccatctgaggtgggaagccagaaaacgagagggaaggcgtccaggaagattacggaggggagatcgcggcccccagagcgatcagagttgtctgtcacaaggccgcgagaacgggggtagggagtgggggatcggggagagaaaaaaagtatgcctgtgtatttcgagcggagggcagcaagaggcctctcctcaagggaaaggtaaacgtggagtaggcagttcccaggaaagggggtgaagaggcgttgggggaggggaagcgtcctgacccaggaaaaacatgaaagggggagttgggtcgcctagattagagggggatctctctccctgggaaaatggggtgttgcaacggtgtgtgcaaggcggcgaggggggtgagaagtggcagcatcctcctaagagcttggggagggccaggcccacgacccaaggagagcgagcgcggggagacggaggaggtgacccttccctcccctggggcccgatcgtgaggttcggtctcttttctgtcggacccttaccttgtcccaggcgctgccggggcctgggcccgggctgcggcgcacggcactcccgggaggcggcaggactcgagttaggcccaacgcggcgccacggcgtttcctggccgggaatggcccgtacccgtgaggtgggggtggggggcagaaaggcggagcgagccaaaggcggggagggggggcagggccagggaaagaggggggccggcactactgtgttggcggactggcgggactggggctgcgtgagtctctgagcgcaggcgggcggcggccgcccctcccccggcagcggcggcggcggcggcggcggcggcggcggcggcagctcactcagcccgctgcccgagcggaaacgccactgaccgcacggggattcccagcgccggcgccaggggcacccgggacacgccccctcccgccgcgccattggcctctccgcccaccgccccgcacccattggcccactcgccgccaatcagcggaagccgccggggccgcctagagaagaggctgtgcttcggcgctccggctcctcagagagcctcggctaggtaggggagcggaactctggtgggaggggaggtgcggtacactggggggatgggtggctaggggggccgtctggtggcttgcgggggttgcctttcccgtgggaagtcgggaacataatgtttgttacgttgggagggaaaggggtggctggatgcaggcgggagggaggcccgccctgcggcaaccggagggggagggagaagggagcggaaaatgctcgaaaccggacggagccattgctctcgcagagggaggagcgcttccggctagcctcttgtcgccgattggccgtttctcctcccgccgtgtgtgaaaacacaaatggcgtattctggttggagtaaagctcctgtcagttacgccgtcgggagtacgcagccgcttagcgactctcgcgttgccccctgggtggggcgggtaggtaggtggggtgtagagatgctgggtgtgcgggcgcggccggcctcctgcggcgggaggggagggtcagtgaaatcggctctggcgcgggcgtcctcccaccctccccttccttcgggggagtcggtttacccgccgcctgcttgtcttcgacacctgattggctgtcgaagctgtgggaccgggcccttgctactggctcgagtctcacatgagcgaaaccactgcgcggggcgcgggggtggcggggaggcgggcgttggtacggtcctccccgaggccgagcgccgcagtgtctggccccgcgcccctgcgcaacgtggcaggaagcgcgcgctggaggcgggggcgggctgccggccgagacttctggatggcggcggccgcggctccgccccgggttcccaccgcctgaagggcgagacaagcccgacctgctacaggcactcgtgggggtgggggaggagcgggggtcggtccggctggtttgtgggtgggaggcgcttgttctccaaaaaccggcgcgagctgcaatcctgagggagctgcggtggaggaggtggagagaaggccgcacccttctgggcagggggaggggagtgccgcaatacctttatgggagttctctgctgcctcccgtcttgtaaggaccgccctgggcctggaagaagccctccctcctttcctcctcgcgtgatctcgtcatcgcctccatgtagggataacagggtaatagtcgcttctcgattatgggcgggattcttttgcctaggcttaaggggctaacttggtccctgggcgttgccctgcaggggagtgagcagctgtaagatttgaggggcgactccgattagtttatcttcccacggactagagttggtgtcgaggttattgtaataagggtggggtagggaaatggagcttagtcattcacctggggctgattttatgcaacgagactgcggattatcactacttatcatttttggagcatttttctagagacagacataaagcatgatcacctgagttttataccatttgagacccttgctgcaccaccaaagtgtagcatcaggttaaatcttaatagaaaaattttagcttttgcttgagaaaccagtgcttccctccctcaccctctctccccaggctctctacccctttgcatccctaccaggcatcttagcaactctcactcatacttgatcccattttccatttgttgtacttgctcctctagtattcagacatagcactagctttctccctctcttgatcttgggtagcctggtgtctcgcgaaaccagacagattggttccaccacaaattaaggcttgagctggggcttgactcttacccagcagtgcttttattcctccctagttcacgttcttaaatgtttatcttgattttcattttatcctttttccttagctgggattctgtccctgaccgtcttcacagtccaggtgatcttgactactgctttacagagaattggatctgaggttaggcaacatctccctttttcttcctctaaatacctctcatttctgttcttaccagttagtaactgatctcagatgcctgtgtgatagcttccaaattgctgtctctgtctttagtgttatcttttgatccatcttacatcttgttaggatgattgtcctaaaggaagatagagcatgaaaatgacaggtgaaactccattactggcttgtactgtttacagcaagttgtctaacccgcgggccgcatgggacccaggacagctttgaatgcgacccaacacaaattcgtaaactttcttaaagttttaggagttttttttgcggggtgttttttttgttgttgttgtctgttttttttttttgctcatcagctgtcctttatatgtggcccaagacagttcttgcagtgtggcccagggaagccgaaagattggacacccctggtttacaggatgaattctgttctcgtgaatgtgatgcataaggtccctggatagactttcctcaatttaacagctttttcatttttcactattttccacatactacacacacaaatatgacctctctcctgaagtcacatggaatgaattgcagttacccaaatcatagagacgtcatatatctttgtggggtggttctttttggaatgctacccttgctgcttttaatcctgcagattcaaatgtcatctcccctttggtcatgccttccatggtctgttgttctatgacccaaactgcccttttaaaaaagcagttttcttctgactcctgagcagtttgtactgcgtgggtcatagtaattctggggttgttgtcattgctgatttaaattaccttaactattctgagctttttgaaggaatagtgtcttgtactcctgagctcctagtatactctgatgtctagccaactttaagtaaatggagtgagcgacatgtaaatgtctttatggtttgacattttattaatcttgagcattggaaaagtggctggtatgactaaagaactgaatttttattttttatctaatttgaattgcaactgtggacagtatagctgtagccccggacttgttctgtggattacagaatacgtggacattgatggttttctctttttactgagcagtaaggaaataaaagctgaagctctctggcccattacatttcatttaactgcttgttttgttttatttattatatatttagctgaaagctaagtccaacaaaccatttttaaagatgtgcctgccttggacaacagtgaaagcaagcgttaatgagtttggatttcaaaaaaaaatgcataatgaattttagggaggtgagattccattaaactgttacaggagctacatagttaaatatatcagaacttgaatgaagaataggtctgtgtgatacaaatgaacccagcctaaatttctgtcctacaagttttcacttcctggaaacacttgattggaggagctctaagcatcatatcttgagttatagaaaacaatcacaaagtaagttttatcctgaggaattataataccaacattttaacaccaaatgctttttatattgctaatacagtatcttacacattttaattatcttttttttttttttgagacggagtcttagtgtcacatactggattgcaatggcatgatcatggctcacgcagccttgacttcctgggctcaagtgatcctcctgcctcagcctcctgagtagctggaactacaggcacacgcttccatgcccagctaatttttttgtgtttttttttttgtagagatgggtttttgctgtgctgcacaggttagaactcctgggctcaagtgatcttcctgccctaagcttcccaaagtcctgggattacaggtgtgagccaccgccctaggccattaaatatactttttaaaaagacccttatgtcagcgatcttcaggcctcctgtgtgtggagacagttgtcttaatgtagattctcagatcacccaaaaattctgaattcttttggggaaggtggaaccccacagggcaggcagtataactcagtatcttcaagttcatttgctgactcggatatgcctatttacctgccaaaaatgattacagtttcagagaaactcttctcaattgtatagtatgtgaccctcaatttttcctcaaatgaactgttttttcctcctcctaaaaaataacagaatgggaattttgagaggcaaagaggaatagtaattaatatttttggaaacttgcaggtcaggt

SEQ ID NO: 99 - DNA, 14851 bp, Artificial Sequence
  Synthetic sequence
gatccccgggtaccgagctcgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttgcatgcctgcaggtcgactctagaggatcgggagtacacactctcctaaaaagctcagcacactccactcagctggatcctccccaatcacaagtatagctatgtaacatctgtccaacaaagaaatgtaaatatgaccttggtcacaaatatgtcatctaaaaacttgtttagtcaactgatgaaggtaattagcacagaacttggggattcgttcagtacctatgtgcaggaaggaaactggagaaatgaacttaaatatagacaaagcccagtggaacccagaggtctgggaaagaaatgagttaccggtgaatacattgcacaagtaacatgagaaagcagaaaatgcaggtcatacacgcacccctgacccagaccagcagagctgactgcagcatccatatccaagagaaagaccactgacgcccaagaagtgagacaagcaaggactctatagaatcaattagcatagaaggggctttcccaacagtttaactttccctctcatgcgatttacctacttgaaccagggctctttcctacactcctcttcacattcccgacttacacgcagagggaaagagaattcataaagggaatatttttctgcctttgaagatattctcacaagatcgttctccacgcccaaggcaagtaaaacgacacaatctggctcaactccaggctcgaaccctacacattcaacgaggctatctcagacacgctgtggcacacgccacggggagccagagaacgtgtggtgggggtggcgaaggtaatgcctttgggaagcagccatctgaggtgggaagccagaaaacgagagggaaggcgtccaggaagattacggaggggagatcgcggcccccagagcgatcagagttgtctgtcacaaggccgcgagaacgggggtagggagtgggggatcggggagagaaaaaaagtatgcctgtgtatttcgagcggagggcagcaagaggcctctcctcaagggaaaggtaaacgtggagtaggcagttcccaggaaagggggtgaagaggcgttgggggaggggaagcgtcctgacccaggaaaaacatgaaagggggagttgggtcgcctagattagagggggatctctctccctgggaaaatggggtgttgcaacggtgtgtgcaaggcggcgaggggggtgagaagtggcagcatcctcctaagagcttggggagggccaggcccacgacccaaggagagcgagcgcggggagacggaggaggtgacccttccctcccctggggcccgatcgtgaggttcggtctcttttctgtcggacccttaccttgtcccaggcgctgccggggcctgggcccgggctgcggcgcacggcactcccgggaggcggcaggactcgagttaggcccaacgcggcgccacggcgtttcctggccgggaatggcccgtacccgtgaggtgggggtggggggcagaaaggcggagcgagccaaaggcggggagggggggcagggccagggaaagaggggggccggcactactgtgttggcggactggcgggactggggctgcgtgagtctctgagcgcaggcgggcggcggccgcccctcccccggcagcggcggcggcggcggcggcggcggcggcggcggcagctcactcagcccgctgcccgagcggaaacgccactgaccgcacggggattcccagcgccggcgccaggggcacccgggacacgccccctcccgccgcgccattggcctctccgcccaccgccccgcacccattggcccactcgccgccaatcagcggaagccgccggggccgcctagagaagaggctgtgcttcggcgctccggctcctcagagagcctcggctaggtaggggagcggaactctggtgggaggggaggtgcggtacactggggggatgggtggctaggggggccgtctggtggcttgcgggggttgcctttcccgtgggaagtcgggaacataatgtttgttacgttgggagggaaaggggtggctggatgcaggcgggagggaggcccgccctgcggcaaccggagggggagggagaagggagcggaaaatgctcgaaaccggacggagccattgctctcgcagagggaggagcgcttccggctagcctcttgtcgccgattggccgtttctcctcccgccgtgtgtgaaaacacaaatggcgtattctggttggagtaaagctcctgtcagttacgccgtcgggagtacgcagccgcttagcgactctcgcgttgccccctgggtggggcgggtaggtaggtggggtgtagagatgctgggtgtgcgggcgcggccggcctcctgcggcgggaggggagggtcagtgaaatcggctctggcgcgggcgtcctcccaccctccccttccttcgggggagtcggtttacccgccgcctgcttgtcttcgacacctgattggctgtcgaagctgtgggaccgggcccttgctactggctcgagtctcacatgagcgaaaccactgcgcggggcgcgggggtggcggggaggcgggcgttggtacggtcctccccgaggccgagcgccgcagtgtctggccccgcgcccctgcgcaacgtggcaggaagcgcgcgctggaggcgggggcgggctgccggccgagacttctggatggcggcggccgcggctccgccccgggttcccaccgcctgaagggcgagacaagcccgacctgctacaggcactcgtgggggtgggggaggagcgggggtcggtccggctggtttgtgggtgggaggcgcttgttctccaaaaaccggcgcgagctgcaatcctgagggagctgcggtggaggaggtggagagaaggccgcacccttctgggcagggggaggggagtgccgcaatacctttatgggagttctctgctgcctcccgtcttgtaaggaccgccctgggcctggaagaagccctccctcctttcctcctcgcgtgatctcgtcatcgcctccatgtagggataagacagatcgacactgctcgaaagttcagatgtgcggcgagttgcgtgactacctacgggtaacagtttcttaccgttcgtataaagtatcctatacgaagttatttatggcagggtgaaacgcaggtcgccagctaccgttcgtataatgtatgctatacgaagttatctttcggcggtgaaattatcgatgagcgtggtggttatgccgatcgcgtctccacgtgcatcaaggcgcgccattgatgcggccgggacagcagagatccactttggcgccggctcgagtggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtgtcgtgacgcggggcaaagaattcccgggtgagccgccaccatggctggagacatgagagctgccaacctttggccaagcccgctcatgatcaaacgctctaagaagaacagcctggccttgtccctgacggccgaccagatggtcagtgccttgttggatgctgagccccccatactctattccgagtatgatcctaccagacccttcagtgaagcttcgatgatgggcttactgaccaacctggcagacagggagctggttcacatgatcaactgggcgaagagggtgccaggctttgtggatttgaccctccatgatcaggtccaccttctagaatgtgcctggctagagatcctgatgattggtctcgtctggcgctccatggagcacccagtgaagctactgtttgctcctaacttgctcttggacaggaaccagggaaaatgtgtagagggcatggtggagatcttcgacatgctgctggctacatcatctcggttccgcatgatgaatctgcagggagaggagtttgtgtgcctcaaatctattattttgcttaattctggagtgtacacatttctgtccagcaccctgaagtctctggaagagaaggaccatatccaccgagtcctggacaagatcacagacactttgatccacctgatggccaaggcaggcctgaccctgcagcagcagcaccagcggctggcccagctcctcctcatcctctcccacatcaggcacatgagtaacaaaggcatggagcatctgtacagcatgaagtgcaagaacgtggtgcccctctatgacctgctgctggaggcggcggacgcccaccgcctacatgcgcccactagccgtggaggggcatccgtggaggagacggaccaaagccacttggccactgcgggctctacttcatcgcattccttgcaaaagtattacatcacgggggaggcagagggtttccctgccacagctgtcgacaatttactgaccgtacaccaaaatttgcctgcattaccggtcgatgcaacgagtgatgaggttcgcaagaacctgatggacatgttcagggatcgccaggcgttttctgagcatacctggaaaatgcttctgtccgtttgccggtcgtgggcggcatggtgcaagttgaataaccggaaatggtttcccgcagaacctgaagatgttcgcgattatcttctatatcttcaggcgcgcggtctggcagtaaaaactatccagcaacatttgggccagctaaacatgcttcatcgtcggtccgggctgccacgaccaagtgacagcaatgctgtttcactggttatgcggcggatccgaaaagaaaacgttgatgccggtgaacgtgcaaaacaggctctagcgttcgaacgcactgatttcgaccaggttcgttcactcatggaaaatagcgatcgctgccaggatatacgtaatctggcatttctggggattgcttataacaccctgttacgtatagccgaaattgccaggatcagggttaaagatatctcacgtactgacggtgggagaatgttaatccatattggcagaacgaaaacgctggttagcaccgcaggtgtagagaaggcacttagcctgggggtaactaaactggtcgagcgatggatttccgtctctggtgtagctgatgatccgaataactacctgttttgccgggtcagaaaaaatggtgttgccgcgccatctgccaccagccagctatcaactcgcgccctggaagggatttttgaagcaactcatcgattgatttacggcgctaaggatgactctggtcagagatacctggcctggtctggacacagtgcccgtgtcggagccgcgcgagatatggcccgcgctggagtttcaataccggagatcatgcaagctggtggctggaccaatgtaaatattgtcatgaactatatccgtaacctggatagtgaaacaggggcaatggtgcgcctgctggaagatggcgatctcgagccatctgctggagacatgagagctgccaacctttggccaagcccgctcatgatcaaacgctctaagaagaacagcctggccttgtccctgacggccgaccagatggtcagtgccttgttggatgctgagccccccatactctattccgagtatgatcctaccagacccttcagtgaagcttcgatgatgggcttactgaccaacctggcagacagggagctggttcacatgatcaactgggcgaagagggtgccaggctttgtggatttgaccctccatgatcaggtccaccttctagaatgtgcctggctagagatcctgatgattggtctcgtctggcgctccatggagcacccagtgaagctactgtttgctcctaacttgctcttggacaggaaccagggaaaatgtgtagagggcatggtggagatcttcgacatgctgctggctacatcatctcggttccgcatgatgaatctgcagggagaggagtttgtgtgcctcaaatctattattttgcttaattctggagtgtacacatttctgtccagcaccctgaagtctctggaagagaaggaccatatccaccgagtcctggacaagatcacagacactttgatccacctgatggccaaggcaggcctgaccctgcagcagcagcaccagcggctggcccagctcctcctcatcctctcccacatcaggcacatgagtaacaaaggcatggagcatctgtacagcatgaagtgcaagaacgtggtgcccctctatgacctgctgctggaggcggcggacgcccaccgcctacatgcgcccactagccgtggaggggcatccgtggaggagacggaccaaagccacttggccactgcgggctctacttcatcgcattccttgcaaaagtattacatcacgggggaggcagagggtttccctgccacagcttgatagcggccgcactcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgccatatgctggctgccatgaacaaaggtggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatgaagatccctcgacctgcgatccccgggtaccgagctgcctgcaggtcgactctagaggatcggccgcatctagaagttcctattccgaagttcctattctctagaaagtataggaacttcgcagaatcatagatctctcgaggttaacgaattctaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacctgcagcccaagcttaccatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgacgcccgccccacgacccgcagcgcccgaccgaaaggagcgcacgaccccatgcatcgataaaataaaagattttatttagtctccagaaaaaggggggaatgaaagaccccacctgtaggtttggcaagctagcgtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgccatatgctggctgccatgaacaaaggtggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatgaagatccctcgacctgaactgttagtagaagttcctattccgaagttcctattctctagaaagtataggaacttcggatcagccgcggcagtctgacgagatcatatcactgtggacgttgatgaaagaatacgttattctttcatcaaatcgtgtcgtggatcagttctggacgagccagggtaatagtcgcttctcgattatgggcgggattcttttgcctaggcttaaggggctaacttggtccctgggcgttgccctgcaggggagtgagcagctgtaagatttgaggggcgactccgattagtttatcttcccacggactagagttggtgtcgaggttattgtaataagggtggggtagggaaatggagcttagtcattcacctggggctgattttatgcaacgagactgcggattatcactacttatcatttttggagcatttttctagagacagacataaagcatgatcacctgagttttataccatttgagacccttgctgcaccaccaaagtgtagcatcaggttaaatcttaatagaaaaattttagcttttgcttgagaaaccagtgcttccctccctcaccctctctccccaggctctctacccctttgcatccctaccaggcatcttagcaactctcactcatacttgatcccattttccatttgttgtacttgctcctctagtattcagacatagcactagctttctccctctcttgatcttgggtagcctggtgtctcgcgaaaccagacagattggttccaccacaaattaaggcttgagctggggcttgactcttacccagcagtgcttttattcctccctagttcacgttcttaaatgtttatcttgattttcattttatcctttttccttagctgggattctgtccctgaccgtcttcacagtccaggtgatcttgactactgctttacagagaattggatctgaggttaggcaacatctccctttttcttcctctaaatacctctcatttctgttcttaccagttagtaactgatctcagatgcctgtgtgatagcttccaaattgctgtctctgtctttagtgttatcttttgatccatcttacatcttgttaggatgattgtcctaaaggaagatagagcatgaaaatgacaggtgaaactccattactggcttgtactgtttacagcaagttgtctaacccgcgggccgcatgggacccaggacagctttgaatgcgacccaacacaaattcgtaaactttcttaaagttttaggagttttttttgcggggtgttttttttgttgttgttgtctgttttttttttttgctcatcagctgtcctttatatgtggcccaagacagttcttgcagtgtggcccagggaagccgaaagattggacacccctggtttacaggatgaattctgttctcgtgaatgtgatgcataaggtccctggatagactttcctcaatttaacagctttttcatttttcactattttccacatactacacacacaaatatgacctctctcctgaagtcacatggaatgaattgcagttacccaaatcatagagacgtcatatatctttgtggggtggttctttttggaatgctacccttgctgcttttaatcctgcagattcaaatgtcatctcccctttggtcatgccttccatggtctgttgttctatgacccaaactgcccttttaaaaaagcagttttcttctgactcctgagcagtttgtactgcgtgggtcatagtaattctggggttgttgtcattgctgatttaaattaccttaactattctgagctttttgaaggaatagtgtcttgtactcctgagctcctagtatactctgatgtctagccaactttaagtaaatggagtgagcgacatgtaaatgtctttatggtttgacattttattaatcttgagcattggaaaagtggctggtatgactaaagaactgaatttttattttttatctaatttgaattgcaactgtggacagtatagctgtagccccggacttgttctgtggattacagaatacgtggacattgatggttttctctttttactgagcagtaaggaaataaaagctgaagctctctggcccattacatttcatttaactgcttgttttgttttatttattatatatttagctgaaagctaagtccaacaaaccatttttaaagatgtgcctgccttggacaacagtgaaagcaagcgttaatgagtttggatttcaaaaaaaaatgcataatgaattttagggaggtgagattccattaaactgttacaggagctacatagttaaatatatcagaacttgaatgaagaataggtctgtgtgatacaaatgaacccagcctaaatttctgtcctacaagttttcacttcctggaaacacttgattggaggagctctaagcatcatatcttgagttatagaaaacaatcacaaagtaagttttatcctgaggaattataataccaacattttaacaccaaatgctttttatattgctaatacagtatcttacacattttaattatcttttttttttttttgagacggagtcttagtgtcacatactggattgcaatggcatgatcatggctcacgcagccttgacttcctgggctcaagtgatcctcctgcctcagcctcctgagtagctggaactacaggcacacgcttccatgcccagctaatttttttgtgtttttttttttgtagagatgggtttttgctgtgctgcacaggttagaactcctgggctcaagtgatcttcctgccctaagcttcccaaagtcctgggattacaggtgtgagccaccgccctaggccattaaatatactttttaaaaagacccttatgtcagcgatcttcaggcctcctgtgtgtggagacagttgtcttaatgtagattctcagatcacccaaaaattctgaattcttttggggaaggtggaaccccacagggcaggcagtataactcagtatcttcaagttcatttgctgactcggatatgcctatttacctgccaaaaatgattacagtttcagagaaactcttctcaattgtatagtatgtgaccctcaatttttcctcaaatgaactgttttttcctcctcctaaaaaataacagaatgggaattttgagaggcaaagaggaatagtaattaatatttttggaaacttgcaggtcaggt

SEQ ID NO: 100 - DNA, 5407 bp, Artificial Sequence
  Synthetic sequence
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagcttggtaccgagctcggatccctacttccaatccaatattggaagtggataatctagagggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcattattattgaagcatttatcagggttattgtctcatgagcggatacatatttgaatgtatttagaaaaataaacaaataggggttccgcgcacatttccccgaaaagtgccacctgacgtc

SEQ ID NO: 101 - DNA, 6101 bp, Artificial Sequence
  Synthetic sequence
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagcttgccgccaccatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgacgcccgccccacgacccgcagcgcccgaccgaaaggagcgcacgaccccatgcatcgataaaataaaagattttatttagtctccagaaaaaggggggaatgaaagaccccacctgtaggtttggcaagctagctctagagggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcattattattgaagcatttatcagggttattgtctcatgagcggatacatatttgaatgtatttagaaaaataaacaaataggggttccgcgcacatttccccgaaaagtgccacctgacgtc

SEQ ID NO: 102 - DNA, 6415 bp, Artificial Sequence
  Synthetic sequence
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagcttgccgccaccatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccggggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagagtagatgccgaccgaacaagtctagagggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcattattattgaagcatttatcagggttattgtctcatgagcggatacatatttgaatgtatttagaaaaataaacaaataggggttccgcgcacatttccccgaaaagtgccacctgacgtc

SEQ ID NO: 103 - DNA, 5786 bp, Artificial Sequence
  Synthetic sequence
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagcttgccgccaccatggccaagcctttgtctcaagaagaatccaccctcattgaaagagcaacggctacaatcaacagcatccccatctctgaagactacagcgtcgccagcgcagctctctctagcgacggccgcatcttcactggtgtcaatgtatatcattttactgggggaccttgtgcagaactcgtggtgctgggcactgctgctgctgcggcagctggcaacctgacttgtatcgtcgcgatcggaaatgagaacaggggcatcttgagcccctgcggacggtgccgacaggtgcttctcgatctgcatcctgggatcaaagccatagtgaaggacagtgatggacagccgacggcagttgggattcgtgaattgctgccctctggttatgtgtgggagggctaagcacaattcgagctcggtactctagagggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcattattattgaagcatttatcagggttattgtctcatgagcggatacatatttgaatgtatttagaaaaataaacaaataggggttccgcgcacatttccccgaaaagtgccacctgacgtc

SEQ ID NO: 104 - DNA, 5814 bp, Artificial Sequence
  Synthetic sequence
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagcttgccgccaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgaatcgataaaataaaagattttatttagtctccagaaaaaggggggaatgaaagaccccacctgtaggtttggtctagagggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcattattattgaagcatttatcagggttattgtctcatgagcggatacatatttgaatgtatttagaaaaataaacaaataggggttccgcgcacatttccccgaaaagtgccacctgacgtc

SEQ ID NO: 105 - DNA, 6101 bp, Artificial Sequence
  Synthetic sequence
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagcttgtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgcactcctctagagggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcattattattgaagcatttatcagggttattgtctcatgagcggatacatatttgaatgtatttagaaaaataaacaaataggggttccgcgcacatttccccgaaaagtgccacctgacgtc

SEQ ID NO: 106 - DNA, 2684 bp, Artificial Sequence
  Synthetic sequence
tatagtgtcacctaaatcgtatgtgtatgatacataaggttatgtattaattgtagccgcgttctaacgacaatatgtccatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcatagggataacagggtaattaactataacggtcctaaggtagcgagggcccatcgattggccatcgcgaatgcatcacgtgctgcagcagctggagctcccgcggcctgcaggtacgtaaggccttggatgtatgttaatatggactaaaggaggcttttgtcgacggatccgatatcggtaccctcctgcttaagggcgcgccggccgcaaattaaagccttcgagcgtcccaaaaccttctcaagcaaggttttcagtataatgttacatgcgtacacgcgtctgtacagaaaaaaaagaaaaatttgaaatataaataacgttcttaatactaacataactataaaaaaataaatagggacctagacttcaggttgtctaactccttccttttcggttagagcggatgtggggggagggcgtgaatgtaagcgtgacataactaattacatgactcgagtctagacctaggcgtacgtgatcaactagtggcgccgcatgccccggggctagccaattgttaattaagcggccgcgttc

SEQ ID NO: 107 - DNA, 2890 bp, Artificial Sequence
  Synthetic sequence
gaacgcggccgcttaattaacaattggctagccccggggcatgcggcgccactagttgatcacgtacgcctaggtctagactcgagtcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcggccggcgcgcccttaagcaggagggtaccgatatcagatctaagcttgaattcgaatttttactaacaaatggtattatttataacagatcttgactgatttttctagggataacagggtaattaactataacggtcctaaggtagcgagggcccatcgattggccatcgcgaatgcatcacgtgctgcagcagctggagctcccgcggcctgcaggtacgtaaggcctaacctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatggacatattgtcgttagaacgcggctacaattaatacataaccttatgtatcatacacatacgatttaggtgacactata

SEQ ID NO: 108 - DNA, 6344 bp, Artificial Sequence
  Synthetic sequence
gaacgcggccgccagctgaagcttcgtacgctgcaggtcgacggatccccgggttaattaagggcccacaaggttttgcattgaggatagtatagaagcaagaatcattgaattacaggaaaaaaaggcaaatatgattcatgctacaataaaccaagatgaagctgccattagcagactaacgccagctgatttacagttcttattcaataactaatattttattctcttattatatattattctcggagtttttaagtgacatcacccgaaaagaagctaagtctttctcctaattcatatttaattattgtacatggacatatcatacgtaatgctcaaccttagctagctagtacggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggagaaaaaaccccggattctagaactagtggatcccccgggctgcaggaattcgatatcaagcttatcgataccgtcgaggggcagagccgatcctgtacactttacttaaaaccattatctgagtgttaaatgtccaatttactgaccgtacaccaaaatttgcctgcattaccggtcgatgcaacgagtgatgaggttcgcaagaacctgatggacatgttcagggatcgccaggcgttttctgagcatacctggaaaatgcttctgtccgtttgccggtcgtgggcggcatggtgcaagttgaataaccggaaatggtttcccgcagaacctgaagatgttcgcgattatcttctatatcttcaggcgcgcggtctggcagtaaaaactatccagcaacatttgggccagctaaacatgcttcatcgtcggtccgggctgccacgaccaagtgacagcaatgctgtttcactggttatgcggcggatccgaaaagaaaacgttgatgccggtgaacgtgcaaaacaggctctagcgttcgaacgcactgatttcgaccaggttcgttcactcatggaaaatagcgatcgctgccaggatatacgtaatctggcatttctggggattgcttataacaccctgttacgtatagccgaaattgccaggatcagggttaaagatatctcacgtactgacggtgggagaatgttaatccatattggcagaacgaaaacgctggttagcaccgcaggtgtagagaaggcacttagcctgggggtaactaaactggtcgagcgatggatttccgtctctggtgtagctgatgatccgaataactacctgttttgccgggtcagaaaaaatggtgttgccgcgccatctgccaccagccagctatcaactcgcgccctggaagggatttttgaagcaactcatcgattgatttacggcgctaaggatgactctggtcagagatacctggcctggtctggacacagtgcccgtgtcggagccgcgcgagatatggcccgcgctggagtttcaataccggagatcatgcaagctggtggctggaccaatgtaaatattgtcatgaactatatccgtaccctggatagtgaaacaggggcaatggtgcgcctgctggaagatggcgattagccattaacgcgtaaatgattgctataattatttgatatttatggtgacatatgagaaaggatttcaacatcgacggaaaatatgtagtgctgtctgtaagcactaatattcagtcgccagccgtcattgtcactgtaaagctgagcgatagaatgcctgatattgactcaatatccgttgcgtttcctgtcaaaagtatgcgtagtgctgaacatttcgtgatgaatgccaccgaggaagaagcacggcgcggttttgctaaagtgatgtctgagtttggcgaactcttgggtaaggttggaattgtcgacctcgagtcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcggccggcgcgccagatctgtttagcttgcctcgtccccgccgggtcacccggccagcgacatggaggcccagaataccctccttgacagtcttgacgtgcgcagctcaggggcatgatgtgactgtcgcccgtacatttagcccatacatccccatgtataatcatttgcatccatacattttgatggccgcacggcgcgaagcaaaaattacggctcctcgctgcagacctgcgagcagggaaacgctcccctcacagacgcgttgaattgtccccacgccgcgcccctgtagagaaatataaaaggttaggatttgccactgaggttcttctttcatatacttccttttaaaatcttgctaggatacagttctcacatcacatccgaacataaacaaccatgggtaccactcttgacgacacggcttaccggtaccgcaccagtgtcccgggggacgccgaggccatcgaggcactggatgggtccttcaccaccgacaccgtcttccgcgtcaccgccaccggggacggcttcaccctgcgggaggtgccggtggacccgcccctgaccaaggtgttccccgacgacgaatcggacgacgaatcggacgacggggaggacggcgacccggactcccggacgttcgtcgcgtacggggacgacggcgacctggcgggcttcgtggtcgtctcgtactccggctggaaccgccggctgaccgtcgaggacatcgaggtcgccccggagcaccgggggcacggggtcgggcgcgcgttgatggggctcgcgacggagttcgcccgcgagcggggcgccgggcacctctggctggaggtcaccaacgtcaacgcaccggcgatccacgcgtaccggcggatggggttcaccctctgcggcctggacaccgccctgtacgacggcaccgcctcggacggcgagcaggcgctctacatgagcatgccctgcccctaatcagtactgacaataaaaagattcttgttttcaagaacttgtcatttgtatagtttttttatattgtagttgttctattttaatcaaatgttagcgtgatttatattttttttcgcctcgacatcatctgcccagatgcgaagttaagtgcgcagaaagtaatatcatgcgtcaatcgtatgtgaatgctggtcgctatactgctgtcgattcgatactaacgccgccatccagtgtcgaaaacgagctccattagtgagtaactctgtgatatctctctataattagcagtttttcactgaaattccaggaaaggtaataaactcagatttttttttatactattggctgcttgttacttatatatcttgaacttctcccagcgggtcttcaaatacatttgggcgatgttcatgttcattaggcaggtatttcgacattgagtcacacgcgaaaaaccgccggaattttttatgtaattgcaagtggaattccgctggcaaaactattgggcccgttaacctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatggacatattgtcgttagaacgcggctacaattaatacataaccttatgtatcatacacatacgatttaggtgacactata

SEQ ID NO: 109 - DNA, 4697 bp, Artificial Sequence
  Synthetic sequence
tatagtgtcacctaaatcgtatgtgtatgatacataaggttatgtattaattgtagccgcgttctaacgacaatatgtccatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcatagggataacagggtaattaactataacggtcctaaggtagcgagggcccatcgattggccatcgcgaatgcatcacgtgctgcagcagctggagctcccgcggcctgcaggtacgtaaggccttggatgtatgttaatatggactaaaggaggcttttgtcgacggatccgatatcggtaccctcctgcttaagggcgcgccggccgcaaattaaagccttcgagcgtcccaaaaccttctcaagcaaggttttcagtataatgttacatgcgtacacgcgtctgtacagaaaaaaaagaaaaatttgaaatataaataacgttcttaatactaacataactataaaaaaataaatagggacctagacttcaggttgtctaactccttccttttcggttagagcggatgtggggggagggcgtgaatgtaagcgtgacataactaattacatgactcgagtctagacctaggcgtacgtgatcaactagtggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagcttgccgccaccatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgacgcccgccccacgacccgcagcgcccgaccgaaaggagcgcacgaccccatgcatcgataaaataaaagattttatttagtctccagaaaaaggggggaatgaaagaccccacctgtaggtttggcaagctagctctagagggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgctagccaattgttaattaagcggccgcgttc

SEQ ID NO: 110 - DNA, 5217 bp, Artificial Sequence
  Synthetic sequence
gaacgcggccgcttaattaacaattggctagcgctggcaagtgtagcggtcacgctgcgcgtaaccaccacacccgccgcgcttaatgcgccgctacagggcgcgtggggataccccctagagccccagctggttctttccgcctcagaagccatagagcccaccgcatccccagcatgcctgctattgtcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagtcgaggctgatcagcgagctctagcatttaggtgacactatagaatagggccctctagacttgttcggtcggcatctactctattcctttgccctcggacgagtgctggggcgtcggtttccactatcggcgagtacttctacacagccatcggtccagacggccgcgcttctgcgggcgatttgtgtacgcccgacagtcccggctccggatcggacgattgcgtcgcatcgaccctgcgcccaagctgcatcatcgaaattgccgtcaaccaagctctgatagagttggtcaagaccaatgcggagcatatacgccccggagccgcggcgatcctgcaagctccggatgcctccgctcgaagtagcgcgtctgctgctccatacaagccaaccacggcctccagaagaagatgttggcgacctcgtattgggaatccccgaacatcgcctcgctccagtcaatgaccgctgttatgcggccattgtccgtcaggacattgttggagccgaaatccgcgtgcacgaggtgccggacttcggggcagtcctcggcccaaagcatcagctcatcgagagcctgcgcgacggacgcactgacggtgtcgtccatcacagtttgccagtgatacacatggggatcagcaatcgcgcatatgaaatcacgccatgtagtgtattgaccgattccttgcggtccgaatgggccgaacccgctcgtctggctaagatcggccgcagcgatcgcatccatggcctccgcgaccggctgcagaacagcgggcagttcggtttcaggcaggtcttgcaacgtgacaccctgtgcacggcgggagatgcaataggtcaggctctcgctgaattccccaatgtcaagcacttccggaatcgggagcgcggccgatgcaaagtgccgataaacataacgatctttgtagaaaccatcggcgcagctatttacccgcaggacatatccacgccctcctacatcgaagctgaaagcacgagattcttcgccctccgagagctgcatcaggtcggagacgctgtcgaacttttcgatcagaaacttctcgacagacgtcgcggtgagttcaggctttttcatggtggcggcaagcttgggtctccctatagtgagtcgtattaatttcgataagccagtaagcagtgggttctctagttagccagagagctctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatagtccacccattgacgtcaatggaaagtccctattggcgttactatgggaacatacgtcattattgacgtcaatgggcgggggtcgttgggcggtcagccaggcgggccatttaccgtaagttatgtaacgcggaactccatatatgggctatgaactaatgaccccgtaattgattactattaataactagtcaataatcaatgtcaacgcgtatatctggcccgtacatcgcgaagcagcgcaaaacgcctaaccctaagcagattcttcatgcaattgtcggtcaagccttgccttgttgtagcttaaattttgctcgcgcactactcagcgacctccaacacacaagcagggagcagatactggcttaactatgcggcatcagagcagattgtactgagagtcgaccataggggatcgggagatctccactagttgatcacgtacgcctaggtctagactcgagtcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcggccggcgcgcccttaagcaggagggtaccgatatcagatctaagcttgaattcgaatttttactaacaaatggtattatttataacagatcttgactgatttttctagggataacagggtaattaactataacggtcctaaggtagcgagggcccatcgattggccatcgcgaatgcatcacgtgctgcagcagctggagctcccgcggcctgcaggtacgtaaggcctaacctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatggacatattgtcgttagaacgcggctacaattaatacataaccttatgtatcatacacatacgatttaggtgacactata

SEQ ID NO: 111 - DNA, 4745 bp, Artificial Sequence
  Synthetic sequence
gaacgcggccgcttaattaacaattggctagcgctggcaagtgtagcggtcacgctgcgcgtaaccaccacacccgccgcgcttaatgcgccgctacagggcgcgtggggataccccctagagccccagctggttctttccgcctcagaagccatagagcccaccgcatccccagcatgcctgctattgtcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagtcgaggctgatcagcgagctctagcatttaggtgacactatagaatagggccctctagagctagcttgccaaacctacaggtggggtctttcattcccccctttttctggagactaaataaaatcttttattttatcgatgcatggggtcgtgcgctcctttcggtcgggcgctgcgggtcgtggggcgggcgtcaggcaccgggcttgcgggtcatgcaccaggtgcgcggtccttcgggcacctcgacgtcggcggtgacggtgaagccgagccgctcgtagaaggggaggttgcggggcgcggaggtctccaggaaggcgggcaccccggcgcgctcggccgcctccactccggggagcacgacggcgctgcccagacccttgccctggtggtcgggcgagacgccgacggtggccaggaaccacgcgggctccttgggccggtgcggcgccaggaggccttccatctgttgctgcgcggccagccgggaaccgctcaactcggccatgcgcgggccgatctcggcgaacaccgcccccgcttcgacgctctccggcgtggtccagaccgccaccgcggcgccgtcgtccgcgacccacaccttgccgatgtcgagcccgacgcgcgtgaggaagagttcttgcagctcggtgacccgctcgatgtggcggtccggatcgacggtgtggcgcgtggcggggtagtcggcgaacgcggcggcgagggtgcgtacggccctggggacgtcgtcgcgggtggcgaggcgcaccgtgggcttgtactcggtcatggtggcggcaagcttgggtctccctatagtgagtcgtattaatttcgataagccagtaagcagtgggttctctagttagccagagagctctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatagtccacccattgacgtcaatggaaagtccctattggcgttactatgggaacatacgtcattattgacgtcaatgggcgggggtcgttgggcggtcagccaggcgggccatttaccgtaagttatgtaacgcggaactccatatatgggctatgaactaatgaccccgtaattgattactattaataactagtcaataatcaatgtcaacgcgtatatctggcccgtacatcgcgaagcagcgcaaaacgcctaaccctaagcagattcttcatgcaattgtcggtcaagccttgccttgttgtagcttaaattttgctcgcgcactactcagcgacctccaacacacaagcagggagcagatactggcttaactatgcggcatcagagcagattgtactgagagtcgaccataggggatcgggagatctccactagttgatcacgtacgcctaggtctagactcgagtcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcggccggcgcgcctaccgttcgtataggatactttatacgaagttataacgtaacctcgaaatacctttgctaggtaccgatatcggatccgtcgacaaaagcctcctttagtccatattaacatacatccaaggccttacgtacctgcaggccgcgggagctccagctgctgcagcacgtgatgcattcgcgatggccaatcgatgggccctcgctaccttaggaccgttatagttaattaccctgttatccctatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatggacatattgtcgttagaacgcggctacaattaatacataaccttatgtatcatacacatacgatttaggtgacactata

SEQ ID NO: 112 - DNA, 5266 bp, Artificial Sequence
  Synthetic sequence
tatagtgtcacctaaatcgtatgtgtatgatacataaggttatgtattaattgtagccgcgttctaacgacaatatgtccatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcaggttaggccttacgtacctgcaggccgcgggagctccagctgctgcagcacgtgatgcattcgcgatggccaatcgatgggccctcgctaccttaggaccgttatagttaattaccctgttatccctagaaaaatcagtcaagatctgttataaataataccatttgttagtaaaaattcgaattcaagcttagatctgatatcggtacctagcaaaggtatttcgaggttacgttttaccgttcgtatagcatacattatacgaagttatggcgcgccggccgcaaattaaagccttcgagcgtcccaaaaccttctcaagcaaggttttcagtataatgttacatgcgtacacgcgtctgtacagaaaaaaaagaaaaatttgaaatataaataacgttcttaatactaacataactataaaaaaataaatagggacctagacttcaggttgtctaactccttccttttcggttagagcggatgtggggggagggcgtgaatgtaagcgtgacataactaattacatgactcgagtctagacctaggcgtacgtgatcaactagtggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagcttgccgccaccatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccggggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagagtagatgccgaccgaacaagtctagagggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgctagccaattgttaattaagcggccgcgttc

SEQ ID NO: 113 - DNA, 6116 bp, Artificial Sequence
  Synthetic sequence
gaacgcggccgcttaattaacaattggctagcgctggcaagtgtagcggtcacgctgcgcgtaaccaccacacccgccgcgcttaatgcgccgctacagggcgcgtggggataccccctagagccccagctggttctttccgcctcagaagccatagagcccaccgcatccccagcatgcctgctattgtcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagtcgaggctgatcagcgagctctagcatttaggtgacactatagaatagggccctctagagctagcttgccaaacctacaggtggggtctttcattcccccctttttctggagactaaataaaatcttttattttatcgatgcatggggtcgtgcgctcctttcggtcgggcgctgcgggtcgtggggcgggcgtcaggcaccgggcttgcgggtcatgcaccaggtgcgcggtccttcgggcacctcgacgtcggcggtgacggtgaagccgagccgctcgtagaaggggaggttgcggggcgcggaggtctccaggaaggcgggcaccccggcgcgctcggccgcctccactccggggagcacgacggcgctgcccagacccttgccctggtggtcgggcgagacgccgacggtggccaggaaccacgcgggctccttgggccggtgcggcgccaggaggccttccatctgttgctgcgcggccagccgggaaccgctcaactcggccatgcgcgggccgatctcggcgaacaccgcccccgcttcgacgctctccggcgtggtccagaccgccaccgcggcgccgtcgtccgcgacccacaccttgccgatgtcgagcccgacgcgcgtgaggaagagttcttgcagctcggtgacccgctcgatgtggcggtccggatcgacggtgtggcgcgtggcggggtagtcggcgaacgcggcggcgagggtgcgtacggccctggggacgtcgtcgcgggtggcgaggcgcaccgtgggcttgtactcggtcatggtggcggcaagcttgggtctccctatagtgagtcgtattaatttcgataagccagtaagcagtgggttctctagttagccagagagctctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatagtccacccattgacgtcaatggaaagtccctattggcgttactatgggaacatacgtcattattgacgtcaatgggcgggggtcgttgggcggtcagccaggcgggccatttaccgtaagttatgtaacgcggaactccatatatgggctatgaactaatgaccccgtaattgattactattaataactagtcaataatcaatgtcaacgcgtatatctggcccgtacatcgcgaagcagcgcaaaacgcctaaccctaagcagattcttcatgcaattgtcggtcaagccttgccttgttgtagcttaaattttgctcgcgcactactcagcgacctccaacacacaagcagggagcagatactggcttaactatgcggcatcagagcagattgtactgagagtcgaccataggggatcgggagatctccactagttgatcacgtacgcctaggtctagactcgagtcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcggccggcgcgcctaccgttcgtataggatactttatacgaagttataacgtaacctcgaaatacctttgctaggtaccgatatcggatccgtcgacaaaagcctcctttagtccatattaacatacatccaaggccttacgtacctgcaggccgcgggagctccagctgctgcagcacgtgatgccatagagcccaccgcatccccagcatgcctgctattgtcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagtcgaggctgatcagcgagctctagcatttaggtgacactatagaatagggccctctagagtaccgagctcgaattgtgcttagccctcccacacataaccagagggcagcaattcacgaatcccaactgccgtcggctgtccatcactgtccttcactatggctttgatcccaggatgcagatcgagaagcacctgtcggcaccgtccgcaggggctcaagatgcccctgttctcatttccgatcgcgacgatacaagtcaggttgccagctgccgcagcagcagcagtgcccagcaccacgagttctgcacaaggtcccccagtaaaatgatatacattgacaccagtgaagatgcggccgtcgctagagagagctgcgctggcgacgctgtagtcttcagagatggggatgctgttgattgtagccgttgctctttcaatgagggtggattcttcttgagacaaaggcttggccatggtggcggcaagcttgggtctccctatagtgagtcgtattaatttcgataagccagtaagcagtgggttctctagttagccagagagctctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatagtccacccattgacgtcaatggaaagtccctattggcgttactatgggaacatacgtcattattgacgtcaatgggcgggggtcgttgggcggtcagccaggcgggccatttaccgtaagttatgtaacgcggaactccatatatgggctatgaactaatgaccccgtaattgattactattaataactagtcaataatcaatgtcattcgcgatggccaatcgatgggccctcgctaccttaggaccgttatagttaattaccctgttatccctatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatggacatattgtcgttagaacgcggctacaattaatacataaccttatgtatcatacacatacgatttaggtgacactata

SEQ ID NO: 114 - DNA, 6144 bp, Artificial Sequence
  Synthetic sequence
gaacgcggccgcttaattaacaattggctagcgctggcaagtgtagcggtcacgctgcgcgtaaccaccacacccgccgcgcttaatgcgccgctacagggcgcgtggggataccccctagagccccagctggttctttccgcctcagaagccatagagcccaccgcatccccagcatgcctgctattgtcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagtcgaggctgatcagcgagctctagcatttaggtgacactatagaatagggccctctagagctagcttgccaaacctacaggtggggtctttcattcccccctttttctggagactaaataaaatcttttattttatcgatgcatggggtcgtgcgctcctttcggtcgggcgctgcgggtcgtggggcgggcgtcaggcaccgggcttgcgggtcatgcaccaggtgcgcggtccttcgggcacctcgacgtcggcggtgacggtgaagccgagccgctcgtagaaggggaggttgcggggcgcggaggtctccaggaaggcgggcaccccggcgcgctcggccgcctccactccggggagcacgacggcgctgcccagacccttgccctggtggtcgggcgagacgccgacggtggccaggaaccacgcgggctccttgggccggtgcggcgccaggaggccttccatctgttgctgcgcggccagccgggaaccgctcaactcggccatgcgcgggccgatctcggcgaacaccgcccccgcttcgacgctctccggcgtggtccagaccgccaccgcggcgccgtcgtccgcgacccacaccttgccgatgtcgagcccgacgcgcgtgaggaagagttcttgcagctcggtgacccgctcgatgtggcggtccggatcgacggtgtggcgcgtggcggggtagtcggcgaacgcggcggcgagggtgcgtacggccctggggacgtcgtcgcgggtggcgaggcgcaccgtgggcttgtactcggtcatggtggcggcaagcttgggtctccctatagtgagtcgtattaatttcgataagccagtaagcagtgggttctctagttagccagagagctctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatagtccacccattgacgtcaatggaaagtccctattggcgttactatgggaacatacgtcattattgacgtcaatgggcgggggtcgttgggcggtcagccaggcgggccatttaccgtaagttatgtaacgcggaactccatatatgggctatgaactaatgaccccgtaattgattactattaataactagtcaataatcaatgtcaacgcgtatatctggcccgtacatcgcgaagcagcgcaaaacgcctaaccctaagcagattcttcatgcaattgtcggtcaagccttgccttgttgtagcttaaattttgctcgcgcactactcagcgacctccaacacacaagcagggagcagatactggcttaactatgcggcatcagagcagattgtactgagagtcgaccataggggatcgggagatctccactagttgatcacgtacgcctaggtctagactcgagtcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcggccggcgcgcctaccgttcgtataggatactttatacgaagttataacgtaacctcgaaatacctttgctaggtaccgatatcggatccgtcgacaaaagcctcctttagtccatattaacatacatccaaggccttacgtacctgcaggccgcgggagctccagctgctgcagcacgtgatgccatagagcccaccgcatccccagcatgcctgctattgtcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagtcgaggctgatcagcgagctctagcatttaggtgacactatagaatagggccctctagaccaaacctacaggtggggtctttcattcccccctttttctggagactaaataaaatcttttattttatcgattcagtcctgctcctcggccacgaagtgcacgcagttgccggccgggtcgcgcagggcgaactcccgcccccacggctgctcgccgatctcggtcatggccggcccggaggcgtcccggaagttcgtggacacgacctccgaccactcggcgtacagctcgtccaggccgcgcacccacacccaggccagggtgttgtccggcaccacctggtcctggaccgcgctgatgaacagggtcacgtcgtcccggaccacaccggcgaagtcgtcctccacgaagtcccgggagaacccgagccggtcggtccagaactcgaccgctccggcgacgtcgcgcgcggtgagcaccggaacggcactggtcaacttggccatggtggcggcaagcttgggtctccctatagtgagtcgtattaatttcgataagccagtaagcagtgggttctctagttagccagagagctctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatagtccacccattgacgtcaatggaaagtccctattggcgttactatgggaacatacgtcattattgacgtcaatgggcgggggtcgttgggcggtcagccaggcgggccatttaccgtaagttatgtaacgcggaactccatatatgggctatgaactaatgaccccgtaattgattactattaataactagtcaataatcaatgtcattcgcgatggccaatcgatgggccctcgctaccttaggaccgttatagttaattaccctgttatccctatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatggacatattgtcgttagaacgcggctacaattaatacataaccttatgtatcatacacatacgatttaggtgacactata

SEQ ID NO: 115 - DNA, 6431 bp, Artificial Sequence
  Synthetic sequence
gaacgcggccgcttaattaacaattggctagcgctggcaagtgtagcggtcacgctgcgcgtaaccaccacacccgccgcgcttaatgcgccgctacagggcgcgtggggataccccctagagccccagctggttctttccgcctcagaagccatagagcccaccgcatccccagcatgcctgctattgtcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagtcgaggctgatcagcgagctctagcatttaggtgacactatagaatagggccctctagagctagcttgccaaacctacaggtggggtctttcattcccccctttttctggagactaaataaaatcttttattttatcgatgcatggggtcgtgcgctcctttcggtcgggcgctgcgggtcgtggggcgggcgtcaggcaccgggcttgcgggtcatgcaccaggtgcgcggtccttcgggcacctcgacgtcggcggtgacggtgaagccgagccgctcgtagaaggggaggttgcggggcgcggaggtctccaggaaggcgggcaccccggcgcgctcggccgcctccactccggggagcacgacggcgctgcccagacccttgccctggtggtcgggcgagacgccgacggtggccaggaaccacgcgggctccttgggccggtgcggcgccaggaggccttccatctgttgctgcgcggccagccgggaaccgctcaactcggccatgcgcgggccgatctcggcgaacaccgcccccgcttcgacgctctccggcgtggtccagaccgccaccgcggcgccgtcgtccgcgacccacaccttgccgatgtcgagcccgacgcgcgtgaggaagagttcttgcagctcggtgacccgctcgatgtggcggtccggatcgacggtgtggcgcgtggcggggtagtcggcgaacgcggcggcgagggtgcgtacggccctggggacgtcgtcgcgggtggcgaggcgcaccgtgggcttgtactcggtcatggtggcggcaagcttgggtctccctatagtgagtcgtattaatttcgataagccagtaagcagtgggttctctagttagccagagagctctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatagtccacccattgacgtcaatggaaagtccctattggcgttactatgggaacatacgtcattattgacgtcaatgggcgggggtcgttgggcggtcagccaggcgggccatttaccgtaagttatgtaacgcggaactccatatatgggctatgaactaatgaccccgtaattgattactattaataactagtcaataatcaatgtcaacgcgtatatctggcccgtacatcgcgaagcagcgcaaaacgcctaaccctaagcagattcttcatgcaattgtcggtcaagccttgccttgttgtagcttaaattttgctcgcgcactactcagcgacctccaacacacaagcagggagcagatactggcttaactatgcggcatcagagcagattgtactgagagtcgaccataggggatcgggagatctccactagttgatcacgtacgcctaggtctagactcgagtcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcggccggcgcgcctaccgttcgtataggatactttatacgaagttataacgtaacctcgaaatacctttgctaggtaccgatatcggatccgtcgacaaaagcctcctttagtccatattaacatacatccaaggccttacgtacctgcaggccgcgggagctccagctgctgcagcacgtgatgccatagagcccaccgcatccccagcatgcctgctattgtcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagtcgaggctgatcagcgagctctagcatttaggtgacactatagaatagggccctctagagctagcttgccaaacctacaggtggggtctttcattcccccctttttctggagactaaataaaatcttttattttatcgatgcatggggtcgtgcgctcctttcggtcgggcgctgcgggtcgtggggcgggcgtcaggcaccgggcttgcgggtcatgcaccaggtgcgcggtccttcgggcacctcgacgtcggcggtgacggtgaagccgagccgctcgtagaaggggaggttgcggggcgcggaggtctccaggaaggcgggcaccccggcgcgctcggccgcctccactccggggagcacgacggcgctgcccagacccttgccctggtggtcgggcgagacgccgacggtggccaggaaccacgcgggctccttgggccggtgcggcgccaggaggccttccatctgttgctgcgcggccagccgggaaccgctcaactcggccatgcgcgggccgatctcggcgaacaccgcccccgcttcgacgctctccggcgtggtccagaccgccaccgcggcgccgtcgtccgcgacccacaccttgccgatgtcgagcccgacgcgcgtgaggaagagttcttgcagctcggtgacccgctcgatgtggcggtccggatcgacggtgtggcgcgtggcggggtagtcggcgaacgcggcggcgagggtgcgtacggccctggggacgtcgtcgcgggtggcgaggcgcaccgtgggcttgtactcggtcatggtggcggcaagcttgggtctccctatagtgagtcgtattaatttcgataagccagtaagcagtgggttctctagttagccagagagctctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatagtccacccattgacgtcaatggaaagtccctattggcgttactatgggaacatacgtcattattgacgtcaatgggcgggggtcgttgggcggtcagccaggcgggccatttaccgtaagttatgtaacgcggaactccatatatgggctatgaactaatgaccccgtaattgattactattaataactagtcaataatcaatgtcattcgcgatggccaatcgatgggccctcgctaccttaggaccgttatagttaattaccctgttatccctatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatggacatattgtcgttagaacgcggctacaattaatacataaccttatgtatcatacacatacgatttaggtgacactata

SEQ ID NO: 116 - DNA, 6431 bp, Artificial Sequence; identical to SEQ ID NO: 115
  Synthetic sequence

SEQ ID NO: 117 - DNA, 6637 bp, Artificial Sequence
  Synthetic sequence
tatagtgtcacctaaatcgtatgtgtatgatacataaggttatgtattaattgtagccgcgttctaacgacaatatgtccatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcaggttaggccttacgtacctgcaggccgcgggagctccagctgctgcagcacgtgatgccatagagcccaccgcatccccagcatgcctgctattgtcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagtcgaggctgatcagcgagctctagcatttaggtgacactatagaatagggccctctagagtaccgagctcgaattgtgcttagccctcccacacataaccagagggcagcaattcacgaatcccaactgccgtcggctgtccatcactgtccttcactatggctttgatcccaggatgcagatcgagaagcacctgtcggcaccgtccgcaggggctcaagatgcccctgttctcatttccgatcgcgacgatacaagtcaggttgccagctgccgcagcagcagcagtgcccagcaccacgagttctgcacaaggtcccccagtaaaatgatatacattgacaccagtgaagatgcggccgtcgctagagagagctgcgctggcgacgctgtagtcttcagagatggggatgctgttgattgtagccgttgctctttcaatgagggtggattcttcttgagacaaaggcttggccatggtggcggcaagcttgggtctccctatagtgagtcgtattaatttcgataagccagtaagcagtgggttctctagttagccagagagctctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatagtccacccattgacgtcaatggaaagtccctattggcgttactatgggaacatacgtcattattgacgtcaatgggcgggggtcgttgggcggtcagccaggcgggccatttaccgtaagttatgtaacgcggaactccatatatgggctatgaactaatgaccccgtaattgattactattaataactagtcaataatcaatgtcattcgcgatggccaatcgatgggccctcgctaccttaggaccgttatagttaattaccctgttatccctagaaaaatcagtcaagatctgttataaataataccatttgttagtaaaaattcgaattcaagcttagatctgatatcggtacctagcaaaggtatttcgaggttacgttttaccgttcgtatagcatacattatacgaagttatggcgcgccggccgcaaattaaagccttcgagcgtcccaaaaccttctcaagcaaggttttcagtataatgttacatgcgtacacgcgtctgtacagaaaaaaaagaaaaatttgaaatataaataacgttcttaatactaacataactataaaaaaataaatagggacctagacttcaggttgtctaactccttccttttcggttagagcggatgtggggggagggcgtgaatgtaagcgtgacataactaattacatgactcgagtctagacctaggcgtacgtgatcaactagtggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagcttgccgccaccatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccggggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagagtagatgccgaccgaacaagtctagagggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgctagccaattgttaattaagcggccgcgttc

SEQ ID NO: 118 - DNA, 6797 bp, Artificial Sequence
  Synthetic sequence
tatagtgtcacctaaatcgtatgtgtatgatacataaggttatgtattaattgtagccgcgttctaacgacaatatgtccatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcaggttaggccttacgtacctgcaggccgcgggagctccagctgctgcagcacgtgatgaggtcgacggtatacagacatgataagatacattgatgagtttggacaaaccacaactagaatgcagtgaaaaaaatgctttatttgtgaaatttgtgatgctattgctttatttgtaaccattataagctgcaataaacaagttggggtgggcgaagaactccagcatgagatccccgcgctggaggatcatccagccggcgtcccggaaaacgattccgaagcccaacctttcatagaaggcggcggtggaatcgaaatctcgtgatggcaggttgggcgtcgcttggtcggtcatttcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagatccgaaaatggatatacaagctcccgggagctttttgcaaaagcctaggcctccaaaaaagcctcctcactacttctggaatagctcagaggcagaggcggcctcggcctctgcataaataaaaaaaattagtcagccatggggcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgcctggggagcctggggactttccacaccctaactgacacacattccacagaattaattcgcgcattcgcgatggccaatcgatgggccctcgctaccttaggaccgttatagttaattaccctgttatccctagaaaaatcagtcaagatctgttataaataataccatttgttagtaaaaattcgaattcaagcttagatctgatatcggtacctagcaaaggtatttcgaggttacgttttaccgttcgtatagcatacattatacgaagttatggcgcgccggccgcaaattaaagccttcgagcgtcccaaaaccttctcaagcaaggttttcagtataatgttacatgcgtacacgcgtctgtacagaaaaaaaagaaaaatttgaaatataaataacgttcttaatactaacataactataaaaaaataaatagggacctagacttcaggttgtctaactccttccttttcggttagagcggatgtggggggagggcgtgaatgtaagcgtgacataactaattacatgactcgagtctagacctaggcgtacgtgatcaactagtggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagcttgccgccaccatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccggggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagagtagatgccgaccgaacaagtctagagggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgctagccaattgttaattaagcggccgcgttc

SEQ ID NO: 119 - DNA, 6637 bp, Artificial Sequence; identical to SEQ ID NO: 117
  Synthetic sequence

SEQ ID NO: 120 - DNA, 6952 bp, Artificial Sequence
  Synthetic sequence
tatagtgtcacctaaatcgtatgtgtatgatacataaggttatgtattaattgtagccgcgttctaacgacaatatgtccatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcaggttaggccttacgtacctgcaggccgcgggagctccagctgctgcagcacgtgatgccatagagcccaccgcatccccagcatgcctgctattgtcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagtcgaggctgatcagcgagctctagcatttaggtgacactatagaatagggccctctagaggagtgcggccgctttacttgtacagctcgtccatgccgagagtgatcccggcggcggtcacgaactccagcaggaccatgtgatcgcgcttctcgttggggtctttgctcagggcggactgggtgctcaggtagtggttgtcgggcagcagcacggggccgtcgccgatgggggtgttctgctggtagtggtcggcgagctgcacgctgccgtcctcgatgttgtggcggatcttgaagttcaccttgatgccgttcttctgcttgtcggccatgatatagacgttgtggctgttgtagttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcggctgaagcactgcacgccgtaggtcagggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcaccatggtggcgacaagcttgggtctccctatagtgagtcgtattaatttcgataagccagtaagcagtgggttctctagttagccagagagctctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatagtccacccattgacgtcaatggaaagtccctattggcgttactatgggaacatacgtcattattgacgtcaatgggcgggggtcgttgggcggtcagccaggcgggccatttaccgtaagttatgtaacgcggaactccatatatgggctatgaactaatgaccccgtaattgattactattaataactagtcaataatcaatgtcattcgcgatggccaatcgatgggccctcgctaccttaggaccgttatagttaattaccctgttatccctagaaaaatcagtcaagatctgttataaataataccatttgttagtaaaaattcgaattcaagcttagatctgatatcggtacctagcaaaggtatttcgaggttacgttttaccgttcgtatagcatacattatacgaagttatggcgcgccggccgcaaattaaagccttcgagcgtcccaaaaccttctcaagcaaggttttcagtataatgttacatgcgtacacgcgtctgtacagaaaaaaaagaaaaatttgaaatataaataacgttcttaatactaacataactataaaaaaataaatagggacctagacttcaggttgtctaactccttccttttcggttagagcggatgtggggggagggcgtgaatgtaagcgtgacataactaattacatgactcgagtctagacctaggcgtacgtgatcaactagtggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagcttgccgccaccatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccggggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagagtagatgccgaccgaacaagtctagagggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgctagccaattgttaattaagcggccgcgttc

SEQ ID NO: 121 - DNA, 16030 bp, Artificial Sequence
  Synthetic sequence
  misc_feature (5798)..(5802): n is a, c, g, or t
  misc_feature (5805)..(5809): n is a, c, g, or t
  misc_feature (5812)..(5816): n is a, c, g, or t
  misc_feature (5819)..(5823): n is a, c, g, or t
  misc_feature (5922)..(5926): n is a, c, g, or t
  misc_feature (5929)..(5933): n is a, c, g, or t
  misc_feature (5936)..(5940): n is a, c, g, or t
  misc_feature (5943)..(5947): n is a, c, g, or t
ctcgtcgtctgattggctctcggggcccagaaaactggcccttgccattggctcgtgttcgtgcaagttgagtccatccgccggccagcgggggcggcgaggaggcgctcccaggttccggccctcccctcggccccgcgccgcagagtctggccgcgcgcccctgcgcaacgtggcaggaagcgcgcgctgggggcggggacgggcagtagggctgagcggctgcggggcgggtgcaagcacgtttccgacttgagttgcctcaagaggggcgtgctgagccagacctccatcgcgcactccggggagtggagggaaggagcgagggctcagttgggctgttttggaggcaggaagcacttgctctcccaaagtcgctctgagttgttatcagtaagggagctgcagtggagtaggcggggagaaggccgcacccttctccggaggggggaggggagtgttgcaatacctttctgggagttctctgctgcctcctggcttctgaggaccgccctgggcctgggagaatcccttccccctcttccctcgtgatctgcaactccagtctttctagaagatgggcgggagtcttctgggcaggcttaaaggctaacctggtgtgtgggcgttgtcctgcaggggaattgaacaggtgtaaaattggagggacaagacttcccacagattttcggttttgtcgggaagttttttaataggggcaaataaggaaaatgggaggataggtagtcatctggggttttatgcagcaaaactacaggttattattgcttgtgatccgcctcggagtattttccatcgaggtagattaaagacatgctcacccgagttttatactctcctgcttgagatccttactacagtatgaaattacagtgtcgcgagttagactatgtaagcagaattttaatcatttttaaagagcccagtacttcatatccatttctcccgctccttctgcagccttatcaaaaggtattttagaacactcattttagccccattttcatttattatactggcttatccaacccctagacagagcattggcattttccctttcctgatcttagaagtctgatgactcatgaaaccagacagattagttacatacaccacaaatcgaggctgtagctggggcctcaacactgcagttcttttataactccttagtacactttttgttgatcctttgccttgatccttaattttcagtgtctatcacctctcccgtcaggtggtgttccacatttgggcctattctcagtccagggagttttacaacaatagatgtattgagaatccaacctaaagcttaactttccactcccatgaatgcctctctcctttttctccatttataaactgagctattaaccattaatggtttccaggtggatgtctcctcccccaatattacctgatgtatcttacatattgccaggctgatattttaagacattaaaaggtatatttcattattgagccacatggtattgattactgcttactaaaattttgtcattgtacacatctgtaaaaggtggttccttttggaatgcaaagttcaggtgtttgttgtctttcctgacctaaggtcttgtgagcttgtattttttctatttaagcagtgctttctcttggactggcttgactcatggcattctacacgttattgctggtctaaatgtgattttgccaagcttcttcaggacctataattttgcttgacttgtagccaaacacaagtaaaatgattaagcaacaaatgtatttgtgaagcttggtttttaggttgttgtgttgtgtgtgcttgtgctctataataatactatccaggggctggagaggtggctcggagttcaagagcacagactgctcttccagaagtcctgagttcaattcccagcaaccacatggtggctcacaaccatctgtaatgggatctgatgccctcttctggtgtgtctgaagaccacaagtgtattcacattaaataaataaatcctccttcttcttcttttttttttttttaaagagaatactgtctccagtagaatttactgaagtaatgaaatactttgtgtttgttccaatatggtagccaataatcaaattactctttaagcactggaaatgttaccaaggaactaatttttatttgaagtgtaactgtggacagaggagccataactgcagacttgtgggatacagaagaccaatgcagactttaatgtcttttctcttacactaagcaataaagaaataaaaattgaacttctagtatcctatttgtttaaactgctagctttacttaacttttgtgcttcatctatacaaagctgaaagctaagtctgcagccattactaaacatgaaagcaagtaatgataattttggatttcaaaaatgtagggccagagtttagccagccagtggtggtgcttgcctttatgcctttaatcccagcactctggaggcagagacaggcagatctctgagtttgagcccagcctggtctacacatcaagttctatctaggatagccaggaatacacacagaaaccctgttggggaggggggctctgagatttcataaaattataattgaagcattccctaatgagccactatggatgtggctaaatccgtctacctttctgatgagatttgggtattattttttctgtctctgctgttggttgggtcttttgacactgtgggctttctttaaagcctccttcctgccatgtggtctcttgtttgctactaacttcccatggcttaaatggcatggctttttgccttctaagggcagctgctgagatttgcagcctgatttccagggtggggttgggaaatctttcaaacactaaaattgtcctttaattttttttttaaaaaatgggttatataataaacctcataaaatagttatgaggagtgaggtggactaatattaaatgagtccctcctagggataagacagatcgacactgctcgaaagttcagatgtgcggcgagttgcgtgactacctacgggtaacagtttcttaccgttcgtataaagtatcctatacgaagttattaagcaggtaccctcctgcttaagggcgcgccggccgcaaattaaagccttcgagcgtcccaaaaccttctcaagcaaggttttcagtataatgttacatgcgtacacgcgtctgtacagaaaaaaaagaaaaatttgaaatataaataacgttcttaatactaacataactataaaaaaataaatagggacctagacttcaggttgtctaactccttccttttcggttagagcggatgtggggggagggcgtgaatgtaagcgtgacataactaattacatgactcgagtctagacctaggcgtacgtgatcaactagtggagatcttcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcgtcgactttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacctgcagcccaagcttaccatgaccgagtacaagcccacggtgcgcctcgccacccgcgacgacgtccccagcatcacgtgctgcagcagctgggagctcccgcggcctgcaggtacgtaaggccttggatgtatgttaatatggactaaaggaggcttttgtcgacggatccnnnnnaannnnnttnnnnnttnnnnnataacttcgtataaagtatcctatacgaacggtattatggcagggtgaaacgcaggtcgccagctaccgttcgtataatgtatgctatacgaagttatnnnnnaannnnnttnnnnnttnnnnntaagcagaatgcgaattcgaatttttactaacaaatggtattatttataacagatcttgactgatttttctagggataacagggtaattaactataacggtcctaaggtagcgagggcccatcgattggccatcgcgaatgattggccatcgcgaatgcaggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgacgcccgccccacgacccgcagcgcccgaccgaaaggagcgcacgaccccatgcatcgataaaataaaagattttatttagtctccagaaaaaggggggaatgaaagaccccacctgtaggtttggcaagctagcgtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgccaagagatttattactccaactagcattccaaggcacagcagtggtgcaaatgagttttccagagcaaccccaaatccccaggagctgttgatcctttaggtatctttccacagccaggattcttgcctggagctgcttgatgccccagactgtgagttgcaacagatgctgttgcgcctcaatagccctcagcaaattgttctgctgctgcactataccagacaataattgtctggcctgtaccgtcagcgtcattgacgctgcgcccatagtgcttcctgctgctcccaagaacccaaggaacaaagctcctattcccactgctcttttttctctctgcaccactcttctctttgccttggtgggtgctactcctaatggttcaatttttactactttatatttatataattcacttctccaattgtccctcatatctcctcctccaggtctgaagatcagcggccgcttgctgtgcggtggtcttacttttgttttgctcttcctctatcttgtctaaagcttccttggtgtcttttatctctatcctttgatgcacacaatagagggttgctactgtattatataatgatctaagttcttctgatcctgtctgaagggatggttgtagctgtcccagtatttgtctacagccttctgatgtttctaacaggccaggattaactgcgaatcgttctagctccctgcttgcccatactatatgttttaatttatattttttctttccccctggccttaaccgaattttttcccatcgcgatctaattctcccccgcttaatactgacgctctcgcacccatctctctccttctagcctccgctagtcaaaatttttggcgtactcaccagtcgccgcccctcgcctcttgccgtgcgcgcttcagcaagccgagtcctgcgtcgagagagctcctctggtttccctttcgctttcaagtccctgttcgggcgccactgctagagattttccacactgactaaaagggtctgagggatctctagttaccagagtcacacaacagacgggcacacactacttgaagcactcaaggcaagctttattgaggcttaagcagtgggttccctagttagccagagagctcccaggctcagatctggtctaaccagagagaccctgctagagattttccacactgactaaaagggtctgagggatctctagttaccagagtcacacaacagacgggcacacactacttgaagcactcaaggcaagctttattgaggcttaagcagtgggttccctagttagccagagagctcccaggctcagatctggtctaaccagagagacccagtacaagcaaaaagcagatcttgtcttcgttgggagtgaattagcccttccagtccccccttttcttttaaaaagtggctaagatctacagctgccttgtaagtcattggtcttaaagtcgacgcggggaggcggcccaaagggagatccgactcgtctgagggcgaaggcgaagacgcggaagaggccgcagagccggcagcaggccgcgggaaggaaggtccgctggattgagggccgaagggacgtagcagaaggacgtcccgcgcagaatccaggtggcaacacaggcgagcagccaagtcaagccttgccttgttgtagcttaaattttgctcgcgcactactcagcgacctccaacacacaagcagggagcagatactggcttaactatgcggcatcagagcagattgtactgagagtcgaccataggggatcgggagatctccactagttgatcacgtacgcctaggtctagactcgagataacttcgtataatgtatgctatacgaacggtactttcggcggtgaaattatcgatgagcgtggtggttatgccgatcgcgtctccacgtgcatcaaggcgcgccattgatgcggccgggacagcagagatccactttggcgccggctcgagtggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtgtcgtgacgcggggcaaagaattcccgggtgagccgccaccatggctggagacatgagagctgccaacctttggccaagcccgctcatgatcaaacgctctaagaagaacagcctggccttgtccctgacggccgaccagatggtcagtgccttgttggatgctgagccccccatactctattccgagtatgatcctaccagacccttcagtgaagcttcgatgatgggcttactgaccaacctggcagacagggagctggttcacatgatcaactgggcgaagagggtgccaggctttgtggatttgaccctccatgatcaggtccaccttctagaatgtgcctggctagagatcctgatgattggtctcgtctggcgctccatggagcacccagtgaagctactgtttgctcctaacttgctcttggacaggaaccagggaaaatgtgtagagggcatggtggagatcttcgacatgctgctggctacatcatctcggttccgcatgatgaatctgcagggagaggagtttgtgtgcctcaaatctattattttgcttaattctggagtgtacacatttctgtccagcaccctgaagtctctggaagagaaggaccatatccaccgagtcctggacaagatcacagacactttgatccacctgatggccaaggcaggcctgaccctgcagcagcagcaccagcggctggcccagctcctcctcatcctctcccacatcaggcacatgagtaacaaaggcatggagcatctgtacagcatgaagtgcaagaacgtggtgcccctctatgacctgctgctggaggcggcggacgcccaccgcctacatgcgcccactagccgtggaggggcatccgtggaggagacggaccaaagccacttggccactgcgggctctacttcatcgcattccttgcaaaagtattacatcacgggggaggcagagggtttccctgccacagctgtcgacaatttactgaccgtacaccaaaatttgcctgcattaccggtcgatgcaacgagtgatgaggttcgcaagaacctgatggacatgttcagggatcgccaggcgttttctgagcatacctggaaaatgcttctgtccgtttgccggtcgtgggcggcatggtgcaagttgaataaccggaaatggtttcccgcagaacctgaagatgttcgcgattatcttctatatcttcaggcgcgcggtctggcagtaaaaactatccagcaacatttgggccagctaaacatgcttcatcgtcggtccgggctgccacgaccaagtgacagcaatgctgtttcactggttatgcggcggatccgaaaagaaaacgttgatgccggtgaacgtgcaaaacaggctctagcgttcgaacgcactgatttcgaccaggttcgttcactcatggaaaatagcgatcgctgccaggatatacgtaatctggcatttctggggattgcttataacaccctgttacgtatagccgaaattgccaggatcagggttaaagatatctcacgtactgacggtgggagaatgttaatccatattggcagaacgaaaacgctggttagcaccgcaggtgtagagaaggcacttagcctgggggtaactaaactggtcgagcgatggatttccgtctctggtgtagctgatgatccgaataactacctgttttgccgggtcagaaaaaatggtgttgccgcgccatctgccaccagccagctatcaactcgcgccctggaagggatttttgaagcaactcatcgattgatttacggcgctaaggatgactctggtcagagatacctggcctggtctggacacagtgcccgtgtcggagccgcgcgagatatggcccgcgctggagtttcaataccggagatcatgcaagctggtggctggaccaatgtaaatattgtcatgaactatatccgtaacctggatagtgaaacaggggcaatggtgcgcctgctggaagatggcgatctcgagccatctgctggagacatgagagctgccaacctttggccaagcccgctcatgatcaaacgctctaagaagaacagcctggccttgtccctgacggccgaccagatggtcagtgccttgttggatgctgagccccccatactctattccgagtatgatcctaccagacccttcagtgaagcttcgatgatgggcttactgaccaacctggcagacagggagctggttcacatgatcaactgggcgaagagggtgccaggctttgtggatttgaccctccatgatcaggtccaccttctagaatgtgcctggctagagatcctgatgattggtctcgtctggcgctccatggagcacccagtgaagctactgtttgctcctaacttgctcttggacaggaaccagggaaaatgtgtagagggcatggtggagatcttcgacatgctgctggctacatcatctcggttccgcatgatgaatctgcagggagaggagtttgtgtgcctcaaatctattattttgcttaattctggagtgtacacatttctgtccagcaccctgaagtctctggaagagaaggaccatatccaccgagtcctggacaagatcacagacactttgatccacctgatggccaaggcaggcctgaccctgcagcagcagcaccagcggctggcccagctcctcctcatcctctcccacatcaggcacatgagtaacaaaggcatggagcatctgtacagcatgaagtgcaagaacgtggtgcccctctatgacctgctgctggaggcggcggacgcccaccgcctacatgcgcccactagccgtggaggggcatccgtggaggagacggaccaaagccacttggccactgcgggctctacttcatcgcattccttgcaaaagtattacatcacgggggaggcagagggtttccctgccacagcttgatagcggccgcactcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgccatatgctggctgccatgaacaaaggtggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatgaagatccctcgacctgcgatccccgggtaccgagctgcctgcaggtcgactctagaggatcggccgcatctagaagttcctattccgaagttcctattctctagaaagtataggaacttcggatcagccgcggcagtctgacgagatcatatcactgtggacgttgatgaaagaatacgttattctttcatcaaatcgtgtcgtggatcagttctggacgagccagggtaatgagctattaaggctttttgtcttatacttaactttttttttaaatgtggtatctttagaaccaagggtcttagagttttagtatacagaaactgttgcatcgcttaatcagattttctagtttcaaatccagagaatccaaattcttcacagccaaagtcaaattaagaatttctgacttttaatgttaatttgcttactgtgaatataaaaatgatagcttttcctgaggcagggtctcactatgtatctctgcctgatctgcaacaagatatgtagactaaagttctgcctgcttttgtctcctgaatactaaggttaaaatgtagtaatacttttggaacttgcaggtcagattcttttataggggacacactaagggagcttgggtgatagttggtaaaatgtgtttcaagtgatgaaaacttgaattattatcaccgcaacctactttttaaaaaaaaaagccaggcctgttagagcatgcttaagggatccctaggacttgctgagcacacaagagtagttacttggcaggctcctggtgagagcatatttcaaaaaacaaggcagacaaccaagaaactacagttaaggttacctgtctttaaaccatctgcatatacacagggatattaaaatattccaaataatatttcattcaagttttcccccatcaaattgggacatggatttctccggtgaataggcagagttggaaactaaacaaatgttggttttgtgatttgtgaaattgttttcaagtgatagttaaagcccatgagatacagaacaaagctgctatttcgaggtctcttggtttatactcagaagcacttctttgggtttccctgcactatcctgatcatgtgctaggcctaccttaggctgattgttgttcaaataaacttaagtttcctgtcaggtgatgtcatatgatttcatatatcaaggcaaaacatgttatatatgttaaacatttgtacttaatgtgaaagttaggtctttgtgggtttgatttttaattttcaaaacctgagctaaataagtcatttttacatgtcttacatttggtggaattgtataattgtggtttgcaggcaagactctctgacctagtaaccctacctatagagcactttgctgggtcacaagtctaggagtcaagcatttcaccttgaagttgagacgttttgttagtgtatactagtttatatgttggaggacatgtttatccagaagatattcaggactatttttgactgggctaaggaattgattctgattagcactgttagtgagcattgagtggcctttaggcttgaattggagtcacttgtatatctcaaataatgctggccttttttaaaaagcccttgttctttatcaccctgttttctacataatttttgttcaaagaaatacttgtttggatctccttttgacaacaatagcatgttttcaagccatattttttttcctttttttttttttttttggtttttcgagacagggtttctctgtatagccctggctgtcctggaactcactttgtagaccaggctggcctcgaactcagaaatccgcctgcctctgcctcctgagtgccgggattaaaggcgtgcaccaccacgcctggctaagttggatattttgttatataactataaccaatactaactccactgggtggatttttaattcagtcagtagtcttaagtggtctttattggcccttcattaaaatctactgttcactctaacagaggctgttggtactagtggcacttaagcaacttcctacggatatactagcagattaagggtcagggatagaaactagtctagcgttttgtatacctaccagctttatactaccttgttctgatagaaatatttcaggacatctagagtgtactataaggttgatggtaagcttataaggaacttgaaagtggagtaactactccatttctctgaggggagaattaaaatttttgaccaagtgttgttgagccactgagaatggtctcagaacataacttcttaaggaaccttcccagattgccctcaacactgcaccacatttggtcctgcttgaacattgccatggctcttaaagtcttaattaagaatattaattgtgtaattattgtttttcctcctttagatcattccttgaggacaggacagtgcttgtttaaggctatatttctgctgtctgagcagcaacaggtcttcgagatcaacatgatgttcataatcccaagatgttgccatttatgttctcagaagcaagcagaggcatgatggtcagtgacagtaatgtcactgtgttaaatgttgctatgcagtttggatttttctaatgtagtgtaggtagaacatatgtgttctgtatgaattaaactcttaagttacaccttgtataatccatgcaatgtgttatgcaattaccattttaagtattgtagctttctttgtatgtgaggataaaggtgtttgtcataaaatgttttgaacatttccccaaagttccaaattataaaaccacaacgttagaacttatttatgaacaatggttgtagtttcatgcttttaaaatgcttaattattcaattaacaccgtttgtgttataatatatataaaactgacatgtagaagtgtttgtccagaacatttcttaaatgtatactgtctttagagagtttaatatagcatgtcttttgcaacatactaacttttgtgttggtgcgagcaatattgtgtagtcattttgaaaggagtcatttcaatgagtgtcagattgttttgaatgttattgaacattttaaatgcagacttgttcgtgttttagaaagcaaaactgtcagaagctttgaactagaaattaaaaagctgaagtatttcagaagggaaataagctacttgctgtattagttgaaggaaagtgtaatagcttagaaaatttaaaaccatatagttgtcattgctgaatatctggcagatgaaaagaaatactcagtggttcttttgag